SEQUENCE LISTING (200 sequences)

SEQ ID NO: 1 - protein, 144 aa, Streptococcus sp.
ENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLS

SEQ ID NO: 2 - protein, 1373 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 3 - protein, 1375 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 4 - protein, 1377 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 5 - protein, 1379 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 6 - protein, 1373 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 7 - protein, 1375 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 8 - protein, 1377 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 9 - protein, 1379 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 10 - protein, 1373 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 11 - protein, 1375 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 12 - protein, 1377 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 13 - protein, 1379 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 14 - protein, 1373 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 15 - protein, 1375 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 16 - protein, 1377 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 17 - protein, 1379 aa, synthetic construct
MGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 18 - protein, 36 aa, synthetic construct
EKSKNDRSKPQPSDDRDRQPPSGEDYPEWKAPGEYE

SEQ ID NO: 19 - protein, 34 aa, synthetic construct
QEPKPQDQSSEVPPPPGSQKPGTKEPHDSKSSGP

SEQ ID NO: 20 - protein, 34 aa, synthetic construct
PDNSSGQKLQLPQPSDKPQDSREKSDSLPSDKRD

SEQ ID NO: 21 - protein, 36 aa, synthetic construct
PDNSTLQTLQLPQPTPSSTDTQQTSDTDPEDTTDVI

SEQ ID NO: 22 - protein, 30 aa, synthetic construct
STSQSDGSSVPADIDQSSDSDQSSSQGQPG

SEQ ID NO: 23 - protein, 14 aa, synthetic construct
AKPDDESQKPPQDD

SEQ ID NO: 24 - protein, 14 aa, synthetic construct
LQLEPGPTTPEYPI

SEQ ID NO: 25 - protein, 14 aa, synthetic construct
IQLPPSDTTPENPI

SEQ ID NO: 26 - protein, 12 aa, synthetic construct
ESNDNSQVPPSL

SEQ ID NO: 27 - protein, 10 aa, synthetic construct
SEQQEYPGSG

SEQ ID NO: 28 - protein, 10 aa, synthetic construct
NNSEQQENPA

SEQ ID NO: 29 - protein, 12 aa, synthetic construct
STDGSGQPKHKP

SEQ ID NO: 30 - protein, 20 aa, synthetic construct
PKPSSESGERYEQQPEPPPP

SEQ ID NO: 31 - protein, 16 aa, synthetic construct
KGGGGEPDEKRPSQSS

SEQ ID NO: 32 - protein, 14 aa, synthetic construct
YAGGTPKEPPPPNS

SEQ ID NO: 33 - protein, 14 aa, synthetic construct
PLVAGGTPFEPPPP

SEQ ID NO: 34 - protein, 14 aa, synthetic construct
PQPDERSQIPDNKE

SEQ ID NO: 35 - protein, 10 aa, synthetic construct
YTDEKPLPRS

SEQ ID NO: 36 - protein, 12 aa, synthetic construct
SHPPQEPPQSNL

SEQ ID NO: 37 - protein, 16 aa, synthetic construct
SESPSKQQPEPKSSKG

SEQ ID NO: 38 - protein, 16 aa, synthetic construct
SESPTNQQPEPQWTTD

SEQ ID NO: 39 - protein, 16 aa, synthetic construct
GGSKGPPPSPPPPQPE

SEQ ID NO: 40 - protein, 16 aa, synthetic construct
GPLPAPPPQPPPPQPN

SEQ ID NO: 41 - protein, 14 aa, synthetic construct
RPLPHDNNKQDYSK

SEQ ID NO: 42 - protein, 61 aa, synthetic construct
  VARIANT 5..61: note = Residue may be present or absent
SGGSGGSGGSGGSGGSGGSGGSGGSGGSGGSGGSGGSGGSGGSGGSGGSGGSGGSGGSGGS

SEQ ID NO: 43 - protein, 4 aa, synthetic construct
SGGS

SEQ ID NO: 44 - protein, 100 aa, synthetic construct
  VARIANT 6..100: note = Residue may be present or absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 45 - protein, 10 aa, synthetic construct
SGGSGGSGGS

SEQ ID NO: 46 - protein, 16 aa, synthetic construct
SGSETPGTSESATPES

SEQ ID NO: 47 - protein, 32 aa, synthetic construct
SGGSSGGSSGSETPGTSESATPESSGGSSGGS

SEQ ID NO: 48 - DNA, 1592 bp, Medicago truncatula
  source 1..1592: mol_type = other DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 49 - DNA, 2000 bp, Zea mays
  source 1..2000: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 50 - protein, 1227 aa, unidentified
  source 1..1227: note = Lachnospiraceae bacterium
SKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 51 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 52 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 53 - protein, 1238 aa, Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 54 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 55 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 56 - protein, 1206 aa, unidentified
  source 1..1206: note = Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 57 - protein, 1233 aa, unidentified
  source 1..1233: note = Lachnospiraceae bacterium
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 58 - protein, 1227 aa, unidentified
  source 1..1227: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 59 - protein, 1264 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 60 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 61 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 62 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 63 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 64 - protein, 1484 aa, unidentified
  VARIANT 1073: note = Xaa can be any naturally occurring amino acid
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 65 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 66 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 67 - DNA, 3987 bp, synthetic construct
  source 1..3987: mol_type = other DNA
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 68 - DNA, 3987 bp, synthetic construct
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 69 - DNA, 3987 bp, synthetic construct
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 70 - DNA, 4101 bp, synthetic construct
  source 1..4101: mol_type = other DNA
gacaagaagtacagcatcgggctggcgatcgggaccaactccgtcggctgggctgtgattaccgacgagtacaaggtgccatccaagaagttcaaggtcctcggcaacactgaccggcacagcattaagaagaacctgattggggcgctgctgttcgattcgggggagactgcggaggcgaccaggctgaagcggactgcgcgccggaggtacaccaggaggaagaatcggatctgctacctccaggagattttctcgaatgagatggccaaggtggacgattccttcttccatcgcctggaggagtcgttcctcgttgaggaggacaagaagcatgagaggcatcccattttcgggaatatcgttgacgaggtggcttaccatgagaagtacccgaccatctaccatctgcggaagaagctcgtcgattcgaccgataaggccgacctgcggctgatctacctggccctcgcgcacatgattaagttccggggccatttcctcatcgagggcgacctcaacccggacaactcggacgtggataagctcttcattcagctcgtgcagacatacaaccagctcttcgaggagaatcccattaacgcctcgggggtcgacgctaaggctattctctcggctcggctgtcgaagtcgcgccggctggagaatctcattgcccagctcccaggcgagaagaagaacggcctcttcggcaacctgattgccctgtcgctggggctcacaccgaatttcaagtcgaacttcgacctcgccgaggacgctaagctccagctcagcaaggatacttacgatgatgacctcgataacctgctcgcccagattggggatcagtacgcggatctgttcctcgcggccaagaatctcagcgatgctattctcctgtcggacattctccgcgtcaacacagagattactaaggccccactgtcggcgagcatgattaagaggtacgatgagcatcatcaggacctgacactgctcaaggcgctggtccggcagcagctccccgagaagtacaaggagattttcttcgatcagtcaaagaatgggtacgcgggctacattgatggcggcgcgtcccaggaggagttctacaagttcattaagcccatcctggagaagatggacgggaccgaggagctgctggtgaagctcaatcgggaggacctgctccggaagcagcgcacattcgacaatggctcgattcctcaccagattcacctgggcgagctgcacgccattctccgcaggcaggaggacttctacccgttcctcaaggacaaccgcgagaagatcgagaagatcctgaccttccggattccatactacgtggggccgctcgcgcgggggaactcccggttcgcgtggatgactcgcaagtccgaagaaacgattacaccgtggaatttcgaggaggtcgtcgacaagggcgctagtgcgcagtcattcattgagaggatgaccaatttcgataagaacctgcctaacgagaaggtgctgccgaagcattcgctgctctacgagtacttcaccgtttacaatgagctgaccaaggtgaagtatgtgactgagggcatgaggaagccagcgttcctgagcggcgagcagaagaaggctatcgtggacctgctcttcaagactaaccggaaggtgactgtgaagcagctcaaggaggactacttcaagaagattgagtgcttcgattccgttgagattagcggggtggaggatcggttcaatgcttcgctcgggacataccacgatctcctgaagatcattaaggataaggacttcctcgacaacgaggagaacgaggacattctcgaagatattgtcctgaccctcaccctcttcgaggatcgggagatgatcgaggagaggctcaagacatacgctcatctgttcgatgataaggtcatgaagcagctgaagcgcaggcggtacacagggtgggggcggctgagccggaagctgatcaacgggattcgggataagcagtccgggaagacaattctcgacttcctcaagtccgacgggttcgctaaccggaacttcatgcagctcattcatgatgactcgctgacattcaaggaggatattcagaaggcgcaggtttcggggcagggcgactcgctccacgagcatattgcgaatctggcgggctcccccgcgattaagaagggcattctgcaaaccgtcaaggtggttgatgagctggtcaaggtcatggggcggcataagccagagaatattgtcatcgagatggcgcgggagaatcagaccacacagaaggggcagaagaactcacgggagcggatgaagcgcatcgaggagggcatcaaggagctggggtcgcagatcctgaaggagcatcccgtggagaacactcagctgcaaaatgagaagctgtacctctactacctccagaacgggagggacatgtatgtggatcaggagctggatattaataggctgagcgattacgatgtcgaccacattgtcccacagtcgttcctgaaggacgacagcattgacaacaaggtgctgacccgctcggataagaacaggggcaagagcgataatgttccaagcgaggaggttgtgaagaagatgaagaactactggcggcagctcctgaacgcgaagctcatcacacagcggaagttcgacaacctcaccaaggctgagcgcgggggcctgagcgagctggacaaggcggggttcattaagaggcagctggtcgagacacggcagattacaaagcatgttgcgcagattctcgattcccggatgaacaccaagtacgatgagaacgataagctgattcgggaggtcaaggtaattaccctgaagtccaagctggtgtccgacttcaggaaggacttccagttctacaaggttcgggagatcaacaactaccaccacgcgcatgatgcctacctcaacgcggtcgtggggaccgctctcatcaagaagtacccaaagctggagtcagagttcgtctacggggattacaaggtttacgacgtgcggaagatgatcgctaagagcgagcaggagattggcaaggctaccgctaagtacttcttctactccaacatcatgaacttcttcaagacagagattaccctcgcgaatggcgagatccggaagaggcccctcatcgagacaaatggggagacaggggagattgtctgggataaggggcgggatttcgcgaccgtccggaaggtcctgtcgatgccccaggttaatattgtcaagaagactgaggtccagactggcggcttctcaaaggagtcgattctcccaaagaggaactccgataagctcattgctcggaagaaggattgggaccccaagaagtacgggggattcgactcccccactgttgcttactctgttctggttgttgctaaggtggagaaggggaagtcgaagaagctgaagagcgtgaaggagctgctcgggattacaattatggagaggtcatccttcgagaagaatcccatcgacttcctggaggccaagggctacaaggaggtgaagaaggacctgattattaagctgcccaagtactcgctcttcgagctggagaatgggcggaagcggatgctggcgtccgcgggggagctgcaaaaggggaacgagctggcgctcccctccaagtatgtgaacttcctctacctggcgtcgcactacgagaagctgaaggggtccccagaggataatgagcagaagcagctcttcgtcgagcagcataagcactacctggacgagattatcgagcagattagcgagttctcgaagcgggtcatcctcgcggatgcgaacctggataaggtgctcagcgcctacaataagcaccgggacaagccgattcgggagcaggcggagaatattattcacctcttcacactcaccaacctcggggcaccagctgcgttcaagtacttcgacactactatcgaccggaagcggtacacctcgacgaaggaggtgctcgacgccaccctcattcaccagtcgatcacaggcctgtacgagacacggattgacctgtcccagctcgggggcgac

SEQ ID NO: 71 - DNA, 4101 bp, synthetic construct
  source 1..4101: mol_type = other DNA
gacaagaagtactccattggcctggcgattgggacaaactcggtggggtgggccgtgattacggatgagtacaaggttccaagcaagaagttcaaggtcctcgggaacacagatcggcattcgattaagaagaatctcattggggcgctcctcttcgactcgggggagacagcggaggctaccaggctcaagcggacagccaggcggcggtacacaaggcggaagaatcgcatctgctacctccaggagattttctcgaatgagatggcgaaggtggacgacagcttcttccatcggctggaggagtccttcctggtggaggaggataagaagcacgagaggcatccaattttcgggaacatcgtggacgaggttgcgtaccatgagaagtaccctacaatctaccatctgcggaagaagctggttgactccacagacaaggcggacctgaggctgatctacctcgctctggcccacatgattaagttccgcgggcatttcctgatcgagggggacctgaatcccgacaattcggatgtggacaagctcttcatccagctggtgcagacctacaaccagctgttcgaggagaatcccatcaatgcgtcgggcgttgacgctaaggccattctgtccgctaggctgtcgaagagcaggaggctggagaacctgatcgcccagctgccaggcgagaagaagaatgggctcttcgggaatctgattgcgctctccctggggctgacaccgaacttcaagagcaatttcgatctggctgaggacgcgaagctccagctctcgaaggacacttacgacgatgacctcgataacctcctcgcgcagatcggggaccagtacgctgatctcttcctcgccgctaagaacctctcggatgctatcctgctctccgacattctccgggttaataccgagattacaaaggccccactgtcggcgtccatgatcaagcggtacgatgagcatcatcaggatctcaccctgctcaaggccctcgtgcggcagcagctgcccgagaagtacaaggagattttcttcgaccagagcaagaatgggtacgctggctacattgacggcggggcctcacaggaggagttctacaagttcatcaagccaatcctggagaagatggatgggacagaggagctgctggtgaagctcaaccgggaggatctgctcaggaagcagcggacgttcgacaacgggtcgattccccatcagatccacctgggggagctgcacgcgatcctgcgccggcaggaggatttctaccctttcctgaaggataatcgggagaagatcgagaagattctcaccttccggattccctactacgtcgggccactcgcgcggggcaatagcaggttcgcctggatgacacggaagagcgaggagacaatcaccccctggaacttcgaggaggttgtcgacaagggggcgtccgcccagtcattcattgagcggatgaccaatttcgacaagaatctgccaaatgagaaggttctcccaaagcatagcctcctctacgagtacttcactgtttacaacgagctgaccaaggtgaagtatgtgaccgagggcatgcggaagcccgcgttcctgtccggcgagcagaagaaggccattgtggacctcctgttcaagaccaatcgcaaggtcacagtcaagcagctcaaggaggattacttcaagaagatcgagtgcttcgactcggttgagattagcggggtggaggatcggttcaacgcgagcctcggcacttaccacgacctcctgaagatcatcaaggataaggacttcctcgacaacgaggagaacgaggatattctggaggacatcgtgctcaccctgacgctgttcgaggatcgggagatgatcgaggagcgcctgaagacctacgctcatctcttcgatgataaggtcatgaagcagctgaagaggaggcggtacaccgggtggggccgcctgagcaggaagctcattaacgggatcagggacaagcagagcggcaagaccatcctggacttcctcaagagcgatggcttcgccaaccggaatttcatgcagctcatccacgacgactccctcaccttcaaggaggacattcagaaggctcaggtcagcggccagggcgactcgctgcatgagcacatcgctaacctggcgggcagcccagccatcaagaagggcatcctccagacagtgaaggtcgtggatgagctggtgaaggtcatgggccggcataagcccgagaatattgtgattgagatggcgcgggagaatcagaccactcagaagggccagaagaactcgcgggagcgcatgaagaggatcgaggaggggattaaggagctgggcagccagattctcaaggagcaccccgtggagaatacccagctccagaacgagaagctgtacctctactacctccagaatgggcgggacatgtatgttgatcaggagctggacatcaatcgcctctcggattacgacgtggaccacatcgtgccccagagcttcctgaaggatgatagcatcgacaataaggtcctgacccgctccgacaagaatcgcggcaagagcgacaacgtgccgagcgaggaggtcgtgaagaagatgaagaactactggcggcagctgctgaacgcgaagctcattacacagcggaagttcgataacctgacgaaggcggagaggggcggcctctccgagctggacaaggcgggcttcattaagaggcagctcgtggagactcgccagatcaccaagcacgtggctcagatcctcgatagccggatgaatacgaagtacgatgagaatgacaagctcatccgggaggtgaaggtaatcaccctgaagtcaaagctcgttagcgatttccggaaggacttccagttctacaaggtgcgggagattaacaactaccatcatgcgcacgatgcgtacctcaatgcggtggtgggcacagccctgattaagaagtaccccaagctggagagcgagttcgtctacggggactacaaggtgtacgatgttcggaagatgatcgccaagagcgagcaggagattgggaaggccaccgctaagtacttcttctactcgaatattatgaatttcttcaagaccgagatcacactcgctaatggggagattcggaagcggcccctcatcgagactaacggggagactggcgagattgtgtgggacaaggggcgcgacttcgctaccgtgcgcaaggtcctctcgatgccccaggttaatattgttaagaagacagaggtgcagacgggcgggttctccaaggagtctatcctgccgaagcggaactcggacaagctgatcgcccgcaagaaggattgggaccccaagaagtacgggggattcgatagcccaaccgtggcttacagcgtcctggtggtcgccaaggttgagaaggggaagtcgaagaagctcaagagcgttaaggagctgctgggcatcaccatcatggagcggtccagcttcgagaagaatcctatcgacttcctggaggctaaggggtacaaggaggtcaagaaggacctgatcattaagctgcccaagtactctctgttcgagctggagaacgggaggaagcggatgctggcgtctgctggcgagctacagaagggcaatgagctggcgctcccctcgaagtatgtcaacttcctctacctggcttcccattacgagaagctgaagggctcgcccgaggataatgagcagaagcagctcttcgtggagcagcacaagcactacctcgacgagatcattgagcagatttcggagttctcgaagcgggtcattctcgcggacgcgaacctcgacaaggtcctctcggcgtacaacaagcaccgggacaagcccatccgggagcaggccgagaacattatccacctcttcacactgaccaacctcggcgctcccgccgcgttcaagtacttcgacaccaccattgaccgcaagagatacacatccaccaaggaggtgctggacgcgaccctcatccaccagagcatcacaggcctctacgagacacggatcgacctctcgcagctcgggggcgat

SEQ ID NO: 72 - DNA, 4092 bp, synthetic construct
  source 1..4092: mol_type = other DNA
gacaagaagtactcgatcggcctggcgattggcacaaacagcgtggggtgggctgtgatcactgatgagtacaaggtgccatcgaagaagttcaaggtgctggggaatacagaccggcattcgatcaagaagaatctcattggcgctctcctcttcgattccggcgagactgctgaggcgacccgcctgaagcgcaccgcccggcggcgctacactcggcggaagaataggatttgctacctccaggagattttctcgaatgagatggccaaggtggatgacagcttcttccaccgcctggaggagtcgttcctggtcgaggaggacaagaagcatgagcggcaccctatcttcgggaatatcgttgatgaggtcgcctaccacgagaagtaccccactatctaccatctccgcaagaagctcgtggacagcacagataaggccgacctccgcctgatctacctcgccctcgcgcacatgattaagttccgggggcacttcctcattgagggggatctgaatcccgataactccgacgtggacaagctgttcatccagctggtgcagacatacaaccagctgttcgaggagaatcccatcaacgcgagcggcgtggacgctaaggccattctgtcggctaggctctcgaagtcgaggcggctggagaacctgattgcgcagctccccggcgagaagaagaacgggctgttcgggaatctcatcgccctctccctcggcctcacaccaaacttcaagagcaatttcgacctggctgaggacgctaagctgcaactctcaaaggatacatacgatgacgacctggacaatctcctggctcagatcggcgaccagtacgctgacctgttcctcgcggccaagaatctgtcggacgcgattctcctcagcgacatcctgcgcgtcaataccgagattacgaaggctccactgtctgcgtcaatgattaagcggtacgatgagcatcaccaggatctgaccctcctgaaggcgctcgtgcggcagcagctgcccgagaagtacaaggagattttcttcgatcagagcaagaatggctacgccggctacatcgacgggggcgcgagccaggaggagttctacaagttcatcaagcccatcctggagaagatggacggcaccgaggagctactcgtgaagctcaatcgggaggatctcctccggaagcagcggacattcgataacgggtctatcccacaccagatccacctcggcgagctgcatgcgattctgcggcggcaggaggatttctaccctttcctgaaggacaaccgggagaagatcgagaagatcctcacattccggattccatactacgtcggccccctggcgaggggcaatagccggttcgcgtggatgacaaggaagtccgaggagactattaccccgtggaatttcgaggaggtggttgacaagggcgcttccgcgcagagcttcattgagcggatgacaaacttcgacaagaatctccccaacgagaaggtcctgccgaagcatagcctcctgtacgagtacttcaccgtctacaatgagctaactaaggtcaagtatgtgacagagggcatgaggaagccagccttcctctcaggcgagcagaagaaggccattgtggacctcctgttcaagacaaaccgcaaggtgacagtgaagcagctgaaggaggattacttcaagaagattgagtgcttcgactcagtggagatttcaggcgtggaggatcggttcaacgcgagcctggggacttaccacgacctgctgaagattattaaggacaaggacttcctggataacgaggagaatgaggacatcctggaggatattgtgctcaccctcaccctgttcgaggacagggagatgattgaggagaggctcaagacctacgcgcacctgttcgatgacaaggtcatgaagcagctgaagaggcggcgctacactgggtggggccgcctgtcgcggaagctgatcaacggcattcgggataagcagtccgggaagaccattctggatttcctgaagtcggacggcttcgccaacaggaatttcatgcagctgatccacgacgactccctcaccttcaaggaggacattcagaaggcccaggttagcggccagggggactcactccacgagcatattgccaatctggccggctctccagctatcaagaagggcatcctgcaaacagttaaggttgttgacgagctggttaaggtcatggggcggcataagcccgagaacattgtcatcgagatggctcgggagaaccagacaactcagaagggccagaagaactccagggagcgcatgaagcggattgaggagggcattaaggagctggggtcccagatcctcaaggagcaccctgtcgagaacactcagctgcaaaacgagaagctctacctgtactacctccagaacgggcgggatatgtatgtggatcaggagctggacatcaacaggctctccgactacgacgtggatcacattgtcccacagtctttcctcaaggatgattccatcgacaacaaggtgctgacgcgcagcgacaagaatagggggaagtcggacaacgttccgagcgaggaggtcgtgaagaagatgaagaattactggaggcagctcctgaatgcgaagctgatcactcagaggaagttcgacaatctgacaaaggcggagaggggcgggctctcggagctggataaggcgggcttcatcaagcggcagctcgttgaaacccggcagatcaccaagcatgtcgcccagatcctcgatagccgcatgaacaccaagtacgatgagaacgacaagctcattcgggaggttaaggtcattacgctgaagtccaagctcgtcagcgacttcaggaaggatttccagttctacaaggttcgggagattaacaactaccaccacgcgcatgatgcgtacctgaacgctgttgtcggcactgctctcatcaagaagtacccaaagctggagtccgagttcgtctacggggactacaaggtctacgatgtccggaagatgatcgccaagtcggagcaggagatcgggaaggctactgcgaagtacttcttctacagcaacattatgaatttcttcaagacggagattacgctggcgaacggggagattaggaagaggcccctcattgagactaatggggagacaggcgagattgtttgggacaagggccgcgacttcgcgactgtgcggaaggtcctgtccatgccacaggtgaatattgttaagaagacagaggtgcagactgggggcttctcgaaggagagcattctcccaaagcggaacagcgataagctcatcgcgcgcaagaaggattgggaccctaagaagtacggcggcttcgattctcccactgtggcctactccgttctcgtggttgccaaggttgagaaggggaagtcgaagaagctgaagtcggtcaaggagctgctcgggattacaatcatggagcggagcagcttcgagaagaaccctattgatttcctggaggccaagggctacaaggaggttaagaaggatctcattatcaagctccctaagtactctctgttcgagctggagaatggccggaagaggatgctggcctcggctggcgagctacagaaggggaatgagctggccctcccgtcgaagtatgtgaatttcctgtacctcgcgtcgcactacgagaagctcaagggcagcccggaggataatgagcagaagcagctcttcgtggagcagcataagcactacctggacgagatcattgagcagatcagcgagttctcgaagcgggttattctggctgatgctaacctggacaaggttctgagcgcctacaataagcatcgcgacaagccgattcgcgagcaggcggagaatattatccacctgttcaccctcactaacctcggggctcccgcggccttcaagtacttcgataccacaatagataggaagcggtacacctcgacgaaggaggtcctcgacgccacactcatccatcagtcgattacaggcctgtacgagacacggattgacctctcgcagctg

SEQ ID NO: 73 - DNA, 4101 bp, synthetic construct
  source 1..4101: mol_type = other DNA
gacaagaagtattccataggcctggctatcggcaccaacagcgtgggctgggccgtcatcaccgacgagtacaaagtgccgagtaaaaagttcaaagtgctcggcaacaccgaccgccactccataaagaaaaacctgatcggggcgctcctgttcgacagcggcgagacggcggaggccacccgcttgaaacgcacggcccgacggcgctacacgcggcgcaagaaccggatctgttacctacaggagattttctctaacgagatggcgaaggtggacgactcgttctttcaccgcctcgaagagtccttcctcgtggaggaggacaagaaacacgagcgccacccgatcttcggcaacatcgtggacgaggtggcctaccacgagaagtacccgaccatctaccacctccggaagaaactcgtggacagcacggacaaggccgacctgaggctcatctacctcgccctggcgcacatgattaagttccggggccacttcctgatcgagggcgacctgaacccggacaacagcgacgtggacaagctgttcatccagctagtccagacctacaaccagcttttcgaggaaaaccccatcaacgccagcggggtggacgcgaaggcgatcctgtccgcccggctgagcaagtcccggcggctggagaacctcatcgcgcagttgcccggcgagaagaagaacgggctgttcgggaacctgatcgccctctccctggggctcaccccgaacttcaagtccaacttcgacctcgccgaggacgccaaactacagctgagcaaggacacctacgacgacgacctcgacaacctgctggcccagatcggggaccagtacgcagacctgttcctcgccgccaagaacctctccgacgccatcctgctgtcggacatcctgcgggtgaacacggagatcacgaaggccccgctctcggcctcgatgattaaacgctacgacgagcaccaccaggacttgaccctcctcaaggcgctggtccgccagcagcttcccgagaagtacaaggaaatctttttcgatcagagcaagaacgggtacgccgggtacatcgacggcggggcgtcccaggaggagttctacaagttcatcaagcccatcctggagaaaatggacgggaccgaggagctgctcgtgaagctcaaccgcgaagatttgctccgcaagcagcgcacgttcgacaacgggtcgatcccgcaccagatccacctgggcgagctgcacgcgatcctcaggcgtcaggaagacttctaccccttcctcaaggacaaccgcgagaagatagagaagattctgaccttcagaattccttattacgtgggcccgctggctcggggcaactcgcgcttcgcctggatgacgcgcaagtccgaggagaccatcaccccgtggaacttcgaggaggtggtggataagggtgcctcggcccagtccttcatcgagcggatgaccaacttcgacaagaacctgccgaacgagaaggtgctccccaagcacagcctgctctacgaatatttcacggtgtacaacgagctgacgaaggtcaagtacgtgaccgagggaatgaggaaacctgcattcctctccggggagcagaagaaagccatagtcgacctcctgttcaagaccaaccggaaggtcaccgtcaagcagctcaaggaggactacttcaagaagatcgagtgcttcgattcagtggagatcagcggcgtcgaggaccggttcaacgccagcctgggcacctaccacgacctgctcaagatcatcaaggacaaggacttcctcgacaacgaggagaacgaggacatcctggaggacatcgtgctgaccctgacgctcttcgaggaccgcgagatgatcgaggagcgcctcaagacctacgcccacctgttcgacgacaaggtgatgaagcagctcaagcggcggagatatactgggtggggccgcctctcccggaagctcattaacggtatcagggataagcagtccgggaagacgatcctcgacttcctcaagtcggacgggttcgccaaccgcaacttcatgcagctcatccacgacgactccctgacgttcaaggaggacatccagaaggcccaagtgtctggtcaaggtgactcgctccacgagcacatcgccaacctcgcgggcagcccggccatcaagaagggaatactccagaccgtcaaggtggtggacgagctggtgaaggtcatgggccgccacaagccggagaacatcgtcatcgagatggcgcgggagaaccagaccacgcagaaggggcagaaaaatagccgtgagcgcatgaagcgcatcgaggaggggattaaggagttgggcagccagatcctcaaggagcaccctgtggagaacacgcagttgcaaaacgagaagctctacctgtactacctccagaacgggagggatatgtacgtggaccaagaactggacatcaaccgcctgtccgactacgacgtggaccacatcgtgccgcagagcttcctcaaggacgacagcatcgacaacaaggtgctcacccggtccgacaagaatcggggcaagtccgacaacgtgcccagcgaggaggtcgtcaaaaagatgaaaaactactggcgacaactactgaacgccaagctcatcacccagcgcaagttcgacaacctcacaaaagccgagcgcggcgggttgagcgagctggacaaggccgggttcatcaagcgccagctcgtcgagacgcgccagatcacgaagcacgtcgcgcagatactcgacagccggatgaacaccaagtacgacgagaacgacaagctcatccgggaggtgaaggtcatcaccctcaagtcgaagctcgtgagcgacttccgcaaggacttccagttctacaaggtccgggagatcaacaactaccaccacgcccacgatgcttatcttaacgccgtggtggggacggccctcattaagaaatacccgaagctggagtcggagttcgtgtacggcgactacaaggtgtacgacgtcaggaagatgatcgccaagtccgaacaggagatcgggaaggccacggcgaaatacttcttctacagcaacatcatgaacttcttcaagaccgagatcaccctcgccaacggcgagatccgcaagcgcccgctcatcgagacgaacggggagaccggcgagatcgtctgggacaaggggcgcgacttcgccactgtgcggaaggtgctgtcgatgccccaggtcaacatcgtcaagaagacggaggtccagacgggcgggttcagcaaggagagcatcctgccgaagcgcaacagcgacaagctgatcgcccgcaaaaaggactgggatccaaaaaagtacggcggcttcgacagccccaccgtcgcctacagcgtcctcgtcgtcgctaaagtcgagaagggcaagtccaaaaagctcaagagcgtcaaggagctgctcgggatcaccatcatggagcggtccagcttcgagaagaacccaattgatttcctggaggcgaagggctacaaggaggtcaagaaagacctcatcataaagctgccgaagtactcactcttcgagctggagaacgggcgcaagcggatgctggcgtcggccggagagctccaaaagggcaacgagctggcgctgccgagcaagtacgtgaacttcctctacctggcgtcccactacgagaagctcaagggcagtccagaggataacgagcagaagcagctattcgtggagcagcacaagcactacctggacgagatcatcgagcagatcagcgagttctccaagcgcgtcatcctggcggacgccaacctggacaaggtgctgtccgcgtacaacaagcaccgcgacaagccgatccgcgagcaagccgagaacatcatccacctgttcaccctcacgaacctcggggcacccgccgccttcaaatatttcgacacgaccatcgaccgcaagcgctacaccagcacgaaggaggtgctcgacgccaccctgatccaccagagcatcaccgggctgtacgagacccgcatcgacctctcgcagctcggcggggac

SEQ ID NO: 74 - DNA, 4101 bp, synthetic construct
  source 1..4101: mol_type = other DNA
gacaagaagtacagtattggattggccatcgggacgaacagcgtgggctgggccgtcatcaccgacgagtacaaggtgccatccaagaagtttaaggttctggggaataccgaccgccactcgatcaagaaaaatctcatcggggcgctgcttttcgacagcggcgagacggcggaagcgacgcggctcaagcggacggctcgtcgccgttacacccggcgtaagaaccgcatctgttacctccaggagatattcagcaacgagatggcgaaggtggacgactcctttttccaccgtcttgaggagtccttcctggtcgaggaggacaagaagcacgagcgccacccgatcttcgggaacatcgtggacgaggtggcctaccacgagaagtaccccacgatctaccacctccgcaaaaaactcgtggactcaactgacaaggccgatttgaggcttatctacctcgccctcgcccacatgattaagttccgtgggcacttcctaatcgagggtgacctcaaccccgacaactctgacgtggacaagctgttcatccagcttgtgcagacctacaatcagctctttgaggagaatccgatcaacgcatctggtgtggacgcaaaggccatcctcagcgcgcggctgagcaagtctaggcggttggagaacctgatcgcccaactgcccggcgagaagaaaaatggcctcttcggcaacctgatcgccctgtcgctggggctcacgccgaacttcaagagtaactttgacctggcggaggacgctaagctccagctatctaaggacacatacgacgacgacctggacaacctgctggcccagatcggcgaccagtacgccgacctcttcctagccgccaagaacctgtccgacgccatcctcctcagcgacatcctgcgcgtgaacacggagatcacgaaggctccgctcagcgcctccatgattaagcggtacgacgagcaccaccaagacctaactttactcaaagccctcgtgcggcagcagcttcccgagaagtacaaagagatattttttgatcagtccaagaacggttatgcgggctacatcgacggcggcgcgagccaggaggagttctacaagttcatcaagcccatcctggagaagatggacggcacggaggagctgctcgtgaagctcaaccgtgaagacctcctgcgaaagcagcgaaccttcgacaacggttcgatcccgcaccagatccacctcggggagctgcacgccatcctgaggcgacaggaggacttctaccctttcctaaaggacaaccgcgagaagattgaaaaaatcctgacgtttcgcataccctactacgtcggcccgctggcgcgcggcaactcccggttcgcctggatgacccgtaagagcgaggagacgatcaccccgtggaacttcgaggaggtcgtggacaagggcgcgagcgcgcagagcttcatcgagcgcatgaccaacttcgacaagaacctcccgaacgagaaggtgctcccaaagcactccctcctgtacgagtatttcaccgtgtacaacgagttgacaaaggtgaagtacgtgacggagggaatgcggaagcctgcgttcctctcgggcgagcagaagaaggcaatcgtggacctgctcttcaagaccaaccggaaggtgacggtgaagcagctcaaggaggactacttcaaaaaaatcgagtgcttcgactccgtggagataagcggcgtggaggaccgattcaacgcctccctcggcacctaccacgacctccttaagatcatcaaggacaaggacttcctggacaacgaggagaacgaggacatcctggaggacatcgtgctcaccctgaccctcttcgaggaccgggagatgatcgaggagcgcctcaagacgtacgcccacttgttcgacgacaaggtgatgaagcagctcaagcggcggcgatacaccgggtggggccgcctatcccgcaaacttatcaacggcatccgcgacaagcagtccggcaagacgatcctggatttcctcaagtcggacgggttcgccaaccggaacttcatgcagctcatccacgacgacagcctcacgttcaaggaggacatccagaaggcccaagtgagcggtcaaggggacagcctccacgagcacattgcgaaccttgctgggagccctgcgatcaagaaggggatattgcaaaccgtgaaggtcgtggacgagttggtgaaggtcatggggcgacacaagcccgagaacatcgtgatcgagatggccagggaaaatcagaccacgcagaagggccaaaaaaacagccgcgagcggatgaagcggatcgaggagggcatcaaggagctggggtcgcagatcctcaaggagcacccggtggagaacacgcagctccagaacgagaagctgtacctctattacctacagaacgggcgggatatgtacgtggaccaggagctagacatcaaccgcctgtccgactacgacgtggaccatatcgtcccgcagtcgttcttgaaggacgacagcatcgacaacaaggtgctcacaagatcggataagaatcgaggcaagtccgacaacgtgccctcggaggaggtggtcaagaaaatgaaaaactactggcggcagttgctgaacgccaagctcattacgcagcggaagttcgacaacctgacgaaggctgaacgtggtgggctcagcgagctagacaaggcggggttcatcaagcggcagctcgtcgagacccggcagatcaccaagcacgtggcgcagatcctggactcgcgcatgaacaccaagtacgacgagaacgacaagctcatccgtgaggtgaaggtcatcacccttaagtctaagctggtcagtgacttccgcaaggacttccagttctacaaggtccgggagatcaacaactaccaccacgcgcacgacgcctacctcaacgcggtggtggggacggcgcttattaagaaatatcccaagctggaaagcgagttcgtttacggcgactacaaggtgtacgacgtccgcaagatgatcgcaaagtcggaacaggaaatcggaaaggcgacggccaaatatttcttttactccaacatcatgaatttttttaagacggagatcaccctggcgaacggggagatccgcaagcggcccctcatcgagaccaacggggagacgggcgagatcgtctgggacaagggccgggacttcgccaccgtgcggaaggtgctttctatgcctcaagtcaatatcgtcaaaaagacagaggtgcagaccggcgggttcagcaaggagtctatcctgccgaagcgcaactcggacaagctcatcgcgcgcaagaaagactgggaccccaaaaaatatggcgggttcgactcgccgaccgtcgcctacagcgtcctcgtggtggctaaggtcgagaagggcaagagcaaaaagctaaagtcggtgaaggagctgctgggcatcaccatcatggagcgctcgtctttcgagaagaatccaatcgacttcctagaggcgaaggggtacaaggaggtcaaaaaggatcttatcatcaaactgccgaagtacagtctgttcgagctggagaacgggcggaagcggatgctggctagtgcgggcgagttgcagaagggcaacgagttggcactgccctccaagtacgtgaacttcctgtacctggcctcccactacgagaagctcaaggggagccccgaggacaacgagcagaagcagctattcgtcgagcagcacaagcactacctggacgagatcatcgagcagatcagtgagttctccaagcgggtcatcctcgcggacgccaacctggacaaggtgctgagcgcgtacaacaagcacagggacaagccaatcagggaacaggccgagaacatcatccacctgttcaccctgaccaacctgggtgcaccggctgccttcaagtactttgacacgaccatcgaccggaagcgctacacctccacgaaggaggtgctggacgccacgctgatccaccagagcatcaccgggctctacgagacacggatcgacctgagccagcttggcggggac

SEQ ID NO: 75 - DNA, 4092 bp, synthetic construct
  source 1..4092: mol_type = other DNA
gacaaaaagtattccattggactcgctatcggcacgaacagcgtcgggtgggcggtcatcactgacgagtacaaggtgccgagcaagaagtttaaggtgctgggaaacaccgacaggcactcgatcaagaaaaatcttatcggggccctactcttcgactccggagaaaccgccgaggccacccggttgaagcgcacggcccgccgtcgctacaccaggcgcaagaaccggatctgctacctccaggagatattcagcaatgagatggcgaaggtggacgactcgttttttcacaggctagaggagtctttcctcgtggaggaggacaagaaacacgagcgccaccccatcttcggcaacatcgtggatgaggtggcatatcacgagaagtacccaaccatctaccacctccgcaaaaagctcgtggactctaccgacaaggccgacctccgtctgatctacctcgcgctggcccacatgattaagttccgaggacactttctgatcgagggcgacctgaacccagacaacagcgacgtggacaagctgttcatccaacttgtccagacctacaatcagctcttcgaggagaaccctatcaacgcctcgggcgtggacgcgaaggccatcctgtccgcccgcctgagcaagtcgcggcggctggagaacctgatcgcccagctccccggcgaaaaaaagaacggcctcttcggcaacctcatcgcgttgtcgctggggctcaccccgaacttcaagtccaacttcgacctggccgaggacgctaaactccagctctcgaaggatacctacgacgacgacctcgacaacctgctggcccagatcggcgaccagtacgcggaccttttcctggcggccaagaacctgagcgacgcgatcctccttagcgacatactccgtgtgaacaccgagatcacgaaggccccgctctccgcgtccatgattaagcgctacgacgagcaccaccaagaccttaccctgcttaaggcgctggtcaggcagcagttaccggagaagtacaaggagatcttttttgatcaatctaagaacggttacgccgggtacatcgacggcggcgcgtcccaggaggagttctacaagttcatcaagccgatcttggagaaaatggacgggaccgaggagctgctcgtgaagctcaaccgcgaagacctcctccgcaagcagcgcaccttcgacaacgggagcatcccgcaccagatccacctgggagagctgcacgcgatcctgcggagacaagaggacttctaccccttcctcaaggacaaccgggagaagattgaaaaaatacttacttttcgtatcccgtactacgtcgggccccttgcgaggggcaactccagattcgcgtggatgacccgcaagtccgaggagaccatcaccccgtggaacttcgaggaggtggtggacaagggcgcgtcggcccagtcgttcatcgagcgcatgaccaacttcgacaagaaccttccgaacgagaaggtgctcccgaagcacagcctgctctacgaatattttactgtgtacaacgagctgacgaaggtcaagtacgttacggaggggatgaggaagcccgccttcctctccggcgagcagaagaaagccattgtggatctcctgttcaagaccaaccgcaaggtgacggtgaaacagctcaaagaggactacttcaagaagatcgagtgcttcgactccgtagagatcagcggggtcgaggaccgcttcaacgcctcgctgggcacgtaccacgacctgctaaagattatcaaggacaaagacttcctagacaatgaggagaacgaggacattctggaggacatcgtgctgactctgacgctgttcgaagaccgcgagatgatcgaggagcggcttaagacgtacgcccacctgttcgacgacaaggtgatgaagcagttgaaacggcggcgctacaccgggtggggccgcctctcccgcaagctcatcaacggcatccgcgacaagcagtcggggaagacgatcctggacttcctcaagagcgacggcttcgccaaccgaaacttcatgcagctaatccacgacgacagcctgacgttcaaggaggacatccagaaggcccaagtgagcggccagggagactcgctacacgagcatatcgccaacctggctggcagcccggcgattaagaaaggaatcctccaaaccgtcaaagtggtggacgagctggtgaaggtgatgggccgccacaagcccgagaacattgtgatcgagatggcgcgggagaaccagacgacgcagaagggccaaaaaaatagcagggaaaggatgaagcgaatagaggaggggatcaaggagctggggagccagattctcaaagagcacccggtcgagaacacacagctccagaacgagaagctgtacctctactacctccaaaacggccgcgatatgtacgtggaccaggaactagacatcaaccggctgagcgactatgacgtggaccacatcgtgccgcagtccttcctcaaggacgactcgattgacaacaaagtgctcactagatccgacaagaacagaggcaagagcgataacgtcccgtcggaggaggtcgtcaagaaaatgaaaaactactggcggcagctcctaaacgccaagctcatcacgcagcgtaagttcgacaacctgacgaaggcggagcggggcgggctgagcgagctggacaaagcggggttcatcaagcggcagctcgttgagacgcggcagatcacaaagcacgtcgcgcaaatcctcgactcccgcatgaacaccaagtacgacgagaacgacaagctcatccgggaggtgaaggtcattacccttaaatcgaagctcgtcagcgactttcgtaaggacttccagttctacaaggtcagagagatcaacaactaccaccacgcccacgacgcctatctgaacgccgtggtgggcaccgcgcttattaagaagtaccccaagctggagtccgagttcgtgtacggcgactacaaggtttatgacgtcaggaagatgatcgccaagtcggaacaggagatcggaaaagctaccgccaaatatttcttctatagcaacatcatgaacttcttcaaaaccgagatcaccctcgccaacggcgagatccggaagcgcccgctcatcgagaccaacggggagaccggggagatcgtctgggacaaggggcgggacttcgctactgtccgaaaggtgctctccatgccacaagtgaatatcgtcaagaaaacagaggtgcagaccggagggttcagtaaggagtccatcctgcccaagcggaactccgacaagctaattgctcgcaaaaaggattgggatcctaaaaaatatggcggcttcgactcgcccacggtcgcctactctgtgctggtcgtggcgaaggtggagaagggcaagtccaagaagctcaagagcgtcaaggagctgctggggatcacgatcatggagcgtagttcgtttgagaagaatcccatcgacttcctggaggctaagggctacaaggaggtcaaaaaggacctcatcattaagctgccgaagtacagcctcttcgagctggagaacgggcggaagcgtatgctcgcctccgctggggagttacaaaaggggaacgagctggcgctgccgtctaagtacgtcaacttcctgtacctggcctcccactacgagaagctcaaggggtcgccggaggacaacgagcagaagcagctcttcgtagagcagcacaagcactacctggacgagatcatcgagcagatttcagagttctcaaagcgggtcatcctcgccgacgccaacctggacaaggtgctctcggcctacaacaagcaccgggacaagccgatccgcgaacaggccgaaaacatcatccacctgttcacgctcaccaacctcggtgccccggcggccttcaagtactttgacacgaccatcgaccggaagcgctatacctcgacgaaggaggtgctggacgccaccctgatccaccagtccatcaccgggctttacgagacccggatcgacctctcgcagcta

SEQ ID NO: 76 - DNA, 4101 bp, synthetic construct
  source 1..4101: mol_type = other DNA
gacaagaagtatagtattggactcgccatcggaaccaactctgtggggtgggctgttattacagatgaatataaggtgccatccaaaaagtttaaagttctgggcaatactgatagacactcaatcaagaagaatctgataggtgcacttctgtttgatagtggagagactgccgaggcaaccagacttaaaaggactgcaagaagaagatataccagaagaaagaataggatttgctatttgcaggaaatcttcagcaacgaaatggccaaggttgatgactcatttttccataggttggaggagagttttcttgtggaggaagataagaagcacgaaagacacccaattttcgggaatatagtggacgaggtggcttatcatgagaagtatcccactatctaccacctgagaaagaaacttgtggactcaaccgataaggctgatcttaggcttatatacttggcccttgcacatatgatcaaattcaggggccattttcttatcgaaggcgatcttaatcccgataactcagatgtggacaagctgtttatacaacttgtgcaaacctacaatcaactcttcgaggagaatcccattaacgcctccggcgtggatgcaaaagccatactgtcagccagactgagcaaaagtaggagactggagaatcttatagcccaactgcccggtgaaaagaagaatgggctcttcggaaatctgatcgctctttcattggggttgacacccaactttaagagtaactttgacttggcagaagatgcaaagttgcagctcagtaaagacacatatgacgatgaccttgacaatctcttggcacaaataggggatcaatacgctgaccttttcctcgctgccaagaacctcagcgacgctatactgttgtccgacattcttagggttaataccgaaattacaaaggcccctcttagtgcaagtatgatcaaaaggtatgatgagcatcaccaagaccttacactgctgaaggctctggttagacagcaactccctgaaaagtataaggaaatattcttcgaccaaagtaagaacgggtacgccggttatattgatgggggcgcaagtcaagaagaattttacaaattcatcaagccaattcttgaaaagatggacgggactgaggaattgctggtgaaactgaatagagaggaccttcttagaaaacagaggacatttgacaatgggtccatcccacaccagattcatctgggggaactccacgcaatattgaggagacaagaagacttttacccattccttaaggataatagagagaaaatcgaaaaaatcctgactttcaggattccttactatgttgggccactggccagggggaactcaagattcgcttggatgacaaggaagtcagaagaaaccataaccccttggaattttgaagaggtggttgataagggggcatcagcccagtctttcatagagaggatgaccaactttgataaaaatcttccaaatgagaaggttttgccaaaacatagtcttttgtacgagtactttactgtttataacgaattgaccaaggtgaagtatgtgaccgagggaatgaggaagccagcatttttgtccggggagcaaaagaaagcaatcgttgatcttctcttcaagaccaacagaaaagtgaccgtgaaacaactgaaggaagactacttcaaaaagatagaatgtttcgattcagtggaaattagcggtgttgaagacaggttcaatgcttcattgggtacttaccacgacctgttgaagataatcaaagacaaggactttctcgataatgaggagaacgaagacatcttggaagacattgtgcttacactcactttgtttgaggacagggaaatgattgaggaaagactcaaaacttacgctcatttgtttgatgataaggttatgaaacaactaaaaagaagaaggtacaccggctggggaagattgagtaggaaactgatcaacggtattagagataaacaatccggaaagactatcctcgatttccttaagagtgatggctttgcaaataggaattttatgcagctgattcatgacgactcacttaccttcaaagaagacatccaaaaagctcaggtgtctgggcaaggcgacagtctgcatgaacatatagctaacttggctgggagtcccgccatcaagaaggggatacttcaaacagttaaagttgtggacgaattggtgaaggtaatgggaaggcacaagcctgaaaatatagtgatagaaatggcaagggaaaatcaaacaacccagaagggacagaagaacagtagggaaaggatgaaaaggatagaagaggggatcaaagagcttggtagccagatcctcaaggaacatccagtggagaatacccaacttcaaaacgagaaactctatttgtactacttgcagaacggaagagatatgtatgtggaccaagagcttgatattaacaggctgagcgattatgacgttgaccacatagtgccccaatcattcctcaaggatgactctattgataataaggtgctgacaaggagtgacaagaatagagggaaatccgacaacgttccatccgaggaagttgtgaagaagatgaagaactactggaggcagttgctgaacgctaagctcattacccagaggaaattcgataacctgaccaaagcagagagaggcgggctgagcgaactcgataaagcaggtttcatcaagagacaactcgtggagactaggcaaattactaagcacgtggctcaaatactcgacagcaggatgaacacaaagtacgacgagaacgacaagctcattagagaggttaaggttattactctgaaaagtaaattggttagcgatttcagaaaggatttccaattctataaggttagagagatcaacaattatcatcatgcacatgatgcctatctgaatgctgtggttggtacagcccttatcaagaagtaccctaagctagagagcgagtttgtgtacggagattataaggtgtatgatgtgaggaaaatgatcgctaaaagtgagcaagagattggaaaggctaccgccaaatacttcttttattccaatattatgaatttcttcaagacagaaatcaccctggctaacggcgagataaggaagaggccgcttatcgaaactaatggggagacaggcgaaatagtgtgggacaaagggagggatttcgcaactgtgaggaaggttttgagcatgcctcaggtgaatatcgttaagaaaaccgaagttcaaactggagggttctctaaggaaagcattctccccaagaggaactccgacaagctgattgctagaaagaaagactgggaccccaagaagtatggcggattcgactcacccactgtggcatatagcgttctcgtggtggcaaaggttgaaaagggtaaatccaaaaaactcaaatccgtgaaggaactccttggcataactattatggaaaggagtagctttgaaaagaatcccatcgactttctcgaagctaagggctataaggaagttaagaaggaccttataatcaaacttccaaaatactccctttttgagttggaaaacggcagaaagagaatgttggccagtgccggggagcttcaaaagggcaacgaactggctctgcctagcaaatatgtgaactttttgtatctggcatcacactacgagaaacttaaaggctctcctgaggacaacgagcaaaaacagctctttgttgaacagcataagcactacctcgacgagattattgagcagatcagcgagttctcaaagagagttattctggctgacgctaatcttgacaaggttttgtccgcttacaacaaacacagggataagccaatcagggagcaggcagaaaacataatccatctctttaccctgacaaacctcggtgcccccgctgctttcaagtattttgatactaccattgacaggaagagatatacttccactaaggaagtgctcgacgcaaccctcatacaccaaagtatcacaggcctctatgaaactaggatagatttgtctcaacttgggggcgat

SEQ ID NO: 77 - DNA, 4101 bp, synthetic construct
  source 1..4101: mol_type = other DNA
gacaaaaagtattccatcgggcttgctatcggaaccaactctgtggggtgggcagttattaccgacgaatacaaggtgcccagcaagaagtttaaggttctggggaacacagatagacatagcataaagaaaaacctgataggcgcactgttgttcgactccggggaaacagccgaagctaccaggctgaagagaactgcaagaagaaggtacaccagaagaaaaaacagaatatgttatctccaagagattttctctaacgagatggccaaggtggacgactcattctttcacagactggaagaatctttccttgtggaagaagataagaaacacgagaggcaccctatttttggcaatatcgtggatgaggtggcttaccacgaaaaataccctacaatataccacctcaggaaaaaattggttgatagtacagacaaggccgacctcaggctcatctatttggccctggcccatatgattaaattcagggggcactttctcatcgagggagatttgaaccccgacaacagtgatgttgataagctctttattcagctcgtgcagacttacaatcagttgtttgaggaaaaccccattaatgcttccggggtggacgccaaggcaatcctttctgcaagactctcaaagtcaaggagactcgaaaatctgatagcacagcttccaggagagaagaagaacgggctctttggaaacctgatcgctctgtcactcggactcacacccaatttcaaaagcaattttgatttggcagaggacgctaagctgcaactcagtaaggatacctacgacgatgacttggataatctgctcgcacaaattggggaccagtatgcagacctgtttctcgcagctaagaacttgagtgacgccatattgctcagtgacatcctcagggttaataccgagattacaaaagctccactctctgcaagcatgatcaagaggtatgacgagcaccatcaagacctgacactccttaaggcgttggttaggcagcaacttcctgaaaagtataaggaaatcttcttcgatcaaagcaaaaacggctacgccggctatatagacgggggagcatcccaagaagaattttataagttcataaaacctatattggagaagatggacgggacagaggaattgctcgtgaaactgaacagggaggatctcctcaggaagcaaaggaccttcgacaatggctccatcccacatcagattcacctcggcgaactgcacgcaatactgagaagacaagaggacttttatcctttcctgaaggacaacagggagaaaatcgagaaaatcttgacattcagaatcccatactacgttgggcctctggccagaggtaacagtaggttcgcctggatgactaggaaatcagaggagactattacaccctggaactttgaagaagttgttgataagggagcttcagcacaatcattcatcgaaagaatgacaaactttgacaaaaatctgcctaatgagaaagtgctcccaaaacattccctgctgtatgagtattttaccgtttataacgagcttaccaaggtgaaatacgttactgaaggtatgagaaagccagcttttctttcaggggagcaaaagaaggctatcgtggatcttctctttaagaccaacagaaaggttaccgtgaagcagcttaaggaagactactttaaaaagatcgagtgttttgactcagtggaaataagcggtgttgaagatagattcaacgcatccttgggaacttatcatgatcttcttaagataatcaaggataaagactttctcgacaacgaggaaaacgaagatatactggaggacatagttctgacacttactttgttcgaggatagggagatgatcgaggaaagactgaaaacatatgctcaccttttcgacgacaaagttatgaaacaactcaagagaaggagatatacagggtgggggagattgagcaggaaactgattaatggtatcagagacaaacagtcaggaaaaacaatactcgactttttgaaatcagacgggttcgcaaataggaatttcatgcagcttatacacgacgattcacttacttttaaagaggacattcaaaaggctcaagttagtggacaaggtgactccctccacgaacacatcgcaaatctcgctggcagccctgcaattaagaagggtatactccagacagttaaggttgttgacgagctggttaaagtgatgggaagacacaaacccgagaacatagtgatagagatggccagggaaaaccaaaccactcaaaaagggcagaaaaattccagagagaggatgaaaaggattgaagaaggtatcaaggagctgggtagccaaattctgaaagaacatcctgtggaaaacactcaactccagaatgagaaactctatctgtactatctgcaaaatgggagagatatgtatgtggaccaggaactggacataaacaggctctcagattacgatgtggatcatatcgtgccacagtcctttcttaaggatgatagcatcgacaataaggtgcttaccaggtccgacaagaacaggggaaagtcagataacgtgccttctgaagaagttgttaaaaagatgaagaactactggagacagctgcttaacgctaagctcataacacagaggaagtttgacaacttgaccaaggccgagagaggcggactctcagaattggataaggcagggttcataaaaaggcagctggtggaaacaaggcagataactaaacatgtggctcagatcctcgatagtaggatgaatacaaaatacgatgagaacgacaagctcataagggaggttaaagtgataactctgaaatccaaactggttagcgattttaggaaggatttccagttttacaaagttagggagatcaacaattatcatcacgcccacgatgcctacttgaacgcagttgtgggtactgcacttatcaaaaagtaccctaagctggaatccgagtttgtttatggagactataaggtgtacgacgttagaaaaatgattgcaaagtcagagcaggagatagggaaagccactgcaaaatatttcttttatagcaatatcatgaatttctttaagacagaaatcacactggccaatggggaaataaggaagaggcccctgatcgaaactaatggcgagacaggggagattgtgtgggataaaggtagggactttgcaacagtgaggaaagtgctgagcatgccccaagttaatatcgttaaaaagaccgaggttcaaacagggggctttagtaaggaaagcattttgcccaagaggaatagtgacaaattgattgctaggaaaaaagattgggaccccaaaaagtatggcggatttgatagccccactgttgcttactccgtgctcgtggttgcaaaggtggagaagggaaagagcaagaaactgaagtcagttaaggaactccttggtatcactatcatggaaagaagctcctttgagaagaaccctattgacttcctggaggctaaagggtacaaagaggttaagaaagaccttatcattaaattgcccaaatatagtcttttcgagcttgaaaacggaagaaagaggatgcttgcatccgctggcgaattgcaaaagggcaatgagcttgctctcccttccaagtatgtgaacttcctttatcttgcctcacactatgaaaaactcaaaggttcacccgaagacaacgaacaaaagcaactatttgtggaacaacacaagcactacctggacgaaatcattgagcaaatttctgagttttcaaaaagggtaatcttggctgacgcaaatctcgacaaagttttgtcagcttacaacaaacatagagataagccaattagagagcaagctgagaatatcatccatctgtttaccctgactaaccttggagcgcctgctgcttttaaatatttcgacaccacaatcgacaggaagaggtacactagcactaaggaagttctcgacgccaccctcatccaccagagtattacaggcctgtacgagacaagaattgatctttctcaacttggtggtgac

SEQ ID NO: 78 - DNA, 4101 bp, synthetic construct
  source 1..4101: mol_type = other DNA
gataagaagtactcaatcggtctggcaatcggaaccaactctgtgggttgggcagtgattacagatgagtataaggtgccaagcaaaaaattcaaggtgctgggtaataccgacagacacagcattaagaagaatttgattggagcactcctctttgactcaggggaaacagcagaggcaacaaggctgaagaggacagcaaggcggaggtacacaaggcggaaaaacaggatatgctacctccaggaaatctttagcaacgagatggctaaagtggatgatagctttttccatagactcgaagaatcctttcttgttgaagaggacaaaaagcatgaaaggcatcccatcttcggcaatatagttgatgaggttgcataccatgagaagtaccccacaatctaccacctcagaaagaaacttgtggactccacagataaagcagacctgaggctcatatacctcgcactcgcacacatgatcaagttcagagggcactttctcatcgaaggtgacctgaatccagataattcagatgtggataaactgtttatacagctggtgcaaacatacaaccaacttttcgaggaaaacccaatcaatgcctccggtgttgatgcaaaggccatcctgtcagcaagactcagcaaaagcaggcggctcgaaaacctcatcgcccagcttcccggtgaaaagaagaacgggctctttggtaatctcatcgcattgagccttggtcttactccaaacttcaagagcaattttgatctggcagaggatgctaaactgcaactctcaaaggacacatatgacgatgaccttgacaatctgttggcccagatcggggaccaatatgcagacctcttcctggccgcaaagaatctgtcagatgcaatcctcttgtccgacatactgagagttaacactgagatcacaaaggcacctctgtccgcctccatgattaagagatacgatgagcatcaccaggatctgactttgctcaaagccctcgttagacagcagttgccagaaaagtacaaagaaatattctttgatcaatcaaaaaacggatatgcagggtacatcgacggtggggcaagccaggaagagttctacaaattcatcaaacctatcctggaaaagatggatgggacagaagagctgctggttaagctgaatagggaagacctcctcagaaagcagaggacatttgataacgggagcatccctcatcaaatccacctcggtgaactccatgctatcctgagaaggcaggaagacttttatccatttttgaaggacaatagggagaaaatcgaaaaaatcctgacattcagaatcccatactacgttggtcctctggcaagaggtaacagtaggttcgcatggatgacaaggaaaagcgaggagacaatcacaccctggaattttgaggaagttgttgacaagggtgccagcgcacaatcctttatcgaaagaatgacaaatttcgacaagaatctgcctaacgaaaaggttctcccaaagcattcactcctgtacgaatattttacagtttataacgaactgactaaagttaaatacgttaccgagggtatgaggaagccagcattcctttccggggaacagaagaaagctattgtggacctcctgttcaagacaaatagaaaagtgacagttaagcaactcaaagaggattacttcaaaaagatcgaatgttttgactctgtggagatcagcggggtggaggatagattcaacgccagcctgggtacatatcatgatctcctgaaaatcattaaagacaaggacttccttgacaacgaggagaacgaggacattctggaagacattgttctgaccctcacactctttgaggatagggagatgattgaggaaagactgaagacctacgcccacctctttgacgataaagtgatgaaacagctcaagagaagaaggtatacaggttgggggagactgagcaggaagttgatcaatgggattagggacaaacagtccgggaaaacaatcctcgattttctgaagtcagacggtttcgcaaacagaaattttatgcagctcattcacgatgacagcttgacattcaaggaagacatccaaaaggctcaagtgagcggccaaggggatagcctccacgagcatattgcaaatctggcaggttcaccagccatcaaaaagggcatacttcagacagttaaggttgtggacgaattggttaaagttatgggcaggcataagccagagaatatcgttatcgaaatggcaagggagaaccaaacaactcaaaaagggcagaaaaatagcagagagaggatgaaaagaatcgaggaagggatcaaggaacttgggtcccaaatcctcaaggagcacccagttgaaaatactcaactgcaaaacgagaagctctatctctactatctccaaaacgggagggatatgtatgttgaccaggagctggatattaacagactgtcagattatgatgttgatcatatcgtgccccagtcattcctgaaggacgattccatcgacaacaaagttctcacaaggtccgataaaaacaggggcaagtccgataacgttccaagcgaagaagtggtgaaaaagatgaaaaactattggagacaacttctgaatgcaaagttgattactcagagaaagtttgacaacctcacaaaagcagaaagaggcgggcttagcgaactcgataaggcagggtttatcaaaagacagctggttgagacaaggcagatcacaaaacatgtggcacagatccttgactcaaggatgaataccaagtatgatgagaatgataagttgatcagggaggttaaagttatcacactcaaatccaaactggtgtcagacttcaggaaagactttcaattttataaggtgagggagatcaataactaccaccatgcacatgacgcctacctgaacgcagtggtgggtacagcattgattaaaaaataccctaagctggagtctgagtttgtgtacggggactacaaggtgtacgacgtgaggaaaatgatagccaagtccgagcaggagatcgggaaagcaacagctaagtatttcttttacagtaatatcatgaatttctttaaaactgagattactctggcaaacggggagatcaggaaaagacccctcatcgagactaatggtgaaacaggtgagatcgtttgggacaaggggagggattttgctactgttagaaaagttctgagtatgccacaagtgaatattgtgaaaaagacagaagttcagacaggtgggttctccaaagaatccatcctgcccaagagaaattcagacaagctcatcgcaagaaagaaggactgggaccctaagaagtacggaggatttgacagccccaccgtggcctattccgtgcttgttgtggcaaaggtggagaaagggaagagcaaaaaactgaaatccgtgaaagaactgctgggaattaccatcatggaaagaagctcctttgagaagaacccaatcgacttcctggaagcaaaaggatataaggaagtgaaaaaggacctcattatcaagctcccaaaatactcacttttcgagttggagaacggtagaaagaggatgctggcaagcgcaggggaacttcagaaaggcaatgagctggcattgccatcaaagtatgtgaacttcctctacttggccagccattacgagaaacttaaaggtagcccagaagataacgagcaaaaacagctctttgtggaacagcataagcattatctggatgagatcatagaacaaatctcagagttttccaagagagttatcctcgcagatgcaaacctggataaggttctctcagcctataataagcatagagacaagccaattagagagcaagcagagaacattatccacttgttcactcttacaaacctgggggcaccagccgccttcaaatatttcgatacaacaatagacagaaagaggtataccagcaccaaagaagttctcgacgccacactgatccatcaatcaatcacaggcctttacgaaactaggatcgacttgtcacaactgggtggggat

SEQ ID NO: 79 - DNA, 3307 bp, synthetic construct
  source 1..3307: mol_type = other DNA
gagcaaggacacctacgacgacgacttggacaacctattggcccagataggtgaccagtatgcagacctcttccttgcggccaagaacttgagtgacgctatactgctcagtgacatcctgagggtgaacactgagatcactaaggcccctctctctgcctcaatgattaagcgttacgacgagcatcaccaggatctcaccctgcttaaggcccttgttcggcagcagctccctgagaagtacaaggagatattttttgaccagtctaagaacggctacgccggttacattgacggtggggcaagccaggaggagttctacaagttcatcaagccgatccttgagaagatggacggcaccgaggagctacttgtcaagttgaaccgggaagacctgctccggaaacagcgtacattcgacaacggcagcatccctcaccagatccacctgggcgaactacacgccatcctccgacgtcaggaggacttctatccattcttgaaagataacagggaaaaaatcgaaaaaatacttacgtttcgaataccttactacgtggggccccttgctcggggaaactccagattcgcatggatgaccaggaagtcagaggagaccatcacaccctggaactttgaggaggtggttgacaaaggtgcttctgcccagtccttcattgagcggatgactaacttcgacaagaacctgcccaacgagaaggtgctgccaaagcacagcctgctctacgaatactttactgtgtacaatgagctgacgaaggtgaagtacgtgacagaggggatgcggaagcccgctttcctgagcggcgagcaaaaaaaagcaatcgtggacctactgttcaagaccaaccgaaaggtgacagtgaagcagctcaaggaggactacttcaaaaaaatcgagtgcttcgactctgttgagataagcggcgtggaggaccgattcaacgcctcattgggaacctatcacgacctgctcaagatcattaaggacaaggacttcctggataatgaggagaatgaggacatcctggaggatattgtgctgacccttactctattcgaggacagggagatgatcgaggagcgactcaagacctacgctcacctgttcgacgacaaggttatgaagcaattgaagcgtaggcgatacacggggtggggaagactctcccgaaaactgataaacggcatcagggacaagcagtcagggaagacgatcttggacttcctgaaatccgacgggttcgccaaccgcaacttcatgcagctcattcacgacgactcactaacgttcaaagaggacattcagaaggctcaagtcagtggacaaggcgactccctgcacgagcacattgcaaaccttgcgggctccccggcgattaaaaagggcattctccaaacggttaaggtggtggacgagctggtgaaggtgatgggccgacacaagcctgagaacatcgtgatcgagatggccagggagaaccagactacccagaagggtcagaagaactctcgggaacgtatgaagcgtattgaggaggggattaaggagttgggctctcaaatcctcaaggagcaccctgtggagaacactcagctccaaaacgagaagctgtacctgtactacctgcaaaacgggcgcgatatgtacgtggatcaggagttggacatcaacaggcttagcgattacgacgtggaccacatcgtgccacagtcattcttaaaggacgacagcatcgacaacaaggttctgacgaggagcgacaagaatcgagggaaaagtgacaatgttccatccgaggaggtggtcaagaaaatgaagaactattggcgtcagcttctgaacgccaagctcatcacccagcggaaattcgacaacctgactaaggctgagcgaggcggactctccgagcttgacaaggctggcttcatcaagcggcagttggtcgaaacccgacagataacgaagcacgttgcccagatacttgactcccgtatgaacaccaagtacgacgagaacgacaagctcatcagggaggtgaaggtcattacccttaagtccaaactcgtcagcgactttcgtaaggacttccagttctacaaggtgcgcgagatcaataactaccaccacgcacacgacgcctacctgaacgcagtggttggaaccgcgttgattaaaaagtaccccaagttggagtcggagttcgtttacggggactacaaggtgtacgacgttcggaagatgatcgccaagtctgaacaggagatcgggaaagcaaccgccaagtatttcttctatagcaacatcatgaacttctttaaaaccgagatcacacttgccaatggcgagatccgtaagaggccgctgatcgagacaaatggggagactggcgagatcgtgtgggacaagggccgcgacttcgcaaccgttcggaaagtcttgtccatgcctcaagtcaacatcgtcaagaagactgaggtgcaaacaggcgggttctcgaaggagtccatactgcccaagaggaactcagacaagctcatagcacgcaaaaaagactgggatccaaagaaatacggcgggttcgactcgccgacagtcgcatactccgtgttagtggtggctaaagtggaaaaggggaagtccaagaagctcaagtccgtcaaggagttgctcgggatcaccattatggaacggtcctcattcgagaagaatcccattgacttcctagaggcgaagggctacaaagaggtcaaaaaggacctaattattaagctccccaagtattcactcttcgaacttgaaaatggtcgtaagcggatgttggcaagcgctggagagcttcagaaggggaacgagcttgcactgccttccaagtacgtgaacttcctgtacctcgcctctcattacgagaagttgaagggctcaccggaggacaacgagcagaagcagttgttcgtggagcagcacaagcactacctcgacgagatcattgagcagataagtgagttcagcaaacgggtgatccttgccgacgctaacctggacaaggtgctgagcgcctacaacaagcacagagacaagccgatccgagagcaagcggagaacatcatacacctgttcaccctcacgaacctcggggctcccgcagccttcaaatattttgacacgaccatcgaccgtaaacgctacactagcacgaaggaggtgctggacgctacccttatccaccagtccatcaccggcctgtacgagacgagaatcgacttgtcgcagctcggtggtgac

SEQ ID NO: 80 - DNA, 4101 bp, synthetic construct
  source 1..4101: mol_type = other DNA
gacaaaaaatactcaattggtctggcaattgggaccaacagtgtcggatgggccgtgattaccgacgagtacaaggtgccgtccaaaaaattcaaggtgcttgggaacaccgaccgccactcgatcaagaaaaacctaatcggtgcgttgcttttcgacagtggggagaccgccgaggcaacacgcttaaaacgcacagctaggaggagatatacacggcgcaagaaccgaatatgctacttacaggagatattctccaatgagatggcgaaggtggacgactctttcttccatcggcttgaggaatccttcctggtcgaggaggacaagaagcacgagcgacacccgatattcgggaacatcgttgatgaggtggcgtaccacgagaagtacccaacgatataccacttacgcaagaagctcgtggactctacggacaaggccgacttgcgccttatctacttggcactggcccacatgattaagttccgaggccacttccttatcgagggtgacctgaaccccgataactccgacgtggacaagctcttcatccaactcgtccagacatacaaccagctattcgaggagaatcctatcaacgcctctggggtggacgctaaagctatcctctcagcccgcctgtcaaagtcgaggaggttggagaacctaatcgcccagcttccaggcgagaagaaaaatgggctgttcggaaaccttatcgcactctcactgggcctaaccccgaacttcaagtccaacttcgacctggcagaggacgcgaaattgcagttgtcgaaagacacctatgacgatgacctggacaacctgttggcccagataggggaccagtacgccgacctgttcctagcggccaagaacctgtccgacgccatcttgctgtcggatatactgcgggtgaacaccgagatcactaaagcacctctctccgccagcatgattaagcgttacgacgagcaccaccaagatttgaccctgctaaaggcacttgtacggcagcagcttcccgagaagtacaaggagatctttttcgaccaaagcaagaacggctacgccgggtacatcgacggaggtgccagccaggaggagttctacaagttcattaagcccatcctggagaagatggacgggactgaggaactacttgtgaagctgaaccgggaagacttactacggaagcagcgtaccttcgacaacggttctatcccacatcagatccatcttggggagttgcacgcgatcctgcgacgccaggaggacttttaccccttcctgaaagacaaccgcgagaaaatcgagaagatactgaccttcagaataccttactacgtcggaccccttgcgcgaggcaactcaagattcgcgtggatgaccaggaaatcagaggagaccatcacaccctggaatttcgaggaggtggttgacaagggtgcctccgcccagtcctttatcgaacgaatgaccaacttcgacaagaacttgcccaacgagaaggtgctccccaaacacagcctcctctacgaatatttcacagtgtacaacgagcttactaaagttaagtatgttactgagggcatgaggaaacccgccttcctgtcaggcgagcagaagaaagctattgtggacctccttttcaagaccaaccggaaggtgacagtgaagcagctcaaggaggactacttcaagaagatagagtgcttcgacagcgtggagatcagcggggtggaggacagattcaatgcctctctcggaacataccacgacttgcttaagatcatcaaggacaaggacttcctcgacaacgaggaaaacgaggatattctggaggatattgttctgactcttaccctgttcgaggaccgggagatgatcgaggagcgtctcaagacctacgcccacctgttcgacgacaaagttatgaagcagctcaagcgtcggagatataccggatggggccgtctgtctcggaagctcatcaacgggatcagggacaagcagtcagggaagacgatcttagacttccttaagtctgacggcttcgccaacaggaacttcatgcagttgatccacgacgacagccttaccttcaaggaggacatccagaaggcccaagtgagtggccagggtgacagcctccacgagcatattgctaatcttgcgggttccccagcgattaaaaagggcatacttcaaaccgttaaggtggtggacgagcttgtcaaggtgatggggcgacacaagcccgagaacatcgtgatcgagatggccagggagaaccagaccacccagaaggggcagaagaatagccgagaacgcatgaagcgcatcgaggaggggattaaggagctagggagccagatcctcaaggaacatcccgtcgagaacacccagctccagaacgagaagctatacctctactacttgcaaaacgggagggatatgtacgtggatcaggagttggacattaaccgcctaagcgactacgacgtagatcacatcgtgcctcagtcattcctcaaagacgacagcattgacaacaaagtcttgacccgatccgacaagaaccgaggaaaatccgacaatgtgccctcagaggaggtcgtcaagaaaatgaagaactattggaggcagctacttaacgccaaactcataacccagcggaagttcgacaacctgacaaaggctgagcggggtgggctcagcgagcttgacaaggctggcttcatcaagcggcagttggtggagacaagacagataacgaagcacgtggctcagatcctggactctcgcatgaacacgaagtacgacgagaacgacaaattgatccgcgaggtcaaggttattacgctcaagagcaaacttgtcagcgatttccgcaaggacttccagttctacaaggtgagggagattaacaactaccaccatgcacatgatgcctacttgaacgcagtggtggggaccgcgcttattaaaaagtaccctaagttggagtcagagttcgtttatggggactacaaggtgtacgacgtccggaagatgattgcaaagtctgaacaggaaatcgggaaggccaccgccaaatatttcttctacagtaacattatgaatttttttaagactgaaattactctcgcaaacggcgagatcaggaagcgtcccctcatcgagacaaacggggagaccggggagatagtctgggacaaggggcgggacttcgctacggtgaggaaggtgctctcgatgccacaagtgaacatcgtcaaaaagacagaggtgcagaccggtggcttctcaaaggagtcaatcctgccaaaacgtaacagcgacaagctcatcgcccgcaagaaagactgggaccctaagaagtatggtgggttcgactcaccgacggtcgcatactccgttctggtcgtggcaaaggtggaaaagggcaagtccaaaaaactgaaatccgtgaaggagttgcttggcattaccatcatggaacgcagcagcttcgagaagaaccccattgacttcctggaggctaaagggtacaaggaggtcaagaaagatttaattattaagctacctaagtacagcttgttcgagctggagaacggccgaaaacgaatgctcgcatccgccggggaacttcaaaagggcaacgagcttgcgctgccctccaagtacgtgaacttcctgtacttggcatcccactacgagaaactcaagggtagcccagaggacaacgagcagaagcagctattcgtggagcagcacaagcactacctcgacgagataatcgagcagatcagtgagttcagtaagcgggtgatactcgcggacgccaacttggacaaggtgcttagtgcctacaacaagcaccgtgacaagcccatccgagaacaggctgagaacatcatccaccttttcactctgacaaacctcggtgctcccgccgccttcaaatacttcgacactaccatcgacaggaagcgctacacatctacgaaggaagttcttgacgctacgcttattcatcagtctatcacagggctgtacgagacaaggatcgaccttagccaactcggcggggat

SEQ ID NO: 81 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 82 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 83 - protein, 228 aa, Rattus norvegicus
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 84 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 85 - DNA, 621 bp, Petromyzon marinus
  source 1..621: mol_type = other DNA
acagatgcagagtatgtgagaattcacgaaaagctggacatctataccttcaagaagcagttctttaacaataagaagtctgtgagccataggtgctacgtgctgttcgagctgaagagaaggggtgaaagaagggcatgtttttgggggtatgctgtgaacaagccccagtctggaactgagagaggcattcacgccgaaattttcagcatcagaaaggtggaggaatacctgagggataaccctggacagtttacaattaattggtattctagctggtctccatgcgctgactgtgccgagaagatcctggaatggtacaaccaggagctgagaggaaatggccataccctgaagatttgggcctgcaagctgtactatgaaaagaacgcaagaaatcagatcggactgtggaacctgagggataatggtgtggggctgaacgtgatggtgtccgagcactatcagtgctgtagaaagattttcattcagtcctcacataatcagctgaacgagaatagatggctggaaaagactctgaagagggctgagaagagaaggtccgaactgtcaattatgatccaggtgaagatcctgcacaccactaagtcacctgccgtg

SEQ ID NO: 86 - protein, 160 aa, synthetic construct
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 87 - protein, 229 aa, Rattus norvegicus [sequence omitted]

SEQ ID NO: 88 - protein, 198 aa, Homo sapiens [sequence omitted]

SEQ ID NO: 89 - protein, 197 aa, synthetic construct
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEDRKAEPEGLRRLHRAGVQIAIMTFKDYFYCWNTFVENHGRTFKAWEGLHENSVRLSRQLRRILLPLYEVDDLRDAFRTCT

SEQ ID NO: 90 - protein, 207 aa, synthetic construct
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 91 - protein, 228 aa, synthetic construct
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 92 - protein, 162 aa, synthetic construct
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 93 - protein, 166 aa, Escherichia coli
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 94 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 95 - protein, 166 aa, synthetic construct
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 96 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 97 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 98 - protein, 1763 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 99 - protein, 1565 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 100 - protein, 1565 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 101 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 102 - protein, 167 aa, synthetic construct
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 103 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 104 - protein, 83 aa, Takahashivirus sp.
  source 1..83: note = Bacillus phage AR9
TNLSDIIEKETGKQLVIQESILMLPEEVEEVIGNKPESDILVHTAYDESTDENVMLLTSDAPEYKPWALVIQDSNGENKIKML

SEQ ID NO: 105 - protein, 1372 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 106 - protein, 1374 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 107 - protein, 1376 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 108 - DNA, 66 bp, Saccharomyces bayanus
  source 1..66: mol_type = other DNA
ttcttgtcgtacttatagatcgctacgttatttcaattttgaaaatctgagtcctgggagtgcgga

SEQ ID NO: 109 - protein, 605 aa, Homo sapiens
MSGWESYYKTEGDEEAEEEQEENLEASGDYKYSGRDSLIFLVDASKAMFESQSEDELTPFDMSIQCIQSVYISKIISSDRDLLAWFYGTEKDKNSVNFKIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPLKEACRAYGLKSGLKKQELLEALTKHFQD

SEQ ID NO: 110 - protein, 482 aa, synthetic construct
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 111 - DNA, 10 bp, Methanobacterium thermoautotrophicum
  source 1..10: mol_type = other DNA
aatttttgga

SEQ ID NO: 112 - protein, 83 aa, Methanobacterium thermoautotrophicum
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 113 - DNA, 25 bp, Emesvirus zinderi
  source 1..25: mol_type = other DNA
gcgcacatgaggatcacccatgtgc

SEQ ID NO: 114 - protein, 116 aa, Emesvirus zinderi
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 115 - DNA, 26 bp, Pepevirus rubrum
  source 1..26: mol_type = other DNA
ataaggagtttatatggaaaccctta

SEQ ID NO: 116 - protein, 127 aa, Pepevirus rubrum
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 117 - DNA, 19 bp, Shigella flexneri
  source 1..19: mol_type = other DNA
ctgaatgcctgcgagcatc

SEQ ID NO: 118 - protein, 62 aa, Shigella flexneri
MKSIRCKNCNKLLFKADSFDHIEIRCPRCKRHIIMLNACEHPTEKHCGKREKITHSDETVRY

SEQ ID NO: 119 - protein, 24 aa, synthetic construct
EELLSKNYHLENEVARLKKGSGSG

SEQ ID NO: 120 - protein, 241 aa, synthetic construct
EEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSG

SEQ ID NO: 121 - protein, 277 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 122 - protein, 11 aa, unidentified
  source 1..11: note = Lachnospiraceae bacterium
GFVTESGEKIK

SEQ ID NO: 123 - DNA, 27 bp, synthetic construct
  source 1..27: mol_type = other DNA
tttaggaatcccttctgcagcacctgg

SEQ ID NO: 124 - RNA, 43 nt, synthetic construct
  source 1..43: mol_type = other RNA
aatttctactaagtgtagatggaatcccttctgcagcacctgg

SEQ ID NO: 125 - protein, 1373 aa, synthetic construct; identical to SEQ ID NO: 10

SEQ ID NO: 126 - protein, 1375 aa, synthetic construct; identical to SEQ ID NO: 11

SEQ ID NO: 127 - protein, 1377 aa, synthetic construct; identical to SEQ ID NO: 12

SEQ ID NO: 128 - protein, 1379 aa, synthetic construct; identical to SEQ ID NO: 13

SEQ ID NO: 129 - protein, 1373 aa, synthetic construct; identical to SEQ ID NO: 14

SEQ ID NO: 130 - protein, 1375 aa, synthetic construct; identical to SEQ ID NO: 15

SEQ ID NO: 131 - protein, 1375 aa, synthetic construct
SKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 132 - protein, 1379 aa, synthetic construct; identical to SEQ ID NO: 17

SEQ ID NO: 133 - DNA, 46 bp, synthetic construct
  source 1..46: mol_type = other DNA
gatgctttaggaatcccttctgcagcacctgggcgcaggtcacgag

SEQ ID NO: 134 - DNA, 99 bp, Homo sapiens
  source 1..99: mol_type = other DNA
cctccggattcctaccccgaaaagacagtggttaggacagggatcaccggggtgacaccccacctcccctgtctattttcatgggtcttggtctcggtg

SEQ ID NO: 135 - DNA, 99 bp, Homo sapiens
  source 1..99: mol_type = other DNA
ggaggcctaaggatggggcttttctgtcaccaatcctgtccctagtggccccactgtggggtggaggggacagataaaagtacccagaaccagagccac

SEQ ID NO: 136 - DNA, 20 bp, Homo sapiens
  source 1..20: mol_type = other DNA
gcattttcaggaggaagcga

SEQ ID NO: 137 - DNA, 23 bp, Homo sapiens
  source 1..23: mol_type = other DNA
caggaggaagcgatggcttcaga

SEQ ID NO: 138 - DNA, 20 bp, Homo sapiens
  source 1..20: mol_type = other DNA
gtcccctccaccccacagtg

SEQ ID NO: 139 - DNA, 23 bp, Homo sapiens
  source 1..23: mol_type = other DNA
tctgtcccctccaccccacagtg

SEQ ID NO: 140 - DNA, 4101 bp, synthetic construct
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 141 - DNA, 4101 bp, synthetic construct
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctggccgacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctgccctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaaggcccctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 142 - DNA, 4101 bp, synthetic construct
  source 1..4101: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 143 - DNA, 4101 bp, synthetic construct
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 144 - DNA, 23 bp, synthetic construct
  source 1..23: mol_type = other DNA
accttggagacggcgactctctg

SEQ ID NO: 145 - DNA, 23 bp, synthetic construct
  source 1..23: mol_type = other DNA
gcggatgttccaatcagtacgca

SEQ ID NO: 146 - DNA, 23 bp, synthetic construct
  source 1..23: mol_type = other DNA
tgtcaccaatcctgtccctagtg

SEQ ID NO: 147 - DNA, 23 bp, synthetic construct
  source 1..23: mol_type = other DNA
tctgtcccctccaccccacagtg

SEQ ID NO: 148 - DNA, 23 bp, synthetic construct
  source 1..23: mol_type = other DNA
tatgagttacaacgaacacctca

SEQ ID NO: 149 - DNA, 23 bp, synthetic construct
  source 1..23: mol_type = other DNA
cacgtctcatatgccccttggca

SEQ ID NO: 150 - RNA, 23 nt, synthetic construct
  source 1..23: mol_type = other RNA
tgtcaccaatcctgtccctagtg

SEQ ID NO: 151 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 152 - protein, 198 aa, synthetic construct
EASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 153 - protein, 17 aa, synthetic construct
SGSETPGTSESATPESM

SEQ ID NO: 154 - protein, 13 aa, synthetic construct
GSPKKKRKVSGGS

SEQ ID NO: 155 - protein, 166 aa, Escherichia coli
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 156 - protein, 397 aa, synthetic construct
GSSETPGTSESATPEGGSGGSGSEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNGGGGSTNLSDIIEKETGKQLVIQESILMLPEEVEEVIGNKPESDILVHTAYDESTDENVMLLTSDAPEYKPWALVIQDSNGENKIKMLSGGSGGSGGSYKLILNGKTLKGETTTEAVDAATAEKVFKQYANDNGVDGEWTYDDATKTFTVTESGGSKRTADGSEFEPKKKRKVGSG

SEQ ID NO: 157 - protein, 1787 aa, synthetic construct
MKRTADGSEFESPKKKRKVSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGGGGSGGGGSGGGGSGPKKKRKVAAAGSEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKSGGSKRTADGSEFEPKKKRKVGSG

SEQ ID NO: 158 - protein, 1787 aa, synthetic construct
MKRTADGSEFESPKKKRKVSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGGGGSGGGGSGGGGSGPKKKRKVAAAGSEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKSGGSKRTADGSEFEPKKKRKVGSG

SEQ ID NO: 159 - protein, 1789 aa, synthetic construct
MKRTADGSEFESPKKKRKVSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGGGGSGGGGSGGGGSGPKKKRKVAAAGSEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKGSGSGGSGSGGSGSGSGGSGSGGSGSGEELLSKNYHLENEVARLKKSGGSKRTADGSEFEPKKKRKVGSG

SEQ ID NO: 160 - protein, 1739 aa, synthetic construct
MKRTADGSEFESPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSTSQSDGSSVPADIDQSSDSDQSSSQGQPGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGSG

SEQ ID NO: 161 - protein, 1739 aa, synthetic construct
MKRTADGSEFESPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSTSQSDGSSVPADIDQSSDSDQSSSQGQPGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGSG

SEQ ID NO: 162 - protein, 1741 aa, synthetic construct
MKRTADGSEFESPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSTSQSDGSSVPADIDQSSDSDQSSSQGQPGSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGSG

SEQ ID NO: 163 - protein, 1734 aa, synthetic construct
MKRTADGSEFESPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLTKYDSGGSKRTADGSEFEPKKKRKVGSG

SEQ ID NO: 164 - protein, 1734 aa, synthetic construct
MKRTADGSEFESPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLTKYDSGGSKRTADGSEFEPKKKRKVGSG

SEQ ID NO: 165 - protein, 1736 aa, synthetic construct
MKRTADGSEFESPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGSPKKKRKVSGGSTNLSDIIEKETGKQLVIQESILMLPEEVEEVIGNKPESDILVHTAYDESTDENVMLLTSDAPEYKPWALVIQDSNGENKIKMLTKYDSGGSKRTADGSEFEPKKKRKVGSG

SEQ ID NO: 166 - protein, 1616 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSKRTADGSEFEPKKKRKVGSG

SEQ ID NO: 167 - protein, 1616 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSKRTADGSEFEPKKKRKVGSG

SEQ ID NO: 168 - protein, 1618 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSGGSKRTADGSEFEPKKKRKVGSG

SEQ ID NO: 169 - protein, 53 aa, Vibrio campbellii
CRVTGVQLKNHLIASHIKPWAVSNNQERLDGHNGLLLAPHVDHLFDKGFISFE

SEQ ID NO: 170 - protein, 137 aa, Bacillus thermoleovorans
MPNRPLKPCNKIGCTNLTRDRYCEQHKHLAEQRQRTRRNDKEYDKHKRNQQARAFYHSREWERVRLAVLARDNYLCQHCLKEKKITRAVIVDHVVPLLVDWSKRLDMDNLQSLCQSCHNRKTAEDKRRYGQGRSGKF

SEQ ID NO: 171 - protein, 49 aa, Okubovirus sp.
  source 1..49: note = Bacillus phage SP01
KGKTFQVHRLVAIHFCEGYEEGLVVDHKDGNKDNNLSTNLRWVTQKINV

SEQ ID NO: 172 - protein, 156 aa, Neisseria meningitidis
SFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNL

SEQ ID NO: 173 - protein, 53 aa, Vibrio campbellii; identical to SEQ ID NO: 169

SEQ ID NO: 174 - protein, 62 aa, Streptomyces coelicolor
SARGAVLKRCQKRCENPECAGHPTELTKAGLPILQVDHVNDLAKGGPDVPWNMIALCPNCHA

SEQ ID NO: 175 - protein, 4 aa, synthetic construct
GSSG

SEQ ID NO: 176 - protein, 6 aa, synthetic construct
GSSGSS

SEQ ID NO: 177 - protein, 8 aa, synthetic construct
GSSGSSGS

SEQ ID NO: 178 - protein, 60 aa, synthetic construct
  VARIANT 4..60: note = Residue may be present or absent
GSSGSSGSSGSSGSSGSSGSSGSSGSSGSSGSSGSSGSSGSSGSSGSSGSSGSSGSSGSS

SEQ ID NO: 179 - protein, 62 aa, synthetic construct
  VARIANT 4..60: note = Residue may be present or absent
GSSGSSGSSGSSGSSGSSGSSGSSGSSGSSGSSGSSGSSGSSGSSGSSGSSGSSGSSGSSGS

SEQ ID NO: 180 - protein, 1228 aa, unidentified
  source 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 181 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 182 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 183 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 184 - protein, 1376 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 185 - protein, 1376 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 186 - protein, 1376 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGSSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 187 - protein, 1372 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 188 - protein, 1372 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 189 - protein, 1372 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 190 - protein, 1374 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 191 - protein, 1374 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 192 - protein, 1374 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGSGENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 193 - protein, 1328 aa, synthetic construct
MAGSKKRRIKQDTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNREKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSKKRRIKQD

SEQ ID NO: 194 - DNA, 3790 bp, synthetic construct
  source 1..3790: mol_type = other DNA
ccatgggcagcaaactggaaaaatttacgaattgttatagcctgtccaagaccctgcgtttcaaagccatccccgttggcaaaacccaggagaatattgataataaacgtctgctggttgaggatgaaaaaagagcagaagactataagggagtcaaaaaactgctggatcggtactacctgagctttataaatgacgtgctgcatagcattaaactgaaaaatctgaataactatattagtctgttccgcaagaaaacccgaacagagaaagaaaataaagagctggaaaacctggagatcaatctgcgtaaagagatcgcaaaagcttttaaaggaaatgaaggttataaaagcctgttcaaaaaagacattattgaaaccatcctgccggaatttctggatgataaagacgagatagcgctcgtgaacagcttcaacgggttcacgaccgccttcacgggctttttcgataacagggaaaatatgttttcagaggaagccaaaagcacctcgatagcgttccgttgcattaatgaaaatttgacaagatatatcagcaacatggatattttcgagaaagttgatgcgatctttgacaaacatgaagtgcaggagattaaggaaaaaattctgaacagcgattatgatgttgaggattttttcgagggggaattttttaactttgtactgacacaggaaggtatagatgtgtataatgctattatcggcgggttcgttaccgaatccggcgagaaaattaagggtctgaatgagtacatcaatctgtataaccaaaagaccaaacagaaactgccaaaattcaaaccgctgtacaagcaagtcctgagcgatcgggaaagcttgagcttttacggtgaaggttataccagcgacgaggaggtactggaggtctttcgcaataccctgaacaagaacagcgaaattttcagctccattaaaaagctggagaaactgtttaagaattttgacgagtacagcagcgcaggtatttttgtgaagaacggacctgccataagcaccattagcaaggatatttttggagagtggaatgttatccgtgataaatggaacgcggaatatgatgacatacacctgaaaaagaaggctgtggtaactgagaaatatgaagacgatcgccgcaaaagctttaaaaaaatcggcagctttagcctggagcagctgcaggaatatgcggacgccgacctgagcgtggtcgagaaactgaaggaaattattatccaaaaagtggatgagatttacaaggtatatggtagcagcgaaaaactgtttgatgcggacttcgttctggaaaaaagcctgaaaaaaaatgatgctgttgttgcgatcatgaaagacctgctcgatagcgttaagagctttgaaaattacattaaagcattctttggcgagggcaaagaaacaaacagagacgaaagcttttatggcgacttcgtcctggcttatgacatcctgttgaaggtagatcatatatatgatgcaattcgtaattacgtaacccaaaagccgtacagcaaagataagttcaaactgtatttccagaacccgcagtttatgggtggctgggacaaagacaaggagacagactatcgcgccactattctgcgttacggcagcaagtactatctcgccatcatggacaaaaaatatgcaaagtgtctgcagaaaatcgataaagacgacgtgaacggaaattacgaaaagattaattataagctgctgccagggcccaacaagatgttaccgaaagtatttttttccaaaaaatggatggcatactataacccgagcgaggatatacagaagatttacaaaaatgggaccttcaaaaagggggatatgttcaatctgaatgactgccacaaactgatcgatttttttaaagatagcatcagccgttatcctaaatggtcaaacgcgtatgattttaatttctccgaaacggagaaatataaagacattgctggtttctatcgcgaagtcgaagaacagggttataaagttagctttgaatcggccagcaagaaagaggttgataaactggtggaggagggtaagctgtatatgtttcagatttataacaaagactttagcgacaaaagccacggtactcctaatctgcatacgatgtactttaaactgctgtttgatgagaataaccacggccaaatccgtctctccggtggagcagaactttttatgcggcgtgcgagcctaaaaaaggaagaactggtggtgcatcccgccaacagcccgattgctaacaaaaatccagataatcctaagaagaccaccacactgtcgtacgatgtctataaggataaacgtttctcggaagaccagtatgaattgcatataccgatagcaattaataaatgcccaaaaaacattttcaaaatcaacactgaagttcgtgtgctgctgaaacatgatgataatccgtatgtgatcggaattgaccgtggggagagaaatctgctgtatattgtagtcgttgatggcaagggcaacatcgttgagcagtatagcctgaatgaaataattaataattttaacggtatacgtattaaaaccgactatcatagcctgctggataaaaaggagaaagagcgttttgaggcacgccaaaattggacgagcatcgaaaacatcaaggaactgaaggcaggatatatcagccaagtagtccataaaatctgtgaactggtggagaagtacgacgctgtcattgccctggaagacctcaatagcggctttaaaaacagccgggtgaaggtggagaaacaggtataccaaaagtttgaaaagatgctcattgataagctgaactatatggttgataaaaagagcaacccgtgcgccactggcggtgcactgaaagggtaccaaattaccaataaatttgaaagctttaaaagcatgagcacgcagaatgggtttattttttatataccagcatggctgacgagcaagattgaccccagcactggttttgtcaatctgctgaaaaccaaatacacaagcattgcggatagcaaaaaatttatttcgagcttcgaccgtattatgtatgttccggaggaagatctgtttgaatttgccctggattataaaaacttcagccgcaccgatgcagattatatcaaaaaatggaagctgtacagttatggtaatcgtatacgtatcttccgtaatccgaagaaaaacaatgtgttcgattgggaagaggtctgtctgaccagcgcgtataaagaactgttcaacaagtacggaataaattatcagcaaggtgacattcgcgcactgctgtgtgaacagtcagataaagcattttatagcagctttatggcgctgatgagcctgatgctccagatgcgcaacagcataaccggtcgcacagatgttgactttctgatcagccctgtgaagaatagcgacggcatcttctacgattccaggaactatgaagcacaggaaaacgctattctgcctaaaaatgccgatgccaacggcgcctataatattgcacggaaggttctgtgggcgattggacagttcaagaaagcggaagatgagaagctggataaggtaaaaattgctattagcaataaggaatggctggagtacgcacagacatcggttaaacacggtagtaaaaggccggcggccacgaaaaaggccggccaggcaaaaaagaaaaagggagcggccgcactcgagcaccaccaccaccaccactgagcggccgc

SEQ ID NO: 195 - DNA, 8986 bp, synthetic construct
  source 1..8986: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcaaactggaaaaatttacgaattgttatagcctgtccaagaccctgcgtttcaaagccatccccgttggcaaaacccaggagaatattgataataaacgtctgctggttgaggatgaaaaaagagcagaagactataagggagtcaaaaaactgctggatcggtactacctgagctttataaatgacgtgctgcatagcattaaactgaaaaatctgaataactatattagtctgttccgcaagaaaacccgaacagagaaagaaaataaagagctggaaaacctggagatcaatctgcgtaaagagatcgcaaaagcttttaaaggaaatgaaggttataaaagcctgttcaaaaaagacattattgaaaccatcctgccggaatttctggatgataaagacgagatagcgctcgtgaacagcttcaacgggttcacgaccgccttcacgggctttttcgataacagggaaaatatgttttcagaggaagccaaaagcacctcgatagcgttccgttgcattaatgaaaatttgacaagatatatcagcaacatggatattttcgagaaagttgatgcgatctttgacaaacatgaagtgcaggagattaaggaaaaaattctgaacagcgattatgatgttgaggattttttcgagggggaattttttaactttgtactgacacaggaaggtatagatgtgtataatgctattatcggcgggttcgttaccgaatccggcgagaaaattaagggtctgaatgagtacatcaatctgtataaccaaaagaccaaacagaaactgccaaaattcaaaccgctgtacaagcaagtcctgagcgatcgggaaagcttgagcttttacggtgaaggttataccagcgacgaggaggtactggaggtctttcgcaataccctgaacaagaacagcgaaattttcagctccattaaaaagctggagaaactgtttaagaattttgacgagtacagcagcgcaggtatttttgtgaagaacggacctgccataagcaccattagcaaggatatttttggagagtggaatgttatccgtgataaatggaacgcggaatatgatgacatacacctgaaaaagaaggctgtggtaactgagaaatatgaagacgatcgccgcaaaagctttaaaaaaatcggcagctttagcctggagcagctgcaggaatatgcggacgccgacctgagcgtggtcgagaaactgaaggaaattattatccaaaaagtggatgagatttacaaggtatatggtagcagcgaaaaactgtttgatgcggacttcgttctggaaaaaagcctgaaaaaaaatgatgctgttgttgcgatcatgaaagacctgctcgatagcgttaagagctttgaaaattacattaaagcattctttggcgagggcaaagaaacaaacagagacgaaagcttttatggcgacttcgtcctggcttatgacatcctgttgaaggtagatcatatatatgatgcaattcgtaattacgtaacccaaaagccgtacagcaaagataagttcaaactgtatttccagaacccgcagtttatgggtggctgggacaaagacaaggagacagactatcgcgccactattctgcgttacggcagcaagtactatctcgccatcatggacaaaaaatatgcaaagtgtctgcagaaaatcgataaagacgacgtgaacggaaattacgaaaagattaattataagctgctgccagggcccaacaagatgttaccgaaagtatttttttccaaaaaatggatggcatactataacccgagcgaggatatacagaagatttacaaaaatgggaccttcaaaaagggggatatgttcaatctgaatgactgccacaaactgatcgatttttttaaagatagcatcagccgttatcctaaatggtcaaacgcgtatgattttaatttctccgaaacggagaaatataaagacattgctggtttctatcgcgaagtcgaagaacagggttataaagttagctttgaatcggccagcaagaaagaggttgataaactggtggaggagggtaagctgtatatgtttcagatttataacaaagactttagcgacaaaagccacggtactcctaatctgcatacgatgtactttaaactgctgtttgatgagaataaccacggccaaatccgtctctccggtggagcagaactttttatgcggcgtgcgagcctaaaaaaggaagaactggtggtgcatcccgccaacagcccgattgctaacaaaaatccagataatcctaagaagaccaccacactgtcgtacgatgtctataaggataaacgtttctcggaagaccagtatgaattgcatataccgatagcaattaataaatgcccaaaaaacattttcaaaatcaacactgaagttcgtgtgctgctgaaacatgatgataatccgtatgtgatcggaattgaccgtggggagagaaatctgctgtatattgtagtcgttgatggcaagggcaacatcgttgagcagtatagcctgaatgaaataattaataattttaacggtatacgtattaaaaccgactatcatagcctgctggataaaaaggagaaagagcgttttgaggcacgccaaaattggacgagcatcgaaaacatcaaggaactgaaggcaggatatatcagccaagtagtccataaaatctgtgaactggtggagaagtacgacgctgtcattgccctggaagacctcaatagcggctttaaaaacagccgggtgaaggtggagaaacaggtataccaaaagtttgaaaagatgctcattgataagctgaactatatggttgataaaaagagcaacccgtgcgccactggcggtgcactgaaagggtaccaaattaccaataaatttgaaagctttaaaagcatgagcacgcagaatgggtttattttttatataccagcatggctgacgagcaagattgaccccagcactggttttgtcaatctgctgaaaaccaaatacacaagcattgcggatagcaaaaaatttatttcgagcttcgaccgtattatgtatgttccggaggaagatctgtttgaatttgccctggattataaaaacttcagccgcaccgatgcagattatatcaaaaaatggaagctgtacagttatggtaatcgtatacgtatcttccgtaatccgaagaaaaacaatgtgttcgattgggaagaggtctgtctgaccagcgcgtataaagaactgttcaacaagtacggaataaattatcagcaaggtgacattcgcgcactgctgtgtgaacagtcagataaagcattttatagcagctttatggcgctgatgagcctgatgctccagatgcgcaacagcataaccggtcgcacagatgttgactttctgatcagccctgtgaagaatagcgacggcatcttctacgattccaggaactatgaagcacaggaaaacgctattctgcctaaaaatgccgatgccaacggcgcctataatattgcacggaaggttctgtgggcgattggacagttcaagaaagcggaagatgagaagctggataaggtaaaaattgctattagcaataaggaatggctggagtacgcacagacatcggttaaacacggtagtaaaaggccggcggccacgaaaaaggccggccaggcaaaaaagaaaaagggagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 196 - DNA, 23 bp, synthetic construct
  source 1..23: mol_type = other DNA
tcatctgtgcccctccctccctg

SEQ ID NO: 197 - DNA, 23 bp, synthetic construct
  source 1..23: mol_type = other DNA
cagcccgctggccctgtaaagga

SEQ ID NO: 198 - DNA, 23 bp, synthetic construct
  source 1..23: mol_type = other DNA
gaacatgaaaacttaaatagaac

SEQ ID NO: 199 - DNA, 23 bp, synthetic construct
  source 1..23: mol_type = other DNA
gtcaggttggctgctgggctggc

SEQ ID NO: 200 - DNA, 23 bp, synthetic construct
  source 1..23: mol_type = other DNA
acagatggggctggacaattttt

(19) United States
(12) Patent Application Publication
(10) Pub. No.: US 2026/0009010 A1
(43) Pub. Date: Jan. 8, 2026

(54) METHODS AND COMPOSITIONS FOR MODIFICATION OF PROTOSPACER ADJACENT MOTIF SPECIFICITY OF CAS12A

(71) Applicant: Pairwise Plants Services, Inc., Durham, NC (US)

(72) Inventors: David Gerhard Schwark, New Hill, NC (US); Joanne Hunt, Wake Forest, NC (US); Joseph Matthew Watts, Cary, NC (US)

(21) Appl. No.: 19/260,726

(22) Filed: Jul. 7, 2025

Related U.S. Application Data
(60) Provisional application No. 63/668,418, filed on Jul. 8, 2024.

(51) Int. Cl. C12N 9/22; C07K 14/47; C12N 9/78; C12N 15/11

(52) U.S. Cl. CPC C12N 9/226; C07K 14/4703; C12N 9/78; C12N 15/111; C07K 2319/80; C12N 2310/20; C12Y 305/04001; C12Y 305/04002

(57) ABSTRACT

This invention relates to variants of Cas12a nucleases and engineered proteins having altered protospacer adjacent motif recognition specificity. The invention further relates to methods of making CRISPR-CAS nuclease variants and methods of modifying nucleic acids using the variants.

### STATEMENT OF PRIORITY

This application claims the benefit, under 35 U.S.C. § 119(e), of U.S. Provisional Application No. 63/668,418 filed on Jul. 8, 2024, the entire contents of which is incorporated by reference herein.

### STATEMENT REGARDING ELECTRONIC FILING OF A SEQUENCE LISTING

A Sequence Listing in XML format, entitled 1499-142PR_ST26.xml, 381,689 bytes in size, generated on Jun. 27, 2024 and filed herewith, is hereby incorporated by reference into the specification for its disclosures.

### FIELD OF THE INVENTION

This invention relates to variants of Cas12a CRISPR-Cas nucleases and engineered proteins having altered protospacer adjacent motif recognition specificity. The invention further relates to methods of making the CRISPR-CAS nuclease variants and methods of modifying nucleic acids using the variants.

### BACKGROUND OF THE INVENTION

Genome editing/modifying is a process that utilizes site-directed nucleases, for example, CRISPR-Cas nucleases, to introduce variation at a targeted genomic location. The most widely utilized nuclease for genome modification, Cas9, can introduce mutations at a genomic region upstream of an NGG motif (e.g., a protospacer adjacent motif (PAM)). Other Cas nucleases have different PAM recognition specificities. When the PAM specificities of these nucleases are particularly stringent, they can reduce the usefulness of the nuclease for genome modification by limiting the number of genomic target sites available for modification by that nuclease.

To address the shortcomings in the art, the present invention provides modified CRISPR-Cas nucleases having improved PAM specificity and methods for designing, identifying and selecting such CRISPR-Cas nucleases.

### SUMMARY OF THE INVENTION

One aspect of the invention provides a CRISPR (Clustered Regularly Interspaced Short Palindromic Repeats) Cas12a polypeptide, wherein the Cas12a polypeptide comprises: (a) an amino acid sequence having at least 95% identity to the amino acid sequence of SEQ ID NO: 180 and a mutation located at one or more than one of the following positions of K135, S168, T246, N263, T307, L324, E330, S345, R359, D367, I369, E379, Y381, I417, F474, E479, E484, D489, L498, N590, K167, Q231, A454, F657, F337, L459, I507, M531, D654, E659, S170, V245, I425, V463, L492, K514, N112, E223, Y426, I455, S117, A239, T295, N100, K387, A404, D405, D423, E539, D572, N211, N527, Y553, Y554, L321, K192, I344 and/or E443, optionally a mutation located at position K135, S168, T246, N263, T307, L324, E330, S345, R359, D367, I369, E379, Y381, I417, F474, E479, E484, D489, L498, and/or N590, and/or a mutation comprising one or more of K135N, S168N, T246S, N263I, T307S, L324M, E330V, S345R, R359S, D367V, I369M, E379G, Y381H, I417F, F474L, E479V, E484V, D489Y, L498M, N590Y, K167I, Q231, A454V, F657, F337L, L459M, I507V, M531I, D654E, E659V, S170L, V245I, I425V, V463A, L492P, K514N, N112D, E223V, Y426D, 1455F, S117C, A239T, T295I, N100S, K387E, A404V, D405G, D423V, E539V, D572G, N211S, N527S, Y553H, Y554N, L321V, K192I, I344T and/or E443D, optionally a mutation comprising one or more of K135N, S168N, T246S, N263I, T307S, L324M, E330V, S345R, R359S, D367V, I369M, E379G, Y381H, I417F, F474L, E479V, E484V, D489Y, L498M, and/or N590Y, optionally the mutation of D489Y, L324M, E484V and/or E330V, with reference to position numbering of SEQ ID NO: 180, wherein the polypeptide exhibits a reduced PAM stringency and increased recognition of new protospacer adjacent motifs (PAMs) as compared to the amino acid sequence of SEQ ID NO: 180, (b) an amino acid sequence having at least 95% identity to the amino acid sequence of SEQ ID NO: 51 and a mutation located at one or more than one of the following positions of E153, S186, R267, I285, Y336, L352, S358, E372, R386, E391, R393, K403, A405, A434, V486, E491, P494, R499, E508, A602, I185, Q252, E465, L698, F365, Q470, A517, A541, L695, P700, A188, S266, I442, L474, G502, K524, T114, P244, L443, I466, D119, Q260, K324, T101, R411, A427, G428, S440, E549, E584, S234, M537, Y563, Y564, A349, R210, L371, and/or K459, optionally a mutation at position E153, S186, R267, I285, Y336, L352, S358, E372, R386, E391, R393, K403, A405, A434, V486, E491, P494, R499, E508, and/or A602, optionally a mutation at position R499, L352, P494 and/or S358, with reference to position numbering of SEQ ID NO: 51, wherein the polypeptide exhibits a reduced PAM stringency and increased recognition of new protospacer adjacent motifs (PAMs) as compared to the amino acid sequence of SEQ ID NO: 51, or (c) an amino acid sequence having at least 95% identity to the amino acid sequence of SEQ ID NO: 55 and a mutation located at one or more than one of the following positions of I162, P196, V290, S308, Q358, L377, K385, T400, Q423, D431, P433, K443, T445, A480, I454, D550, E559, V564, A573, N666, I195, Q275, A525, F741, Y392, L529, I582, A606, G738, D743, S198, F289, I488, T533, E567, K589, S116, A267, F489, A525, N121, T283, E343, T104, L451, R467, D468, P486, E614, E648, T243, N602, Y628, Y629, L374, S220, L399, and/or Q507, optionally a mutation located at I162, P196, V290, S308, Q358, L377, K385, T400, Q423, D431, P433, K443, T445, A480, I454, D550, E559, V564, A573, and/or N666, optionally a mutation at position V564, L377, E559 and/or K385, with reference to position numbering of SEQ ID NO: 55, wherein the polypeptide exhibits a reduced PAM stringency and increased recognition of new protospacer adjacent motifs (PAMs) as compared to the amino acid sequence of SEQ ID NO: 55.

A second aspect of the invention provides a Type V Clustered Regularly Interspaced Short Palindromic Repeats (CRISPR)-associated (Cas) (CRISPR-Cas) system comprising a guide nucleic acid (CRISPR RNA, CRISPR DNA, crRNA, crDNA) comprising a spacer sequence and a repeat sequence and (a) a fusion protein comprising the Cas12a polypeptide of the invention and a polypeptide of interest; or (b) a nucleic acid encoding the Cas12a polypeptide of the invention, and a nucleic acid encoding the polypeptide of interest, wherein the guide nucleic acid is capable of forming a complex with the Cas12a polypeptide or the fusion protein and the spacer sequence is capable of hybridizing to a target nucleic acid, thereby guiding the Cas12a polypeptide and the polypeptide of interest to the target nucleic acid, whereby the target nucleic acid is modified (e.g., cleaving or editing) or modulated (e.g., modulating transcription).

A third aspect of the invention provides a method of modifying a target nucleic acid comprising contacting the target nucleic acid with: (a)(i) a Cas12a polypeptide of the invention, or the fusion protein of the invention, and (ii) a guide nucleic acid (e.g., CRISPR RNA, CRISPR DNA, crRNA, crDNA); (b) a complex of the invention and a guide nucleic acid; (c) a composition comprising (i) a Cas12a polypeptide of the invention, or a fusion protein of the invention, and (ii) a guide nucleic acid; and/or (d) a system of the invention, thereby modifying the target nucleic acid.

A fourth aspect of the invention provides a method of modifying a target nucleic acid, comprising contacting a cell or a cell free system comprising the target nucleic acid with: (a)(i) a polynucleotide encoding a Cas12a polypeptide of the invention or an expression cassette or vector comprising the same, and (ii) a guide nucleic acid, or an expression cassette or vector comprising (a)(i) and (ii); and/or (b) a nucleic acid construct encoding a (i) complex comprising a Cas12a polypeptide or a fusion protein of the invention, and (ii) a guide nucleic acid, or an expression cassette or vector comprising the same, thereby modifying the target nucleic acid.

A fifth aspect of the invention provides a method of editing a target nucleic acid, comprising: contacting the target nucleic acid with: (a)(i) a fusion protein of the invention, and (a)(ii) a guide nucleic acid; (b) a complex comprising a fusion protein of the invention, and a guide nucleic acid; (c) a composition comprising a fusion protein of the invention and a guide nucleic acid; and/or (d) a system of the invention, thereby editing the target nucleic acid.

A sixth aspect of the invention provides a method of editing a target nucleic acid, comprising contacting a cell or a cell free system comprising the target nucleic acid with: (a)(i) a polynucleotide encoding a fusion protein of the invention, or an expression cassette or vector comprising the same, and (a)(ii) a guide nucleic acid, or an expression cassette or vector comprising (a)(i) and (a)(ii); and/or (b) a nucleic acid construct encoding a complex comprising a fusion protein of the invention, and a guide nucleic acid, or an expression cassette or vector comprising the same; and/or (c) a system of the invention, thereby editing the target nucleic acid.

A seventh aspect of the invention provides an engineered protein comprising: a first polypeptide that is a first portion of a modified protein, wherein the modified protein comprises an amino acid sequence having at least 80%, 85%, 90%, or 95% identity to the amino acid sequence of SEQ ID NO: 180 (LbCas12a) and, with reference to the amino acid sequence of SEQ ID NO: 180, comprises a mutation at one or more of the following positions selected from the group consisting of: N100, K116, K120, K121, D122, E125, T148, T149, T152, D156, E159, N211, N263, T295, E330, K387, A404, D405, D423, E484, L498, N527, Q529, G532, D535, K538, E539, D541, Y542, Y553, Y554, D572, L585, K591, M592, K595, V596, S599, K600, K601, Y616, Y646, W649, N112, S117, K135, K167, S168, S170, K192, E223, Q231, A239, V245, T246, T307, L321, L324, F337, I344, S345, R359, D367, I369, E379, Y381, I417, I425, Y426, E443, A454, I455, L459, V463, F474, E479, D489, L492, I507, K514, M531, N590, F657, D654, E659 and any combination thereof with reference to position numbering of SEQ ID NO: 180; and a second polypeptide that is heterologous to the first polypeptide and is not a Type V CRISPR-Cas effector polypeptide, wherein the first polypeptide and second polypeptide are different from each other, and wherein the engineered protein comprises the mutation.

An eighth aspect of the invention provides an engineered protein comprising: an amino acid sequence having at least 80%, 85%, 90%, or 95% identity to the amino acid sequence of SEQ ID NO: 107 (SYN3298); and a mutation at one or more of the following positions selected from the group consisting of: N100, K116, K120, K121, D122, E125, T148, T149, T152, D156, E159, N211, N263, T443, E478, K535, A552, D553, D571, E632, L646, N675, Q677, G680, D683, K686, E687, D689, Y690, Y701, Y702, D720, L733, K739, M740, K743, V744, S747, K748, K749, Y764, Y794, W797, N112, S117, K135, K167, S168, S170, K192, E223, Q231, A239, V245, T246, T455, L469, L472, F485, I492, S493, R507, D515, I517, E527, Y529, I565, I573, Y574, E591, A602, I603, L607, V611, F622, E627, D637, L640, I655, K662, M679, N738, D802, F805, E807 and any combination thereof with reference to residue position numbering of SEQ ID NO: 107 (SYN3298).

The invention further provides complexes and nucleic acid molecules comprising the engineered proteins of the invention. The invention additionally provides expression cassettes and/or vectors comprising polynucleotides encoding CRISPR-Cas nucleases and/or fusion proteins and/or engineered proteins of the invention and/or cells comprising polynucleotides, polypeptides, fusion proteins and/or engineered proteins of the invention and/or kits comprising the same. Further provided are methods of using the CRISPR-Cas nucleases and/or fusion proteins and/or engineered proteins of the invention.

These and other aspects of the invention are set forth in more detail in the description of the invention below.

### BRIEF DESCRIPTION OF THE SEQUENCES

SEQ ID NO: 1 and 169-174 are exemplary HNH domain polypeptide sequences useful with this invention.

SEQ ID NOs: 2-17, 105-107, 125-132, 157-168, and 181-193 are exemplary engineered protein polypeptide sequences useful with this invention.

SEQ ID NOs: 18-47, 153-154, and 175-179 are exemplary linker amino acid sequences useful with this invention.

SEQ ID NO: 48-49 are exemplary regulatory sequences encoding a promoter and intron.

SEQ ID NOs: 50-66 and 180 are exemplary Cas12a polypeptide sequences useful with this invention.

SEQ ID NOs: 67-69 are exemplary Cas12a polynucleotide sequences useful with this invention.

SEQ ID NOs: 70-80 and 140-143 are exemplary Cas9 polynucleotide sequences useful with this invention.

SEQ ID NOs: 81-82 are exemplary Cas9 polypeptide sequences useful with this invention.

SEQ ID NOs: 83-84, 86-92, 152 and 156 are exemplary cytosine deaminase polypeptide sequences useful with this invention.

SEQ ID NO: 85 is an exemplary cytosine deaminase polynucleotide sequence useful with this invention.

SEQ ID NOs: 93-103 and 155 are exemplary adenine deaminase polypeptide sequences useful with this invention.

SEQ ID NO: 104 is an exemplary uracil-DNA glycosylase inhibitor (UGI) sequence useful with this invention.

SEQ ID NOs: 108-118 provide example RNA recruiting motifs and corresponding affinity polypeptides useful with this invention.

SEQ ID NOs: 119-121 provide example peptide tags and an affinity polypeptide useful with this invention.

SEQ ID NO: 122 is an exemplary RuvC domain sequence useful with this invention.

SEQ ID NOs: 123 and 133-135 are exemplary target sites and substrates useful with this invention.

SEQ ID NOs: 124, 136-139, 144-150, and 196-200 are example CRISPR RNA and spacer sequences for nucleic acid guides useful with this invention.

SEQ ID NO: 151 is an exemplary Cas12b polypeptide sequences useful with this invention.

SEQ ID NO: 194-195 are exemplary expression vector polynucleotide sequences useful with this invention.

### BRIEF DESCRIPTION OF THE DRAWINGS

FIG. 1 is an illustration depicting the mechanism of action for Type II CRISPR endonucleases.

FIG. 2 is an illustration depicting the mechanism of action for Type V CRISPR endonucleases.

FIG. 3 is the crystal structure of SpCas9 (PDB ID 4UN3) bound to a single guide RNA (sgRNA) and target DNA. Domains shown are as follows: RuvC, bridge helix, Rec1, Rec2, HNH, and PAM-interacting.

FIG. 4 is a diagram of Cas12a domains viewed facing the REC lobe. From this view, a portion of the crRNA/target DNA duplex is visibly exposed to the surface of Cas12a.

FIG. 5 is an overlay of the HNH domain from SpCas9 onto the candidate insertion site in LbCas12a.

FIG. 6 is an illustration depicting the soluble fraction lysed Escherichia coli expressing HNH-3287, 3288, 3289, 3290, 3296, 3297, 3298, and 3299.

FIG. 7 is an illustration depicting the nicking activity of purified HNH-3287, 3288, 3289, 3290, 3296, 3297, and 3298.

FIG. 8 is an image of a gel that indicates that nickases according to some embodiments of the present invention were solubly expressed in E. coli.

FIG. 9 is an image of a gel that indicates that nickases according to some embodiments of the present invention can nick a DNA substrate.

FIG. 10 is an image of a gel that indicates that nickases according to some embodiments of the present invention can be RNA-dependent.

FIG. 11 is an image of a gel that indicates that nickases according to some embodiments of the present invention can act as a DNA nickase.

FIG. 12 is an illustration showing a labeled target strand.

FIG. 13 is an illustration showing a labeled non-target strand.

FIG. 14 is an image of a gel including samples incubated with a labeled target strand.

FIG. 15 is an image of a gel including samples incubated with a labeled non-target strand.

FIG. 16 is an image of the entire gel showing the lanes of FIG. 14 and FIG. 15 along with the lanes for the controls.

FIG. 17 is an illustration showing editing efficiencies for respective enzyme pairs according to some embodiments of the present invention.

FIGS. 18-21 are graphs showing the percentage of C to T editing for various target regions corresponding to the respective spacers: FANCF spacer 1 (FIG. 18), FANCF spacer 2 (FIG. 19), AAVS1 spacer 1 (FIG. 20), and AAVS1 spacer 2 (FIG. 21).

FIGS. 22-23 are graphs showing the percentage of A to G editing for various target regions corresponding to the respective spacers: RNF2 spacer 1 (FIG. 22) and RNF2 spacer 2 (FIG. 23).

FIG. 24 is a graph showing the indel generation frequency of RR-LbCas12a and three synthetic enzymes (3287 RR, 3288 RR, and 3298RR), which each included G532R and K595R (RR) mutations with reference to position numbering of SEQ ID NO: 180 (LbCas12a), at known, non-native protospacer adjacent motif (PAM) sequences.

FIG. 25 is a graph showing the indel generation frequency of LbCas12a, RR-LbCas12a, 3287 RR, 3288 RR, and 3298 RR at native TTTV PAM sequences.

FIG. 26 is a graph showing the indel generation frequency of RVR-LbCas12a (having G532R, K538V, and Y542R (RVR) mutations) and RVR 3287, RVR 3288, and RVR 3298, which each included G532R, K538V, and Y542R (RVR) mutations with reference to position numbering of SEQ ID NO: 180 (LbCas12a), at known, non-native PAM sequences.

FIG. 27 is a graph showing the indel generation frequency of LbCas12a, RVR-LbCas12a (having G532R, K538V, and Y542R (RVR) mutations) and 3287 RVR, 3288 RVR, and 3298 RVR, which each included G532R, K538V, and Y542R (RVR) mutations with reference to position numbering of SEQ ID NO: 180 (LbCas12a), at native TTTV PAM sequences.

FIGS. 28-31 are graphs showing biological replicates of the indel generation frequency of LbCas12a, 3287, 3288, and 3298; as well as 3287 D156R, 3288 D156R, and 3298 D156R, which included a D156R mutation with reference to position numbering of SEQ ID NO: 180 (LbCas12a), at native TTTV PAM sequences.

FIG. 32 is a graph showing the indel generation frequency of RVR-LbCas12a, RVR 3287, RVR 3288, and RVR 3298 at known, non-native PAM sequences.

FIG. 33 is a graph showing the indel generation frequency LbCas12a, RVR-LbCas12a, 3287 RVR, 3288 RVR, and 3298 RVR at native TTTV PAM sequences.

FIG. 34. Average Indel percentages for Round 1 directed evolution variants (nucleases) in HEK293 Ts. The average Indel percentage is across 3 spacers.

FIG. 35. Average indel percentages for Round 2 directed evolution variants (using K595Y nuclease as the base enzyme) in HEK293T cells. Five spacers were assayed for each variant; however, not all spacers returned data. Values presented are the average of the spacers that returned data and error bars are the standard deviation.

FIG. 36 Amino acid mutations identified in rounds 1 and 2 were transferred to the RR version of LbCas12a (LbCas12a-RR). The figure shows percent indels when these RR variants were tested as nucleases in HEK293T cells. Five spacers were assayed for each variant, not all spacers returned data. These values are the average of the spacers tested with error bars representing the SD.

FIG. 37 Amino acid mutations identified in rounds 1 and 2 were transferred to the RVR version of LbCas12a (LbCas12a-RVR). The figure shows percent indels when these RR variants were tested as nucleases in HEK293T cells. Five spacers were assayed for each variant, not all spacers returned data. These values are the average of the spacers tested with error bars representing the SD.

FIG. 38. Amino acid mutations identified in round 2 were transferred LbCas12a-RR, LbCas12a-RVR, and SHARC. The figure shows percent indels when these RR variants were tested as nucleases in HEK293T cells. Five spacers were assayed for each variant, not all spacers returned data. These values are the average of the spacers tested with error bars representing the SD.

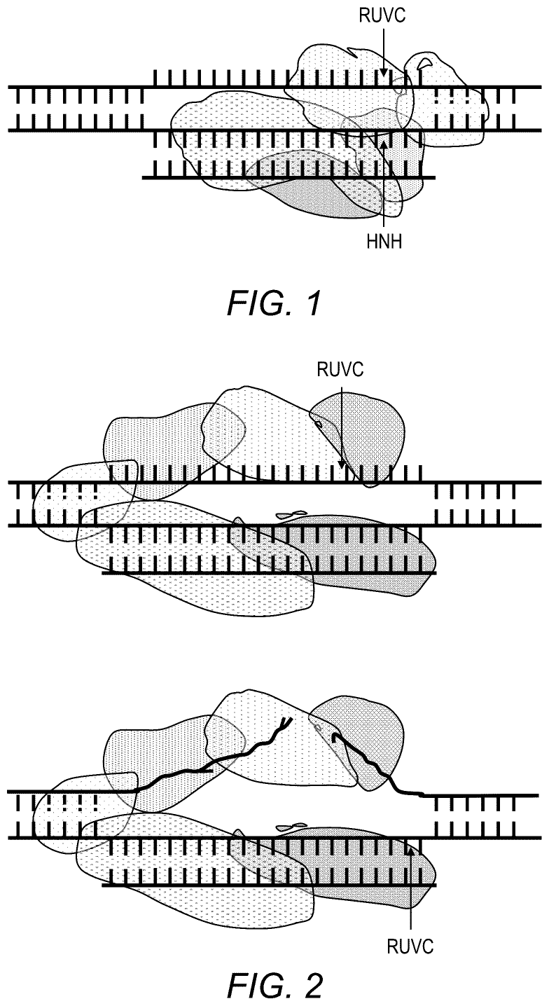

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Except where defined differently for a specific aspect, the term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more as compared to a control.

or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In particular embodiments, the reduction can result in no or essentially no (i.e., an insignificant amount, e.g., less than about 10% or even 5%) detectable activity or amount.

A control plant is typically the same plant as the edited plant, but the control plant has not been similarly edited and therefore is devoid of the mutation. A control plant maybe an isogenic plant and/or a wild type plant. Thus, a control plant can be the same breeding line, variety, or cultivar as the subject plant into which a mutation as described herein is introgressed, but the control breeding line, variety, or cultivar is free of the mutation. In some embodiments, a comparison between a plant of the invention and a control plant is made under the same growth conditions, e.g., the same environmental conditions (soil, hydration, light, heat, nutrients, and the like).

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

As used herein, the term “heterologous” refers to a nucleotide/polypeptide that originates from a foreign species, or, if from the same species, is substantially modified from its native form in composition and/or genomic locus by deliberate human intervention. A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “native nucleic acid” is a nucleic acid that is naturally occurring in or endogenous to a reference organism. A “homologous” nucleic acid sequence is a nucleotide sequence naturally associated with a host cell into which it is introduced.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue. A truncation can include a truncation at the C-terminal end of a polypeptide or at the N-terminal end of a polypeptide. A truncation of a polypeptide can be the result of a deletion of the corresponding 5′ end or 3′ end of the gene encoding the polypeptide. A frameshift mutation can occur when deletions or insertions of one or more base pairs are introduced into a gene, optionally resulting in an out-of-frame mutation or an in-frame mutation. Frameshift mutations in a gene can result in the production of a polypeptide that is longer, shorter or the same length as the wild type polypeptide depending on when the first stop codon occurs following the mutated region of the gene. As an example, an out-of-frame mutation that produces a premature stop codon can produce a polypeptide that is shorter that the wild type polypeptide, or, in some embodiments, the polypeptide may be absent/undetectable. A DNA inversion is the result of a rotation of a genetic fragment within a region of a chromosome.

A “null allele” is a nonfunctional allele caused by a genetic mutation that results in a complete lack of production of the corresponding protein or produces a protein that is non-functional.

A “knock-out mutation” is a mutation that results in a non-functional protein, but which may have a detectable transcript or protein.

A “recessive mutation” is a mutation in a gene that produces a phenotype when homozygous but the phenotype is not observable when the locus is heterozygous.

A “dominant mutation” is a mutation in a gene that produces a mutant phenotype in the presence of a non-mutated copy of the gene. A dominant mutation may be a loss or a gain of function mutation, a hypomorphic mutation, a hypermorphic mutation or a weak loss of function or a weak gain of function.

A “dominant negative mutation” is a mutation that produces an altered gene product (e.g., having an aberrant function relative to wild type), which gene product adversely affects the function of the wild-type allele or gene product. For example, a “dominant negative mutation” may block a function of the wild type gene product. A dominant negative mutation may also be referred to as an “antimorphic mutation.”

A “semi-dominant mutation” refers to a mutation in which the penetrance of the phenotype in a heterozygous organism is less than that observed for a homozygous organism.

A “weak loss-of-function mutation” is a mutation that results in a gene product having partial function or reduced function (partially inactivated) as compared to the wild type gene product.

A “hypomorphic mutation” is a mutation that results in a partial loss of gene function, which may occur through reduced expression (e.g., reduced protein and/or reduced RNA) or reduced functional performance (e.g., reduced activity), but not a complete loss of function/activity. A “hypomorphic” allele is a semi-functional allele caused by a genetic mutation that results in production of the corresponding protein that functions at anywhere between 1% and 99% of normal efficiency.

A “hypermorphic mutation” is a mutation that results in increased expression of the gene product and/or increased activity of the gene product.

A “gain-of-function” allele or mutation is a mutation that confers a new function on the encoded gene product and/or confers a new gene expression pattern. In some embodiments, a gain-of-function mutation may be dominant or semi-dominant.

As used herein, a “non-natural mutation” refers to a mutation that is generated through human intervention and differs from mutations found in the same gene that have occurred in nature (e.g., occurred naturally and not as a result of a modification made by a human).

A “locus” is a position on a chromosome where a gene or marker or allele is located. In some embodiments, a locus may encompass one or more nucleotides.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype.—In some embodiments of this invention, the phrase “desired allele,” “target allele” or “allele of interest” refers to an allele(s) that is associated with increased yield under non-water stress conditions in a plant relative to a control plant not having the target allele or alleles.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity; e.g., substantial complementarity) to the comparator nucleotide sequence.

A “portion” or “fragment” of a nucleotide sequence of the invention will be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950 or 1000 or more nucleotides or any range or value therein) to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. As an example, a repeat sequence of guide nucleic acid of this invention may comprise a portion of a wild type CRISPR-Cas repeat sequence (e.g., a wild-type Type V CRISPR Cas repeat, e.g., a repeat from the CRISPR Cas system that includes, but is not limited to, Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c, and the like). Similarly, a portion of a polypeptide may be included in a larger polypeptide of which it is a constituent.

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, a “portion” may be related to the number of amino acids that are deleted from a polypeptide. In some embodiments, a polypeptide fragment comprises, consists essentially of or consists of at least about 5, 6, 7, 8, 9, 10, 11, 12, 13, 14, 15, 20, 21, 22, 23, 24, 25, 26, 27, 28, 29, 30, 35, 40, 45, 46, 47, 48, 49, 50, 55, 60, 65, 70, 75, 80, 85, 90, 95, 100, 125, 150, 175, 200, 225, 250, 300, 350, 400 or more consecutive amino acids of a reference polypeptide. A “region” of a polynucleotide or a polypeptide refers to a portion of consecutive nucleotides or consecutive amino acid residues of that polynucleotide or a polypeptide, respectively.

As used herein with respect to nucleic acids, the term “functional fragment” refers to nucleic acid that encodes a functional fragment of a polypeptide. A “functional fragment” with respect to a polypeptide is a fragment of a polypeptide that retains one or more of the activities of the native reference polypeptide.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA) as well as web-based alignment programs such as Clustal Omega, EMBOSS Needle, EMBOSS Stretcher, EMBOSS Water, LALIGN, GGSEARCH2SEQ, EMBOS Cons, Kalign, MAFFT, MUSCLE, and T-Coffee. In some embodiments, an “optimal alignment” of two sequences (e.g., two polypeptide sequences) is the highest scoring alignment, optionally from an alignment conducted by a tool such as the local homology algorithm of Smith and Waterman, the homology alignment algorithm of Needleman and Wunsch, the search for similarity method of Pearson and Lipman, GAP, BESTFIT, FASTA, and TFASTA available as part of the GCG® Wisconsin Package® (Accelrys Inc., San Diego, CA), Clustal Omega, EMBOSS Needle, EMBOSS Stretcher, EMBOSS Water, LALIGN, GGSEARCH2SEQ, EMBOS Cons, Kalign, MAFFT, MUSCLE, and/or T-Coffee. In some embodiments, an “optimal alignment” of two sequences (e.g., two polypeptide sequences) may be the alignment that provides the highest percent sequence identity, optionally allowing for one or more gap(s) to be introduced into one or both sequences. An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more sequences may be to a full-length sequence or a portion thereof, or to a longer sequence. For purposes of this invention “percent identity” and/or optimal alignment may be determined using Basic Local Alignment Search Tool (BLAST) provided by the National Center for Biotechnology Information such as BLASTX, for translated nucleotide sequences, BLASTN for polynucleotide sequences, and BLASTP for polypeptide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

Any nucleotide sequence, polynucleotide and/or recombinant nucleic acid construct of this invention can be optimized for expression in any organism of interest. Optimization for expression is well known in the art and involves, for example, modification of a nucleotide sequence for codon usage bias using species specific codon usage tables. Also relevant for optimization of expression is the editing efficiency of the target gene. Thus, the ability to generate a specific edit with a frequency that produces a sufficient number of edited alleles to enable selection of a commercially relevant edited allele of a target gene is a factor to be considered when optimizing for expression. In some embodiments, a polynucleotide, nucleic acid construct, expression cassette, and/or vector of the present invention (e.g., that comprises/encodes a polypeptide of the present invention (e.g., an engineered protein), a nucleic acid binding polypeptide (e.g., a DNA binding polypeptide such as a sequence-specific DNA binding domain from a polynucleotide-guided endonuclease, a zinc finger nuclease, a transcription activator-like effector nuclease (TALEN), an Argonaute protein, and/or a CRISPR-Cas effector protein), a guide nucleic acid, a cytosine deaminase and/or adenine deaminase) may be optimized for expression in an organism (e.g., an animal such as a human, a plant, a fungus, an archaeon, or a bacterium). In some embodiments, the optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acid constructs, polynucleotides, expression cassettes, and/or vectors but which have not been optimized for expression.

To modify a nucleotide sequence for codon usage, codon usage tables are generated based on a sequence analysis of the most highly expressed genes for a organism/species of interest. When the nucleotide sequences are to be expressed in the nucleus, the codon usage tables are generated based on a sequence analysis of highly expressed nuclear genes for the species of interest. The modifications of the nucleotide sequences are determined by comparing the species specific codon usage table with the codons present in the native polynucleotide sequences. As is understood in the art, codon optimization of a nucleotide sequence results in a nucleotide sequence having less than 100% identity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9%, and any range or value therein) to the native nucleotide sequence but which still encodes a polypeptide having the same function as that encoded by the original, native nucleotide sequence. Thus, in some embodiments of the invention, the polynucleotides, nucleic acid constructs, expression cassettes, and/or vectors of the invention (e.g., comprising/encoding a polypeptide, fusion protein, complex of the invention, e.g., a modified CRISPR-Cas nuclease) are codon optimized for expression in a particular species of interest, e.g., a particular plant species, a particular bacterial species, a particular animal species, and the like. In some embodiments, a codon optimized nucleic acid construct, polynucleotide, expression cassette, and/or vector of the invention have about 70% to about 99.9% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the polynucleotides, nucleic acid constructs, expression cassettes, and/or vectors of the invention that have not been codon optimized

In any of the embodiments, a polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” or “fused” in reference to polypeptides, refers to the attachment of one polypeptide to another. A polypeptide may be linked or fused to another polypeptide (at the N-terminus or the C-terminus) directly (e.g., via a peptide bond) or through a linker (e.g., a peptide linker).

The term “linker” in reference to polypeptides is art-recognized and refers to a chemical group, or a molecule linking two molecules or moieties, e.g., two polypeptides or domains of a fusion protein, such as, for example, a CRISPR-Cas effector protein and a peptide tag and/or a polypeptide of interest. A linker may be comprised of a single linking molecule (e.g., a single amino acid) or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide (e.g., a peptide linker).

In some embodiments, a peptide linker may be about 4 to about 100 or more amino acids in length, for example, about 2, 3, 4, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker. In some embodiments, the peptide linker is a GS linker having 1, 2, 3, or 4 amino acid residues, optionally 2 or 4 amino acid residues. In some embodiments, the peptide linker has one of the amino acid sequences of SEQ ID NOs: 18-47 or 175-179. In some embodiments, the peptide linker may comprise an amino acid sequence of (GGS)ₙ, GS, SG, GSSG (SEQ ID NO: 175), GSSGSS (SEQ ID NO: 176), GSSGSSGS (SEQ ID NO: 177), (GSS)ₙ (SEQ ID NO: 178), (GSS)ₙGS (SEQ ID NO: 179), S(GGS)ₙ (SEQ ID NO: 42), SGGS (SEQ ID NO: 43), or (GGGGS)n (SEQ ID NO: 44), wherein n is an integer of 1-20 (e.g., 1, 2, 3, 4, 5, 6, 7, 8, 9, 10, 11, 12, 13, 14, 15, 16, 17, 18, 19, or 20). In some embodiments, the peptide linker may comprise the amino acid sequence: SGGSGGSGGS (SEQ ID NO: 45). In some embodiments, the peptide linker may comprise the amino acid sequence: SGSETPGTSESATPES (SEQ ID NO: 46), also referred to as the XTEN linker. In some embodiments, the peptide linker may comprise the amino acid sequence: SGGSSGGSSGSETPGTSESATPESSGGSSGGS (SEQ ID NO: 47), also referred to as the GS-XTEN-GS linker.

As used herein, the term “linked,” or “fused” in reference to polynucleotides, refers to the attachment of one polynucleotide to another polynucleotide. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). In some embodiments, a promoter region may comprise at least one intron (e.g., SEQ ID NO: 48 or SEQ ID NO: 49).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)).

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79:87-94), maize (Christensen et al., 1989. Plant Molec. Biol. 12:619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231:150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP U.S. Pat. No. 255,378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9 (5): 297-306 (2015)), ZmSTK2_USP from maize (Wang et al. Genome 60 (6): 485-495 (2017)), LAT52 and LAT59 from tomato (Twell et al. Development 109 (3): 705-713 (1990)), Zm13 (U.S. Pat. No. 10,421,972), PLA2-0 promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37 (8): 1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof. As a non-limiting example, a nucleic acid construct of the present invention may encode a base editor comprising a CRISPR-Cas nuclease (e.g., SEQ ID NOs: 180 and/or 51-66, or 193 (amino acid sequences) or SEQ ID NOs: 67-69 (nucleotide sequences)), which has been modified as described herein and a deaminase, wherein the nucleic acid construct further comprises a promoter comprising/associated with an intron. As a further non-limiting example, a nucleic acid construct of the present invention may encode a base editor comprising an CRISPR-Cas nuclease (e.g., SEQ ID NOs: 180 and/or 51-66, or 193 or SEQ ID NOs: 67-69) which has been modified as described herein and a deaminase, wherein the nuclease and/or the deaminase comprises one or more introns and optionally, the nucleic acid construct further comprises a promoter comprising/associated with an intron.

An “editing system” as used herein refers to any site-specific (e.g., sequence-specific) nucleic acid editing system now known or later developed, which system can introduce a modification (e.g., a mutation) in a nucleic acid in target specific manner. For example, an editing system (e.g., a site- and/or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which may comprise one or more polypeptide(s) and/or one or more polynucleotide(s) that when present and/or expressed together (e.g., as a system) in a composition and/or cell can modify (e.g., mutate) a target nucleic acid in a sequence specific manner. In some embodiments, an editing system (e.g., a site- and/or sequence-specific editing system) can comprise one or more polynucleotide(s) and/or one or more polypeptide(s), including but not limited to a nucleic acid binding polypeptide (e.g., a DNA binding domain), a nuclease, another polypeptide, and/or a polynucleotide. In some embodiments, a CRISPR-Cas editing system is provided and/or is used that comprises an engineered protein of the present invention.

In some embodiments, an editing system comprises one or more sequence-specific nucleic acid binding polypeptide(s) (e.g., a DNA binding domain) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system comprises one or more cleavage polypeptide(s) (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN).

A “nucleic acid binding polypeptide” as used herein refers to a polypeptide or domain that binds and/or is capable of binding a nucleic acid (e.g., a target nucleic acid). A DNA binding domain is an exemplary nucleic acid binding polypeptide and may be a site- and/or sequence-specific nucleic acid binding domain. In some embodiments, a nucleic acid binding polypeptide may be a sequence-specific nucleic acid binding polypeptide such as, but not limited to, a sequence-specific binding domain from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, a nucleic acid binding polypeptide comprises a cleavage domain (e.g., a nuclease domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, the nucleic acid binding polypeptide is a polypeptide associates with and/or is capable of associating with (e.g., forms a complex with) with one or more nucleic acid molecule(s) (e.g., forms a complex with a guide nucleic acid as described herein), which may direct and/or guide the nucleic acid binding polypeptide to a specific target nucleotide sequence (e.g., a gene locus of a genome) that is complementary to the one or more nucleic acid molecule(s) (or a portion or region thereof), thereby causing the nucleic acid binding polypeptide to bind to the nucleotide sequence at the specific target site. In some embodiments, the nucleic acid binding polypeptide is a CRISPR-Cas effector protein as described herein.

In some embodiments, an editing system comprises or is a ribonucleoprotein such as an assembled ribonucleoprotein complex (e.g., a ribonucleoprotein that comprises a CRISPR-Cas effector protein, a guide nucleic acid, and optionally a deaminase). In some embodiments, a ribonucleoprotein of an editing system may be assembled together (e.g., a pre-assembled ribonucleoprotein including a CRISPR-Cas effector protein, a guide nucleic acid, and optionally a deaminase) such as when contacted to a target nucleic acid or when introduced into a cell (e.g., a mammalian cell or a plant cell). In some embodiments, a ribonucleoprotein of an editing system may assemble into a complex (e.g., a covalently and/or non-covalently bound complex) while a portion of the ribonucleoprotein is contacting a target nucleic acid and/or may assemble after and/or during introduction into a plant cell. In some embodiments, an editing system may be assembled (e.g., into a covalently and/or non-covalently bound complex) when introduced into a plant cell. In some embodiments, a ribonucleoprotein may comprise an engineered protein of the present invention, a guide nucleic acid, and optionally a deaminase.

The terms “transgene” or “transgenic” as used herein refer to at least one nucleic acid sequence that is taken from the genome of one organism or produced synthetically, and which is then introduced into a host cell (e.g., a plant cell) or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation into the genome and thus without inheritance of the respective at least one molecule introduced into the genome of a cell. The term “transgene-free” refers to a condition in which a transgene is not present or found in the genome of a host cell or tissue or organism of interest.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a nucleic acid construct of the invention (e.g., encoding a Cas12a of the invention, a polynucleotide encoding an engineered protein of the present invention, a polynucleotide encoding a nuclease, a polynucleotide encoding a cytosine deaminase, a polynucleotide encoding an adenine deaminase, a polynucleotide encoding a deaminase fusion protein, a polynucleotide encoding a peptide tag, a polynucleotide encoding an affinity polypeptide, a polynucleotide encoding a glycosylase, and/or a polynucleotide comprising a guide nucleic acid), wherein the nucleic acid construct is operably associated with at least a control sequence (e.g., a promoter). Thus, some embodiments of the invention provide expression cassettes designed to express, for example, a nucleic acid construct of the invention. When an expression cassette comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter, or they may be different promoters. Thus, a polynucleotide encoding a sequence specific nucleic acid binding domain, a polynucleotide encoding a nuclease protein/domain, a polynucleotide encoding a CRISPR-Cas effector protein/domain, a polynucleotide encoding an deaminase protein/domain, a polynucleotide encoding a reverse transcriptase polypeptide/domain (e.g., RNA-dependent DNA polymerase), and/or a polynucleotide encoding a 5′-3′ exonuclease polypeptide/domain, a guide nucleic acid, an extended guide nucleic acid and/or RT template when comprised in a single expression cassette may each be operably linked to a single promoter, or separate promoters in any combination. As a further example, a polynucleotide encoding an engineered protein, a polynucleotide encoding a deaminase (e.g., an adenine deaminase), and a polynucleotide comprising a guide nucleic acid comprised in an expression cassette may each be operably associated with a single promoter or one or more of the polynucleotide(s) may be operably associated with separate promoters (e.g., two or three promoters) in any combination, which may be the same or different from each other.

In some embodiments, an expression cassette comprising the polynucleotides/nucleic acid constructs of the invention may be optimized for expression in an organism (e.g., an animal, a plant, a bacterium and the like).

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. The termination region and/or the enhancer region may be native to the transcriptional initiation region, may be native to a gene encoded by a nucleic acid construct of the invention, e.g., a CRISPR-Cas effector protein or a gene encoding a deaminase, may be native to a gene encoding a polypeptide of the present invention, may be native to a host cell, or may be native to another source (e.g., foreign or heterologous to the promoter, to a gene encoding the CRISPR-Cas effector protein or a gene encoding the deaminase, to a host cell, or any combination thereof). The enhancer region may be native to a gene encoded by a nucleic acid construct of the invention, e.g., a CRISPR-Cas effector protein or a gene encoding a deaminase, may be native to a gene encoding a polypeptide of the present invention, may be native to a host cell, or may be native to another source (e.g., foreign or heterologous to the promoter, to a gene encoding the CRISPR-Cas effector protein or a gene encoding the deaminase, to a host cell, or any combination thereof).

An expression cassette of the invention also can include a nucleotide sequence encoding a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or whether the marker is simply a trait that one can identify through observation or testing, such as by screening (e.g., fluorescence). Many examples of suitable selectable markers are known in the art and can be used in the expression cassettes described herein.

The expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid construct of this invention and/or expression cassettes comprising the same may be comprised in vectors as described herein and as known in the art. In some embodiments, the vector may be a high copy number vector (e.g., a high copy number E. coli vector; e.g., pUC, pBluescript, pGEM and the like). Thus, for example, a library of the present invention may be constructed using a high copy number vector.

As used herein, “contact”, “contacting”, “contacted,” and grammatical variations thereof, refers to placing the components of a desired reaction together under conditions suitable for carrying out the desired reaction (e.g., transformation, transcriptional control, genome editing, nicking, and/or cleavage). Thus, for example, a target nucleic acid may be contacted with (a) a polynucleotide and/or nucleic acid construct of the invention encoding a Cas12a nuclease of the invention and (b) a guide nucleic acid, under conditions whereby the polynucleotide/nucleic acid construct is expressed and the Cas12a nuclease is produced, wherein the nuclease forms a complex with the guide nucleic acid and the complex hybridizes to the target nucleic acid, thereby modifying the target nucleic acid. In some embodiments, a target nucleic acid may be contacted with (a) a Cas12a nuclease of the invention and/or a fusion protein comprising the same (e.g., the Cas12a nuclease of the invention and a polypeptide of interest (e.g., a deaminase)) and (b) a guide nucleic acid, wherein the Cas12a nuclease forms a complex with the guide nucleic acid and the complex hybridizes to the target nucleic acid, thereby modifying the target nucleic acid. As described herein, the target nucleic acid may be contacted with the polynucleotides/nucleic acid constructs/polypeptides of the invention prior to, concurrently with, or after contact with the guide nucleic acid. In some embodiments, a target nucleic acid may be contacted with a nucleic acid construct of the invention encoding, for example, a nucleic acid binding polypeptide (e.g., a DNA binding domain such as a sequence-specific DNA binding protein (e.g., a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein)), a guide nucleic acid, a polynucleotide encoding a polypeptide of the present invention, and optionally a cytosine deaminase and/or adenine deaminase under conditions whereby the nucleic acid binding polypeptide (e.g., a CRISPR-Cas effector protein) is expressed, and the nucleic acid binding polypeptide forms a complex with the guide nucleic acid, the complex hybridizes to the target nucleic acid, and optionally the polypeptide of the present invention, cytosine deaminase, and/or adenine deaminase is/are recruited to the nucleic acid binding polypeptide (and thus, to the target nucleic acid) or is/are fused to the nucleic acid binding polypeptide, thereby modifying the target nucleic acid. In some embodiments, the polypeptide of the present invention, cytosine deaminase, and/or adenine deaminase and the nucleic acid binding polypeptide localize at the target nucleic acid, optionally through covalent and/or non-covalent interactions.

In some embodiments, a target nucleic acid may be contacted with a nucleic acid construct of the invention encoding an engineered protein of the present invention, a guide nucleic acid, and optionally a cytosine deaminase and/or adenine deaminase under conditions whereby the engineered protein is expressed, or a target nucleic acid may be contacted with an engineered protein of the present invention, a guide nucleic acid, and optionally a cytosine deaminase and/or adenine deaminase. The engineered protein can form a complex with the guide nucleic acid, and the complex can hybridize to the target nucleic acid, and optionally the cytosine deaminase and/or adenine deaminase is/are recruited to the engineered protein (and thus, to the target nucleic acid) or the cytosine deaminase and/or adenine deaminase are fused to the engineered protein, thereby modifying the target nucleic acid. The cytosine deaminase and/or adenine deaminase and the engineered protein may localize at the target nucleic acid, optionally through covalent and/or non-covalent interactions.

As used herein, “modifying” or “modification” in reference to a target nucleic acid includes editing (e.g., mutating), covalent modification, exchanging/substituting nucleic acids/nucleotide bases, deleting, cleaving, nicking, and/or transcriptional control of a target nucleic acid. In some embodiments, a modification may include one or more single base changes (SNPs) of any type. In some embodiments, a modification comprises a SNP. In some embodiments, a modification comprises exchanging and/or substituting one or more (e.g., 1, 2, 3, 4, 5, or more) nucleotides. In some embodiments, an insertion or deletion may be about 1 base to about 30,000 bases or more in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000 bases in length or more, or any value or range therein). Thus, in some embodiments, an insertion or deletion may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 bases in length, or any range or value therein; about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 bases to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 bases or more in length, or any value or range therein; about 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 bases to about 2500, 3000, 3500, 4000, 4500, 5000, 5500, 6000, 6500, 7000, 7500, 8000, 8500, 9000, 9500, or 10,000 bases or more in length, or any value or range therein; or about 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 bases to about 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, or 5000 bases or more in length, or any value or range therein. In some embodiments, an insertion or deletion may be about 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, or 10,000 bases to about 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, or 30,000 bases or more in length, or any value or range therein.

“Recruit,” “recruiting” or “recruitment” as used herein refer to attracting one or more polypeptide(s) or polynucleotide(s) to another polypeptide or polynucleotide (e.g., to a particular location in a genome) using protein-protein interactions, nucleic acid protein interactions (e.g., RNA-protein interactions), and/or chemical interactions. Protein-protein interactions can include, but are not limited to, peptide tags (epitopes, multimerized epitopes) and corresponding affinity polypeptides, RNA recruiting motifs and corresponding affinity polypeptides, and/or chemical interactions. Example chemical interactions that may be useful with polypeptides and polynucleotides for the purpose of recruitment can include, but are not limited to, rapamycin-inducible dimerization of FRB-FKBP; Biotin-streptavidin interaction; SNAP tag (Hussain et al. Curr Pharm Des.19 (30): 5437-42 (2013)); Halo tag (Los et al. ACS Chem Biol. 3 (6): 373-82 (2008)); CLIP tag (Gautier et al. Chemistry & Biology 15:128-136 (2008)); DmrA-DmrC heterodimer induced by a compound (Tak et al. Nat Methods 14 (12): 1163-1166 (2017)); Bifunctional ligand approaches (fuse two protein-binding chemicals together) (Voß et al. Curr Opin Chemical Biology 28:194-201 (2015)) (e.g. dihyrofolate reductase (DHFR) (Kopyteck et al. Cell Chem Biol 7 (5): 313-321 (2000)).

“Introducing,” “introduce,” “introduced” (and grammatical variations thereof) in the context of a polynucleotide of interest or editing system means presenting a nucleotide sequence of interest (e.g., polynucleotide, a nucleic acid construct, and/or a guide nucleic acid) and/or editing system (e.g., a polynucleotide, polypeptide, and/or ribonucleoprotein) to a host organism or cell of said organism (e.g., host cell; e.g., a plant cell) in such a manner that the nucleotide sequence or editing system gains access to the interior of a cell. Thus, for example, a polynucleotide of the invention encoding a Cas12a nuclease as described herein and guide nucleic acid or a nucleic acid construct of the invention encoding an engineered protein of the present invention, a guide nucleic acid, and a cytosine deaminase and/or adenine deaminase may be introduced into a cell of an organism, thereby transforming the cell with the Cas12a nuclease and guide nucleic acid or transforming the cell with the engineered protein, a guide nucleic acid, and a cytosine deaminase and/or adenine deaminase. In some embodiments, an engineered protein and/or a guide nucleic acid may be introduced into a cell of an organism, optionally wherein the engineered protein and guide nucleic acid may be comprised in a complex (e.g., a ribonucleoprotein). In some embodiments, the organism is a eukaryote (e.g., a mammal such as a human).

The term “transformation” or “transfection” may be used interchangeably and as used herein refer to the introduction of a heterologous nucleic acid into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a polynucleotide/nucleic acid construct of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, and/or nucleic acid constructs of the invention and/or expression cassettes and/or vectors comprising the same may be expressed transiently and/or they can be stably incorporated into the genome of the host organism. Thus, in some embodiments, a nucleic acid construct of the invention may be transiently introduced into a cell of the organism along with a guide nucleic acid and as such none of the DNA of the nucleic acid constructs, nucleotide sequences, and/or polynucleotides of the invention and/or expression cassettes and/or vectors comprising the same is maintained in the cell.

A polynucleotide/nucleic acid construct, polypeptide, and/or ribonucleoprotein of the invention can be introduced into a cell by any method known to those of skill in the art. In some embodiments, transformation methods include, but are not limited to, transformation via bacterial-mediated nucleic acid delivery (e.g., via Agrobacteria), viral-mediated nucleic acid delivery, silicon carbide and/or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the cell (e.g., a plant cell or an animal cell), including any combination thereof. In some embodiments of the invention, transformation of a cell comprises nuclear transformation. In some embodiments, transformation of a cell comprises plastid transformation (e.g., chloroplast transformation). In some embodiments, a recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

A nucleotide sequence therefore can be introduced into a host organism or its cell in any number of ways that are well known in the art. The methods of the invention do not depend on a particular method for introducing one or more nucleotide sequences into the organism, only that they gain access to the interior of at least one cell of the organism. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, and/or in separate transformation events, or, alternatively, where relevant, a nucleotide sequence may be incorporated into a plant, for example, as part of a breeding protocol. In some embodiments, the cell is a eukaryotic cell (e.g., a plant cell or a mammalian such as a human cell). In some embodiments, the cell is not a mammalian cell. In some embodiments, the cell is not a human cell.

In some embodiments, a nucleic acid construct of the invention (e.g., a polynucleotide encoding a Cas12a polypeptide of the invention, a polynucleotide encoding an engineered protein of the present invention, a polynucleotide encoding a deaminase, and/or a guide nucleic acid and/or expression cassettes and/or vectors comprising the same) may be operably linked to at least one regulatory sequence, optionally, wherein the at least one regulatory sequence may be optimized for expression in a plant. In some embodiments, the at least one regulatory sequence may be, for example, a promoter, an operon, a terminator, or an enhancer. In some embodiments, the at least one regulatory sequence may be a promoter. In some embodiments, the regulatory sequence may be an intron. In some embodiments, the at least one regulatory sequence may be, for example, a promoter operably associated with an intron or a promoter region comprising an intron. In some embodiments, the at least one regulatory sequence may be, for example a ubiquitin promoter and its associated intron (e.g., Medicago truncatula and/or Zea mays and their associated introns). In some embodiments, the at least one regulatory sequence may be a terminator nucleotide sequence and/or an enhancer nucleotide sequence.

In some embodiments, a nucleic acid construct of the invention may be operably associated with a promoter region, wherein the promoter region comprises an intron, optionally wherein the promoter region may be a ubiquitin promoter and intron (e.g., a Medicago or a maize ubiquitin promoter and intron, e.g., SEQ ID NO: 48 or SEQ ID NO: 49). In some embodiments, the nucleic acid construct of the invention that is operably associated with a promoter region comprising an intron may be optimized for expression in a plant.

In some embodiments, a nucleic acid construct of the invention may encode one or more (e.g., 1, 2, 3, 4, or more) polypeptide(s) of interest. The one or more polypeptides of interest may be optimized for expression in a eukaryote (e.g., a human or a plant). In some embodiments, an engineered protein may comprise to one or more (e.g., 1, 2, 3, 4, or more) polypeptide(s) of interest. For example, the heterologous polypeptide of an engineered protein may comprise or be a polypeptide of interest.

A polypeptide of interest useful with this invention can include, but is not limited to, a polypeptide or protein domain having deaminase activity, nickase activity, recombinase activity, transposase activity, methylase activity, glycosylase (DNA glycosylase) activity, glycosylase inhibitor activity (e.g., uracil-DNA glycosylase inhibitor (UGI)), a reverse transcriptase, a peptide tag (e.g., a GCN4 peptide tag), demethylase activity, transcription activation activity, transcription repression activity, transcription release factor activity, histone modification activity, nuclease activity, single-strand RNA cleavage activity, double-strand RNA cleavage activity, restriction endonuclease activity (e.g., Fok1), nucleic acid binding activity, methyltransferase activity, DNA repair activity, DNA damage activity, dismutase activity, alkylation activity, depurination activity, oxidation activity, pyrimidine dimer forming activity, integrase activity, transposase activity, polymerase activity, ligase activity, helicase activity, a nuclear localization sequence or activity, an affinity polypeptide, a peptide tag, and/or photolyase activity. In some embodiments, the polypeptide of interest is a Fok1 nuclease, or a uracil-DNA glycosylase inhibitor. When encoded in a nucleic acid (polynucleotide, expression cassette, and/or vector) the encoded polypeptide or protein domain may be optimized for expression in an organism. In some embodiments, a polypeptide of interest may be linked to an engineered protein of the present invention or CRISPR-Cas effector protein domain to provide a CRISPR-Cas fusion protein. In some embodiments, a CRISPR-Cas fusion protein that comprises a CRISPR-Cas effector protein domain linked to a peptide tag may also be linked to a polypeptide of interest (e.g., a CRISPR-Cas effector protein domain may be, for example, linked to both a peptide tag (or an affinity polypeptide) and, for example, a polypeptide of interest.

In some embodiments, an editing system of the present invention comprises a CRISPR-Cas effector protein. As used herein, a “CRISPR-Cas effector protein” is a protein or polypeptide that cleaves, cuts, or nicks a nucleic acid; binds a nucleic acid (e.g., a target nucleic acid and/or a guide nucleic acid); and/or that identifies, recognizes, or binds a guide nucleic acid as defined herein. In some embodiments, a CRISPR-Cas effector protein may be an enzyme (e.g., a nuclease, endonuclease, nickase, etc.) and/or may function as an enzyme. In some embodiments, a CRISPR-Cas effector protein refers to a CRISPR-Cas nuclease. In some embodiments, a CRISPR-Cas effector protein comprises nuclease activity and/or nickase activity, comprises a nuclease domain whose nuclease activity and/or nickase activity has been reduced or eliminated, comprises single stranded DNA cleavage activity (ss DNAse activity) or which has ss DNAse activity that has been reduced or eliminated, and/or comprises self-processing RNAse activity or which has self-processing RNAse activity that has been reduced or eliminated. A CRISPR-Cas effector protein may bind to a target nucleic acid. A CRISPR-Cas effector protein may be a Type I, II, III, IV, V, or VI CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be a Type II CRISPR-Cas effector protein, for example, a Cas9 effector protein. In some embodiments, a CRISPR-Cas effector protein may be Type V CRISPR-Cas effector protein, for example, a Cas12 effector protein. In some embodiments, a CRISPR-Cas effector protein may be Cas12a and optionally may have an amino acid sequence of any one of SEQ ID NOs: 50-66 or 180 and/or a nucleotide sequence of any one of SEQ ID NOs: 67-69. In some embodiments, a CRISPR-Cas effector protein may be an active Cas12a and optionally may have an amino acid sequence of SEQ ID NO: 58 or 180. In some embodiments, a CRISPR-Cas effector protein may be an inactive (i.e., dead) Cas12a and optionally may have an amino acid sequence of SEQ ID NO: 50. In some embodiments, a CRISPR-Cas effector protein may be Cas12b and optionally may have an amino acid sequence of SEQ ID NO: 151.

Exemplary CRISPR-Cas effector proteins include, but are not limited to, a Cas9, C2c1, C2c3, Cas12a (also referred to as Cpf1), Cas12b, Cas12c, Cas12d, Cas12e, Cas13a, Cas13b, Cas13c, Cas13d, Casl, CasIB, Cas2, Cas3, Cas3′, Cas3″, Cas4, Cas5, Cas6, Cas7, Cas8, Cas9 (also known as Csnl and Csx12), Cas10, Csy1, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csxl4, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site and/or nuclease domain (e.g., RuvC, HNH, e.g., a RuvC site of a Cas12a nuclease domain; e.g., a RuvC site and/or HNH site of a Cas9 nuclease domain). A CRISPR-Cas effector protein having a mutation in its nuclease active site and/or nuclease domain, and therefore, no longer comprising nuclease activity, is commonly referred to as “inactive” or “dead,” e.g., dCas9. In some embodiments, a CRISPR-Cas effector protein having a mutation in its nuclease active site and/or nuclease domain may have impaired activity or reduced activity (e.g., nickase activity) as compared to the same CRISPR-Cas effector protein without the mutation.

A CRISPR Cas9 effector protein or Cas9 useful with this invention may be any known or later identified Cas9 nuclease. In some embodiments, a Cas9 of the present invention may be a protein from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. In some embodiments, a CRISPR-Cas effector protein may be a Cas9 and optionally may have a nucleotide sequence of any one of SEQ ID NOs: 70-80 or 140-143 and/or an amino acid sequence of any one of SEQ ID NOs: 81-82.

In some embodiments, the CRISPR-Cas effector protein may be a Cas9 derived from Streptococcus pyogenes and/or may recognize the PAM sequence motif NGG, NAG, NGA (Mali et al, Science 2013; 339 (6121): 823-826). In some embodiments, the CRISPR-Cas effector protein may be a Cas9 derived from Streptococcus thermophiles and/or may recognize the PAM sequence motif NGGNG and/or NNAGAAW (W=A or T) (See, e.g., Horvath et al, Science, 2010; 327 (5962): 167-170, and Deveau et al, J Bacteriol 2008; 190 (4): 1390-1400). In some embodiments, the CRISPR-Cas effector protein may be a Cas9 derived from Streptococcus mutans and/or may recognize the PAM sequence motif NGG and/or NAAR (R=A or G) (See, e.g., Deveau et al, J Bacteriol 2008; 190 (4): 1390-1400). In some embodiments, the CRISPR-Cas effector protein may be a Cas9 derived from Streptococcus aureus and/or may recognize the PAM sequence motif NNGRR (R=A or G). In some embodiments, the CRISPR-Cas effector protein may be a Cas9 derived from S. aureus and/or may recognize the PAM sequence motif N GRRT (R=A or G). In some embodiments, the CRISPR-Cas effector protein may be a Cas9 derived from S. aureus and/or may recognize the PAM sequence motif N GRRV (R=A or G). In some embodiments, the CRISPR-Cas effector protein may be a Cas9 that is derived from Neisseria meningitidis and/or may recognize the PAM sequence motif N GATT or N GCTT (R=A or G, V=A, G or C) (See, e.g., Hou et ah, PNAS 2013, 1-6). In the aforementioned embodiments in this paragraph, N in the PAM sequence motif can be any nucleotide residue, e.g., any of A, G, C or T. In some embodiments, the CRISPR-Cas effector protein may be a Cas13a derived from Leptotrichia shahii and/or may recognize a protospacer flanking sequence (PFS) (or RNA PAM (rPAM)) sequence motif of a single 3′ A, U, or C, which may be located within the target nucleic acid.

A Type V CRISPR-Cas effector protein useful with embodiments of the invention may be any Type V CRISPR-Cas nuclease. Exemplary Type V CRISPR-Cas effector proteins include, but are not limited, to Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c nuclease. In some embodiments, a Type V CRISPR-Cas effector protein may be a Cas12a. In some embodiments, a Type V CRISPR-Cas effector protein may be a nickase, optionally, a Cas12a nickase. In some embodiments, a Type V CRISPR-Cas effector protein may be a Cas12b (e.g., SEQ ID NO: 151).

In some embodiments, the CRISPR-Cas effector protein may be a Type V Clustered Regularly Interspaced Short Palindromic Repeats (CRISPR)-Cas nuclease. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein useful with this invention may be any known or later identified Cas12a (previously known as Cpf1) (see, e.g., U.S. Pat. No. 9,790,490, which is incorporated by reference for its disclosures of Cpf1 (Cas12a) sequences). The term “Cas12a” refers to an RNA-guided protein that can have nuclease activity, the protein comprising a guide nucleic acid binding domain and an active, inactive, or partially active DNA cleavage domain, thereby the RNA-guided nuclease activity of the Cas12a may be active, inactive or partially active, respectively. In some embodiments, a Cas12a useful with the invention may comprise a mutation in the nuclease active site (e.g., a RuvC site of the Cas12a domain). A Cas12a having a mutation in its nuclease domain and/or nuclease active site, and therefore, no longer comprising nuclease activity, is commonly referred to as deadCas12a (e.g., dCas12a). In some embodiments, a Cas12a having a mutation in its nuclease domain and/or nuclease active site may have impaired activity, e.g., may have reduced nickase activity.

In some embodiments, a Cas12a domain that may be modified to alter PAM specificity as described herein can include, but is not limited to, the amino acid sequence of any one of SEQ ID NOs: 180, SEQ IS NOs: 51-66 or SEQ ID NO: 193 (e.g., SEQ ID NOs: 180, 51, 52, 53, 54, 55, 56, 57, 58, 59, 60, 61, 62, 63, 64, 65 and/or 66 and/or 193) or a polynucleotide encoding the same (e.g., SEQ ID NOs: 27-29). In some embodiments, a Cas12a domain that may be modified to alter PAM specificity as described herein can include, but is not limited to, the amino acid sequence of any one of SEQ ID NO: 180, SEQ ID NO: 51 and/or SEQ ID NO: 55.

In some embodiments, a CRISPR-Cas effector protein may be optimized for expression in an organism, for example, in an animal (e.g., a mammal such as a human), a plant, a fungus, an archaeon, or a bacterium. In some embodiments, a CRISPR-Cas effector protein (e.g., Cas12a polypeptide/domain or a Cas9 polypeptide/domain) may be optimized for expression in a plant.

Any deaminase domain/polypeptide useful for base editing may be used with this invention. A “cytosine deaminase” and “cytidine deaminase” as used herein refer to a polypeptide or domain thereof that catalyzes or is capable of catalyzing cytosine deamination in that the polypeptide or domain catalyzes or is capable of catalyzing the removal of an amine group from a cytosine base. Thus, a cytosine deaminase may result in conversion of cytosine to a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid. In some embodiments, a cytosine deaminase encoded by a polynucleotide of the invention generates a C to T, G, or A conversion in the complementary strand in the genome.

A cytosine deaminase useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally-occurring cytosine deaminase, including, but not limited to, a primate (e.g., a human, monkey, chimpanzee, gorilla), a dog, a cow, a rat or a mouse. Thus, in some embodiments, an cytosine deaminase useful with the invention may be about 70% to about 100% identical to a wild-type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same. Evolved deaminases are disclosed in, for example, U.S. Pat. No. 10,113,163, Gaudelli et al. Nature 551 (7681): 464-471 (2017)) and Thuronyi et al. (Nature Biotechnology 37:1070-1079 (2019)), each of which are incorporated by reference herein for their disclosure of deaminases and evolved deaminases. In some embodiments, the cytosine deaminase may be an APOBEC1 deaminase having the amino acid sequence of SEQ ID NO: 83. In some embodiments, the cytosine deaminase may be an APOBEC3A deaminase having the amino acid sequence of SEQ ID NO: 84. In some embodiments, the cytosine deaminase may be an CDA1 deaminase, optionally a CDA1 having the nucleotide sequence of SEQ ID NO: 85. In some embodiments, the cytosine deaminase may be a FERNY deaminase, optionally a FERNY having the amino acid sequence of SEQ ID NO: 86. In some embodiments, the cytosine deaminase may be a rAPOBEC1 deaminase, optionally a rAPOBEC1 deaminase having the amino acid sequence of SEQ ID NO: 87. In some embodiments, the cytosine deaminase may be a hAID deaminase, optionally a hAID having the amino acid sequence of SEQ ID NO: 88 or SEQ ID NO: 89. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., “evolved deaminases”) (see, e.g., SEQ ID NO: 90, SEQ ID NO: 91, SEQ ID NO: 92). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of any one of SEQ ID NOs: 83, 84, or 86-92 or to the nucleotide sequence of SEQ ID NO: 85 (e.g., at least 80%, at least 85%, at least 90%, at least 92%, at least 95%, at least 96%, at least 97%, at least 98%, at least 99%, or at least 99.5% identical to the amino acid sequence of any one of SEQ ID NOs: 83, 84, or 86-92 or to the nucleotide sequence of SEQ ID NO: 85). In some embodiments, a polynucleotide encoding a cytosine deaminase may be codon optimized for expression in a plant and the codon optimized polypeptide may be about 70% to 99.5% identical to the reference polynucleotide.

An “adenine deaminase” and “adenosine deaminase” as used herein refer to a polypeptide or domain thereof that catalyzes or is capable of catalyzing the hydrolytic deamination (e.g., removal of an amine group from adenine) of adenine or adenosine. In some embodiments, an adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid. An adenine deaminase useful with this invention may be any known or later identified adenine deaminase from any organism (see, e.g., U.S. Pat. No. 10,113,163, which is incorporated by reference herein for its disclosure of adenine deaminases).

In some embodiments, an adenosine deaminase may be a variant of a naturally-occurring adenine deaminase. Thus, in some embodiments, an adenosine deaminase may be about 70% to 100% identical to a wild-type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild-type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA). In some embodiments, a TadA polypeptide or TadA domain does not comprise an N-terminal methionine. In some embodiments, a wild-type E. coli TadA comprises the amino acid sequence of SEQ ID NO: 93. In some embodiments, a mutated/evolved E. coli TadA* comprises the amino acid sequence of any one of SEQ ID NOs: 94-97. In some embodiments, a polynucleotide encoding a TadA/TadA* may be codon optimized for expression in a plant. In some embodiments, an adenine deaminase may comprise all or a portion of an amino acid sequence of any one of SEQ ID NOs: 98-103. In some embodiments, an adenine deaminase may comprise all or a portion of an amino acid sequence of any one of SEQ ID NOs: 93-103.

In some embodiments, a nucleic acid construct of this invention may further encode a glycosylase inhibitor (e.g., a uracil glycosylase inhibitor (UGI) such as uracil-DNA glycosylase inhibitor). In some embodiments, the invention provides fusion proteins comprising an engineered protein and a UGI and/or one or more polynucleotides encoding the same, optionally wherein the one or more polynucleotides may be codon optimized for expression in a plant.

A “uracil glycosylase inhibitor” useful with the invention may be any protein or polypeptide that is capable of inhibiting a uracil-DNA glycosylase base-excision repair enzyme. In some embodiments, a UGI domain comprises a wild-type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO: 104 or a polypeptide having about 70% to about 99.5% identity to the amino acid sequence of SEQ ID NO: 104 (e.g., at least 80%, at least 85%, at least 90%, at least 92%, at least 95%, at least 96%, at least 97%, at least 98%, at least 99%, or at least 99.5% identical to the amino acid sequence of SEQ ID NO: 104). For example, in some embodiments, a UGI domain may comprise a fragment of the amino acid sequence of SEQ ID NO: 104 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO: 104. In some embodiments, a UGI domain may be a variant of a known UGI (e.g., SEQ ID NO: 104) having about 70% to about 99.5% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% identity, and any range or value therein) to the known UGI. In some embodiments, a polynucleotide encoding a UGI may be optimized for expression in a plant (e.g., a plant) and the codon optimized polypeptide may be about 70% to about 99.5% identical to the reference polynucleotide.

In some embodiments, a polypeptide of interest for use with this invention may comprise at least one polypeptide or protein domain having glycosylase inhibitor activity. In some embodiments, the polypeptide of interest may be a uracil-DNA glycosylase inhibitor (UGI) polypeptide/domain. In some embodiments, a nucleic acid construct encoding a Cas12a nuclease of this invention and a cytosine deaminase domain (e.g., encoding a fusion protein comprising a Cas12a nuclease of the invention and a cytosine deaminase domain) may further encode a uracil-DNA glycosylase inhibitor (UGI), wherein the UGI is optimized for expression in an organism. In some embodiments, the invention provides a fusion protein comprising a Cas12a nuclease modified as described herein, a cytosine deaminase domain, and a UGI and/or one or more polynucleotides encoding the same, optionally wherein the one or more polynucleotides may be optimized for expression in an organism.

The present invention provides Cas12a nucleases modified to include recognition of non-natural PAM recognition sites/sequences (e.g., a Cas12a nuclease or engineered protein that comprises non-natural or altered PAM recognition specificity in addition to or instead of the natural PAM recognition specificity for that particular Cas12 or Cas12a nuclease). The present invention further provides engineered proteins that have altered PAM specificity as compared to a Cas12 or Cas12a nuclease. For example, an engineered protein of the present invention may be a nuclease that comprises non-natural PAM recognition specificity (e.g., altered binding affinity, altered PAM recognition) in addition to or instead of the natural PAM recognition specificity (e.g., wild-type binding affinity) for a Cas12a. In addition, the present invention is directed to methods for designing, identifying and selecting Cas12a nucleases and/or engineered proteins having desirable characteristics including improved PAM recognition specificity.

As used herein, a “non-natural PAM recognition site, a “non-native PAM recognition site,” “non-natural PAM sequence,” and/or “non-native PAM sequence,” refer to PAM sequences that are recognized by a modified Cas nuclease or engineered protein as described herein, but which are not recognized by the corresponding wild type Cas nuclease.

As used herein in reference to a modified protein, a modified Cas12a polypeptide, engineered protein, and/or nuclease, “altered PAM specificity” means that the PAM specificity of the modified protein, modified Cas12a polypeptide, engineered protein, and/or nuclease is altered from that of the wild-type nuclease (e.g., non-native PAM sequences are recognized in addition to and/or instead of the native PAM sequence(s)). For example, a modified Cas12a nuclease (e.g., a modified protein) would be altered in its PAM specificity if it recognizes a PAM sequence other than and/or in addition to the native Cas12a PAM sequence of TTTV, wherein V is A, C or G (natural or wild type PAM recognition sequence for Cas12a). In some embodiments, an engineered protein of the present invention comprises a polypeptide that is a portion of Cas12a and the engineered protein has altered PAM specificity in that it recognizes a PAM sequence other than and/or in addition to the native Cas12a PAM sequence of TTTV, wherein V is A, C or G. In some embodiments, a modified protein of the present invention may be as described in U.S. Patent Application Publication No. 2021/0115421 and/or a nuclease or portion thereof (e.g., polypeptide thereof) may be modified and/or altered as described in U.S. Patent Application Publication No. 2021/0115421, which is incorporated herein by reference in its entirety. In some embodiments, an engineered protein of the present invention recognizes a native PAM sequence (e.g., a PAM sequence of TTTV, wherein V is A, C or G) and/or recognizes a non-native PAM sequence (e.g., a PAM sequence of CCCC, TCCA, TCCC, TCCG, TTCA, TTCC, TATA, TATC, and TATG, and/or TTCG; e.g., a PAM sequence of TTTT, TTCA, TTCC, TTCG, ATTC, CTTA, CTTC, CTTG, GTTC, TATA, TATC, CTCC, TCCG, TACA, TCCG, TACA, TCCG, TCCC, TCCA, and/or TATG).

Accordingly, the present invention provides Cas12a nucleases having modified PAM recognition specificities. In some embodiments, the present invention provides a CRISPR (Clustered Regularly Interspaced Short Palindromic Repeats) Cas12a polypeptide, wherein the Cas12a polypeptide comprises (a) an amino acid sequence having at least 95% identity (e.g., about 95, 96, 97, 98, 99, 100% identity; e.g., about 95% to about 100%) to the amino acid sequence of SEQ ID NO: 180 and a mutation located at one or more than one (e.g., 2, 3, 4, 5, 6, 7, 8, 9, 10, 11, 12, 13 or more) of the following positions of K135, S168, T246, N263, T307, L324, E330, S345, R359, D367, 1369, E379, Y381, I417, F474, E479, E484, D489, L498, N590, K167, Q231, A454, F657, F337, L459, 1507, M531, D654, E659, S170, V245, I425, V463, L492, K514, N112, E223, Y426, I455, S117, A239, T295, N100, K387, A404, D405, D423, E539, D572, N211, N527, Y553, Y554, L321, K192, I344 and/or E443, optionally a mutation located at position K135, S168, T246, N263, T455, L324, E330, S345, R359, D367, I369, E379, Y381, I417, F474, E479, E484, D489, L498, and/or N590, and/or a mutation comprising one or more of K135N, S168N, T246S, N263I, T455S, L324M, E330V, S345R, R359S, D367V, I369M, E379G, Y381H, I417F, F474L, E479V, E484V, D489Y, L498M, N590Y, K167I, Q231, A454V, F657, F337L, L459M, I507V, M531I, D654E, E659V, S170L, V245I, I425V, V463A, L492P, K514N, N112D, E223V, Y426D, 1455F, S117C, A239T, T295I, N100S, K387E, A404V, D405G, D423V, E539V, D572G, N211S, N527S, Y553H, Y554N, L321V, K192I, I344T and/or E443D, optionally a mutation comprising one or more of K135N, S168N, T246S, N263I, T307S, L324M, E330V, S345R, R359S, D367V, I369M, E379G, Y381H, I417F, F474L, E479V, E484V, D489Y, L498M, and/or N590Y, optionally the mutation of D489Y, L324M, E484V and/or E330V, with reference to position numbering of SEQ ID NO: 180, wherein the polypeptide exhibits a reduced PAM stringency and increased recognition of new protospacer adjacent motifs (PAMs) as compared to the amino acid sequence of SEQ ID NO: 180. Thus, in some embodiments, a Cas12a polypeptide of this invention may comprise a single mutation located at, for example, the position of K135, S168, T246, N263, T455, L324, E330, S345, R359, D367, I369, E379, Y381, I417, F474, E479, E484, D489, L498 or N590 with reference to the position numbering of SEQ ID NO: 180, or a single mutation of K135N, S168N, T246S, N263I, T455S, L324M, E330V, S345R, R359S, D367V, I369M, E379G, Y381H, 1417F, F474L, E479V, E484V, D489Y, L498M, or N590Y with reference to the position numbering of SEQ ID NO: 180 or a Cas12a polypeptide of this invention may comprise a combination of two or more mutations located at, for example, any two or more positions of K135, S168, T246, N263, T455, L324, E330, S345, R359, D367, I369, E379, Y381, I417, F474, E479, E484, D489 and/or L498 with reference to the position numbering of SEQ ID NO: 180, and/or a combination of two or more mutations of K135N, S168N, T246S, N263I, T307S, L324M, E330V, S345R, R359S, D367V, I369M, E379G, Y381H, I417F, F474L, E479V, E484V, D489Y, L498M, and/or N590Y with reference to the position numbering of SEQ ID NO: 180.

In some embodiments, a mutation of a Cas12a polypeptide at the positions of K135, S168, T246, N263, T307, L324, E330, S345, R359, D367, I369, E379, Y381, I417, F474, E479, E484, D489 or L498 with reference to the position numbering of SEQ ID NO: 180 may comprise, consist essentially of, or consist of the amino acid mutation of K135N, S168N, T246S, N263I, T307S, L324M, E330V, S345R, R359S, D367V, I369M, E379G, Y381H, I417F, F474L, E479V, E484V, D489Y, or L498M with reference to position numbering of SEQ ID NO: 180. In some embodiments, a Cas12a polypeptide of this invention may comprise a mutation of N590Y.

In some embodiments, the present invention provides a CRISPR (Clustered Regularly Interspaced Short Palindromic Repeats) Cas12a polypeptide, wherein the Cas12a polypeptide comprises (b) an amino acid sequence having at least 95% identity (e.g., about 95, 96, 97, 98, 99, 100% identity; e.g., about 95% to about 100%) to the amino acid sequence of SEQ ID NO: 51 and a mutation at one or more than one (e.g., 2, 3, 4, 5, 6, 7, 8, 9, 10, 11, 12, 13 or more) of the following positions E153, S186, R267, I285, Y336, L352, S358, E372, R386, E391, R393, K403, A405, A434, V486, E491, P494, R499, E508, A602, I185, Q252, E465, L698, F365, Q470, A517, A541, L695, P700, A188, S266, I442, L474, G502, K524, T114, P244, L443, I466, D119, Q260, K324, T101, R411, A427, G428, S440, E549, E584, S234, M537, Y563, Y564, A349, R210, L371, and/or K459, optionally a mutation at position E153, S186, R267, I285, Y336, L352, S358, E372, R386, E391, R393, K403, A405, A434, V486, E491, P494, R499, E508, and/or A602, optionally a mutation at position R499, L352, P494 and/or S358, with reference to position numbering of SEQ ID NO: 51, wherein the polypeptide exhibits a reduced PAM stringency and increased recognition of new protospacer adjacent motifs (PAMs) as compared to the amino acid sequence of SEQ ID NO: 51, wherein the Cas12a polypeptide may comprise a mutation located at any combination of the above positions. Thus, in some embodiments, a Cas12a polypeptide of this invention may comprise a single mutation, for example, located at the position of E153, S186, R267, I285, Y336, L352, S358, E372, R386, E391, R393, K403, A405, A434, V486, E491, P494, R499, E508, and/or A602 with reference to the position numbering of SEQ ID NO: 51, or a Cas12a polypeptide of this invention may comprise a combination of two or more mutations located at, for example, any two or more positions of E153, S186, R267, I285, Y336, L352, S358, E372, R386, E391, R393, K403, A405, A434, V486, E491, P494, R499, E508, and/or A602 with reference to the position numbering of SEQ ID NO: 51.

In some embodiments, the present invention provides a CRISPR (Clustered Regularly Interspaced Short Palindromic Repeats) Cas12a polypeptide of (c), wherein the Cas12a polypeptide (c) comprises an amino acid sequence having at least 95% identity (e.g., about 95, 96, 97, 98, 99, 100% identity; e.g., about 95% to about 100%) to the amino acid sequence of SEQ ID NO: 55 and a mutation located at one or more than one (e.g., 2, 3, 4, 5, 6, 7, 8, 9, 10, 11, 12, 13 or more) of the following positions of I162, P196, V290, S308, Q358, L377, K385, T400, Q423, D431, P433, K443, T445, A480, I454, D550, E559, V564, A573, N666, I195, Q275, A525, F741, Y392, L529, I582, A606, G738, D743, S198, F289, I488, T533, E567, K589, S116, A267, F489, A525, N121, T283, E343, T104, L451, R467, D468, P486, E614, E648, T243, N602, Y628, Y629, L374, S220, L399, and/or Q507, optionally a mutation located at I162, P196, V290, S308, Q358, L377, K385, T400, Q423, D431, P433, K443, T445, A480, I454, D550, E559, V564, A573, and/or N666, optionally a mutation at position V564, L377, E559 and/or K385, with reference to position numbering of SEQ ID NO: 55, wherein the polypeptide exhibits a reduced PAM stringency and increased recognition of new protospacer adjacent motifs (PAMs) as compared to the amino acid sequence of SEQ ID NO: 55, wherein the Cas12a polypeptide may comprise a mutation located at any combination of the above positions. Thus, in some embodiments, a Cas12a polypeptide (c) of this invention may comprise a single mutation located at a position, for example, of I162, P196, V290, S308, Q358, L377, K385, T400, Q423, D431, P433, K443, T445, A480, I454, D550, E559, V564, A573, and/or N666 with reference to the position numbering of SEQ ID NO: 55, or a Cas12a polypeptide of this invention may comprise a combination of two or more mutations located at any two or more positions of, for example, I162, P196, V290, S308, Q358, L377, K385, T400, Q423, D431, P433, K443, T445, A480, 1454, D550, E559, V564, A573, and/or N666 with reference to the position numbering of SEQ ID NO: 55.

In some embodiments, in addition to a mutation at one or more than one of the following positions of K135, S168, T246, N263, T307, L324, E330, S345, R359, D367, I369, E379, Y381, I417, F474, E479, E484, D489, L498, N590, K167, Q231, A454, F657, F337, L459, I507, M531, D654, E659, S170, V245, I425, V463, L492, K514, N112, E223, Y426, I455, S117, A239, T295, N100, K387, A404, D405, D423, E539, D572, N211, N527, Y553, Y554, L321, K192, I344 and/or E443 with reference to the position numbering of SEQ ID NO: 180, and/or one or more of the mutations of K135N, S168N, T246S, N263I, T307S, L324M, E330V, S345R, R359S, D367V, I369M, E379G, Y381H, I417F, F474L, E479V, E484V, D489Y, L498M, N590Y, K167I, Q231, A454V, F657, F337L, L459M, I507V, M531I, D654E, E659V, S170L, V245I, I425V, V463A, L492P, K514N, N112D, E223V, Y426D, 1455F, S117C, A239T, T295I, N100S, K387E, A404V, D405G, D423V, E539V, D572G, N211S, N527S, Y553H, Y554N, L321V, K192I, I344T and/or E443D with reference to the position numbering of SEQ ID NO: 180, a Cas12a polypeptide of (a) may further comprise, consist of, or consist essentially of a mutation located at one or more than one of the following positions of K116, K120, K121, D122, E125, T148, T149, T152, D156, E159, Q529, D535, K538, D541, Y542, L585, N590, K591, M592, K595, V596, S599, K600, K601, Y616, Y646, and/or W649 with reference to position numbering of SEQ ID NO: 180, optionally wherein the mutation at the location of K116, K120, K121, D122, E125, T148, T149, T152, D156, E159, Q529, D535, K538, D541, Y542, L585, N590, K591, M592, K595, V596, S599, K600, K601, Y616, Y646, and/or W649 may comprise, consist of, or consist essentially of the mutations of K116R, K116N, K116D, K120R, K120H, K120N, K120T, K120Y, K120Q, K121S, K121T, K121H, K121R, K121G, K121D, K121Q, D122R, D122K, D122H, D122E, D122N, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D1561, D156V, D156L, D156E, E159K, E159R, E159H, E159Y, E159Q, Q529N, Q529T, Q529H, Q529A, Q529F, Q529G, Q529G, Q529S, Q529P, Q529W, Q529D, G532D, G532N, G532S, G532H, G532F, G532K, G532R, G532Q, G532A, G532L, G532C, D535N, D535H, D535V, D535T, D535, S D535A, D535W, D535K, K538R K538V, K538Q, K538W, K538Y, K538F, K538H, K538L, K538M, K538C, K538G, K538A, K538P, D541N, D541H, D541R, D541K, D541Y, D5411, D541A, D541S, D541E, Y542R, Y542K, Y542H, Y542Q, Y542F, Y542L, Y542M, Y542P, Y542V, Y542N, Y542T, L585G, L585H, L585F, N590Y, K591W, K591F, K591Y, K591H, K591R, K591S, K591A, K591G, K591P, M592R, M592K, M592Q, M592E, M592A, K595R, K595Q, K595Y, K595L, K595W, K595H, K595E, K595S, K595D, K595M, V596T, V596H, V596G, V596A, S599G, S599H, S599N, S599D, K600R, K600H, K600G, K601R, K601H, K601Q, K601T, Y616K, Y616R, Y616E, Y616F, Y616H, Y646R, Y646E, Y646K, Y646H, Y646Q, Y646W, Y646N, W649H, W649K, W649Y, W649R, W649E, W649S, W649V, and/or W649T with reference to position numbering of SEQ ID NO: 180.

In some embodiments, in addition to a mutation at one or more than one of the following positions of E153, S186, R267, I285, Y336, L352, S358, E372, R386, E391, R393, K403, A405, A434, V486, E491, P494, R499, E508, A602, I185, Q252, E465, L698, F365, Q470, A517, A541, L695, P700, A188, S266, I442, L474, G502, K524, T114, P244, L443, I466, D119, Q260, K324, T101, R411, A427, G428, S440, E549, E584, S234, M537, Y563, Y564, A349, R210, L371, and/or K459 with reference to the position numbering of SEQ ID NO: 51, a Cas12a polypeptide of (b) may further comprise, consist of, or consist essentially of a mutation located at one or more than one of the following positions of E127, K548, N552, F598, A602 and/or K607 with reference to position numbering of SEQ ID NO: 51.

In some embodiments, in addition to a mutation at one or more than one of the following positions of I162, P196, V290, S308, Q358, L377, K385, T400, Q423, D431, P433, K443, T445, A480, 1454, D550, E559, V564, A573, N666, I195, Q275, A525, F741, Y392, L529, I582, A606, G738, D743, S198, F289, I488, T533, E567, K589, S116, A267, F489, A525, N121, T283, E343, T104, L451, R467, D468, P486, E614, E648, T243, N602, Y628, Y629, L374, S220, L399, and/or Q507 with reference to the position numbering of SEQ ID NO: 55, a Cas12a polypeptide of (c) may further comprise, consist of, or consist essentially of a mutation located at one or more than one of the following positions of D129, K613, N617, L661, N666 and/or K671 with reference to position numbering of SEQ ID NO: 55.

As would be understood, any single Cas12a polypeptide having two or more mutations would comprise only a single mutation at any given position. Thus, for example, a polypeptide may have mutation at position D535 that may be only one of the mutations of D535A, D535H, D535K, D535N, D535S, D535T, or D535V.

In some embodiments, (a) a Cas12a polypeptide of the invention may comprise, consist of, or consist essentially of any one of the following combinations of mutations of (1) D156R, D489Y and K595Y, (2) D156R, L324M, E484V and K595Y, (3) D156R, E330V and D489Y, (4) D156R, D367V, E379G, Y381H, N590Y and K595Y, (5) D156R, I417F and D489Y, (6) D156R, I417F, K538N and K595Y, (7) D156R, E330V, I417F, and K538N, (8) D156R and E330V, (9) D156R and 1417F, (10) D156R, N263I and L498M, (11) K135N, D156R, T307S, S345R, D489V and K595Y, and/or (12) D156R, S168N, T246S, R359S, I369M, F474L, E479V, K538N, Y542N and K595Y with reference to position numbering of SEQ ID NO: 180; (b) a Cas12a polypeptide of the invention may comprise, consist of, or consist essentially of mutations at any one of the following combinations of locations of (1) E174, R499 and K607, (2) E174, L352, P494 and K607, (3) E174, S358 and R499, (4) E174, E391, K403, A405, A602 and K607, (5) E174, A434 and R499, (6) E174, A434, K548 and K607, (7) E174, S358, A434 and K548, (8) E174 and S358, (9) E174 and A434, (10) E174, I285 and E508, (11) E153, E174, Y336, E372, R499 and K607, and/or (12) E174, S186, R267, R386, R393, V486, E491, K548, N552 and K607 with reference to position numbering of SEQ ID NO: 51; and/or (c) a Cas12a polypeptide of the invention may comprise, consist of, or consist essentially of mutations at any one of the following combinations of locations of (1) E184, V564 and K671, (2) E184, L377, E559 and K671, (3) E184, K385 and V564, (4) E184, D431, K443, T445, N666 and K671, (5) E184, A480 and V564, (6) E184, A480, K613 and K671, (7) E184, K385, A480, and K613, (8) E184 and K385, (9) E184 and A480, (10) E184, S308, and A573, (11) I162, E184, Q358, T400, V564 and K671, or (12) E184, P196, V290, Q423, P433, I545, D550, K613, N617 and K671 with reference to position numbering of SEQ ID NO: 55.

In some embodiments, a mutation in a LBCas12a polypeptide of the invention does not comprise of a mutation of D156R, G532R, K538R, K538V, Y542R or K595R with reference to position numbering of SEQ ID NO: 180. In some embodiments, a mutation in a LBCas12a polypeptide does not comprise of a mutation of N263, T295, E330, E484, L498, N100, K387, A404, D405, D423, E539, D572, N211, N527, Y553, and/or Y554 with reference to position numbering of SEQ ID NO: 180. In some embodiments, the mutation in a Cas12a polypeptide of the invention does not comprise the combination of mutations of (a) G532R and K595R, (b) G532R, K538V and Y542R, or (c) D156R, G532R and K532R with reference to position numbering of SEQ ID NO: 180. In some embodiments, a mutation in a LBCas12a polypeptide of the invention does not comprise of a mutation of E174, S542, K548, N552 or K607 with reference to position numbering of SEQ ID NO: 51. In some embodiments, a mutation in a LBCas12a polypeptide does not comprise of a mutation of 1285, K324, S358, P494, E508, T101, R411, A427, G428, S440, E549, E584, S234, M537, Y563, and/or Y564 with reference to position numbering of SEQ ID NO: 51. In some embodiments, the mutation in a Cas12a polypeptide of the invention does not comprise the combination of mutations of (a) S542 and K607, (b) S542, K548 and N552, or (c) E174 and S542 with reference to position numbering of SEQ ID NO: 51. In some embodiments, a mutation in a LBCas12a polypeptide of the invention does not comprise of a mutation of E184, N607, K613, N617 or K671 with reference to position numbering of SEQ ID NO: 55. In some embodiments, a mutation in a LBCas12a polypeptide does not comprise of a mutation of S308, E343, K385, E559, A573, T104, L451, R467, D468, P486, E614, E648, T243, N602, Y628, and/or Y629 with reference to position numbering of SEQ ID NO: 55. In some embodiments, the mutation in a Cas12a polypeptide of the invention does not comprise the combination of mutations of (a) N607 and K671, (b) N607, K613 and K613, or (c) E184 and N607 with reference to position numbering of SEQ ID NO: 55.

In some embodiments, a Cas12a polypeptide of the invention exhibits an altered protospacer adjacent motif (PAM) specificity as compared to wild type LbCas12a (e.g., SEQ ID NO: 180, SEQ ID NO: 51, SEQ ID NO: 55), wherein the altered PAM specificity may include, but is not limited to, NNNG, NNNT, NNNA, NNNC, NNG, NNT, NNC, NNA, NG, NT, NC, NA, NN, NNN, NNNN, wherein each N of each sequence is independently selected from any of T, C, G, or A. In some embodiments, an altered PAM specificity of a Cas12a of the invention exhibits access at PAMs having the sequences of NNBV, wherein N=A/G/C/T, B=G/C/T and V=A/G/C. In some embodiments, the altered PAM specificity may include, but is not limited to, TTTT, TTCA, TTCC, TTCG, ATTC, CTTA, CTTC, CTTG, GTTC, TATA, TATC, CTCC, TCCG, TACA, TCCG, TACA, TCCG, TCCC, TCCA, and/or TATG.

In addition to having an altered PAM recognition specificity a Cas12a nuclease may further comprise a mutation in the nuclease active site (e.g., RuvC domain) (e.g., deadLbCas12a, dLbCas12a). Such modifications may result in the Cas12a polypeptide having reduced nuclease activity (e.g., nickase activity) or no nuclease activity.

In some embodiments, a Type V Clustered Regularly Interspaced Short Palindromic Repeats (CRISPR)-associated (Cas) (CRISPR-Cas) system is provided comprising a guide nucleic acid (CRISPR RNA, CRISPR DNA, crRNA, crDNA) comprising a spacer sequence and a repeat sequence and (a) a fusion protein comprising the Cas12a polypeptide of the invention and a polypeptide of interest; or (b) a nucleic acid encoding the Cas12a polypeptide of the invention, and a nucleic acid encoding the polypeptide of interest, wherein the guide nucleic acid is capable of forming a complex with the Cas12a polypeptide or the fusion protein and the spacer sequence is capable of hybridizing to a target nucleic acid, thereby guiding the Cas12a polypeptide and the polypeptide of interest to the target nucleic acid, whereby the target nucleic acid is modified (e.g., cleaving or editing) or modulated (e.g., modulating transcription). In some embodiments, the Cas12a polypeptide comprises a mutation in the nuclease domain, e.g., the RuvC domain. In some embodiments, the system comprises a polypeptide of interest linked to the C-terminus and/or the N-terminus of the mCas12a nuclease (e.g., a fusion protein), optionally via a peptide linker. In some embodiments, a polypeptide of interest may comprise at least one polypeptide or protein domain having, for example, deaminase (deamination) activity, nickase activity, recombinase activity, transposase activity, methylase activity, glycosylase (DNA glycosylase) activity, glycosylase inhibitor activity (e.g., uracil-DNA glycosylase inhibitor (UGI)). demethylase activity, transcription activation activity, transcription repression activity, transcription release factor activity, histone modification activity, nuclease activity, single-strand RNA cleavage activity, double-strand RNA cleavage activity, restriction endonuclease activity (e.g., Fok1), nucleic acid binding activity, methyltransferase activity, DNA repair activity, DNA damage activity, dismutase activity, alkylation activity, depurination activity, oxidation activity, pyrimidine dimer forming activity, integrase activity, transposase activity, polymerase activity, ligase activity, helicase activity, and/or photolyase activity, optionally wherein the polypeptide of interest comprises at least one polypeptide or protein domain having deaminase activity. In some embodiments, a polypeptide of interest may comprise a uracil-DNA glycosylase inhibitor (UGI).

Additionally, provided herein are fusion proteins that comprise a Cas12a nuclease of the invention. In some embodiments, the invention provides a fusion protein comprising the Cas12a polypeptide of invention, and a linker, optionally a peptide linker. In some embodiments, a peptide linker may be linked to the C-terminus and/or the N-terminus of the Cas12a polypeptide of the invention. In some embodiments, a fusion protein may comprise a polypeptide of interest linked to the C-terminus and/or to the N-terminus of the Cas12a of the invention. In some embodiments, the present invention provides a fusion protein comprising a Cas12a of the invention and an optional intervening linker linking the polypeptide of interest.

In some embodiments, the invention provides a polynucleotide encoding a Cas12a polypeptide of the invention or a fusion protein comprising the Cas12a polypeptide of the invention. In some embodiments, a polynucleotide may be optimized, for example, optimized for expression in an organism, e.g., in the cell of an organism, optionally wherein the organism may include but is not limited to, an animal, a plant, a fungus, an archaeon, or a bacterium. In some embodiments, the invention provides a cell comprising the polynucleotide of the invention, the nucleic acid construct of the invention, the expression cassette or vector of the invention, and/or the system of the invention.

Any linker known in the art or later identified that does not interfere with the activity of the fusion protein may be used. A linker that does not “interfere” with the activity of a fusion protein is a linker that does not reduce or eliminate the activity of the polypeptides of the fusion protein (e.g., the nuclease and/or the polypeptide of interest); that is, the nuclease activity, nucleic acid binding activity, editing activity, and/or any other activity of the nuclease or peptide of interest is maintained in a fusion protein in which the nuclease and the polypeptide of interest are tethered to one another via the linker. In some embodiments, a peptide linker may be linked (e.g., at its N-terminus) to the C-terminus of a Cas12a of the invention, optionally wherein the fusion protein may further comprise a polypeptide of interest linked to the C-terminus of the linker. In some embodiments, a peptide linker may be linked (e.g., at its C-terminus) to the N-terminus of a Cas12a of the invention, optionally wherein the fusion protein may further comprise a polypeptide of interest linked to the N-terminus of the linker. In some embodiments, a Cas12a of the invention may be linked at both its C-terminus and N-terminus to a linker and/or a polypeptide of interest (directly or via a linker).

A polypeptide of interest useful with this invention can include, but is not limited to, a polypeptide or protein domain having deaminase (deamination) activity, nickase activity, recombinase activity, transposase activity, methylase activity, glycosylase (DNA glycosylase) activity, glycosylase inhibitor activity (e.g., uracil-DNA glycosylase inhibitor (UGI)). demethylase activity, transcription activation activity, transcription repression activity, transcription release factor activity, histone modification activity, nuclease activity, single-strand RNA cleavage activity, double-strand RNA cleavage activity, restriction endonuclease activity (e.g., Fok1), nucleic acid binding activity, methyltransferase activity, DNA repair activity, DNA damage activity, dismutase activity, alkylation activity, depurination activity, oxidation activity, pyrimidine dimer forming activity, integrase activity, transposase activity, polymerase activity, ligase activity, helicase activity, and/or photolyase activity.

In some embodiments, a polypeptide of interest may comprise at least one polypeptide or protein domain having deaminase activity. In some embodiments, the at least one polypeptide or protein domain may be an adenine deaminase domain. An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, a Cas12a nuclease of the invention may comprise a mutation in its nuclease active site (e.g., RuvC). A Cas12a nuclease having a mutation in its nuclease active site(s) and no longer comprising nuclease activity are commonly referred to as “dead,” e.g., dCas12a. In some embodiments, a Cas12a domain or polypeptide of the invention having a mutation in its nuclease active site(s) may have impaired activity or reduced activity (e.g., nickase activity) as compared to the same Cas12a nuclease without the mutation.

Cas12a nucleases of the invention may be used in combination with a guide RNA (gRNA, CRISPR array, CRISPR RNA, crRNA), designed to function with the Cas12a nuclease, to modify a target nucleic acid. A guide nucleic acid useful with this invention comprises at least a spacer sequence and a repeat sequence. The guide nucleic acid is capable of forming a complex with the Cas12a nuclease domain encoded and expressed by a polynucleotide/nucleic acid construct of the invention encoding a Cas12a nuclease and the spacer sequence is capable of hybridizing to a target nucleic acid, thereby guiding the nucleic acid construct (e.g., a Cas12a nuclease of the invention (and/or a polypeptide of interest)) to the target nucleic acid, wherein the target nucleic acid may be modified (e.g., cleaved or edited) or modulated (e.g., modulating transcription) by the Cas12a nuclease (and/or an encoded deaminase domain and/or polypeptide of interest). As an example, a nucleic acid construct encoding aCas12a domain linked to a cytosine deaminase domain (e.g., a fusion protein) may be used in combination with guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas12a domain (e.g., a Cas12a nuclease modified as described herein) linked to an adenine deaminase domain (e.g., a fusion protein) may be used in combination with a guide nucleic acid to modify a target nucleic acid, wherein the adenine deaminase domain of the fusion protein deaminates an adenosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target nucleic acid (e.g., protospacer), and at least one repeat sequence (e.g., a repeat of a Type V Cas12a CRISPR-Cas system, or a fragment or portion thereof), wherein the repeat sequence may be linked to the 5′ end and/or the 3′ end of the spacer sequence. The design of a gRNA of this invention may be based on a Type V Cas12a system. In some embodiments, a “guide nucleic acid,” as used herein means a nucleic acid that comprises at least one spacer sequence, which is complementary to (and hybridizes to) a target nucleic acid (e.g., a target DNA and/or a protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas12f, Cas12i, Cas13a, Cas13b, Cas13c, Cas13d, Casl, CasIB, Cas2, Cas3, Cas3′, Cas3″, Cas4, Cas5, Cas6, Cas7, Cas8, Cas9 (also known as Csn1 and Csx12), Cas10, Csyl, Csy2, Csy3, Cse1, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csxl4, Csx10, Csx16, CsaX, Csx3, Csxl, Csxl5, Csfl, Csf2, Csf3, Csf4 (dinG), and/or Csf5, or a fragment thereof), wherein the repeat sequence may be linked to the 5′ end and/or the 3′ end of the spacer sequence. In some embodiments, the guide nucleic acid comprises DNA. In some embodiments, the guide nucleic acid comprises RNA (e.g., is a guide RNA). The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one “repeat sequence-spacer” sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

In some embodiments, a “repeat sequence” refers to, for example, any repeat sequence of a wild-type Cas12a locus (e.g., a LbCas12a locus, an AsCas12a locus, a FnCas12a locus) or a repeat sequence of a synthetic crRNA that is functional with the Cas12a nuclease encoded by the nucleic acid constructs of the invention. A repeat sequence useful with this invention can be any known or later identified repeat sequence of a Cas12a locus or it can be a synthetic repeat designed to function in a Cas12a Type V CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from a wild-type V CRISPR-Cas locus (e.g., a wild type Cas12a locus). A repeat sequence from a wild-type Cas12a locus may be determined through established algorithms, such as using the CRISPRfinder offered through CRISPRdb (see, Grissa et al. Nucleic Acids Res. 35 (Web Server issue): W52-7). In some embodiments, a repeat sequence or portion thereof is linked at its 3′ end to the 5′ end of a spacer sequence, thereby forming a repeat-spacer sequence (e.g., guide RNA, crRNA). In some embodiments, a “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35 (Web Server issue): W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide RNA comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100, or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more consecutive nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, and any range or value therein)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have about 70% complementarity to a target nucleic acid. In other embodiments, the spacer nucleotide sequence can have about 80% complementarity to a target nucleic acid. In still other embodiments, the spacer nucleotide sequence can have about 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., about 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that may be at least about 15 nucleotides to about 30 nucleotides in length. In some embodiments, the spacer may be about 20, 21, 22, 23, 24, or 25 nucleotides in length. In some embodiments, the spacer may be 23 nucleotides in length.

In some embodiments, the 5′ region of a spacer sequence of a guide nucleic acid may be fully complementary to a target nucleic acid, while the 3′ region of the spacer may be substantially complementary to the target nucleic acid (such as for a spacer in a Type V CRISPR-Cas system), or the 3′ region of a spacer sequence of a guide nucleic acid may be fully complementary to a target nucleic acid, while the 5′ region of the spacer may be substantially complementary to the target nucleic acid (such as for a spacer in a Type II CRISPR-Cas system), and therefore, the overall complementarity of the spacer sequence to the target nucleic acid may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence, may be 100% complementary to the target nucleic acid, while the remaining nucleotides in the 3′ region of the spacer sequence may be substantially complementary (e.g., at least about 70% complementary) to the target nucleic acid. In some embodiments, the first 1 to 8 nucleotides (e.g., the first 1, 2, 3, 4, 5, 6, 7, 8, nucleotides, and any range therein) of a 5′ end of the spacer sequence may be 100% complementary to the target nucleic acid, while the remaining nucleotides in the 3′ region of the spacer sequence may be substantially complementary (e.g., at least about 50% complementary (e.g., about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleic acid.

As a further example, in a guide nucleic acid for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target nucleic acid, while the remaining nucleotides in the 5′ region of the spacer sequence are substantially complementary (e.g., at least about 70% complementary) to the target nucleic acid. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target nucleic acid, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target nucleic acid. A recruiting guide RNA further comprises one or more recruiting motifs as described herein, which may be linked to the 5′ end of the guide or the 3′ end or it may be inserted into the recruiting guide nucleic acid (e.g., within the hairpin loop).

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refer to a region of an organism's genome that is fully complementary (100% complementary) or substantially complementary (e.g., at least 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, and any range or value therein)) to a spacer sequence in a guide nucleic acid as defined herein. A target nucleic acid is targeted by an editing system (or a component thereof) as described herein. In some embodiments, a target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome, an animal (e.g., human) genome, a bacterial genome). In some embodiments, a target region may be selected from any at least 15 consecutive nucleotides (e.g., 15, 16, 17, 18, 19, 20, 21, 22, 23, 24, 25, 26, 27, 28, 29, 30 or more nucleotides, and any range or value therein; e.g., about 19 to about 25 nucleotides, about 20 to about 24 nucleotides in length, and the like) located immediately adjacent to a PAM sequence.

A “protospacer sequence” refers to the target nucleic acid and specifically to the portion of the target nucleic acid (e.g., or target region in the genome) that is fully or substantially complementary (and hybridizes) to the spacer sequence of the CRISPR repeat-spacer sequences (e.g., guide RNAs, CRISPR arrays, crRNAs).

In the case of Type V CRISPR-Cas Cas12a systems, the protospacer sequence is flanked (immediately adjacent to) a protospacer adjacent motif (PAM). The PAM is located at the 5′ end on the non-target strand and at the 3′ end of the target strand (see below, as an example).

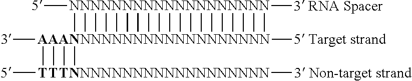

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′.

The polypeptides, fusion proteins and/or systems of the invention may be encoded by polynucleotides or nucleic acid constructs. In some embodiments, a polynucleotide/nucleic acid construct encoding the polypeptides, fusion proteins and/or systems of the invention may be operably associated with regulatory elements (e.g., promotors, terminators and the like) for expression in an organism of interest and/or a cell of an organism of interest as described herein. In some embodiments, a polynucleotide/nucleic acid construct encoding a polypeptide, fusion protein and/or system of the invention may be optimized for expression in an organism.

In some embodiments, the present invention provides a complex comprising (a) a Cas12a polypeptide of the invention or a fusion protein of the invention and (b) a guide nucleic acid (e.g., CRISPR RNA, CRISPR DNA, crRNA, crDNA).

In some embodiments, the present invention provides a composition comprising (a) a Cas12a polypeptide of the invention or a fusion protein of the invention and (b) a guide nucleic acid.

In some embodiments, expression cassettes and/or vectors are provided comprising the polynucleotides/nucleic acid constructs of the invention. In some embodiments, expression cassettes and/or vectors comprising the polynucleotides/nucleic acid constructs of the invention and/or optionally one or more guide nucleic acids may be provided. In some embodiments, a nucleic acid construct encoding a modified CRISPR-Cas nuclease and/or a fusion protein comprising a CRISPR-Cas nuclease modified as described herein may be comprised in the same or a separate expression cassette or vector from that comprising the guide nucleic acid. When the nucleic acid construct is comprised in a separate expression cassette or vector from that comprising the guide nucleic acid, a target nucleic acid may be contacted with (e.g., provided with) the expression cassette or vector comprising the nucleic acid construct of the invention prior to, concurrently with, or after the expression cassette comprising the guide nucleic acid is provided (e.g., contacted with the target nucleic acid).

In some embodiments, the present invention provides expression cassettes and/or vectors encoding compositions and/or complexes of the invention or comprising systems of the invention.

In some embodiments, the polynucleotides, nucleic acid constructs, expression cassettes and/or vectors of the invention that are optimized for expression in an organism may be about 70% to about 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%, and any value or range therein) to the polynucleotides, nucleic acid constructs, expression cassettes and/or vectors encoding the same modified CRISPR-Cas nuclease or fusion protein of the invention but which has not been optimized for expression in an organism. An organism for which a polynucleotide or nucleic acid construct may be optimized may include but is not limited to, an animal, a plant, a fungus, an archaeon, or a bacterium. In some embodiments, a polynucleotide or nucleic acid construct of the invention is optimized for expression in a plant.

In some embodiments, the invention provides cells comprising one or more polynucleotides, guide nucleic acids, nucleic acid constructs, systems, expression cassettes and/or vectors of the invention.

The nucleic acid constructs of the invention (e.g., encoding a modified CRISPR-Cas nuclease of the invention and/or a fusion protein comprising a modified CRISPR-Cas nuclease of the invention) and expression cassettes/vectors comprising the same may be used for modifying target nucleic acids and/or their expression in vivo (e.g., in an organism or the cell of an organism; e.g., a plant) and in vitro (e.g., in a cell or a cell free system).

The present invention further provides methods for altering the PAM specificity of a Cas12a polypeptide. In some embodiments, a method for altering PAM specificity is provided that comprises introducing a mutation into the Cas12a polypeptide wherein the mutation is located at amino acid residue of any one or more of K135, S168, T246, N263, T307, L324, E330, S345, R359, D367, I369, E379, Y381, I417, F474, E479, E484, D489, L498, N590, K167, Q231, A454, F657, F337, L459, I507, M531, D654, E659, S170, V245, I425, V463, L492, K514, N112, E223, Y426, I455, S117, A239, T295, N100, K387, A404, D405, D423, E539, D572, N211, N527, Y553, Y554, L321, K192, I344 and/or E443 with reference to the position numbering of SEQ ID NO: 180. In some embodiments, the mutation at amino acid residue K135, S168, T246, N263, T307, L324, E330, S345, R359, D367, I369, E379, Y381, I417, F474, E479, E484, D489, L498, N590, K167, Q231, A454, F657, F337, L459, I507, M531, D654, E659, S170, V245, I425, V463, L492, K514, N112, E223, Y426, I455, S117, A239, T295, N100, K387, A404, D405, D423, E539, D572, N211, N527, Y553, Y554, L321, K192, I344 and/or E443 that is introduced into the Cas12a polypeptide is K116R, K116N, K116D, K120R, K120H, K120N, K120T, K120Y, K120Q, K121S, K121T, K121H, K121R, K121G, K121D, K121Q, D122R, D122K, D122H, D122E, D122N, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D1561, D156V, D156L, D156E, E159K, E159R, E159H, E159Y, E159Q, Q529N, Q529T, Q529H, Q529A, Q529F, Q529G, Q529G, Q529S, Q529P, Q529W, Q529D, G532D, G532N, G532S, G532H, G532F, G532K, G532R, G532Q, G532A, G532L, G532C, D535N, D535H, D535V, D535T, D535,S D535A, D535W, D535K, K538R K538V, K538Q, K538W, K538Y, K538F, K538H, K538L, K538M, K538C, K538G, K538A, K538P, D541N, D541H, D541R, D541K, D541Y, D5411, D541A, D541S, D541E, Y542R, Y542K, Y542H, Y542Q, Y542F, Y542L, Y542M, Y542P, Y542V, Y542N, Y542T, L585G, L585H, L585F, K591W, K591F, K591Y, K591H, K591R, K591S, K591A, K591G, K591P, M592R, M592K, M592Q, M592E, M592A, K595R, K595Q, K595Y, K595L, K595W, K595H, K595E, K595S, K595D, K595M, V596T, V596H, V596G, V596A, S599G, S599H, S599N, S599D, K600R, K600H, K600G, K601R, K601H, K601Q, K601T, Y616K, Y616R, Y616E, Y616F, Y616H, Y646R, Y646E, Y646K, Y646H, Y646Q, Y646W, Y646N, W649H, W649K, W649Y, W649R, W649E, W649S, W649V, and/or W649T with reference to position numbering of SEQ ID NO: 180. In some embodiments, the mutation that is introduced into the Cas12a polypeptide may comprise, consist of, or consist essentially of one or more of the amino acid residue positions of K135, S168, T246, N263, T307, L324, E330, S345, R359, D367, I369, E379, Y381, I417, F474, E479, E484, D489, L498, and/or N590 with reference to position numbering of SEQ ID NO: 180. In some embodiments, the mutation may comprise, consist of, or consist essentially of any one or more of K135N, S168N, T246S, N263I, T307S, L324M, E330V, S345R, R359S, D367V, I369M, E379G, Y381H, I417F, F474L, E479V, E484V, D489Y, L498M, N590Y, K167I, Q231, A454V, F657, F337L, L459M, I507V, M531I, D654E, E659V, S170L, V245I, I425V, V463A, L492P, K514N, N112D, E223V, Y426D, 1455F, S117C, A239T, T295I, N100S, K387E, A404V, D405G, D423V, E539V, D572G, N211S, N527S, Y553H, Y554N, L321V, K192I, I344T and/or E443D with reference to position numbering of SEQ ID NO: 180, optionally the mutation may comprise, consist of, or consist essentially of one or more of K135N, S168N, T246S, N263I, T307S, L324M, E330V, S345R, R359S, D367V, I369M, E379G, Y381H, I417F, F474L, E479V, E484V, D489Y, L498M, and/or N590Y with reference to position numbering of SEQ ID NO: 180, optionally the mutation may comprise, consist of, or consist essentially of one or more of D489Y, L324M, E484V and/or E330V with reference to position numbering of SEQ ID NO: 180.

In some embodiments, a method for altering PAM specificity is provided that comprises introducing a mutation into a Cas12a polypeptide wherein the mutation is located at one or more than one of the following positions of E153, S186, R267, I285, Y336, L352, S358, E372, R386, E391, R393, K403, A405, A434, V486, E491, P494, R499, E508, A602, I185, Q252, E465, L698, F365, Q470, A517, A541, L695, P700, A188, S266, I442, L474, G502, K524, T114, P244, L443, I466, D119, Q260, K324, T101, R411, A427, G428, S440, E549, E584, S234, M537, Y563, Y564, A349, R210, L371, and/or K459, optionally a mutation at position E153, S186, R267, I285, Y336, L352, S358, E372, R386, E391, R393, K403, A405, A434, V486, E491, P494, R499, E508, and/or A602, optionally a mutation at position R499, L352, P494 and/or S358, with reference to position numbering of SEQ ID NO: 51, wherein the polypeptide exhibits a reduced PAM stringency and increased recognition of new protospacer adjacent motifs (PAMs) as compared to the amino acid sequence of SEQ ID NO: 51. In some embodiments, the mutation may comprise, consist of, or consist essentially of any one or more of E153, S186, R267, I285, Y336, L352, S358, E372, R386, E391, R393, K403, A405, A434, V486, E491, P494, R499, E508, A602, I185, Q252, E465, L698, F365, Q470, A517, A541, L695, P700, A188, S266, I442, L474, G502, K524, T114, P244, L443, I466, D119, Q260, K324, T101, R411, A427, G428, S440, E549, E584, S234, M537, Y563, Y564, A349, R210, L371, and/or K459 with reference to position numbering of SEQ ID NO: 51, optionally the mutation may comprise, consist of, or consist essentially of one or more of E153, S186, R267, I285, Y336, L352, S358, E372, R386, E391, R393, K403, A405, A434, V486, E491, P494, R499, E508, and/or A602 with reference to position numbering of SEQ ID NO: 51, optionally the mutation may comprise, consist of, or consist essentially of one or more of R499, L352, P494 and/or S358 with reference to position numbering of SEQ ID NO: 51.

In some embodiments, a method for altering PAM specificity is provided that comprises introducing a mutation into a Cas12a polypeptide wherein the mutation is located at one or more than one of the following positions of I162, P196, V290, S308, Q358, L377, K385, T400, Q423, D431, P433, K443, T445, A480, I454, D550, E559, V564, A573, N666, I195, Q275, A525, F741, Y392, L529, I582, A606, G738, D743, S198, F289, I488, T533, E567, K589, S116, A267, F489, A525, N121, T283, E343, T104, L451, R467, D468, P486, E614, E648, T243, N602, Y628, Y629, L374, S220, L399, and/or Q507, optionally a mutation located at I162, P196, V290, S308, Q358, L377, K385, T400, Q423, D431, P433, K443, T445, A480, I454, D550, E559, V564, A573, and/or N666, optionally a mutation at position V564, L377, E559 and/or K385, with reference to position numbering of SEQ ID NO: 55, wherein the polypeptide exhibits a reduced PAM stringency and increased recognition of new protospacer adjacent motifs (PAMs) as compared to the amino acid sequence of SEQ ID NO: 55. In some embodiments, the mutation may comprise, consist of, or consist essentially of any one or more I162, P196, V290, S308, Q358, L377, K385, T400, Q423, D431, P433, K443, T445, A480, I454, D550, E559, V564, A573, N666, I195, Q275, A525, F741, Y392, L529, I582, A606, G738, D743, S198, F289, I488, T533, E567, K589, S116, A267, F489, A525, N121, T283, E343, T104, L451, R467, D468, P486, E614, E648, T243, N602, Y628, Y629, L374, S220, L399, and/or Q507 with reference to position numbering of SEQ ID NO: 55, optionally the mutation may comprise, consist of, or consist essentially of one or more of I162, P196, V290, S308, Q358, L377, K385, T400, Q423, D431, P433, K443, T445, A480, I454, D550, E559, V564, A573, and/or N666 with reference to position numbering of SEQ ID NO: 55, optionally the mutation may comprise, consist of, or consist essentially of one or more of V564, L377, E559 and/or K385 with reference to position numbering of SEQ ID NO: 55.

In some embodiments, in addition to the introduction of a mutation located at amino acid residue of any one or more of K135, S168, T246, N263, T307, L324, E330, S345, R359, D367, I369, E379, Y381, I417, F474, E479, E484, D489, L498, N590, K167, Q231, A454, F657, F337, L459, I507, M531, D654, E659, S170, V245, I425, V463, L492, K514, N112, E223, Y426, I455, S117, A239, T295, N100, K387, A404, D405, D423, E539, D572, N211, N527, Y553, Y554, L321, K192, I344 and/or E443 with reference to the position numbering of SEQ ID NO: 180, the method for altering PAM specificity may further comprise introducing into the Cas12a polypeptide a mutation at one or more than one of the following positions of K116, K120, K121, D122, E125, T148, T149, T152, D156, E159, Q529, D535, K538, D541, Y542, L585, K591, N590, M592, K595, V596, S599, K600, K601, Y616, Y646, and/or W649 with reference to position numbering of SEQ ID NO: 180. In some embodiments, in addition to the introduction of a mutation located at one or more than one of the following positions of E153, S186, R267, I285, Y336, L352, S358, E372, R386, E391, R393, K403, A405, A434, V486, E491, P494, R499, E508, A602, I185, Q252, E465, L698, F365, Q470, A517, A541, L695, P700, A188, S266, I442, L474, G502, K524, T114, P244, L443, I466, D119, Q260, K324, T101, R411, A427, G428, S440, E549, E584, S234, M537, Y563, Y564, A349, R210, L371, and/or K459 with reference to position numbering of SEQ ID NO: 51, the method for altering PAM specificity may further comprise introducing a mutation at one or more than one of the following positions of E127, K548, N552, F598, A602 and/or K607 with reference to position numbering of SEQ ID NO: 51. In some embodiments, in addition to the introduction of a mutation located at one or more than one of the following positions of I162, P196, V290, S308, Q358, L377, K385, T400, Q423, D431, P433, K443, T445, A480, I454, D550, E559, V564, A573, N666, I195, Q275, A525, F741, Y392, L529, I582, A606, G738, D743, S198, F289, I488, T533, E567, K589, S116, A267, F489, A525, N121, T283, E343, T104, L451, R467, D468, P486, E614, E648, T243, N602, Y628, Y629, L374, S220, L399, and/or Q507 with reference to position numbering of SEQ ID NO: 55, the method for altering PAM specificity may further comprise introducing into a mutation at one or more than one of the following positions of D129, K613, N617, L661, N666 and/or K671 with reference to position numbering of SEQ ID NO: 55. In some embodiments, the mutation located at K116, K120, K121, D122, E125, T148, T149, T152, D156, E159, Q529, D535, K538, D541, Y542, L585, K591, N590, M592, K595, V596, S599, K600, K601, Y616, Y646, and/or W649 with reference to position numbering of SEQ ID NO: 180 may be K116R, K116N, K116D, K120R, K120H, K120N, K120T, K120Y, K120Q, K121S, K121T, K121H, K121R, K121G, K121D, K121Q, D122R, D122K, D122H, D122E, D122N, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D1561, D156V, D156L, D156E, E159K, E159R, E159H, E159Y, E159Q, Q529N, Q529T, Q529H, Q529A, Q529F, Q529G, Q529G, Q529S, Q529P, Q529W, Q529D, G532D, G532N, G532S, G532H, G532F, G532K, G532R, G532Q, G532A, G532L, G532C, D535N, D535H, D535V, D535T, D535,S D535A, D535W, D535K, K538R K538V, K538Q, K538W, K538Y, K538F, K538H, K538L, K538M, K538C, K538G, K538A, K538P, D541N, D541H, D541R, D541K, D541Y, D5411, D541A, D541S, D541E, Y542R, Y542K, Y542H, Y542Q, Y542F, Y542L, Y542M, Y542P, Y542V, Y542N, Y542T, L585G, L585H, L585F, K591W, K591F, K591Y, K591H, K591R, K591S, K591A, K591G, K591P, M592R, M592K, M592Q, M592E, M592A, K595R, K595Q, K595Y, K595L, K595W, K595H, K595E, K595S, K595D, K595M, V596T, V596H, V596G, V596A, S599G, S599H, S599N, S599D, K600R, K600H, K600G, K601R, K601H, K601Q, K601T, Y616K, Y616R, Y616E, Y616F, Y616H, Y646R, Y646E, Y646K, Y646H, Y646Q, Y646W, Y646N, W649H, W649K, W649Y, W649R, W649E, W649S, W649V, and/or W649T with reference to position numbering of SEQ ID NO: 180.

In some embodiments, the method for altering PAM specificity may further comprise introducing into a Cas12a polypeptide of any one of the following combination of mutations of (a) (1) D156R, D489Y and K595Y, (2) D156R, L324M, E484V and K595Y, (3) D156R, E330V and D489Y, (4) D156R, D367V, E379G, Y381H, N590Y and K595Y, (5) D156R, I417F and D489Y, (6) D156R, 1417F, K538N and K595Y, (7) D156R, E330V, I417F, and K538N, (8) D156R and E330V, (9) D156R and 1417F, (10) D156R, N263I and L498M, (11) K135N, D156R, T307S, S345R, D489V and K595Y, and/or (12) D156R, S168N, T246S, R359S, I369M, F474L, E479V, K538N, Y542N and K595Y with reference to position numbering of SEQ ID NO: 180; (b) (1) E174, R499 and K607, (2) E174, L352, P494 and K607, (3) E174, S358 and R499, (4) E174, E391, K403, A405, A602 and K607, (5) E174, A434 and R499, (6) E174, A434, K548 and K607, (7) E174, S358, A434 and K548, (8) E174 and S358, (9) E174 and A434, (10) E174, I285 and E508, (11) E153, E174, Y336, E372, R499 and K607, and/or (12) E174, S186, R267, R386, R393, V486, E491, K548, N552 and K607 with reference to position numbering of SEQ ID NO: 51; and/or (c) (1) E184, V564 and K671, (2) E184, L377, E559 and K671, (3) E184, K385 and V564, (4) E184, D431, K443, T445, N666 and K671, (5) E184, A480 and V564, (6) E184, A480, K613 and K671, (7) E184, K385, A480, and K613, (8) E184 and K385, (9) E184 and A480, (10) E184, S308, and A573, (11) I162, E184, Q358, T400, V564 and K671, or (12) E184, P196, V290, Q423, P433, I545, D550, K613, N617 and K671 with reference to position numbering of SEQ ID NO: 55.

A mutation that is introduced may be a single mutation or it may be a combination of two or more mutations. As would be understood, any single Cas12a polypeptide having two or more mutations would comprise only a single mutation at any given position. In some embodiments, the Cas12a polypeptide that is altered in PAM specificity by the methods of this invention is a LbCas12a polypeptide (Lachnospiraceae bacterium). In some embodiments, the Cas12a polypeptide that is altered in PAM specificity by the methods of this invention is a AsCas12a polypeptide (Acidaminococcus sp.). In some embodiments, the Cas12a polypeptide that is altered in PAM specificity by the methods of this invention is a FnCas12a polypeptide (Francisella tularensis).

Cas12a polypeptides or nucleases of the invention modified as described herein may be used for modifying a target nucleic acid in a cell or a cell free system (e.g., altering the target nucleic acid, altering the genome of the cell/organism). Accordingly, in some embodiments, a method of modifying a target nucleic acid is provided, the method comprising: contacting the target nucleic acid with: (a)(i) a Cas12a polypeptide of the invention, or a fusion protein of the invention (e.g., a Cas12a polypeptide of the invention and a polypeptide of interest (e.g., a deaminase)), and (ii) a guide nucleic acid; (b) a complex of the invention comprising (i) a Cas12a polypeptide or a fusion protein of the invention, and (ii) a guide nucleic acid; (c) a composition comprising (i) a Cas12a polypeptide of the invention, or a fusion protein of the invention, and (ii) a guide nucleic acid; and/or (d) a system of the invention, thereby modifying the target nucleic acid. Further provided is a method of modifying a target nucleic acid, comprising contacting a cell or a cell free system comprising the target nucleic acid with: (a)(i) a polynucleotide encoding a Cas12a polypeptide of the invention, or an expression cassette or vector comprising the same, and (ii) a guide nucleic acid, or an expression cassette or vector comprising the same; and/or (b) a nucleic acid construct of the invention (e.g., encoding a Cas12a polypeptide of the invention), or an expression cassette or vector comprising the same, thereby modifying the target nucleic acid.

In some embodiments, a method of modifying/altering the genome of a cell or organism is provided, the method comprising: contacting a target nucleic acid in the genome of the cell/organism with: (a)(i) a Cas12a polypeptide of the invention, or a fusion protein of the invention (e.g., a Cas12a polypeptide of the invention and a polypeptide of interest (e.g., a deaminase)), and (ii) a guide nucleic acid; (b) a complex of the invention comprising (i) a Cas12a polypeptide or a fusion protein of the invention, and (ii) a guide nucleic acid; (c) a composition comprising (i) a modified CRISPR-Cas nuclease of the invention, or a fusion protein of the invention, and (ii) a guide nucleic acid; and/or (d) a system of the invention, thereby modifying/altering the genome of a cell or organism. In some embodiments, the cell or organism is a plant cell or a plant.

In some embodiments, a method of modifying a target nucleic acid is provided, the method comprising: contacting a cell or a cell free system comprising the target nucleic acid with: (a)(i) a polynucleotide of the invention (e.g., encoding a Cas12a polypeptide of the invention, or encoding fusion protein comprising a Cas12a polypeptide of the invention and a polypeptide of interest (e.g., a deaminase)), or an expression cassette or vector comprising the same, and (ii) a guide nucleic acid, or an expression cassette and/or vector comprising the same; and/or (b) a nucleic acid construct encoding a complex of the invention comprising a Cas12a polypeptide of the invention, or fusion protein comprising a Cas12a polypeptide of the invention and a polypeptide of interest (e.g., a deaminase), or an expression cassette and/or vector comprising the same, wherein the contacting is carried out under conditions whereby the polynucleotide and/or nucleic acid construct is/are expressed and the Cas12a polypeptide and/or fusion protein is/are produced, which forms a complex with the guide nucleic acid, thereby modifying the target nucleic acid. In some embodiments, a method of modifying/altering the genome of a cell and/or organism is provided, the method comprising: contacting a cell and/or cell in an organism comprising the target nucleic acid with: (a)(i) a polynucleotide of the invention (e.g., encoding a Cas12a polypeptide of the invention, or encoding fusion protein comprising a Cas12a polypeptide of the invention and a polypeptide of interest (e.g., a deaminase)), or an expression cassette or vector comprising the same, and (ii) a guide nucleic acid, or an expression cassette and/or vector comprising the same; and/or (b) a nucleic acid construct encoding a complex of the invention comprising a Cas12a polypeptide of the invention, or fusion protein comprising a Cas12a polypeptide of the invention and a polypeptide of interest (e.g., a deaminase), or an expression cassette and/or vector comprising the same, wherein the contacting is under conditions whereby the polynucleotide and/or nucleic acid construct is/are expressed and the Cas12a polypeptide and/or fusion protein is/are produced, which form(s) a complex with the guide nucleic acid, thereby modifying the target nucleic acid.

In some embodiments, the present invention provides a method of editing a target nucleic acid, the method comprising: contacting the target nucleic acid with: (a)(i) a fusion protein of the present invention (comprising a Cas12a polypeptide of the invention and a polypeptide of interest (e.g., a deaminase), and (a)(ii) a guide nucleic acid; (b) a complex comprising a fusion protein of the present invention, and a guide nucleic acid; (c) a composition comprising the fusion protein of the present invention and a guide nucleic acid; and/or (d) a system of the invention, thereby editing the target nucleic acid.

In some embodiments, the present invention provides a method of editing a target nucleic acid, the method comprising: contacting a cell or a cell free system comprising the target nucleic acid with: (a)(i) a polynucleotide encoding a fusion protein of the invention (e.g., a Cas12a polypeptide of the invention and a polypeptide of interest (e.g. a deaminase)) or an expression cassette and/or vector comprising the same, and (a)(ii) a guide nucleic acid, or an expression cassette and/or vector comprising the same; (b) a nucleic acid construct encoding a complex comprising a fusion protein of the present invention, and a guide nucleic acid, or an expression cassette and/or vector comprising the same; and/or (c) a system of the present invention wherein the contacting is carried out under conditions whereby the polynucleotide and/or nucleic acid construct is/are expressed and a CRISPR-Cas nuclease modified as described herein and/or fusion protein is/are produced, which form(s) a complex with the guide nucleic acid, thereby editing the target nucleic acid.

The CRISPR-Cas nucleases having modified PAM recognition specificities may be utilized in many ways including, but not limited to, creating indels (NHEJ), in homology directed repair, as a genome recognition element without a nuclease function (dead Cas12a), as a genome recognition element with a partially functional nuclease (nickase Cas12a), in fusion proteins for catalytic editing of genomic DNA (DNA base editors), in fusions proteins for catalytic editing of RNA (RNA base editors), for targeting of other macromolecules to specific genomic regions; for targeting of small chemicals to specific genomic regions, for labeling of specific genomic regions and/or for CRISPR-directed genomic recombination strategies.

When provided in different nucleic acid constructs, expression vectors, and/or vectors, a nucleic acid construct of the invention may be contacted with a target nucleic acid prior to, concurrently with or after contacting the target nucleic acid with a guide nucleic acid.

The modified CRISPR-Cas nucleases of the invention and polypeptides and nucleic acid constructs encoding the same may be used for modifying a target nucleic acid in any organism, including but not limited to, an animal, a plant, a fungus, an archaeon, or a bacterium. An animal can include, but is not limited to, a mammal, an insect, a fish, a bird, and the like. Exemplary mammals for which this invention may be useful include, but are not limited to, primates (human and non-human (e.g., a chimpanzee, baboon, monkey, gorilla, etc.)), cats, dogs, mice, rats, ferrets, gerbils, hamsters, cows, pigs, horses, goats, donkeys, or sheep In some embodiments, the methods of the invention are not used for modifying a target nucleic acid in an animal. In some embodiments, the methods of the invention are not used for modifying a target nucleic acid in a mammal. In some embodiments, the methods of the invention are not used for modifying a target nucleic acid in a human.

The present invention further provides an engineered protein, the engineered protein comprising: a first polypeptide that is a first portion of a modified protein, wherein the modified protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more identity to the amino acid sequence of SEQ ID NO: 180 (LbCas12a) and, relative to the amino acid sequence of SEQ ID NO: 180, comprises a mutation at one or more of the following positions selected from the group consisting of: N100, K116, K120, K121, D122, E125, T148, T149, T152, D156, E159, N211, N263, T295, E330, K387, A404, D405, D423, E484, L498, N527, Q529, G532, D535, K538, E539, D541, Y542, Y553, Y554, D572, L585, K591, M592, K595, V596, S599, K600, K601, Y616, Y646, W649, N112, S117, K135, K167, S168, S170, K192, E223, Q231, A239, V245, T246, T307, L321, L324, F337, I344, S345, R359, D367, I369, E379, Y381, I417, I425, Y426, E443, A454, I455, L459, V463, F474, E479, D489, L492, I507, K514, M531, N590, F657, D654, E659 and any combination thereof with reference to position numbering of SEQ ID NO: 180; and a second polypeptide that is heterologous to the first polypeptide and is not a Type V CRISPR-Cas effector polypeptide, wherein the first polypeptide and second polypeptide are different from each other, and wherein the engineered protein comprises the mutation. In some embodiments, one or more (e.g., 1, 2, 3, 4, 5, or more) portion(s) of the engineered protein are a portion of the modified protein and the one or more portion(s) may be the same or different from each other. In some embodiments, the engineered protein comprises at least two different portions of the modified protein and the first portion and/or second portion may comprise one or more mutation(s) as described herein for the modified protein. In some embodiments, the first polypeptide comprises the mutation and/or the second polypeptide comprises the mutation. In some embodiments, a Type V CRISPR-Cas effector polypeptide of the engineered protein comprises the one or more mutation(s). In some embodiments, the first polypeptide comprises the mutation relative to the amino acid sequence of SEQ ID NO: 180 and the mutation is at one or more of the following positions selected from the group consisting of: N100, K116, K120, K121, D122, E125, T148, T149, T152, D156, E159, N211, N263, T295, E330, K387, A404, D405, D423, E484, L498, N527, Q529, G532, D535, K538, E539, D541, Y542, Y553, Y554, D572, L585, K591, M592, K595, V596, S599, K600, K601, Y616, Y646, W649, N112, S117, K135, K167, S168, S170, K192, E223, Q231, A239, V245, T246, T307, L321, L324, F337, I344, S345, R359, D367, I369, E379, Y381, I417, I425, Y426, E443, A454, I455, L459, V463, F474, E479, D489, L492, I507, K514, M531, N590, F657, D654, E659, and any combination thereof with reference to position numbering of SEQ ID NO: 180. For example, the first polypeptide, optionally when optimally aligned to the amino acid sequence of SEQ ID NO: 180, has at least one mutation at the position(s) of N100, K116, K120, K121, D122, E125, T148, T149, T152, D156, E159, N211, N263, T295, E330, K387, A404, D405, D423, E484, L498, N527, Q529, G532, D535, K538, E539, D541, Y542, Y553, Y554, D572, L585, K591, M592, K595, V596, S599, K600, K601, Y616, Y646, W649, N112, S117, K135, K167, S168, S170, K192, E223, Q231, A239, V245, T246, T307, L321, L324, F337, I344, S345, R359, D367, I369, E379, Y381, I417, I425, Y426, E443, A454, I455, L459, V463, F474, E479, D489, L492, I507, K514, M531, N590, F657, D654, E659, and any combination thereof with reference to position numbering of SEQ ID NO: 180. In some embodiments, the engineered protein comprises a third polypeptide that is a portion of the modified protein. The third polypeptide, optionally when optimally aligned to the amino acid sequence of SEQ ID NO: 180, may have at least one mutation at the position(s) of: N100, K116, K120, K121, D122, E125, T148, T149, T152, D156, E159, N211, N263, T295, E330, K387, A404, D405, D423, E484, L498, N527, Q529, G532, D535, K538, E539, D541, Y542, Y553, Y554, D572, L585, K591, M592, K595, V596, S599, K600, K601, Y616, Y646, W649, N112, S117, K135, K167, S168, S170, K192, E223, Q231, A239, V245, T246, T307, L321, L324, F337, I344, S345, R359, D367, I369, E379, Y381, I417, I425, Y426, E443, A454, I455, L459, V463, F474, E479, D489, L492, I507, K514, M531, N590, F657, D654, E659, and any combination thereof with reference to position numbering of SEQ ID NO: 180. In some embodiments, the modified protein of the engineered protein, optionally when optimally aligned to the amino acid sequence of SEQ ID NO: 180, may comprise one or more amino acid mutations comprising, consisting of, or consisting essentially of: N100S, K116D, K116R, K116N, K120R, K120H, K120N, K120T, K120Y, K120Q, K121S, K121T, K121H, K121R, K121G, K121D, K121Q, D122R, D122K, D122H, D122E, D122N, E125G, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D1561, D156V, D156L, D156E, E159K, E159R, E159H, E159Y, E159Q, N211S, N263I, T295I, E330V, K387E, A404V, D405G, D423V, E484D, L498M, N527S, Q529N, Q529T, Q529H, Q529A, Q529F, Q529G, Q529S, Q529P, Q529W, Q529D, G532D, G532N, G532S, G532H, G532F, G532K, G532R, G532Q, G532A, G532L, G532C, D535N, D535H, D535V, D535T, D535S, D535A, D535W, D535K, K538R, K538V, K538Q, K538W, K538Y, K538F, K538H, K538L, K538M, K538C, K538G, K538A, K538P, E539V, D541N, D541H, D541R, D541K, D541Y, D5411, D541A, D541S, D541E, Y542R, Y542K, Y542H, Y542Q, Y542F, Y542L, Y542M, Y542P, Y542V, Y542N, Y542T, Y553H, Y554N, D572G, L585Q, L585G, L585H, L585F, K591W, K591F, K591Y, K591H, K591R, K591S, K591A, K591G, K591P, M592R, M592K, M592Q, M592E, M592A, K595R, K595Q, K595Y, K595L, K595W, K595H, K595E, K595S, K595D, K595M, V596T, V596H, V596G, V596A, S599G, S599H, S599N, S599D, K600R, K600H, K600G, K601R, K601H, K601Q, K601T, Y616K, Y616R, Y616E, Y616F, Y616H, Y646R, Y646E, Y646K, Y646H, Y646Q, Y646W, Y646N, W649H, W649K, W649Y, W649R, W649E, W649S, W649V, W649T, N112D, S117C, K135N, K167I, S168N, S170L, K192I, E223V, Q231, A239T, V245I, T246S, T307S, L321V, L324M, F337L, 1344T, S345R, R359S, D367V, I369M, E379G, Y381H, I417F, 1425V, Y426D, E443D, A454V, 1455F, L459M, F474L, E479V, D489Y, L492P, 1507V, K514N, M531I, N590Y, D654E, F657, E659V, V463A and any combination thereof with reference to the position numbering of SEQ ID NO: 180. In some embodiments, a mutation in the modified protein of the engineered protein, optionally when optimally aligned to the amino acid sequence of SEQ ID NO: 180, may comprise, consist of, or consist essentially of any one or more of: N100S, K116D, E125G, T152R, D156E, N211S, N263I, T295I, E330V, K387E, A404V, D405G, D423V, E484D, L498M, N527S, G532R, K538V, E539V, Y542R, Y553H, Y554N, D572G, L585Q, K595R, K595Y, N112D, S117C, K135N, K167I, S168N, S170L, K192I, E223V, Q231, A239T, V245I, T246S, T307S, L321V, L324M, F337L, 1344T, S345R, R359S, D367V, I369M, E379G, Y381H, I417F, 1425V, Y426D, E443D, A454V, 1455F, L459M, V463A F474L, E479V, D489Y, L492P, I507V, K514N, M531I, N590Y, D654E, F657, E659V and any combination thereof with reference to the position numbering of SEQ ID NO: 180.

In some embodiments, modified protein of the engineered protein, optionally when optimally aligned to the amino acid sequence of SEQ ID NO: 180, may comprise, consist of, or consist essentially of any one of the following combinations of mutations: (1) D156R, D489Y and K595Y, (2) D156R, L324M, E484V and K595Y, (3) D156R, E330V and D489Y, (4) D156R, D367V, E379G, Y381H, N590Y and K595Y, (5) D156R, I417F and D489Y, (6) D156R, I417F, K538N and K595Y, (7) D156R, E330V, I417F, and K538N, (8) D156R and E330V, (9) D156R and 1417F, (10) D156R, N263I and L498M, (11) K135N, D156R, T307S, S345R, D489V and K595Y, and/or (12) D156R, S168N, T246S, R359S, I369M, F618L, E479V, K538N, Y686N and K595Y with reference to the position numbering of SEQ ID NO: 180.

As would be understood, an engineered protein having two or more mutations would comprise only a single mutation at any given position. Thus, for example, a polypeptide may have mutation at position K120 that may be only one of the mutations of K120R, K120H, K120N, K120T, K120Y, or K120Q.

In some embodiment, the invention further provides an engineered protein comprising: an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more identity to the amino acid sequence of SEQ ID NO: 107 (SYN3298); and the engineered protein comprises a mutation relative to the amino acid sequence of SEQ ID NO: 107 (SYN3298) (e.g., optimally aligned to SEQ ID NO: 107) a mutation at one or more of the following positions selected from the group consisting of: N100, K116, K120, K121, D122, E125, T148, T149, T152, D156, E159, N211, N263, T443, E478, K535, A552, D553, D571, E632, L646, N675, Q677, G680, D683, K686, E687, D689, Y690, Y701, Y702, D720, L733, K739, M740, K743, V744, S747, K748, K749, Y764, Y794, W797, N112, S117, K135, K167, S168, S170, K192, E223, Q231, A239, V245, T246, T455, L469, L472, F485, I492, S493, R507, D515, I517, E527, Y529, I565, I573, Y574, E591, A602, I603, L607, V611, F622, E627, D637, L640, I655, K662, M679, N738, D802, F805, E807 and any combination thereof with reference to residue position numbering of SEQ ID NO: 107 (SYN3298). For example, an engineered protein of the present invention, optionally when optimally aligned to the amino acid sequence of SEQ ID NO: 107, has at least one mutation at the position(s) of N100, K116, K120, K121, D122, E125, T148, T149, T152, D156, E159, N211, N263, T443, E478, K535, A552, D553, D571, E632, L646, N675, Q677, G680, D683, K686, E687, D689, Y690, Y701, Y702, D720, L733, K739, M740, K743, V744, S747, K748, K749, Y764, Y794, W797, N112, S117, K135, K167, S168, S170, K192, E223, Q231, A239, V245, T246, T455, L469, L472, F485, I492, S493, R507, D515, I517, E527, Y529, I565, I573, Y574, E591, A602, I603, L607, V611, F622, E627, D637, L640, I655, K662, M679, N738, D802, F805, E807, and any combination thereof with reference to position numbering of SEQ ID NO: 107. In some embodiments, the engineered protein comprises a first polypeptide that is a Type V CRISPR-Cas effector polypeptide and that is devoid of a nuclease domain, and a second polypeptide that is heterologous to the first polypeptide and that is not a Type V CRISPR-Cas effector polypeptide. The engineered protein may further comprise a third polypeptide that is a Type V CRISPR-Cas effector polypeptide. Any portion of the engineered protein may include the one or more (e.g., 1, 2, 3, 4, 5, or more) mutation(s). In some embodiments, one or more (e.g., 1, 2, 3, 4, 5, or more) portions of the engineered protein are a portion of the amino acid sequence of SEQ ID NO: 107 (SYN3298) and the one or more portions may be the same or different from each other. In some embodiments, the engineered protein comprises at least two different portions of the amino acid sequence of SEQ ID NO: 107 (SYN3298) and a first portion and/or a second portion may comprise the one or more mutation(s) as described herein. In some embodiments, the engineered protein comprises at least three different portions of the amino acid sequence of SEQ ID NO: 107 (SYN3298) and a first portion and/or a second portion may comprise the one or more mutation(s) as described herein and a third portion (e.g., a heterologous polypeptide) may not comprise the one or more mutation(s). In some embodiments, a Type V CRISPR-Cas effector polypeptide of the engineered protein comprises the one or more mutation(s).

In some embodiments, a mutation in the modified protein of the engineered protein, optionally when optimally aligned to the amino acid sequence of SEQ ID NO: 107, may comprise, consist of, or consist essentially of any one or more of: N100S, K116D, K116R, K116N, K120R, K120H, K120N, K120T, K120Y, K120Q, K121S, K121T, K121H, K121R, K121G, K121D, K121Q, D122R, D122K, D122H, D122E, D122N, E125G, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D1561, D156V, D156L, D156E, E159K, E159R, E159H, E159Y, E159Q, N211S, N263I, T4431, E478V, K535E, A552V, D553G, D571V, E632D, L646M, N675S, Q677N, Q677T, Q677H, Q677A, Q677F, Q677G, Q677S, Q677P, Q677W, Q677D, G680D, G680N, G680S, G680H, G680F, G680K, G680R, G680Q, G680A, G680L, G680C, D683N, D683H, D683V, D683T, D683S, D683A, D683W, D683K, K686R, K686V, K686Q, K686W, K686Y, K686F, K686H, K686L, K686M, K686C, K686G, K686A, K686P, E687V, D689N, D689H, D689R, D689K, D689Y, D6891, D689A, D689S, D689E, Y690R, Y690K, Y690H, Y690Q, Y690F, Y690L, Y690M, Y690P, Y690V, Y690N, Y690T, Y701H, Y702N, D720G, L733Q, L733G, L733H, L733F, K739W, K739F, K739Y, K739H, K739R, K739S, K739A, K739G, K739P, M740R, M740K, M740Q, M740E, M740A, K743R, K743Q, K743Y, K743L, K743W, K743H, K743E, K743S, K743D, K743M, V744T, V744H, V744G, V744A, S747G, S747H, S747N, S747D, K748R, K748H, K748G, K749R, K749H, K749Q, K749T, Y764K, Y764R, Y764E, Y764F, Y764H, Y794R, Y794E, Y794K, Y794H, Y794Q, Y794W, Y794N, W797H, W797K, W797Y, W797R, W797E, W797S, W797V, W797T, N112D, S117C, K135N, K167I, S168N, S170L, K192I, E223V, Q231, A239T, V245I, T246S, T455S, L321V, L324M, F337L, 1344T, S345R, R359S, D367V, I369M, E379G, Y381H, I417F, 1425V, Y426D, E443D, A454V, 1455F, L459M, V463A, F474L, E479V, D489Y, L492P, I507V, K514N, M531I, N590Y, D654E, F657, E659V, and any combination thereof with reference to the position numbering of SEQ ID NO: 107. In some embodiments, the engineered protein, optionally when optimally aligned to the amino acid sequence of SEQ ID NO: 107, comprises one or more amino acid mutation(s) comprising, consisting essentially of, consisting of: N100S, K116D, E125G, T152R, D156E, N211S, N263I, T4431, E478V, K535E, A552V, D553G, D571V, E632D, L646M, N675S, G680R, K686V, E687V, Y690R, Y701H, Y702N, D720G, L733Q, K743R, K743Y, N112D, S117C, K135N, K167I, S168N, S170L, K192I, E223V, Q231, A239T, V245I, T246S, T455S, L321V, L324M, F337L, 1344T, S345R, R359S, D367V, I369M, E379G, Y381H, I417F, 1425V, Y426D, E443D, A454V, 1455F, L459M, V463A, F474L, E479V, D489Y, L492P, I507V, K514N, M531I, N590Y, D654E, F657, E659V, and any combination thereof with reference to the position numbering of SEQ ID NO: 107.

In some embodiments, modified protein of the engineered protein, optionally when optimally aligned to the amino acid sequence of SEQ ID NO: 107, may comprise, consist of, or consist essentially of any one of the following combinations of mutations: (1) D156R, D637Y and K743Y, (2) D156R, L472M, E632V and K743Y, (3) D156R, E478V, D637Y, (4) D156R, D515V, E527G, Y529H, N738Y and K743Y, (5) D156R, 1565F and D637Y, (6) D156R, I565F, K686N and K743Y, (7) D156R, E478V V, I565F, and K686N, (8) D156R and E478V, (9) D156R and I565F, (10) D156R, N263I and L646M, (11) K135N, D156R, T455S, S493R, D637V and K743Y, and/or (12) D156R, S168N, T246S, R507S, I517M, F474L, E627V, K686N, Y542N and K743Y with reference to the amino acid residue positions of SEQ ID NO: 107.

In some embodiment, the invention further provides is an engineered protein comprising: an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more identity to the amino acid sequence of SEQ ID NO: 105 (SYN3287); and the engineered protein comprises a mutation relative to the amino acid sequence of SEQ ID NO: 105 (SYN3287) at one or more of the following positions selected from the group consisting of: N100, K116, K120, K121, D122, E125, T148, T149, T152, D156, E159, N211, N263, T439, E474, K531, A548, D549, D567, E628, L642, N671, Q673, G676, D679, K682, E683, D685, Y686, Y697, Y698, D716, L729, K735, M736, K739, V740, S743, K744, K745, Y760, Y790, W793, N112, S117, K135, K167, S168, S170, K192, E223, Q231, A239, V245, T246, T451, L465, L468, F481, 1488, S489, R503, D511, 1513, E523, Y525, 1561, 1569, Y570, E587, A598, 1599, L603, V607, F618, E623, D633, L636, 1651, K658, M675, N734, D798, F801, E803 and any combination thereof with reference to amino acid residue position numbering of SEQ ID NO: 105 (SYN3287). In some embodiments, the engineered protein comprises a first polypeptide that is a Type V CRISPR-Cas effector polypeptide and that is devoid of a nuclease domain, and a second polypeptide that is heterologous to the first polypeptide and that is not a Type V CRISPR-Cas effector polypeptide. The engineered protein may further comprise a third polypeptide that is a Type V CRISPR-Cas effector polypeptide. Any portion of the engineered protein may include the one or more (e.g., 1, 2, 3, 4, 5, or more) mutation(s). In some embodiments, one or more (e.g., 1, 2, 3, 4, 5, or more) portions of the engineered protein are a portion of the amino acid sequence of SEQ ID NO: 105 (SYN3287) and the one or more portions may be the same or different from each other. In some embodiments, the engineered protein comprises at least two different portions of the amino acid sequence of SEQ ID NO: 105 (SYN3287) and a first portion and/or a second portion may comprise the one or more mutation(s) as described herein. In some embodiments, the engineered protein comprises at least three different portions of the amino acid sequence of SEQ ID NO: 105 (SYN3287) and a first portion and/or a second portion may comprise the one or more mutation(s) as described herein and a third portion (e.g., a heterologous polypeptide) may not comprise the one or more mutation(s). In some embodiments, a Type V CRISPR-Cas effector polypeptide of the engineered protein comprises the one or more mutation(s). In some embodiments, the engineered protein, optionally when optimally aligned to the amino acid sequence of SEQ ID NO: 105 may comprise, consist of, or consist essentially of any one or more of the amino acid mutations of: N100S, K116D, K116R, K116N, K120R, K120H, K120N, K120T, K120Y, K120Q, K121S, K121T, K121H, K121R, K121G, K121D, K121Q, D122R, D122K, D122H, D122E, D122N, E125G, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D1561, D156V, D156L, D156E, E159K, E159R, E159H, E159Y, E159Q, N211S, N263I, T4391, E474V, K531E, A548V, D549G, D567V, E628D, L642M, N671S, Q673N, Q673T, Q673H, Q673A, Q673F, Q673G, Q673S, Q673P, Q673W, Q673D, G676D, G676N, G676S, G676H, G676F, G676K, G676R, G676Q, G676A, G676L, G676C, D679N, D679H, D679V, D679T, D679S, D679A, D679W, D679K, K682R, K682V, K682Q, K682W, K682Y, K682F, K682H, K682L, K682M, K682C, K682G, K682A, K682P, E683V, D685N, D685H, D685R, D685K, D685Y, D6851, D685A, D685S, D685E, Y686R, Y686K, Y686H, Y686Q, Y686F, Y686L, Y686M, Y686P, Y686V, Y686N, Y686T, Y697H, Y698N, D716G, L729Q, L729G, L729H, L729F, K735W, K735F, K735Y, K735H, K735R, K735S, K735A, K735G, K735P, M736R, M736K, M736Q, M736E, M736A, K739R, K739Q, K739Y, K739L, K739W, K739H, K739E, K739S, K739D, K739M, V740T, V740H, V740G, V740A, S743G, S743H, S743N, S743D, K744R, K744H, K744G, K745R, K745H, K745Q, K745T, Y760K, Y760R, Y760E, Y760F, Y760H, Y790R, Y790E, Y790K, Y790H, Y790Q, Y790W, Y790N, W793H, W793K, W793Y, W793R, W793E, W793S, W793V, W793T, N112D, S117C, K135N, K167I, S168N, S170L, K192I, E223V, Q231, A239T, V245I, T451S, L465V, L468M, F481L, 1488T, S489R, R503S, D511V, 1513M, E523G, Y525H, 1561F, 1569V, Y570D, E587D, A598V, 1599F, L603M, V607A, F618L, E623V, D633Y, L636P, 1651V, K658N, M6751, N734Y, D798E, F801, E803V, and any combination thereof with reference to the position numbering of SEQ ID NO: 105. In some embodiments, the mutation may be one or more amino acid mutations selected from the group consisting of: N100S, K116D, E125G, T152R, D156E, N211S, N263I, T4391, E474V, K531E, A548V, D549G, D567V, E628D, L642M, N671S, G676R, K682V, E683V, Y686R, Y697H, Y698N, D716G, L729Q, K739R, K739Y, N112D, S117C, K135N, K167I, S168N, S170L, K192I, E223V, Q231, A239T, V245I, T451S, L465V, L468M, F481L, 1488T, S489R, R503S, D511V, 1513M, E523G, Y525H, 1561F, 1569V, Y570D, E587D, A598V, 1599F, L603M, V607A, F618L, E623V, D633Y, L636P, 1651V, K658N, M6751, N734Y, D798E, F801, E803V, and any combination thereof with reference to the position numbering of SEQ ID NO: 105.

In some embodiments, modified protein of the engineered protein, optionally when optimally aligned to the amino acid sequence of SEQ ID NO: 105, may comprise, consist of, or consist essentially of any one of the following combinations of mutations: (1) D156R, D633Y and K739Y, (2) D156R, L468M, E628V and K739Y, (3) D156R, E474V and D633Y, (4) D156R, D511V, E523G, Y525H, N734Y and K739Y, (5) D156R, 1561F and D633Y, (6) D156R, 1561F, K682N and K739Y, (7) D156R, E474V, 1561F, and K682N, (8) D156R and E474V, (9) D156R and I563F, (10) D156R, N263I and L642M, (11) K135N, D156R, T451S, S489R, D633V and K739Y, and/or (12) D156R, S168N, T246S, R503S, 1513M, F620L, E623V, K682N, Y688N and K739Y with reference to the amino acid residue positions of SEQ ID NO: 105.

In some embodiment, the invention further provides is an engineered protein comprising: an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more identity to the amino acid sequence of SEQ ID NO: 106 (SYN3288); and the engineered protein comprises a mutation relative to the amino acid sequence of SEQ ID NO: 106 (SYN3288) at one or more of the following positions selected from the group consisting of: N100, K116, K120, K121, D122, E125, T148, T149, T152, D156, E159, N211, N263, T441, E476, K533, A550, D551, D569, E630, L644, N673, Q675, G678, D681, K684, E685, D687, Y688, Y699, Y700, D718, L731, K737, M738, K741, V742, S745, K746, K747, Y762, Y792, W795, N112, S117, K135, K167, S168, S170, K192, E223, Q231, A239, V245, T246, T453, L467, L470, F483, I490, S491, R505, D513, I515, E525, Y527, 1563, 1571, Y572, E589, A600, I601, L605, V609, F620, E625, D635, L638, I653, K660, M677, N736, D800, F803, E805 and any combination thereof with reference to residue position numbering of SEQ ID NO: 106 (SYN3288). In some embodiments, the engineered protein comprises a first polypeptide that is a Type V CRISPR-Cas effector polypeptide and that is devoid of a nuclease domain, and a second polypeptide that is heterologous to the first polypeptide and that is not a Type V CRISPR-Cas effector polypeptide. The engineered protein may further comprise a third polypeptide that is a Type V CRISPR-Cas effector polypeptide. Any portion of the engineered protein may include the one or more (e.g., 1, 2, 3, 4, 5, or more) mutation(s). In some embodiments, one or more (e.g., 1, 2, 3, 4, 5, or more) portions of the engineered protein are a portion of the amino acid sequence of SEQ ID NO: 106 (SYN3288) and the one or more portions may be the same or different from each other. In some embodiments, the engineered protein comprises at least two different portions of the amino acid sequence of SEQ ID NO: 106 (SYN3288) and a first portion and/or a second portion may comprise the one or more mutation(s) as described herein. In some embodiments, the engineered protein comprises at least three different portions of the amino acid sequence of SEQ ID NO: 106 (SYN3288) and a first portion and/or a second portion may comprise the one or more mutation(s) as described herein and a third portion (e.g., a heterologous polypeptide) may not comprise the one or more mutation(s). In some embodiments, a Type V CRISPR-Cas effector polypeptide of the engineered protein comprises the one or more mutation(s). In some embodiments, a mutation in the modified protein of the engineered protein, optionally when the engineered protein is optimally aligned to the amino acid sequence of SEQ ID NO: 106, may comprise, consist of, or consist essentially of any one or more of: N100S, K116D, K116R, K116N, K120R, K120H, K120N, K120T, K120Y, K120Q, K121S, K121T, K121H, K121R, K121G, K121D, K121Q, D122R, D122K, D122H, D122E, D122N, E125G, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D1561, D156V, D156L, D156E, E159K, E159R, E159H, E159Y, E159Q, N211S, N263I, T4411, E476V, K533E, A550V, D551G, D569V, E630D, L644M, N673S, Q675N, Q675T, Q675H, Q675A, Q675F, Q675G, Q675S, Q675P, Q675W, Q675D, G678D, G678N, G678S, G678H, G678F, G678K, G678R, G678Q, G678A, G678L, G678C, D681N, D681H, D681V, D681T, D681S, D681A, D681W, D681K, K684R, K684V, K684Q, K684W, K684Y, K684F, K684H, K684L, K684M, K684C, K684G, K684A, K684P, E685V, D687N, D687H, D687R, D687K, D687Y, D6871, D687A, D687S, D687E, Y688R, Y688K, Y688H, Y688Q, Y688F, Y688L, Y688M, Y688P, Y688V, Y688N, Y688T, Y699H, Y700N, D718G, L731Q, L731G, L731H, L731F, K737W, K737F, K737Y, K737H, K737R, K737S, K737A, K737G, K737P, M738R, M738K, M738Q, M738E, M738A, K741R, K741Q, K741Y, K741L, K741W, K741H, K741E, K741S, K741D, K741M, V742T, V742H, V742G, V742A, S745G, S745H, S745N, S745D, K746R, K746H, K746G, K747R, K747H, K747Q, K747T, Y762K, Y762R, Y762E, Y762F, Y762H, Y792R, Y792E, Y792K, Y792H, Y792Q, Y792W, Y792N, W795H, W795K, W795Y, W795R, W795E, W795S, W795V, W795T, N112D, S117C, K135N, K167I, S168N, S170L, K192I, E223V, Q231, A239T, V245I, T246S, T453S, L467V, L470M, F483L, 1490T, S491R, R505S, D513V, I515M, E525G, Y527H, I563F, 1571V, Y572D, E589D, A600V, 1601F, L605M, V609A, F620L, E625V, D635Y, L638P, 1653V, K660N, M6771, N736Y, D800E, F803, E805V, and any combination thereof with reference to the position numbering of SEQ ID NO: 106. In some embodiments, the mutation in the engineered protein may be one or more amino acid mutations selected from the group consisting of: N100S, K116D, E125G, T152R, D156E, N211S, N263I, T4411, E476V, K533E, A550V, D551G, D569V, E630D, L644M, N673S, G678R, K684V, E685V, Y688R, Y699H, Y700N, D718G, L731Q, K741R, K741Y, N112D, S117C, K135N, K167I, S168N, S170L, K192I, E223V, Q231, A239T, V245I, T246S, T453S, L467V, L470M, F483L, 1490T, S491R, R505S, D513V, I515M, E525G, Y527H, I563F, 1571V, Y572D, E589D, A600V, I601F, L605M, V609A, F620L, E625V, D635Y, L638P, 1653V, K660N, M6771, N736Y, D800E, F803, E805V, and any combination thereof with reference to the position numbering of SEQ ID NO: 106.

In some embodiments, modified protein of the engineered protein, optionally when optimally aligned to the amino acid sequence of SEQ ID NO: 106, may comprise, consist of, or consist essentially of any one of the following combinations of mutations: (1) D156R, D635Y and K741Y, (2) D156R, L470M, E630V and K741Y, (3) D156R, E476V and D635Y, (4) D156R, D513V, E525G, Y527H, N736Y and K741Y, (5) D156R, I563F and D635Y, (6) D156R I563F, K684N and K741Y, (7) D156R, E476V, I563F, and K684N, (8) D156R and E476V, (9) D156R and I563F, (10) D156R, N263I and L644M, (11) K135N, D156R, T453S, S491R, D635V and K741Y, and/or (12) D156R, S168N, T246S, R505S, I515M, F622L, E625V, K684N, Y690N and K741Y with reference to the amino acid residue positions of SEQ ID NO: 106.

In some embodiments, a mutation in a modified protein of the engineered protein of the invention does not comprise of a mutation of D156R, G676R, K682R, K682V, Y686R or K739R with reference to position numbering of SEQ ID NO: 105. In some embodiments, a mutation in a LBCas12a polypeptide does not comprise of a mutation of N263, T439, E474, E628, L642, N100, K531, A548, D549, D567, E683, D716, N211, N671, Y697, and/or Y698 with reference to position numbering of SEQ ID NO: 105. In some embodiments, the mutation in a Cas12a polypeptide of the invention does not comprise the combination of mutations of (a) G676R and K739R, (b) G676R, K682V and Y686R, or (c) D156R, G676R and K676R with reference to position numbering of SEQ ID NO: 105.

In some embodiments, a mutation in a modified protein of the engineered protein of the invention does not comprise of a mutation of D156R, G678R, K684R, K684V, Y688R or K741R with reference to position numbering of SEQ ID NO: 106. In some embodiments, a mutation in a LBCas12a polypeptide does not comprise of a mutation of N263, T441, E476, E630, L644, N100, K533, A550, D551, D569, E685, D718, N211, N673, Y699, and/or Y700 with reference to position numbering of SEQ ID NO: 106. In some embodiments, the mutation in a Cas12a polypeptide of the invention does not comprise the combination of mutations of (a) G678R and K741R, (b) G678R, K684V and Y688R, or (c) D156R, G678R and K678R with reference to position numbering of SEQ ID NO: 106.

In some embodiments, a mutation in a modified protein of the engineered protein of the invention does not comprise of a mutation of D156R, G680R, K686R, K686V, Y690R or K743R with reference to position numbering of SEQ ID NO: 107. In some embodiments, a mutation in a LBCas12a polypeptide does not comprise of a mutation of N263, T443, E478, E632, L646, N100, K535, A552, D553, D571, E687, D720, N211, N675, Y701, and/or Y702 with reference to position numbering of SEQ ID NO: 107. In some embodiments, the mutation in a Cas12a polypeptide of the invention does not comprise the combination of mutations of (a) G680R and K743R, (b) G680R, K686V and Y690R, or (c) D156R, G680R and K680R with reference to position numbering of SEQ ID NO: 107.

In some embodiments, an engineered protein of the invention may exhibit an altered PAM (protospacer adjacent motif) specificity compared to the PAM specificity for LbCas12a.

In some embodiments, an engineered protein of the invention may further comprise a mutation in a nuclease active site (e.g., RuvC domain) (e.g., deadLbCas12a, dLbCas12a), optionally wherein the modified protein comprises an amino acid sequence having at least 80%, 85%, 90%, or 95% identity to the amino acid sequence of SEQ ID NO: 50.

In some embodiments, the first polypeptide of an engineered protein of the invention may comprise an amino acid residue 1 to about amino residue 250, 300, or 350 with reference to the position numbering of the modified protein, optionally wherein the first polypeptide comprises the mutation and/or the first polypeptide comprises amino acid residue 1 to about amino residue 282, 283, 284, 285, 286, 287, 288, 289, 290, 291, 292, or 293 with reference to the position numbering of the modified protein (e.g., SEQ ID NO: 180).

In some embodiments, the second polypeptide has a length of about 10, 50, 100, or 150 to about 200, 250, or 300 amino acids and/or the first polypeptide has a length of about 100, 200, or 250 to about 300, 350, or 400 amino acids, optionally wherein the second polypeptide has a length of about 140 or 150 to about 160, 170, 180, 190, 200, 210, 220, 230, 240, or 250 amino acids and/or the first polypeptide has a length of about 250 or 275 to about 300 or 350 amino acids.

In some embodiments, the second polypeptide comprises a first nuclease domain or a portion thereof. In some embodiments, the second polypeptide comprises a target strand nickase domain or a portion thereof, optionally wherein the second polypeptide comprises a target strand specific nickase domain, a nontarget strand specific nickase domain, or a target and nontarget strand nickase domain.

In some embodiments, the engineered protein of the invention further comprises a third polypeptide that comprises a second nuclease domain or a portion thereof, optionally wherein the third polypeptide comprises the mutation and/or wherein the first polypeptide and the third polypeptide are non-continuous (i.e., are separated from each (optionally by at least 10, 50, 100, or more amino acids) and are not directly attached to each other).

In some embodiments, the second polypeptide may be heterologous to the third polypeptide.

In some embodiments, the third polypeptide may be a Type V CRISPR-Cas effector polypeptide, optionally wherein the third polypeptide may be a second portion of the modified protein and may be different than the first polypeptide.

In some embodiments, the second nuclease domain or a portion thereof may be a nontarget and target strand nickase domain or a portion thereof.

In some embodiments, the second nuclease domain may be active. In some embodiments, the second nuclease domain may be inactive.

In some embodiments, the second polypeptide may comprise a HNH domain, optionally wherein the HNH domain comprises a mutation that modifies the activity of the HNH domain (e.g., a H840A mutation).

In some embodiments, the first and third polypeptides are each a portion of the modified protein and the second polypeptide is between and/or linked to (e.g., directly or indirectly) two amino acids that are two consecutive or nonconsecutive amino acids of the modified protein.

In some embodiments, the second polypeptide may be positioned in the engineered protein in a location that corresponds to an interdomain linker region of the modified protein.

In some embodiments, the second polypeptide comprises an amino acid sequence having at least 70%, 75%, 80%, 85%, 90%, 95%, 96%, 97%, 98%, 99%, or more sequence identity to one or more of SEQ ID NOs: 1 or 169-174, optionally wherein the second polypeptide comprises an amino acid sequence of any one of SEQ ID NOs: 1 or 169-174.

In some embodiments, the engineered protein comprises, in the amino terminal to carboxy terminal direction, the first polypeptide, the second polypeptide, and the third polypeptide, optionally wherein the third polypeptide has a length of about 800 or 850 to about 900, 1,000, or 1,100 amino acids (e.g., about 900 to about 950 or 1,000 amino acids).

In some embodiments, the engineered protein may further comprise all or a portion of a wedge domain, a Rec1 domain, a Rec2 domain, a PAM-interacting domain, a RuvC domain, a bridge helix, and/or a Nuc domain of the modified protein, optionally wherein the engineered protein comprises all or a portion of a wedge domain, a Rec1 domain, a Rec2 domain, a PAM-interacting domain, a RuvC domain, a bridge helix, and/or a Nuc domain of LbCas12a. In some embodiments, the engineered protein may comprise a Rec1 domain and a Rec2 domain and the second polypeptide may be located between the Rec1 domain and the Rec2 domain.

In some embodiments, an engineered protein of the invention may be devoid of at least a portion of the modified protein, optionally wherein the engineered protein may be devoid of at least a portion of LbCas12a.

In some embodiments, an engineered protein of the invention may further comprise a first linker between the first polypeptide and the second polypeptide and/or a second linker between the second polypeptide and the third polypeptide, optionally wherein the first linker and/or second comprises an amino acid sequence of (GGS), wherein n is an integer of 1-20; GS; SG; and/or an amino acid sequence of one of SEQ ID NOs: 18-47 or 176-179. In some embodiments, the first linker and/or the second linker may comprise 1 to 10 amino acids, optionally wherein the first linker and/or the second linker may comprise 1, 2, 3, or 4 amino acids. In some embodiments, the first linker and/or second linker may comprise glycine and/or serine.

In some embodiments, an engineered protein of the invention may comprise an amino acid sequence having about 70%, 75%, 80%, 85%, 90%, 95%, 96%, 97%, 98%, 99%, or more sequence identity to the amino acid sequence of a wild-type CRISPR-Cas effector protein, optionally wherein the engineered protein comprises an amino acid sequence having at least 70%, 75%, 80%, 85%, 90%, 95%, 96%, 97%, 98%, 99%, or more sequence identity to one or more of SEQ ID NOs: 50-66 151, or 180.

In some embodiments, when the modified protein of an engineered protein is optimally aligned to any one of the amino acid sequences of SEQ ID NO: 50, 58, or 180, the second polypeptide may be between any one of the following sets of amino acid residues that is present in the engineered protein: amino acid residues 290 and 291 of SEQ ID NO: 50, amino acid residues 291 and 292 of SEQ ID NO: 50, amino acid residues 291 and 292 of SEQ ID NO: 58, or amino acid residues 292 and 293 of SEQ ID NO: 58, amino acid residues 290 and 291 of SEQ ID NO: 180, amino acid residues 291 and 292 of SEQ ID NO: 180, or amino acid residues 292 and 293 of SEQ ID NO: 180.

In some embodiments, the engineered protein may be a nuclease, optionally wherein the engineered protein is a target strand nickase, a nontarget strand nickase, or a target and nontarget strand nickase.

In some embodiments, the engineered protein of the invention has increased efficiency in nicking the target strand and/or nontarget strand of a target nucleic acid compared to a CRISPR-Cas effector protein (e.g., a wild-type CRISPR-Cas effector protein and/or a protein having a sequence of one of SEQ ID NOs: 50-66 or 151, or 180).

In some embodiments, the engineered protein recognizes a non-natural protospacer adjacent motif (PAM) site and/or sequence (e.g., the engineered protein comprises an altered PAM specificity compared to wild-type LbCas12a). As used herein, a “non-natural PAM site” is any PAM site that is not recognized by a wild type LbCas12 nuclease. Thus, for a wild type LbCas12a nuclease this would be any PAM sequence other than the PAM sequence of TTTV, wherein V is A, C or G. In some embodiments, a “non-natural PAM site” may include, but is not limited to, a PAM sequence of TTTT, TTCA, TTCC, TTCG, ATTC, CTTA, CTTC, CTTG, GTTC, TATA, TATC, CTCC, TCCG, TACA, TCCG, TACA, TCCG, TCCC, TCCA, and/or TATG.

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encodes an engineered protein, and/or a deaminase, and each may be comprised on the same or on a separate expression cassette or vector from that comprising the one or more guide nucleic acids. When the nucleic acid construct encoding an engineered protein or the components of an editing system is/are comprised on separate expression cassette(s) or vector(s) from that comprising the guide nucleic acid, a target nucleic acid may be contacted with (e.g., provided with) the expression cassette(s) or vector(s) encoding the engineered protein or components of an editing system in any order from one another and the guide nucleic acid, e.g., prior to, concurrently with, or after the expression cassette comprising the guide nucleic acid is provided (e.g., contacted with the target nucleic acid).

Methods of recruiting one or more components of an editing system to each other and/or to a target nucleic acid are known in the art and may include the use of a peptide tag or an affinity polypeptide that interacts with the peptide tag. In some embodiments, a guide nucleic acid may be linked to an RNA recruiting motif and a deaminase may be linked to an affinity polypeptide capable of interacting with the RNA recruiting motif, thereby recruiting the deaminase to the target nucleic acid. Alternatively, chemical interactions may be used to recruit a polypeptide (e.g., a deaminase) to a target nucleic acid.

A “recruiting motif” as used herein refers to one half of a binding pair that may be used to recruit a compound to which the recruiting motif is bound to another compound that includes the other half of the binding pair (i.e., a “corresponding motif”). The recruiting motif and corresponding motif may bind covalently and/or noncovalently. In some embodiments, a recruiting motif is an RNA recruiting motif (e.g., an RNA recruiting motif that is capable of binding and/or configured to bind to an affinity polypeptide), an affinity polypeptide (e.g., an affinity polypeptide that is capable of binding and/or configured to bind an RNA recruiting motif and/or a peptide tag), or a peptide tag (e.g., a peptide tag that is capable of binding and/or configured to bind an affinity polypeptide). For example, when a recruiting motif is an RNA recruiting motif, the corresponding motif for the RNA recruiting motif may be an affinity polypeptide that binds the RNA recruiting motif. A further example is that when a recruiting motif is a peptide tag, the corresponding motif for the peptide tag may be an affinity polypeptide that binds the peptide tag. Thus, a compound comprising a recruiting motif (e.g., an affinity polypeptide) may be recruited to another compound (e.g., a guide nucleic acid) comprising a corresponding motif for the recruiting motif (e.g., an RNA recruiting motif).

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26 (5): 910-924 (2017)); Gilbreth (Curr Opin Struc Biol 22 (4): 413-420 (2013)), U.S. Pat. No. 9,982,053, each of which are incorporated by reference in their entireties for the teachings relevant to affibodies, anticalins, monobodies and/or DARPins.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases).

In some embodiments of the invention, a guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF). Exemplary RNA recruiting motifs and corresponding affinity polypeptides that may be useful with this invention can include, but are not limited to, SEQ ID NOs: 108-118.

In some embodiments, the components for recruiting polypeptides and nucleic acids may include those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together; e.g., dihyrofolate reductase (DHFR)).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

As described herein, a “peptide tag” may be employed to recruit one or more polypeptides. A peptide tag may be any polypeptide that is capable of being bound by a corresponding motif such as an affinity polypeptide. A peptide tag may also be referred to as an “epitope” and when provided in multiple copies, a “multimerized epitope.” Example peptide tags can include, but are not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. In some embodiments, a peptide tag may also include phosphorylated tyrosines in specific sequence contexts recognized by SH2 domains, characteristic consensus sequences containing phosphoserines recognized by 14-3-3 proteins, proline rich peptide motifs recognized by SH3 domains, PDZ protein interaction domains or the PDZ signal sequences, and an AGO hook motif from plants. Peptide tags are disclosed in WO2018/136783 and U.S. Patent Application Publication No. 2017/0219596, which are incorporated by reference for their disclosures of peptide tags. Peptide tags that may be useful with this invention can include, but are not limited to, SEQ ID NO: 119 and SEQ ID NO: 120. An affinity polypeptide useful with peptide tags includes, but is not limited to, SEQ ID NO: 121.

A peptide tag may comprise or be present in one copy or in 2 or more copies of the peptide tag (e.g., multimerized peptide tag or multimerized epitope) (e.g., about 2, 3, 4, 5, 6, 7, 8, 9, 10, 11, 12, 13, 14, 15, 16, 17, 18, 9, 20, 21, 22, 23, 24, or 25 or more peptide tags). When multimerized, the peptide tags may be fused directly to one another or they may be linked to one another via one or more amino acids (e.g., 1, 2, 3, 4, 5, 6, 7, 8, 9, 10, 11, 12, 13, 14, 15, 16, 17, 18, 19, 20), or more amino acids), optionally about 3 to about 10, about 4 to about 10, about 5 to about 10, about 5 to about 15, or about 5 to about 20 amino acids, and the like, and any value or range therein. Thus, in some embodiments, a CRISPR-Cas effector protein of the invention may comprise a CRISPR-Cas effector protein domain fused to one peptide tag or to two or more peptide tags, optionally wherein the two or more peptide tags are fused to one another via one or more amino acid residues. In some embodiments, a peptide tag useful with the invention may be a single copy of a GCN4 peptide tag or epitope or may be a multimerized GCN4 epitope comprising about 2 to about 25 or more copies of the peptide tag (e.g., about 2, 3, 4, 5, 6, 7, 8, 9, 10, 11, 12, 13, 14, 15, 16, 17, 18, 19, 20, 21, 22, 23, 24, 25 or more copies of a GCN4 epitope or any range therein).

In some embodiments, a peptide tag may be fused to a CRISPR-Cas polypeptide or domain. In some embodiments, a peptide tag may be fused or linked to the C-terminus of a CRISPR-Cas effector protein to form a CRISPR-Cas fusion protein. In some embodiments, a peptide tag may be fused or linked to the N-terminus of a CRISPR-Cas effector protein to form a CRISPR-Cas fusion protein. In some embodiments, a peptide tag may be fused within a CRISPR-Cas effector protein (e.g., a peptide tag may be in a loop region of a CRISPR-Cas effector protein). In some embodiments, peptide tag may be fused to a cytosine deaminase and/or to an adenine deaminase.

An “affinity polypeptide” (e.g., “recruiting polypeptide”) refers to any polypeptide that is capable of binding to its corresponding peptide tag, peptide tag, or RNA recruiting motif. An affinity polypeptide for a peptide tag may be, for example, an antibody and/or a single chain antibody that specifically binds the peptide tag, respectively. In some embodiments, an antibody for a peptide tag may be, but is not limited to, an scFv antibody. In some embodiments, an affinity polypeptide may be fused or linked to the N-terminus of a deaminase (e.g., a cytosine deaminase or an adenine deaminase). In some embodiments, the affinity polypeptide is stable under the reducing conditions of a cell or cellular extract.

The nucleic acid constructs of the invention and/or guide nucleic acids may be comprised in one or more expression cassettes as described herein. In some embodiments, a nucleic acid construct of the invention may be comprised in the same or in a separate expression cassette or vector from that comprising a guide nucleic acid and/or a recruiting guide nucleic acid.

In some embodiments, a nucleic acid construct, expression cassette, or vector of the invention that is optimized for expression in an organism (e.g., a human or plant) may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to a nucleic acid construct, expression cassette or vector comprising the same polynucleotide(s) but which have not been codon optimized for expression in the organism.

When used in combination with a guide nucleic acid, a nucleic acid construct of the invention (and a expression cassette and/or a vector comprising the same) may be used to modify a target nucleic acid and/or its expression. A target nucleic acid may be contacted with a nucleic acid construct of the invention and/or expression cassettes and/or vectors comprising the same prior to, concurrently with or after contacting the target nucleic acid with the guide nucleic acid/recruiting guide nucleic acid (and/or expression cassettes and vectors comprising the same.

According to embodiments of the present invention, provided herein are engineered proteins. An “engineered protein” as used herein refers to a polypeptide or protein that is not found naturally in nature. In some embodiments, the engineered protein refers to a polypeptide that comprises a first polypeptide from a first protein and a second polypeptide from a second protein, wherein the first and second proteins are different from each other. In some embodiments, an engineered protein of the present invention comprises a polypeptide from a CRISPR-Cas effector protein (i.e., a CRISPR-Cas effector polypeptide) and a polypeptide that is heterologous to the CRISPR-Cas effector polypeptide (i.e., a heterologous polypeptide). A polypeptide from a CRISPR-Cas effector protein is referred to herein as a “CRISPR-Cas effector polypeptide” and a “CRISPR-Cas effector polypeptide” is all or a portion of a CRISPR-Cas effector protein. In some embodiments, a “CRISPR-Cas effector polypeptide” does not include all of a CRISPR-Cas effector protein and, thus, has a reduced number of amino acids compared to the number of amino acids for the CRISPR-Cas effector protein. In some embodiments, the CRISPR-Cas effector polypeptide is a Type V CRISPR-Cas effector polypeptide (i.e., all or a portion of a Type V CRISPR-Cas effector protein). In some embodiments, a Type V CRISPR-Cas effector polypeptide is a portion of a Type V CRISPR-Cas effector protein. In some embodiments, an engineered protein of the present invention is a target strand nickase that has about 50%, 55%, 60%, 65%, 70%, 75%, 80%, 85%, 90%, 95%, 96%, 97%, 98%, 99%, or more sequence identity to one or more wild-type CRISPR-Cas effector protein(s) (e.g., a Type V CRISPR-Cas effector protein) and/or to an amino acid sequence of one or more of SEQ ID NOs: 50-66, 151, and/or 180. In some embodiments, an engineered protein of the present invention is a target strand nickase that has at least about 70%, 75%, 80%, 85%, 90%, 95%, 96%, 97%, 98%, 99%, or more sequence identity to one or more wild-type CRISPR-Cas effector protein(s) (e.g., a Type V CRISPR-Cas effector protein) and/or to an amino acid sequence of one or more of SEQ ID NOs: 50-66, 151, and/or 180.

In some embodiments, an engineered protein and/or modified protein of the present invention may comprise an altered protospacer adjacent motif (PAM) specificity as compared to wild type LbCas12a (e.g., SEQ ID NO: 180), LbCas12a comprising the mutations S542R/K607R (e.g., LbCas12a-RR; e.g., SEQ ID NO: 182), and/or LbCas12a comprising the mutations S542R/K548V/N552R (e.g., LbCas12a-RVR; e.g., SEQ ID NO: 183). An engineered protein and/or modified protein of the present invention may have an altered PAM specificity, wherein the altered PAM specificity includes, but is not limited to, NNNG, NNNT, NNNA, NNNC, NNG, NNT, NNC, NNA, NG, NT, NC, NA, NN, NNN, NNNN, wherein each N of each sequence is independently selected from any of T, C, G, or A. In some embodiments, the altered PAM specificity may include, but is not limited to, TTTA, TTTC, TTTG, TTTT, TTCA, TTCC, TTCG, TATA, TATC, TCCG, TCCC, TOCA, and/or TATG. In some embodiments, the altered PAM specificity may include, but is not limited to, TTTT, TTCA, TTCC, TTCG, ATTC, CTTA, CTTC, CTTG, GTTC, TATA, TATC, CTCC, TCCG, TACA, TCCG, TACA, TCCG, TCCC, TCCA, and/or TATG. In some embodiments, the altered PAM specificity is not TTTA, TTTC, TTTG, TTTT, TTCA, TTCC, TTCG, TATA, TATC, TCCG, TCCC, TCCA, and/or TATG. In some embodiments, the altered PAM specificity may be NNNN, wherein each N of each sequence is independently selected from any of T, C, G, or A.

In addition to having an altered PAM recognition specificity an engineered protein and/or modified protein of the present invention may further comprise a mutation in the nuclease active site (e.g., RuvC domain) (e.g., deadLbCas12a, dLbCas12a). Such modifications may result in the engineered protein and/or modified protein having reduced nuclease activity (e.g., nickase activity) or no nuclease activity.

In some embodiments, a CRISPR-Cas effector polypeptide (e.g., a Type V CRISPR-Cas effector polypeptide) of an engineered protein of the present invention is devoid of a nuclease domain (e.g., devoid of a RuvC domain). The polypeptide that is heterologous to the CRISPR-Cas effector polypeptide may be referred to herein as a heterologous polypeptide. The heterologous polypeptide may be a polypeptide of interest as described herein. In some embodiments, an engineered protein comprises all or a portion of a deaminase domain (e.g., a cytosine deaminase and/or adenine deaminase), which may be linked to any portion of the engineered protein. For example, in some embodiments, all or a portion of a deaminase domain is linked to the N- or C-terminus of the CRISPR-Cas effector polypeptide and/or to the N- or C-terminus of the engineered protein. In some embodiments, all or a portion of a deaminase domain is between two portions of an engineered protein. In some embodiments, an engineered protein comprises all or a portion of a polypeptide of interest, which may be linked to any portion of the engineered protein.

An engineered protein may cleave, cut, or nick a nucleic acid; bind a nucleic acid (e.g., a target nucleic acid and/or a guide nucleic acid); and/or identify, recognize, or bind a guide nucleic acid as defined herein. In some embodiments, an engineered protein or a portion thereof may be an enzyme (e.g., a nuclease, endonuclease, nickase, etc.) and/or may function as an enzyme. In some embodiments, an engineered protein of the present invention is an RNA-guided DNA-binding protein. In some embodiments, an engineered protein is present in and/or forms a complex with a guide nucleic acid that is a single guide nucleic acid (e.g., a gRNA, CRISPR array, and/or crRNA), optionally wherein the guide nucleic acid is a single crRNA. In some embodiments, a complex comprises an engineered protein and a guide nucleic acid and the guide nucleic acid and/or complex consists of a single guide nucleic acid (e.g., a single crRNA). In some embodiments, an engineered protein binds a single guide nucleic acid (e.g., a single crRNA), recognizes and/or binds a target nucleic acid, and has nuclease activity, optionally wherein the engineered protein cleaves the target strand of the target nucleic acid.

In some embodiments, an engineered protein comprises a first CRISPR-Cas effector polypeptide and a heterologous polypeptide. The first CRISPR-Cas effector polypeptide may be referred to herein as a first polypeptide and may be a Type V CRISPR-Cas effector polypeptide. The heterologous polypeptide may be referred to herein as a second polypeptide. The first polypeptide may be devoid of a nuclease domain, optionally devoid of a RuvC domain. The second polypeptide may be linked to the N- or C-terminus of the first polypeptide, optionally with or without a linker (e.g., a peptide linker). In some embodiments, the second polypeptide is linked to the N- or C-terminus of the first polypeptide with a peptide linker, optionally wherein the peptide linker is a GS linker. In some embodiments, the peptide linker is a GS linker having 1, 2, 3, or 4 amino acid residues, optionally 2 or 4 amino acid residues. In some embodiments, the peptide linker has one of the amino acid sequences of SEQ ID NOs: 18-47 or 175-179. In some embodiments, the peptide linker may comprise an amino acid sequence of (GGS)ₙ, GS, SG, GSSG (SEQ ID NO: 175), GSSGSS (SEQ ID NO: 176), GSSGSSGS (SEQ ID NO: 177), (GSS)ₙ (SEQ ID NO: 178), (GSS)ₙGS (SEQ ID NO: 179), S(GGS)ₙ (SEQ ID NO: 42), SGGS (SEQ ID NO: 43), or (GGGGS)n (SEQ ID NO: 44), wherein n is an integer of 1-20 (e.g., 1, 2, 3, 4, 5, 6, 7, 8, 9, 10, 11, 12, 13, 14, 15, 16, 17, 18, 19, or 20).

In some embodiments, the first polypeptide is a portion of a first CRISPR-Cas effector protein (e.g., a portion of a Type V CRISPR-Cas effector protein such as a portion of a Cas12a). In some embodiments, the heterologous polypeptide (e.g., second polypeptide) comprises a nuclease domain, optionally a HNH domain (e.g., an HNH domain comprising a sequence having at least 70%, 75%, 80%, 85%, 90%, 95%, 96%, 97%, 98%, 99%, or 100% sequence identity to the amino acid sequence of one or more of SEQ ID NOs: 1 or 169-174). In some embodiments, the second polypeptide comprises a HNH domain that is from a CRISPR-Cas effector protein and/or comprises a sequence having at least 70%, 75%, 80%, 85%, 90%, 95%, 96%, 97%, 98%, 99%, or 100% sequence identity to the amino acid sequence of any one of SEQ ID NOs: 1 or 172. In some embodiments, the second polypeptide comprises a HNH domain that is not from a CRISPR-Cas effector protein and/or comprises a sequence having at least 70%, 75%, 80%, 85%, 90%, 95%, 96%, 97%, 98%, 99%, or 100% sequence identity to the amino acid sequence of any one of SEQ ID NOs: 169-171 or 173-174. In some embodiments, the second polypeptide is a polypeptide from a CRISPR-Cas effector protein, optionally wherein the second polypeptide is from a different type of CRISPR-Cas effector protein (e.g., a Type II CRISPR-Cas effector protein) than the type of the first CRISPR-Cas effector protein (e.g., a Type V CRISPR-Cas effector protein) from which the first CRISPR-Cas effector polypeptide is a portion of.

In some embodiments, an engineered protein comprises a first polypeptide, a second polypeptide, and a third polypeptide, which may be linked together in any order. In some embodiments, the first polypeptide may be devoid of a RuvC domain. The second polypeptide may be linked to the N- or C-terminus of the first polypeptide, optionally with or without a linker (e.g., a peptide linker), and/or the second polypeptide may be linked to the N- or C-terminus of the third polypeptide, optionally with or without a linker (e.g., a peptide linker). In some embodiments, the second polypeptide is between the first polypeptide and the third polypeptide. In some embodiments, the first polypeptide is a portion of a first CRISPR-Cas effector protein (e.g., a portion of a Type V CRISPR-Cas effector protein such as a portion of a Cas12a) and the third polypeptide is a portion of a second CRISPR-Cas effector protein (e.g., a portion of a Type V CRISPR-Cas effector protein such as a portion of a Cas12a), wherein the first CRISPR-Cas effector protein and second CRISPR-Cas effector protein may be the same protein or different proteins. In some embodiments, the first CRISPR-Cas effector protein and the second CRISPR-Cas effector protein are the same, thereby the first polypeptide and third polypeptide are portions from the same protein, but may be different portions of the CRISPR-Cas effector protein. The first polypeptide and third polypeptide may have different sequences. In some embodiments, the first polypeptide and third polypeptide may comprise a sequence that is the same. In some embodiments, the first polypeptide and third polypeptide together provide the full sequence of the CRISPR-Cas effector protein. In some embodiments, the first polypeptide and third polypeptide together do not make up the full sequence of the CRISPR-Cas effector protein (i.e., a portion of the sequence of the CRISPR-Cas effector protein is not present in the two sequences of the first polypeptide and third polypeptide); for example, 1 or 5 to 10, 15, 20, 25, 30, or more amino acids (e.g., 1, 2, 3, 4, 5, 6, 7, 8, 9, 10, 15, 20, 25, 30, or more amino acid(s)) of the CRISPR-Cas effector protein may not be present in the sequences of the first and third effector polypeptides. In some embodiments, the second polypeptide comprises a nuclease domain, optionally a HNH domain (e.g., an HNH domain from a Type II CRISPR-Cas effector protein). In some embodiments, the second polypeptide comprises a HNH domain that is not from a CRISPR-Cas effector protein. In some embodiments, the second polypeptide is a polypeptide from a CRISPR-Cas effector protein, optionally wherein the second polypeptide is from a different type of CRISPR-Cas effector protein (e.g., a Type II CRISPR-Cas effector protein) than the type of the first CRISPR-Cas effector protein (e.g., a Type V CRISPR-Cas effector protein) from which the first polypeptide is a portion of and/or than the type of the second CRISPR-Cas effector protein (e.g., a Type V CRISPR-Cas effector protein) from which the third polypeptide is a portion of. In some embodiments, the second polypeptide is from a Type II CRISPR-Cas effector protein (e.g., is a portion (e.g., the HNH domain or a portion thereof) of the Type II CRISPR-Cas effector protein), the first polypeptide is a portion of a Type V CRISPR-Cas effector protein, and the third polypeptide is a portion of a Type V CRISPR-Cas effector protein, wherein the first and second CRISPR-Cas effector polypeptides are different. In some embodiments, the second polypeptide is heterologous to one of the first polypeptide and third polypeptide. In some embodiments, the second polypeptide is heterologous to both the first polypeptide and the third polypeptide.

“Heterologous polypeptide” as used herein refers to a non-naturally occurring polypeptide compared to a CRISPR-Cas effector polypeptide present in an engineered protein. Accordingly, a heterologous polypeptide of an engineered protein is not found in nature in at least one CRISPR-Cas effector polypeptide of the engineered protein, so the heterologous polypeptide is non-naturally occurring with respect the at least one CRISPR-Cas effector polypeptide. For example, an engineered protein of the present invention may include a CRISPR-Cas effector polypeptide that is a portion of a CRISPR-Cas effector protein and a heterologous polypeptide (that is optionally from a CRISPR-Cas effector protein), and the heterologous polypeptide is non-naturally occurring compared to the CRISPR-Cas effector polypeptide in the absence of the heterologous polypeptide (e.g., the CRISPR-Cas effector polypeptide without or prior to including (e.g., insertion or fusion of) the heterologous polypeptide and the CRISPR-Cas effector polypeptide); in some embodiments, the heterologous polypeptide is heterologous to the CRISPR-Cas effector protein from which the CRISPR-Cas effector polypeptide is a portion of. In some embodiments, an engineered protein includes a heterologous polypeptide, a first CRISPR-Cas effector polypeptide that is a portion of a first CRISPR-Cas effector protein, and a second CRISPR-Cas effector polypeptide that is a portion of a second CRISPR-Cas effector protein, and the heterologous polypeptide is non-naturally occurring in (i.e., heterologous to) the first CRISPR-Cas effector polypeptide, the first CRISPR-Cas effector protein, the second CRISPR-Cas effector, and the second CRISPR-Cas effector protein. Similarly, a nucleotide sequence encoding a heterologous polypeptide is heterologous to (i.e., non-naturally occurring compared to) a nucleotide sequence encoding a CRISPR-Cas effector polypeptide of an engineered protein.

In some embodiments, the heterologous polypeptide comprises a polypeptide or domain from a different type of protein than a CRISPR-Cas effector polypeptide of the engineered protein (e.g., a Type V CRISPR-Cas effector polypeptide). For example, a heterologous polypeptide of an engineered protein of the present invention may be a portion of a first CRISPR-Cas effector protein (e.g., a Type II CRISPR-Cas effector polypeptide) and the heterologous polypeptide is heterologous to another CRISPR-Cas effector polypeptide that is present in the engineered protein (e.g., a Type V CRISPR-Cas effector polypeptide). In some embodiments, an engineered protein comprises one or more (e.g., 1, 2, 3, or more) portion(s) of (i.e., one or more CRISPR-Cas effector polypeptide(s) from) a Type V CRISPR-Cas effector protein (e.g., Cas 12a) and one or more (e.g., 1, 2, 3, or more) polypeptide(s) from a different type of CRISPR-Cas effector protein such as a Type II CRISPR-Cas effector protein. When two or more portions or polypeptides are from the same protein and each are present in an engineered protein, the two or more portions or polypeptides may be separated from each other in the engineered protein by a linker and/or a heterologous polypeptide (i.e., the two or more portions or polypeptides may not be directly linked) or may be in a different order than that of the protein from which they are from (e.g., a wild-type protein and/or CRISPR-Cas effector protein). In some embodiments, an engineered protein comprises one or more (e.g., 1, 2, 3, or more) portion(s) of (i.e., one or more CRISPR-Cas effector polypeptide(s) from) a Type V CRISPR-Cas effector protein (e.g., Cas 12a) and at least one polypeptide from and/or portion of a Type II CRISPR-Cas effector protein (e.g., Cas 9). In some embodiments, an engineered protein comprises a first CRISPR-Cas effector polypeptide that is a portion of a Type V CRISPR-Cas effector protein (e.g., Cas 12a) and a heterologous polypeptide from a Type II CRISPR-Cas effector protein (e.g., Cas 9). In some embodiments, an engineered protein comprises a first CRISPR-Cas effector polypeptide that is a portion of a Type V CRISPR-Cas effector protein (e.g., Cas 12a), a heterologous polypeptide from a Type II CRISPR-Cas effector protein (e.g., Cas 9), and a second CRISPR-Cas effector polypeptide that is a portion of a Type V CRISPR-Cas effector protein (e.g., Cas 12a), optionally wherein the first and second CRISPR-Cas effector polypeptides are different portions from the same Type V CRISPR-Cas effector protein (e.g., Cas 12a). In some embodiments, an engineered protein comprises a first CRISPR-Cas effector polypeptide that is a portion of a Type V CRISPR-Cas effector protein (e.g., Cas 12a), a heterologous polypeptide that comprises a HNH domain or a portion thereof, and a second CRISPR-Cas effector polypeptide that is a portion of a Type V CRISPR-Cas effector protein (e.g., Cas 12a), optionally wherein the first and second CRISPR-Cas effector polypeptides are different portions from the same Type V CRISPR-Cas effector protein (e.g., Cas 12a).

In some embodiments, an engineered protein comprises one or more (e.g., 1, 2, 3, or more) domain(s) or a portion thereof from a Type V CRISPR-Cas effector protein (e.g., Cas 12a) and one or more (e.g., 1, 2, 3, or more) domain(s) or a portion thereof from a different type of CRISPR-Cas effector protein such as a Type II CRISPR-Cas effector protein. In some embodiments, an engineered protein comprises one or more (e.g., 1, 2, 3, or more) domain(s) or a portion thereof from a Type V CRISPR-Cas effector protein (e.g., Cas 12a) and at least one domain or a portion thereof from a Type II CRISPR-Cas effector protein (e.g., Cas 9). In some embodiments, the heterologous polypeptide of an engineered protein does not interfere with or adversely affect the activity of a CRISPR-Cas effector polypeptide and/or of one or more domain(s) of the CRISPR-Cas effector polypeptide (e.g., a RuvC domain).

The second polypeptide and/or heterologous polypeptide of an engineered protein of the present invention may have a length of about 10 to about 300 amino acids such as about 10, 20, 30, 40, 50, 60, 70, 80, 90, or 100 amino acids to about 110, 125, 150, 175, 200, 225, 250, 275, or 300 amino acids. In some embodiments, the second polypeptide and/or heterologous polypeptide has a length of about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 amino acids. In some embodiments, the second polypeptide and/or heterologous polypeptide has a length of about 120, 125, 130, 135, or 140 amino acids to about 145, 150, 155, or 160 amino acids. In some embodiments, the second polypeptide and/or heterologous polypeptide has a length of 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, or 160 amino acids. In some embodiments, the second polypeptide and/or heterologous polypeptide is between a first CRISPR-Cas effector polypeptide and a third CRISPR-Cas effector polypeptide and the second polypeptide and/or heterologous polypeptide is heterologous to one or both of the first and third CRISPR-Cas effector polypeptides.

In some embodiments, a CRISPR-Cas effector polypeptide (e.g., a first and/or third polypeptide) of an engineered protein of the present invention has a length of about 100, 150, 200, or 250 amino acids to about 300, 350, or 400 amino acids. In some embodiments, a CRISPR-Cas effector polypeptide has a length of about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 amino acids. In some embodiments, a CRISPR-Cas effector polypeptide has a length of about 800, 850, or 900 amino acids to about 950, 1,000, 1,050, or 1,100 amino acids. In some embodiments, a CRISPR-Cas effector polypeptide has a length of about 800, 810, 820, 830, 840, 850, 860, 870, 880, 890, 900, 910, 920, 930, 940, 950, 960, 970, 980, 990, 1,000, 1,010, 1,020, 1,030, 1,040, 1,050, 1,060, 1,070, 1,080, 1,090, 1,100 amino acids. In some embodiments, an engineered protein comprises a first CRISPR-Cas effector polypeptide having a length of about 100, 150, 200, or 250 amino acids to about 300, 350, or 400 amino acids, a heterologous polypeptide having a length of about 10, 50, 100, or 140 amino acids to about 160, 200, 250, or 300 amino acids; and a second CRISPR-Cas effector polypeptide having a length of about 100, 200, 300, 400, 500, 600, 700, 800, 850, or 900 amino acids to about 950, 1,000, 1,050, or 1,100 amino acids.

In some embodiments, the heterologous polypeptide (e.g., a second polypeptide) comprises a nuclease domain or a portion thereof, which can be referred to herein as a “heterologous nuclease domain or a portion thereof” since the nuclease domain or a portion thereof from the heterologous polypeptide is heterologous to one or more CRISPR-Cas effector polypeptide(s) present in the engineered protein. The heterologous polypeptide may be a DNA nuclease domain or a portion thereof. In some embodiments, the heterologous nuclease domain or a portion thereof is from a CRISPR-Cas effector protein. In some embodiments, the heterologous nuclease domain or a portion thereof is not from a CRISPR-Cas effector protein. In some embodiments, the heterologous nuclease domain or a portion thereof is from a bacterial protein, optionally wherein the heterologous nuclease domain or a portion thereof is from a restriction endonuclease, homing endonuclease, colicin, pyocin, reverse transcriptase, DNase, and/or a standalone HNH domain. In some embodiments, an engineered protein comprises a heterologous polypeptide that includes a nuclease domain or a portion thereof (i.e., a heterologous nuclease domain or a portion thereof), and the engineered protein is a nuclease that optionally cleaves the target strand of a target nucleic acid and/or the non-target stand of a target nucleic acid. In some embodiments, the engineered protein cleaves the target strand of a target nucleic acid and the non-target stand of the target nucleic acid and provides either a blunt-ended double strand break of the target nucleic acid or a staggered double-strand break of the target nucleic acid. In some embodiments, the engineered protein cleaves the target strand of a target nucleic acid and the non-target stand of the target nucleic acid and the distance (e.g., number of nucleotides) between the cut sites is 0, 1, 2, 3, 4, or 5 nucleotides to about 6, 7, 8, 9, 10, 11, 12, 13, 14, 15, 16, 17, 18, 19, or 20 nucleotides.

In some embodiments, the heterologous nuclease domain or a portion thereof may be a target strand nickase domain or a portion thereof. A “target strand nickase domain or a portion thereof” as used herein refers to a polypeptide that has nickase activity to the target strand of a target nucleic acid when the domain or portion thereof is in its native protein. That is, a target strand nickase domain or a portion thereof can or is capable of nicking (e.g., cleaving or breaking) the target strand (also referred to as the sense (e.g., “+”; template) strand) of the target nucleic acid when the domain or portion thereof is in its native protein. For example, the HNH domain of Cas9 nicks and/or has nickase activity to the target strand of a target nucleic acid. “Nickase activity” as used herein refers to a single-strand break in a nucleic acid.

In some embodiments, a target strand nickase domain or a portion thereof, when present in an engineered protein, may have nickase activity to the target strand of a target nucleic acid. In some embodiments, a target strand nickase domain or a portion thereof, when present in an engineered protein, may have nickase activity to the nontarget strand (also referred to as the antisense (e.g., “−”, complementary) strand) of a target nucleic acid. When a target strand nickase domain or a portion thereof in an engineered protein has nickase activity to both the target strand and nontarget strand, the target strand nickase domain or a portion thereof may cleave both strands sequentially. In some embodiments, a target strand nickase domain or a portion thereof in an engineered protein has more activity (e.g., enzymatic activity) towards the target strand than the nontarget strand of a target nucleic acid. For example, when present in an engineered protein, the target strand nickase domain or a portion thereof may prefer or cleave faster the target strand of a target nucleic acid than the nontarget strand of the target nucleic acid.

A “target strand specific nickase domain” as used herein refers to a polypeptide that has nickase activity only to the target strand of a target nucleic acid and does not nick the nontarget strand of the target nucleic acid. A “nontarget strand specific nickase domain” as used herein refers to a polypeptide that has nickase activity only to the nontarget strand of a target nucleic acid and does not nick the target strand of the target nucleic acid. A “target and nontarget strand nickase domain” as used herein refers to a polypeptide that has nickase activity to both the target strand and the nontarget strand of a target nucleic acid. In some embodiments, an engineered protein comprises a target strand nickase domain or a portion thereof and the target strand nickase domain or a portion thereof is a target strand specific nickase domain in the engineered protein. In some embodiments, an engineered protein comprises a target strand nickase domain or a portion thereof and the target strand nickase domain or a portion thereof is a nontarget strand specific nickase domain in the engineered protein. In some embodiments, an engineered protein comprises a target strand nickase domain or a portion thereof and the target strand nickase domain or a portion thereof is a target and nontarget strand nickase domain in the engineered protein.

An engineered protein may comprise a heterologous polypeptide (e.g., a second polypeptide) that comprises target strand nickase domain or a portion thereof. Accordingly, the engineered protein may have nickase activity to the target strand of a target nucleic acid and/or to the nontarget strand of the target nucleic acid. Thereby, the engineered protein may be a target strand nickase and/or a nontarget strand nickase. A “target strand nickase” as used herein in reference to an engineered protein refers to an engineered protein that can or is capable of cleaving the target strand of a target nucleic acid. A “nontarget strand nickase” as used herein in reference to an engineered protein refers to an engineered protein that can or is capable of cleaving the nontarget strand of a target nucleic acid. A “target and nontarget strand nickase” as used herein in reference to an engineered protein refers to an engineered protein that can or is capable of cleaving both the target and nontarget strand of a target nucleic acid in any order (e.g., sequentially or simultaneously). In some embodiments, the engineered protein is a target strand nickase and/or has nickase activity to the target strand of a target nucleic acid. In some embodiments, the engineered protein is a nontarget strand nickase and/or has nickase activity to the nontarget strand of a target nucleic acid. In some embodiments, the engineered protein is a target and nontarget strand nickase and/or has nickase activity to the target strand and nontarget strand of a target nucleic acid.

In some embodiments, the heterologous polypeptide of an engineered protein comprises a target strand nickase domain or a portion thereof and the target strand nickase domain or a portion thereof of the engineered protein has nickase activity to the target strand of a target nucleic acid, thereby the engineered protein is a target strand nickase. In some embodiments, the heterologous polypeptide of an engineered protein comprises a target strand nickase domain or a portion thereof and the target strand nickase domain or a portion thereof of the engineered protein has nickase activity to the nontarget strand of a target nucleic acid, thereby the engineered protein is a nontarget strand nickase. In some embodiments, the heterologous polypeptide of an engineered protein comprises a target strand nickase domain or a portion thereof and the target strand nickase domain or a portion thereof of the engineered protein has nickase activity to the both the target and nontarget strand of a target nucleic acid, thereby the engineered protein is a target and nontarget strand nickase. In some embodiments, the heterologous polypeptide of an engineered protein comprises a target strand nickase domain or a portion thereof and the target strand nickase domain or a portion thereof of the engineered protein has nickase activity to at least the target strand of a target nucleic acid and a CRISPR-Cas effector polypeptide of the engineered protein comprises a nuclease domain or a portion thereof that has nickase activity to at least the nontarget strand of the target nucleic acid, thereby the engineered protein is a target and nontarget strand nickase. In some embodiments, the CRISPR-Cas effector polypeptide of the engineered protein comprises a nuclease domain or portion thereof that is a target and nontarget strand nickase domain or portion thereof, but the nuclease domain or portion thereof is inactivated so that nuclease activity to the target strand is inactivated, thereby the target strand of the target nucleic acid is not nicked by the nuclease domain or portion thereof.

In some embodiments, an engineered protein may comprise one or more (e.g., 1, 2, or more) nuclease domain(s) or a portion thereof. In some embodiments, an engineered protein comprises at least two different nuclease domains or a portion thereof. In some embodiments, an engineered protein may comprise a native nuclease domain, optionally one or more (e.g., 1, 2, or more) native nuclease domain(s). A “native nuclease domain” as used herein refers to a nuclease domain that is naturally present in a CRISPR-Cas effector protein. In some embodiments, an engineered protein comprises a first heterologous nuclease domain (e.g., from and/or present in the heterologous polypeptide) and a second nuclease domain. The second nuclease domain may be from and/or present in CRISPR-Cas effector protein. In some embodiments, the first nuclease domain may be a native nuclease domain and/or the second nuclease domain may be a native nuclease domain. In some embodiments, the second nuclease domain is a target and nontarget strand nickase domain or a portion thereof. A “nontarget and target strand nickase domain or a portion thereof” as used herein refers to a polypeptide that has nickase activity to the nontarget strand of a target nucleic acid and to the target strand of the target nucleic acid when the domain or portion thereof is in its native protein, and cleaves the nontarget strand before the target strand or prefers or cleaves faster the nontarget strand than the target strand. A nontarget and target strand nickase domain or a portion thereof may provide a staggered double strand break in the target nucleic acid. In some embodiments, the second nuclease domain is active. In some embodiments, the second nuclease domain is deactivated (i.e., dead, inactive, or devoid of nickase activity). In some embodiments, the second nuclease domain only nicks the nontarget strand of a target nucleic acid and/or comprises a mutation that inactivates nickase activity to the target strand of a target nucleic acid. A nuclease domain or portion thereof in an engineered protein may be deactivated by a mutation in the nuclease domain or portion thereof that removes or inactivates nickase activity. In some embodiments, an engineered protein comprises a nuclease domain or portion thereof from a Type V CRISPR-Cas effector protein such as a Cas12a (e.g., from one of SEQ ID NO: 50-66 or 180) or Cas12b (e.g., from SEQ ID NO: 151). In some embodiments, the nuclease domain is a RuvC domain from a Type V CRISPR-Cas effector protein such as a Cas12a or Cas12b. An engineered protein may comprise one or more nuclease domain(s) that provide a blunt-ended double strand break of a target nucleic acid or a staggered double-strand break of a target nucleic acid.

In some embodiments, the heterologous polypeptide (e.g., a second polypeptide) of an engineered protein comprises all or a portion of a HNH domain of a CRISPR-Cas effector protein. The heterologous polypeptide and/or HNH domain may comprise and/or form a zinc finger motif. In some embodiments, the heterologous polypeptide and/or HNH domain has a length of about 10, 20, 30, 40, 50, 60, 70, 80, 90, or 100 amino acids to about 110, 125, 150, 175, 200, 225, 250, 275, or 300 amino acids. The heterologous polypeptide and/or HNH domain may comprise about 25 or 30 to about 40 or 45 amino acids and/or may comprise one or at least two histidines and an asparagine that are optionally in a nucleic acid binding and cleavage site. In some embodiments, the heterologous polypeptide and/or HNH domain may comprise about 25 or 30 to about 40 or 45 amino acids that include two histidines and one asparagine that are present in and/or form a zinc finger motif. The heterologous polypeptide and/or HNH domain may comprise and/or form two antiparallel beta-strands that are linked by a loop and/or may comprise an alpha helix, optionally wherein a histidine is present in at least one of the beta-strands, an asparagine is present in the loop, and/or a histidine or asparagine is present in the alpha-helix. The heterologous polypeptide may comprise all or a portion of a HNH domain having a structure as described in Pediaditakis M, et al. Journal of Bacteriology 194 (22); 6184-6194. In some embodiments, the heterologous polypeptide of an engineered protein comprises all or a portion of a HNH domain of a Type II CRISPR-Cas effector protein such as a Cas9 HNH domain. The heterologous polypeptide of an engineered protein may comprise all or a portion of a HNH domain (e.g., a Cas9 HNH domain) that is inactive. The HNH domain or a portion thereof may have an inactivating mutation (e.g., a mutation that removes nickase activity). In some embodiments, the heterologous polypeptide of an engineered protein comprises all or a portion of an HNH domain that has an inactivating mutation and/or the HNH domain is inactive (e.g., does not have nickase activity). In some embodiments, the heterologous polypeptide of an engineered protein comprises all or a portion of an inactivated HNH domain that has a H840A mutation. In some embodiments, the heterologous polypeptide of an engineered protein comprises an amino acid sequence having at least 70%, 75%, 80%, 85%, 90%, 95%, 96%, 97%, 98%, 99%, or 100% sequence identity to the amino acid sequence of one or more of SEQ ID NOs: 1 or 169-174. In some embodiments, the heterologous polypeptide of an engineered protein comprises the amino acid sequence of any one of SEQ ID NOs: 1 or 169-174.

In some embodiments, the heterologous polypeptide of an engineered protein comprises an amino acid sequence that has an amino acid residue that is not a histidine residue at a position corresponding to amino acid residue number 839 of SEQ ID NO: 81, when the amino acid sequence of the heterologous polypeptide and SEQ ID NO: 81 are optimally aligned. In some embodiments, the heterologous polypeptide of an engineered protein comprises an amino acid sequence that has an amino acid residue that is not a histidine residue at a position corresponding to amino acid residue number 75 of SEQ ID NO: 1, when the amino acid sequence of the heterologous polypeptide and SEQ ID NO: 1 are optimally aligned. In some embodiments, the heterologous polypeptide of an engineered protein comprises an amino acid sequence that has an alanine residue at a position corresponding to amino acid residue number 839 of SEQ ID NO: 81, when the amino acid sequence of the heterologous polypeptide and SEQ ID NO: 81 are optimally aligned. In some embodiments, the heterologous polypeptide of an engineered protein comprises an amino acid sequence that has an alanine residue at a position corresponding to amino acid residue number 75 of SEQ ID NO: 1, when the amino acid sequence of the heterologous polypeptide and SEQ ID NO: 1 are optimally aligned.

In some embodiments, the heterologous polypeptide of an engineered protein may be between and/or linked to (e.g., directly or indirectly) two consecutive or nonconsecutive amino acids that are present in a CRISPR-Cas effector protein. In some embodiments, the engineered protein is prepared by inserting a heterologous polypeptide between two consecutive or nonconsecutive amino acids of a CRISPR-Cas effector protein or a portion thereof. In some embodiments, an engineered protein may comprise in the amino terminal to carboxy terminal direction, a first CRISPR-Cas effector polypeptide, a heterologous polypeptide, and a second CRISPR-Cas effector polypeptide, with the first and second CRISPR-Cas effector polypeptides being from the same CRISPR-Cas effector protein.

In some embodiments, a CRISPR-Cas effector polypeptide comprises a portion of a Type V CRISPR-Cas effector protein such as Cas12a or Cas12b. The CRISPR-Cas effector polypeptide may comprise all or a portion of a nucleic acid binding domain such as a nucleic acid binding domain from a Type V CRISPR-Cas effector protein (e.g., Cas12a or Cas12b). In some embodiments, a CRISPR-Cas effector polypeptide of an engineered protein comprises an amino acid sequence having at least 70%, 75%, 80%, 85%, 90%, 95%, 96%, 97%, 98%, 99%, or 100% sequence identity to a portion of the amino acid sequence of one or more of SEQ ID NOs: 50-66, 151, or 180. In some embodiments, a CRISPR-Cas effector polypeptide comprises a portion of the amino acid sequence of any one of SEQ ID NOs: 50-66, 151, or 180. In some embodiments, an engineered protein comprises two or more (e.g., 2, 3, 4, or more) separate portions of the amino acid sequence of any one of SEQ ID NOs: 50-66, 151, or 180.

In some embodiments, an engineered protein of the present invention may be devoid of about 5, 10, 15, 20, 25, 30, 35, 40, 45, 50, 55, 60, 65, 70, 75, 80, 85, 90, 95, 100 or more amino acids that are present in a CRISPR-Cas effector protein such as one having a sequence of any one of SEQ ID NOs: 50-66, 151, or 180. In some embodiments, an engineered protein of the present invention may be devoid of 0, 1, 2, 3, 4, 5, 6, 7, 8, 9, 10, 11, 12, 13, 14, or 15 amino acids that are present in a CRISPR-Cas effector protein such as one having a sequence of any one of SEQ ID NOs: 50-66, 151, or 180. For example, an engineered protein may be devoid of one or more amino acids from amino acid residue 283 to amino acid residue 293 of SEQ ID NO: 50 or SEQ ID NO: 58 or SEQ ID NO: 180; from amino acid residue 331 to amino acid residue 341 of SEQ ID NO: 55; from amino acid residue 312 to amino acid residue 322 of SEQ ID NO: 51; or from corresponding amino acid residues for a sequence that is optimally aligned to one of SEQ ID NOs: 50, 51, 58, 55, or 180 (e.g., from amino acid residues that correspond to amino acid residues 283-293 when a sequence (e.g., SEQ ID NO: 52) is optimally aligned to SEQ ID NO: 50). In some embodiments, an engineered protein is devoid of one or more (e.g., 1, 2, 3, 4, or more) interdomain linker region(s) (e.g., a region that is between two domains such as two adjacent domains) that are present in a CRISPR-Cas effector protein (e.g., one having a sequence of any one of SEQ ID NOs: 50-66, 151, or 180) from which a CRISPR-Cas effector is a portion of and that is present in the engineered protein.

In some embodiments, the heterologous polypeptide of an engineered protein may be between and/or linked to (e.g., directly or indirectly) two consecutive or nonconsecutive amino acids of a CRISPR-Cas effector protein (e.g., a CRISPR-Cas effector protein having an amino acid sequence of any one of SEQ ID NOs: 50-66, 151, or 180). For example, an engineered protein may comprise, from the N- to C-terminus, a first CRISPR-Cas effector polypeptide, an HNH domain, and a second CRISPR-Cas effector polypeptide, wherein the first and second CRISPR-Cas effector polypeptides are each a portion of a CRISPR-Cas effector protein and the last amino acid residue at the C-terminus of the first CRISPR-Cas effector polypeptide and the first amino acid reside at the N-terminus of the second CRISPR-Cas effector polypeptide are two consecutive or nonconsecutive amino acid residues of the a CRISPR-Cas effector protein. The heterologous polypeptide may be linked directly to one or both of the two consecutive or nonconsecutive amino acids of the CRISPR-Cas effector protein (i.e., no linker is used to attach one terminus of the heterologous polypeptide to a terminus of a CRISPR-Cas effector polypeptide that is a portion of the CRISPR-Cas effector protein). In some embodiments, the heterologous polypeptide may be linked indirectly (e.g., via a linker such as a peptide linker) to one or both of the two consecutive or nonconsecutive amino acids of the CRISPR-Cas effector protein. In some embodiments, the heterologous polypeptide of an engineered protein may be between and/or linked to (e.g., directly or indirectly) two consecutive amino acids of a CRISPR-Cas effector protein (e.g., a CRISPR-Cas effector protein having an amino acid sequence of any one of SEQ ID NOs: 50-66, 151, or 180). In some embodiments, the heterologous polypeptide of an engineered protein may be between and/or linked to (e.g., directly or indirectly) two nonconsecutive amino acids of a CRISPR-Cas effector protein (e.g., a CRISPR-Cas effector protein having an amino acid sequence of any one of SEQ ID NOs: 50-66, 151, or 180).

In some embodiments, the two consecutive or nonconsecutive amino acids are two of the amino acid residues from amino acid residue 250, 260, 270, or 280 to amino acid residue 290, 300, 310, 320, 330, 340, or 350 that are consecutive or nonconsecutive, respectively. In some embodiments, the heterologous polypeptide may be between and/or linked to (e.g., directly or indirectly) two consecutive or nonconsecutive amino acids that are two of the following amino acid residues: amino acid residues 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, and 350 of a CRISPR-Cas effector protein (e.g., a CRISPR-Cas effector protein having an amino acid sequence of any one of SEQ ID NOs: 50-66, 151, or 180). In some embodiments, the heterologous polypeptide of an engineered protein may be between and/or linked to (e.g., directly or indirectly) two nonconsecutive amino acids of a CRISPR-Cas effector protein (e.g., a CRISPR-Cas effector protein having an amino acid sequence of any one of SEQ ID NOs: 50-66, 151, or 180), wherein one of the two nonconsecutive amino acid residues is amino acid residue 270, 271, 272, 273, 274, 275, 276, 277, 278, 279, 280, 281, 282, 283, 284, or 285 and the other of the two nonconsecutive amino acid residues is amino acid residue 286, 287, 288, 289, 290, 291, 292, 293, 294, 295, 296, 297, 298, 299, 300, 301, 302, 303, 304, or 305, optionally of SEQ ID NOs: 50-66, 151, or 180. In some embodiments, the heterologous polypeptide is between and/or linked to (e.g., directly or indirectly) amino acid residues 290 and 291, amino acid residues 291 and 292, amino acid residues 292 and 293, amino acid residues 293 and 294, amino acid residues 320 and 321, amino acid residues 321 and 322, amino acid residues 339 and 340, or amino acid residues 340 and 341 of a CRISPR-Cas effector protein such as a CRISPR-Cas effector protein having an amino acid sequence of any one of SEQ ID NOs: 50-66, 151, or 180. For example, in some embodiments, the heterologous polypeptide may be between and/or linked to (e.g., directly or indirectly) amino acid residues 290 and 291 of SEQ ID NO: 50; amino acid residues 291 and 292 of SEQ ID NO: 50; amino acid residues 291 and 292 of SEQ ID NO: 58; amino acid residues 292 and 293 of SEQ ID NO: 58; amino acid residues 320 and 321 of SEQ ID NO: 51; amino acid residues 321 and 322 of SEQ ID NO: 51; amino acid residues 322 and 323 of SEQ ID NO: 51; amino acid residues 339 and 340 of SEQ ID NO: 55; amino acid residues 340 and 341 of SEQ ID NO: 55; amino acid residues 290 and 291 of SEQ ID NO: 180; amino acid residues 291 and 292 of SEQ ID NO: 180; amino acid residues 292 and 293 of SEQ ID NO: 180; or corresponding amino acid residues for a sequence that is optimally aligned to one of SEQ ID NOs: 50, 51, 58, 55, or 180 (e.g., amino acid residues that correspond to amino acid residues 291 and 292 when a sequence (e.g., SEQ ID NO: 52) is optimally aligned to SEQ ID NO: 50). In some embodiments, the heterologous polypeptide of an engineered protein may be in an interdomain linker region (e.g., a region that is between two domains such as two adjacent domains) of a CRISPR-Cas effector protein. In some embodiments, the heterologous polypeptide may be positioned in an engineered protein such that it is adjacent to an exposed portion of the target strand of a target nucleic acid.

In some embodiments, an engineered protein comprises all or a portion of a wedge domain, a Rec1 domain, a Rec2 domain, a PAM-interacting domain, a RuvC domain, a bridge helix, and/or a Nuc domain each of which may be from a Type V CRISPR-Cas effector protein such as Cas12a, Cas12b, and/or a protein having a sequence of any one of SEQ ID NOs: 50-66, 151, or 180. In some embodiments, an engineered protein comprises all or a portion of a Cas12a domain having a structure as described in Yamano, Takashi, et al., Mol Cell 67:633-645 (2017). In some embodiments, an engineered protein comprises all or a portion of an alpha-helical recognition (REC) lobe, optionally wherein the domains of all or a portion of the REC lobe in the engineered protein may be in a different order and/or structure than in a Type V CRISPR-Cas effector protein. A REC lobe may contain a Rec1 domain and Rec2 domain. A Rec1 domain may comprise 13 alpha helices and/or a Rec2 domain may comprise 10 alpha helices and two beta strands that may form a small antiparallel sheet. In some embodiments, the heterologous polypeptide of an engineered protein may be between a polypeptide for all or a portion of a Rec1 domain and a polypeptide for all or a portion of a Rec2 domain each of which may be from a Type V CRISPR-Cas effector protein such as Cas12a, Cas12b, and/or a protein having a sequence of any one of SEQ ID NOs: 50-66, 151, or 180. In some embodiments, all or a portion of the heterologous polypeptide is at an exposed surface or interface of the engineered protein. In some embodiments, the CRISPR-Cas effector polypeptide of an engineered protein comprises all or a portion of a RuvC domain. As one of skill in the art would understand, some domains (e.g., the wedge and RuvC domains of Cas12a) are not continuous in sequence and may be split into two or more (e.g., 2, 3, 4, or more) non-continuous sequences. For example, the polypeptide for Cas12 may have the following from the N- to C-terminus: first portion of the wedge domain (WED-1), Rec1 domain, Rec2 domain, second portion of the wedge domain (WED-2), PAM-interacting domain (PI), third portion of the wedge domain (WED-3), first portion of the RuvC domain (RuvC-1), bridge helix, second portion of the RuvC domain (RuvC-2), Nuc domain, and third portion of the RuvC domain (RuvC-3). In some embodiments, an engineered protein comprises all or a portion of an active RuvC domain. In some embodiments, an engineered protein comprises all or a portion of an inactivated RuvC domain, optionally all or a portion of an inactivated RuvC domain that has a D10A mutation. In some embodiments, an engineered protein comprises all or a portion of an inactivated RuvC domain and the polypeptide comprising all or a portion of the inactivated RuvC domain has an alanine at a position corresponding to amino acid residue 831 of SEQ ID NO: 50 when the polypeptide is optimally aligned to SEQ ID NO: 50, optionally wherein the mutation is referred to as a D10A and/or D832A mutation.

A CRISPR-Cas effector polypeptide may comprise a nuclease, optionally a RuvC like nuclease. In some embodiments, a CRISPR-Cas effector polypeptide comprises a RuvC domain or a portion thereof. In some embodiments, a CRISPR-Cas effector polypeptide comprises a nuclease in the Rnase H superfamily. In some embodiments, a CRISPR-Cas effector polypeptide comprises RNase H-like enzyme having a catalytic core that may include a β-sheet comprising five β-strands, ordered 32 145, optionally where the β-strand 2 is antiparallel to the other β-strands. On both sides the central β-sheet may be flanked by α-helices, the number of which may differ between related enzymes. In some embodiments, a CRISPR-Cas effector polypeptide comprises a RNase H-like catalytic core where the active site residues include one or more of aspartic acid, glutamic acid and histidine. In some embodiments, a CRISPR-Cas effector polypeptide comprising a RNase H-like catalytic core may include negatively charged side chains in the active sites of the RNase H-like polypeptide that, directly or through a water molecule, are involved in coordinating a divalent metal ion. In some embodiments, a CRISPR-Cas effector polypeptide comprises RNase H-like catalytic core that uses a two ion-dependent mechanism of catalysis, optionally wherein the ion is Mg²⁺ and/or Mn²⁺. In some embodiments, a CRISPR-Cas effector polypeptide comprises nuclease and/or RNase H-like nuclease as described in Majorek K A, et al. Nucleic Acids Res. 2014; 42 (7): 4160-4179, which is incorporated herein by reference in its entirety.

In some embodiments, a CRISPR-Cas effector polypeptide comprises one or more (e.g., 1, 2, 3, 4 or more) mutations. The one or more mutations may be to improve or modify the activity of a heterologous polypeptide and/or the activity of a CRISPR-Cas effector polypeptide. In some embodiments, a CRISPR-Cas effector polypeptide may comprise an inactivating mutation such as a D10A mutation in the RuvC domain. In some embodiments, a CRISPR-Cas effector polypeptide comprises all or a portion of a Rec1 domain that comprises one or more (e.g., 1, 2, 3, 4 or more) mutations such as in one or more of amino acid residue(s) 243-253 of a CRISPR-Cas effector protein (e.g., a CRISPR-Cas effector protein having an amino acid sequence of any one of SEQ ID NOs: 50-66, 151, or 180) and/or in the sequence GFVTESGEKIK (SEQ ID NO: 122). In some embodiments, a CRISPR-Cas effector polypeptide comprises a hairpin and/or the sequence GFVTESGEKIK (SEQ ID NO: 122), and one or more of the amino acid residue(s) in the hairpin and/or sequence may be mutated. In some embodiments, a CRISPR-Cas effector polypeptide comprises a hairpin and/or the sequence GFVTESGEKIK (SEQ ID NO: 122), and all or a portion of the hairpin and/or sequence is deleted. In some embodiments, a CRISPR-Cas effector polypeptide comprises a hairpin and/or the sequence GFVTESGEKIK (SEQ ID NO: 122), and 1, 2, 3, 4, 5, or more amino acid residues are added to one or both ends of the hairpin and/or sequence.

In some embodiments, an engineered protein comprises, from the N- to C-terminus, a first CRISPR-Cas effector polypeptide, an HNH domain, and a second CRISPR-Cas effector polypeptide, wherein the first and second CRISPR-Cas effector polypeptides are each a portion of deactivated LbCas12a (e.g., LbCas12a having a sequence of SEQ ID NO: 50) and the last amino acid residue at the C-terminus of the first CRISPR-Cas effector polypeptide and the first amino acid reside at the N-terminus of the second CRISPR-Cas effector polypeptide are two consecutive amino acid residues of the deactivated LbCas12a. The HNH domain may be from Streptococcus pyogenes Cas9 (SpCas9) and/or may have a sequence comprising SEQ ID NO: 1. In some embodiments, the HNH domain may have a sequence of any one of SEQ ID NOs: 1 or 169-174. The HNH domain may be positioned in the engineered protein such that it is adjacent to an exposed portion of the target strand of a target nucleic acid. The engineered protein may be a target strand nickase. In some embodiments, the engineered protein only nicks the target DNA strand. In some embodiments, the engineered protein is a target and nontarget strand nickase.

One or more (e.g., 1, 2, 3, 4, or more) linker(s) may be present in an engineered protein. For example, a linker may be present between a CRISPR-Cas effector polypeptide and a heterologous polypeptide (e.g., a second polypeptide). In some embodiments, a linker may be present between a first CRISPR-Cas effector polypeptide and a heterologous polypeptide and a linker may be present between the heterologous polypeptide and a second CRISPR-Cas effector polypeptide. Exemplary linkers include, but are not limited to, those described herein. In some embodiments, the linker comprises 1 to 2, 3, 4, 5, 6, 7, 8, 9, or 10 amino acids and/or comprises glycine and/or serine. In some embodiments, the linker comprises 1, 2, 3, or 4 amino acids that are glycine and/or serine (i.e., a GS linker). In some embodiments, the engineered protein is devoid of a linker between a CRISPR-Cas effector polypeptide and a heterologous polypeptide. In some embodiments, the heterologous polypeptide is indirectly linked to an amino acid residue at the N-terminus of a CRISPR-Cas effector polypeptide via a linker and/or the heterologous polypeptide is indirectly linked to an amino acid residue at the C-terminus of a CRISPR-Cas effector polypeptide via a linker. Two or more peptide linkers present in an engineered protein of the present invention may be independently selected from each other and may be the same or different from each other. In some embodiments, an engineered protein comprises a peptide linker that is a GS linker having 1, 2, 3, or 4 amino acid residues, optionally 2 or 4 amino acid residues. In some embodiments, an engineered protein comprises a peptide linker that has an amino acid sequence of (GGS)ₙ wherein n is an integer of 1-20; GS; SG; and/or an amino acid sequence of any one of SEQ ID NOs: 18-47 or 176-179.

In some embodiments, the heterologous polypeptide is directly linked (i.e., without a linker) to an amino acid residue at the N-terminus of a CRISPR-Cas effector polypeptide and/or the heterologous polypeptide is directly linked (i.e., without a linker) to an amino acid residue at the C-terminus of a CRISPR-Cas effector polypeptide.

An engineered protein may comprise an amino acid sequence having at least 70%, 75%, 80%, 85%, 90%, 95%, 96%, 97%, 98%, 99%, or more sequence identity to any one of SEQ ID NOs: 2-17, 125-132, or 157-168. In some embodiments, an engineered protein comprises and/or has an amino acid sequence of any one of SEQ ID NOs: 2-17, 125-132, or 157-168. An engineered protein may have at least 70%, 75%, 80%, 85%, 90%, 95%, 96%, 97%, 98%, 99%, or more sequence identity to all or a portion of an amino acid sequence of a wild-type CRISPR-Cas effector protein. In some embodiments, the engineered protein has at least 70%, 75%, 80%, 85%, 90%, 95%, 96%, 97%, 98%, 99%, or more sequence identity to all or a portion of the amino acid sequence of any one of SEQ ID NOs: 50-66, 151, or 180. In some embodiments, the engineered protein has about 70%, 75%, or 80% to about 85%, 90%, 95%, or 98% sequence identity to all or a portion of the amino acid sequence of any one of SEQ ID NOs: 50-66, 151, or 180.

An engineered protein may have increased efficiency compared to a CRISPR-Cas effector protein (e.g., Cas12a, a CRISPR-Cas effector protein having a sequence of SEQ ID NOs: 50-66, 151, or 180, and/or a wild-type CRISPR-Cas effector protein) such as increased efficiency in nicking the target strand and/or nontarget strand of a target nucleic acid. In some embodiments, an engineered protein may have increased efficiency compared to a CRISPR-Cas effector protein (e.g., Cas12a, a CRISPR-Cas effector protein having a sequence of SEQ ID NOs: 50-66, 151, or 180, and/or a wild-type CRISPR-Cas effector protein) in nicking the target strand of a target nucleic acid. In some embodiments, an engineered protein may provide for an increased number of target strand breaks in a target nucleic acid compared to the number of target strand breaks in the target nucleic acid with a CRISPR-Cas effector protein (e.g., Cas12a, a CRISPR-Cas effector protein having a sequence of SEQ ID NOs: 50-66, 151, or 180, and/or a wild-type CRISPR-Cas effector protein). In some embodiments, an engineered protein may have increased efficiency compared to a CRISPR-Cas effector protein (e.g., Cas12a, a CRISPR-Cas effector protein having a sequence of SEQ ID NOs: 50-66, 151, or 180, and/or a wild-type CRISPR-Cas effector protein) in modifying a target nucleic acid.

Compositions, complexes, and systems comprising an engineered protein may be provided according to embodiments of the present invention. In some embodiments, a composition, complex and/or system comprising an engineered protein may be a base editing composition, complex, and/or system. A composition, complex, and/or system of the present invention may include a guide nucleic acid (e.g., a guide RNA) and/or a deaminase (e.g., a cytosine deaminase and/or an adenine deaminase). In some embodiments, an engineered protein, guide nucleic acid, and optionally deaminase form a complex or are comprised in a complex (e.g., a ribonucleoprotein). The engineered protein, guide nucleic acid, and optionally deaminase may not naturally occur together and/or a complex comprising the engineered protein, guide nucleic acid, and optionally deaminase may not naturally occur together. In some embodiments, an engineered protein comprises and/or is fused to a deaminase (e.g., an adenine deaminase and/or a cytosine deaminase).

Also provided herein are nucleic acid molecules encoding an engineered protein of present invention along with an expression cassettes and/or vector comprising a nucleic acid molecule of the present invention.

According to some embodiments, a method is provided that comprises contacting a target nucleic acid with: an engineered protein of the present invention, a guide nucleic acid (e.g., a guide RNA), and optionally a deaminase. In some embodiments, the engineered protein, the guide nucleic acid, and/or deaminase form a complex or are comprised in a complex. In some embodiments, the method may modify the target nucleic acid and/or may provide one or more single strand breaks in the target nucleic acid.

In some embodiments, a composition, system, method, and/or complex comprising an engineered protein may have increased efficiency compared to a composition, system, method, and/or complex comprising a CRISPR-Cas effector protein (e.g., Cas12a, a CRISPR-Cas effector protein having a sequence of SEQ ID NOs: 50-66, 151, or 180, and/or a wild-type CRISPR-Cas effector protein). In some embodiments, a composition, system, method, and/or complex comprising an engineered protein that is a target strand nickase may have increased efficiency compared to a composition, system, method, and/or complex comprising a CRISPR-Cas effector protein (e.g., Cas12a, a CRISPR-Cas effector protein having a sequence of SEQ ID NOs: 50-66, 151, or 180, and/or a wild-type CRISPR-Cas effector protein). This may be because nicking the target strand may increase the efficiency of genome editing tools such as base editors and/or base diversifiers. In some embodiments, a composition, system, method, and/or complex comprising an engineered protein may provide for an increased number of target strand breaks in a target nucleic acid compared to the number of target strand breaks in the target nucleic acid with a composition, system, method, and/or complex comprising a CRISPR-Cas effector protein (e.g., Cas12a, a CRISPR-Cas effector protein having a sequence of SEQ ID NOs: 50-66, 151, or 180, and/or a wild-type CRISPR-Cas effector protein).

An engineered protein and/or a composition, system, method, and/or complex comprising an engineered protein may provide improved or altered insertion and/or deletion (e.g., indel) size and/or composition, improved or altered deletion size in a target nucleic acid, improved or altered nicking ability on either strand (i.e., target or nontarget strand of a target nucleic acid), and/or increased nuclease activity compared to a CRISPR-Cas effector protein (e.g., Cas12a, a CRISPR-Cas effector protein having a sequence of SEQ ID NOs: 50-66, 151, or 180, and/or a wild-type CRISPR-Cas effector protein) and/or to a composition, system, method, and/or complex comprising a CRISPR-Cas effector protein (e.g., Cas12a, a CRISPR-Cas effector protein having a sequence of SEQ ID NOs: 50-66, 151, or 180, and/or a wild-type CRISPR-Cas effector protein). In some embodiments, an engineered protein and/or a composition, system, method, and/or complex comprising an engineered protein imparts nuclease function onto a catalytically inactivated CRISPR-Cas effector protein. In some embodiments, an engineered protein and/or a composition, system, method, and/or complex comprising an engineered protein provides a different editing profile and/or a different cleavage pattern for a target nucleic acid compared to the editing profile and/or a different cleavage pattern of the target nucleic acid for a Cas effector protein ((e.g., Cas12a, a CRISPR-Cas effector protein having a sequence of SEQ ID NOs: 50-66, 151, or 180, and/or a wild-type CRISPR-Cas effector protein) and/or a composition, system, method, and/or complex comprising a CRISPR-Cas effector protein (e.g., Cas12a, a CRISPR-Cas effector protein having a sequence of SEQ ID NOs: 50-66, 151, or 180, and/or a wild-type CRISPR-Cas effector protein).

In some embodiments, a method of the present invention may have increased efficiency in modifying a target nucleic acid compared to the efficiency of a control method (e.g., a method comprising contacting the target nucleic acid with a CRISPR-Cas effector protein (e.g., Cas12a, a CRISPR-Cas effector protein having a sequence of SEQ ID NOs: 50-66, 151, or 180, and/or a wild-type CRISPR-Cas effector protein) and/or that is devoid of an engineered protein).

As described herein, the polypeptides, engineered proteins, nucleic acids, expression cassettes, and/or vectors of the present invention may be codon optimized for expression in an organism. An organism useful with this invention may be any organism or cell thereof for which nucleic acid modification may be useful. An organism can include, but is not limited to, any animal (e.g., a mammal), any plant, any fungus, any archaeon, or any bacterium. In some embodiments, the organism may be a plant or cell thereof. In some embodiments, the organism is an animal such as a mammal (e.g., a human).

The target nucleic acid may be a genomic sequence from any organism (e.g., eukaryote such as a mammal or a plant). In some embodiments, the target nucleic acid is a genomic sequence from a model organism such as, but not limited to, Escherichia coli, an immortalized human cell line (e.g., HEK293, HeLa, etc.), Caenorhabditis elegans, and/or Drosophila Melanogaster. In some embodiments, the target nucleic acid is a genomic sequence from a non-model organism. Exemplary non-model organisms include, but are not limited to crop plants (e.g., fruit crop plants, vegetable crop plants, and/or field crop plants) and/or animals such as humans, primates and/or mice. In some embodiments, the non-model organism is a crop plant such as corn, soybean, wheat, or canola. In some embodiments, the non-model organism is an animal for testing and/or use of a human therapeutic.

A target nucleic acid of any plant or plant part may be modified and/or edited (e.g., mutated, e.g., base edited, cleaved, nicked, and the like) using the nucleic acid constructs of the invention. Any plant (or groupings of plants, for example, into a genus or higher order classification) may be modified using the nucleic acid constructs of this invention including an angiosperm, a gymnosperm, a monocot, a dicot, a C3, C4, CAM plant, a bryophyte, a fern and/or fern ally, a microalgae, and/or a macroalgae. A plant and/or plant part useful with this invention may be a plant and/or plant part of any plant species/variety/cultivar. The term “plant part,” as used herein, includes but is not limited to, embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell can be in the form of an isolated single cell or can be a cultured cell or can be a part of a higher-organized unit such as, for example, a plant tissue or a plant organ.

Non-limiting examples of plants useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, Chinese cabbage, bok choy), cardoni, carrots, napa, okra, onions, celery, parsley, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas (e.g., field peas, snow peas, snap peas), soybeans, garbanzo beans (chickpeas)), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, sunflower, raspberry, blackberry, black raspberry and/or cherry.

The present invention further comprises a kit to carry out methods of this invention. A kit of this invention can comprise reagents, buffers, and/or apparatus for mixing, measuring, sorting, labeling, etc., as well as instructions and the like as would be appropriate for modifying a target nucleic acid.

In some embodiments, the invention provides a kit comprising one or more polynucleotides and/or nucleic acid constructs of the invention, and/or expression cassettes and/or vectors comprising the same, with optional instructions for the use thereof. In some embodiments, a kit may further comprise a polypeptide of interest and/or polynucleotide encoding the same and expression cassette and/or vector comprising the same. In some embodiments, a guide nucleic acid may be provided on the same expression cassette and/or vector as a nucleic acid construct of the invention. In some embodiments, a guide nucleic acid may be provided on a separate expression cassette or vector from that comprising the nucleic acid construct of the invention.

Accordingly, in some embodiments, kits are provided comprising a nucleic acid construct comprising (a) a polynucleotide(s) as provided herein and (b) a promoter that drives expression of the polynucleotide(s) of (a). In some embodiments, the kit may further comprise a nucleic acid construct encoding a guide nucleic acid, wherein the construct comprises a cloning site for cloning into the backbone of the guide nucleic acid a nucleic acid sequence that is identical or complementary to a target nucleic acid sequence.

In some embodiments, the kit may comprise a nucleic acid construct comprising/encoding one or more nuclear localization signals, wherein the nuclear localization signals are fused to the CRISPR-Cas nuclease. In some embodiments, kits are provided comprising a nucleic acid construct of the invention encoding a modified CRISPR-Cas nuclease of the invention or, and/or an expression cassette and/or vector comprising the same, wherein the nucleic acid constructs, expression cassettes and/or vectors may further encode one or more selectable markers useful for identifying transformants (e.g., a nucleic acid encoding an antibiotic resistance gene, herbicide resistance gene, and the like). In some embodiments, the nucleic acid construct may be an mRNA that encodes one or more introns within the encoded CRISPR-Cas nuclease. In some embodiments, a kit may comprise promoters and promoters with introns for use in expression of the polypeptides and nucleic acid constructs of the invention.

A polypeptide, polynucleotide, nucleic acid construct, expression cassette, vector, composition, kit, system and/or cell of the present invention may comprise all or a portion of a sequence of one or more of SEQ ID NOs: 1-192. In some embodiments, a polypeptide, polynucleotide, nucleic acid construct, expression cassette, vector, composition, kit, system and/or cell of the present invention may comprise at least about 20%, 25%, 30%, 35%, 40%, 45%, 50%, 55%, 60%, 65%, 70%, 75%, 80%, 85%, 90%, 95%, 96%, 97%, 98%, 99%, or more consecutive amino acids of a sequence of one or more of SEQ ID NOs: 1-192.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

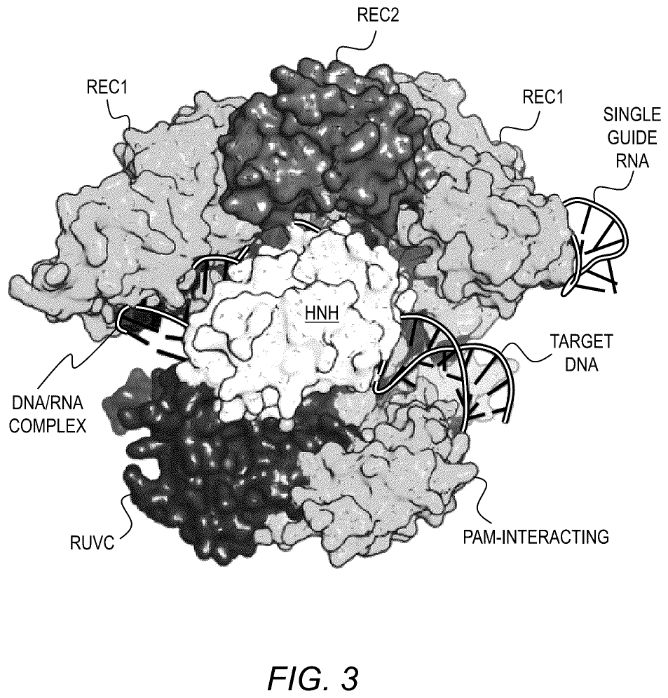

Using existing domain annotations along with visual inspection of the SpCas9 crystal structure (PDB ID 4UN3) in PyMOL (The PyMOL Molecular Graphics System, Version 2.0. Schrodinger, LLC), the full HNH domain from SpCas9 (FIG. 3) was first identified and its residue boundaries determined. The domain is largely resolved in the crystal structure, but several residues connecting the N terminus of the HNH domain to the Rec1 domain are unresolved in the crystal structure. The location of Cas9 target DNA strand cleavage site relative to the HNH domain was also noted. This relative orientation was mimicked in subsequent rational positioning of the HNH domain relative to the target DNA strand of Cas12a.

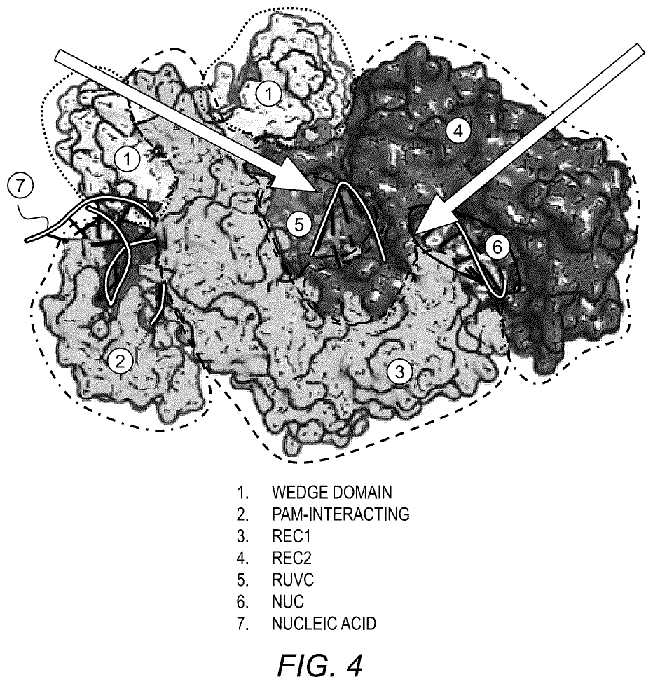

The crystal structure of the LbCas12a ternary complex (PDB ID 5XUS) was next examined to locate an accessible region of the target DNA strand. Although the side of the target DNA/crRNA duplex closest to the RuvC domain is heavily shielded by other Cas12a domains, there is an exposed portion of the target DNA (indicated by the left arrow in FIG. 4) on the opposite side of the protein at the interface between the Rec1 and Rec2 domains (FIG. 4). A linker (indicated by the right arrow in FIG. 4) connecting the two domains sits adjacent to this exposed site and has few interactions with other residues in LbCas12a; this linker was chosen as a candidate site for domain insertion.

To determine the precise placement of the SpCas9 HNH domain relative to LbCas12a, the exposed DNA bases in the groove between the Rec1 and Rec2 domains of LbCas12a were next identified. Then, treating the HNH domain and its target DNA (four bases with two on each side of the cleavage site) as a unit, alignments of the HNH target DNA to the exposed target strand of LbCas12a were tested in a sliding window using PyMOL until an alignment was identified that would place the HNH domain near the insertion loop and would minimize clashes with other domains of LbCas12a. The position of the HNH domain was then adjusted manually using PyMOL to minimize clashes between HNH and Cas12a.

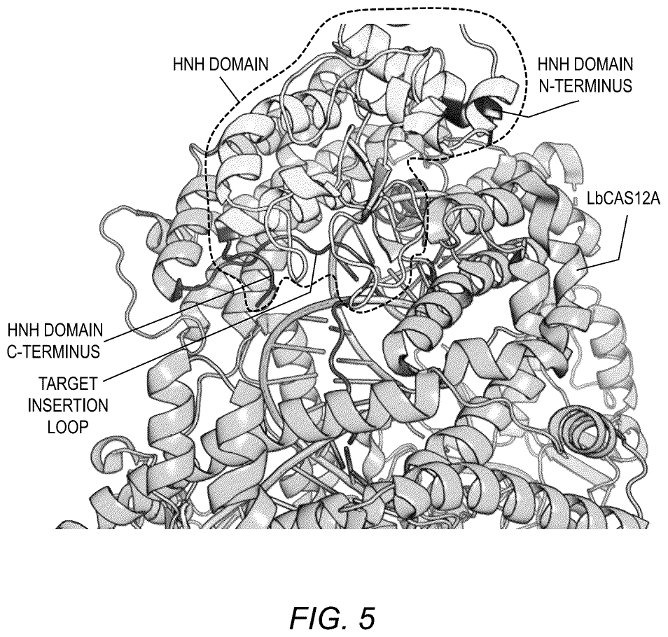

The final selected position of the HNH domain is shown in FIG. 5. While the C terminus of the HNH domain is very close to the C terminal end of the insertion loop, the HNH N terminus is relatively far from the insertion loop; however, this structure does not include the unstructured residues linking the SpCas9 Rec1 and HNH domains. A highly conserved hairpin in this region that interacts with the target DNA/crRNA duplex was further identified as a potential site for later design.

To prepare the Cas12a-HNH fusion structure for computational linker modeling, the N terminus of the HNH domain was initially extended by appending residues from SpCas9 that connect the Rec1 and HNH domains (and which are unresolved in the SpCas9 crystal structure) using PyMOL. The resulting structure was exported and prepared for linker modeling using custom Python scripts that inserted the HNH domain residues into possible insertion sites throughout the insertion loop as shown in Table 1.

TABLE 1
Results of preliminary computational screening
of possible insertion sites in Cas12a.

| | # | |
|---|---|---|
| | successful | |
| Insertion | closures/ | |
| Site | 10 attempts | Observations |
| 282 | 0 | |
| 283 | 0 | |
| 284 | 0 | |
| 285 | 3 | |
| 286 | 0 | |
| 287 | 1 | |
| 288 | 3 | When loop closure does occur, it would likely |
| | | offer little flexibility for the N-terminal |
| | | linker. The C-terminal linker seems long |
| | | enough to be flexible. |
| 289 | 2 | |
| 290 | 0 | |
| 291 | 9 | Insertion in a flexible location between glycine |
| | | and glutamate residues |
| 292 | 9 | Insertion in a flexible location between |
| | | glutamate and glycine residues |
| 293 | 6 | Insertion would be immediately before a |
| | | tyrosine that interacts with other Cas12a |
| | | residues |

A rapid computational screen was then performed to test the ability of the HNH domain termini to connect to the two ends of the linker cut site using the Rosetta Remodel protocol (Huang P. S. et al. (2011) PLoS ONE 6 (8): e24109. https://doi.org/10.1371/journal.pone.002410) included in the Rosetta macromolecular modeling software package. For each insertion point, ten iterations of loop closure (with no sequence design or insertions) were performed. The number of times that the linkers were able to successfully connect out of those ten iterations were tallied and compared (Table 1). Two of these insertion sites were then selected for more thorough linker modeling, including variations in linker lengths, based on a combination of their rate of successful loop closure and manual inspection (shown in bold in Table 1).

For the two selected insertion sites, fine-grained testing was then performed with small (2 to 4 residue) glycine-serine insertions or deletions in the N-terminal and C-terminal linkers and with more thorough sampling (100 iterations each). Possible residues for deletion were selected based on manual inspection of the sequence. Based on the linker modeling results, eight designs (four for each insertion site) were selected for experimental testing including extensions of 0, 2, or 4 residues of the N-terminal linker and extensions of 0 or 2 residues of the C-terminal linker.

### Example 2

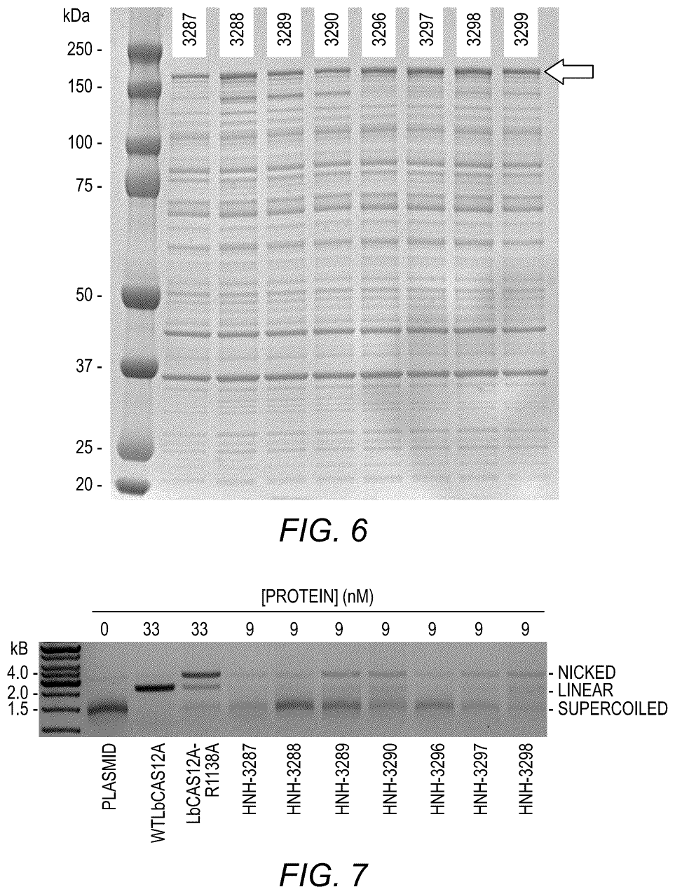

DNA coding regions for 8 LbCas12a-HNH constructs (HNH-3287, HNH-3288, HNH-3289, HNH-3290, HNH-3296, HNH-3297, HNH-3298, and HNH-3299) were synthesized with a 6-Histidine tag using solid state synthesis. The coding regions were coned into a pET28a plasmid (Novagen) behind an inducible T7 promoter and transfected into BL21 (DE3)-Star cells (Invitrogen) and plated on kanamycin. Single colonies were grown in 30 ml of Luria Broth at 37° C. to an A600 optical density of 0.5. 500 mM IPTG was added and the temperature was lowered to 18° C. for 18 hours of expression. Cells were pelleted and lysed with BugBuster Master Mix (Millipore) according to the manufacturer's directions. Cell debris was pelleted and a soluble fraction was imaged on a 4-12% Bis-Tis PAGE gel (Invitrogen) under reducing conditions, and visualized using Coomassie staining. All eight HNH constructs showed soluble protein expression at the approximate MW of 160 kDa (FIG. 6, arrow).

Soluble protein expression for all eight constructs containing HNH nuclease in the middle of the Cas12a protein speaks to the quality of the fusion designs. Large domain insertions into the middle of proteins often results in insoluble protein expression or no expression in Escherichia coli. The observation of all eight proteins being highly expressed suggests the chimeric protein is folding properly and has not led to the disruption of either protein folds.

The expression protocol was repeated to generate proteins suitable for nuclease assays. After pelleting the eight constructs, the E. coli cells were frozen, thawed, and were suspended in Buffer A (20 mM HEPES-KOH PH 7.5, 500 mM NaCl, 10% glycerol, 2 mM TCEP, and 10 mM Imidazole pH 7.5). 0.3 mg/ml lysozyme was added, and the cells were incubated at room temperature for 20 minutes, followed by sonication (QSonica) with a ⅛-inch tip, 25% power, 10 second bursts followed by 30 second rests for 2.25 minutes. Cell debris was pelleted, and the supernatant was loaded onto Ni-NTA Agarose (Bio-Rad), washed with 20 mM imidazole in buffer A, and eluted with 300 mM imidazole in Buffer A. Approximate concentrations of proteins were 0.5 to 2 mg/ml (estimated by NanoDrop A280 absorbances) in a total eluate of 200 μL.

### Example 3

A plasmid-based assay was used to assess nicking activity by purified HNH-3287, HNH-3288, HNH-3289, HNH-3290, HNH-3296, HNH-3297, and HNH-3298. Plasmid nicking assays work on the principle that supercoiled plasmids extracted from bacteria run smaller on agarose gels than linearized, double-cut plasmid. Furthermore, if only one strand is nicked the plasmid runs even larger than linearized plasmid. This assay has been used extensively in the CRISPR field to assess if an enzyme is a double-stranded nuclease or a single-stranded nuclease (Jinek et al., Science. 2012 Aug. 17; 337 (6096): 816-21) (Zetsche et al., Cell. 2015 Oct. 22; 163 (3): 759-71).

The sequence 5′-TTTAGGAAT CCCTTCTGC AGCACCTGG-3′ (SEQ ID NO: 123), where the protospacer-adjacent motif (PAM) is in bold, was synthesized and cloned into a pUC18 plasmid. The plasmid was expressed in DH5a cells and purified using plasmid miniprep kits (Qiagen). CRISPR RNA molecules were synthesized (Synthego) without any chemical modifications with the sequence 5′-AAUUUCUACU AAGUGUAGAU GGAAUCCCUU CUGCAGCACC UGG-3′ (SEQ ID NO: 124) where the portion complimentary to the plasmid is emboldened. 30 μL reactions were assembled with a 10:10:1 RNA:Protein:Plasmid ratio, incubated for 15 minutes at 37° C., heat-inactivated at 85° C. for 2 minutes, and loaded on a 1% agarose gel containing 1/100 v/v SYBR-Safe stain (Invitrogen).

Proteins tested were wildtype LbCas12a (wtLbCas12a), LbCas12a-R1138A, and the various chimeric HNH proteins. The R1138A is a point mutation in LbCas12a which corresponds to a known non-template strand nickase mutation for AsCas12a (R1226A) (Yamano T, et al. Cell. 2016 May 5; 165 (4): 949-62). Concentrations tested were 33 nM for wtLbCas12a and LbCas12a-R1138. A lower 9 nM was used for the various HNH constructs to distinguish the most active nucleases by being somewhat near the expected Kd rather than generating complete nicking.

The resulting gel (FIG. 7) indicates that HNH-3287, HNH-3288, HNH-3289, HNH-3290, HNH-3296, HNH-3297, and HNH-3298 are all nickases with percentages of nicking from ˜25% efficiency to ˜75% efficiency (upper bands, the nicked plasmids, compared to lower bands, the supercoiled plasmids) at these low 9 nM protein concentrations. Longer incubations or higher concentrations result in complete nicking, but do not allow for comparing relative mutant activities. Chimera HNH-3298 appears to have the highest percentage of nicking activity with 9 nM [protein] for 15 minutes at 37° C.

### Example 4

### Methods

### Protein Expression and Purification

For initial testing of expression and activity, His-tagged proteins, SYN3287 (SEQ ID NO: 105; SEQ ID NO: 125), SYN3288 (SEQ ID NO: 106; SEQ ID NO: 126), SYN3289 (SEQ ID NO: 127), SYN3290 (SEQ ID NO: 128), SYN3296 (SEQ ID NO: 129), SYN3297 (SEQ ID NO: 130), SYN3298 (SEQ ID NO: 107; SEQ ID NO: 131), and SYN3299 (SEQ ID NO: 132), were expressed in BL21 cells in 30 mL cultures. Each of the proteins included an active HNH domain and an inactivated RuvC domain. Cells were pelleted, frozen overnight, and lysed by sonication. Proteins were then crudely purified from the lysate using HisPur™ Ni-NTA Spin Columns.

For assays of SYN3298 and SYN3289, proteins were expressed in the same way with the following changes: proteins were expressed in 1 L cultures and purified using HisTrap-HF columns by FPLC. Fractions containing the protein of interest were further purified by cation exchange and stored in 50% glycerol.

### Plasmid Nickase Assay

To determine the activity of purified proteins as nickases or nucleases, 30 μL reactions were prepared containing 1×NEBuffer 3.1, 100 femtomoles of the DNA substrate, and equal parts purified protein and an appropriate guide RNA (1 picomole of each unless otherwise noted). Reactions were incubated at 37° C. for 30 minutes, stopped by a 20 minute Proteinase K digestion at room temperature, and separated on a 1% agarose gel. The target site for the plasmid nickase assay had a sequence of SEQ ID NO: 133.

### Fluorescent Nickase Assay

DNA substrates were produced by annealing one labeled (SEQ ID NO: 134) and one unlabeled (SEQ ID NO: 135) DNA strand to produce substrates labeled with Cy5 either on the PAM-containing or non-PAM-containing strand. The spacer for this assay included a sequence of SEQ ID NO: 150. Nicking reactions were prepared as described for the plasmid nickase assay an incubated at 37° C. for 30 minutes. Reactions were stopped by digesting samples with Proteinase K for 10 minutes. All samples were then mixed with urea loading buffer to 1× concentration and heated to 90° C. for 5 minutes to denature the substrates. Samples were separated by running on a 6% TBE Urea gel at 4° C. and 100V.

### HEK293T Cell Transfection

Eukaryotic HEK293T (ATCC CRL-3216) cells were cultured in Dulbecco's Modified Eagle's Medium plus GlutaMax (ThermoFisher) supplemented with 10% (v/v) FBS (FBS), at 37° C. with 5% CO2. Protein components were synthesized using gene synthesis and subsequently cloned into plasmids with a CMV promoter. Guide RNAs were cloned with a human U6 promoter. HEK293T cells were seeded on 48-well collagen-coated BioCoat plates (Corning). Cells were transfected at about 70% confluency. 375 ng of CRISPR plasmid and 125 ng of guide RNA expression plasmids were transfected using 1.5 μl of Lipofectamine 3000 (ThermoFisher Scientific) per well according to the manufacturer's protocol. Genomic DNA from transfected cells were obtained after 3 days and indels were detected and quantified using high-throughput Illumina amplicon sequencing.

To determine which strand the designed proteins preferentially nick, pairs of guides were designed such that a Cas9 guide and a guide for the designed proteins on the same strand would cut close to each other (within ˜10 bp). Each tested design was paired with either a nuclease-dead SpCas9, a SpCas9 D10A target strand nickase, or a SpCas9 H840A nontarget strand nickase. If the synthetic nickase and its paired Cas9 nickase cut opposite strands, then a greater editing frequency was expected than if they cut the same strand due to the production of double-stranded breaks.

### Results

His-Tagged Designed Synthetic Nickases were Successfully Expressed in BL21 E. coli.

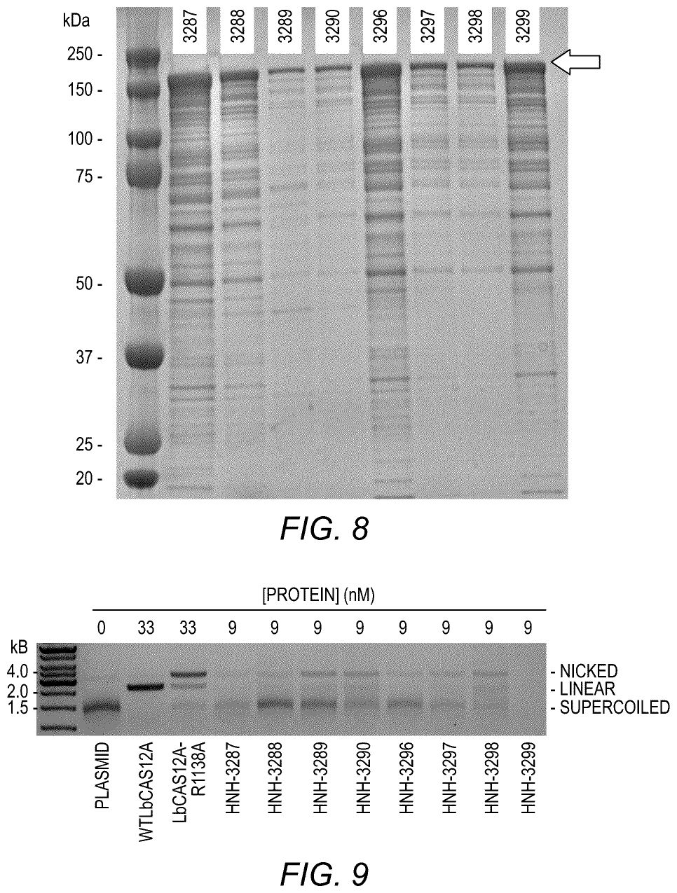

After crude purification of the designed nickases as described above, all samples showed a band at the expected size (˜160 kDa) as shown in FIG. 8, indicating that the nickases were solubly expressed in E. coli.

Initial Plasmid Nicking Activity Observed from Crude Purifications of Synthetic Nickases.

Plasmid nickase assays were performed as described in the methods section above using the crude nickase purifications shown in FIG. 8. Due to low yields from some of the purifications, all designed nickases were tested at low concentrations so that they could be compared directly. As can be seen in FIG. 9, all but one of the designs showed a band indicating the presence of nicked plasmid that was more prominent than in the negative control sample, suggesting that the designs are capable of nicking a DNA substrate.

### RNA Dependence of Plasmid Nicking Using Crudely Purified Synthetic Nickases.

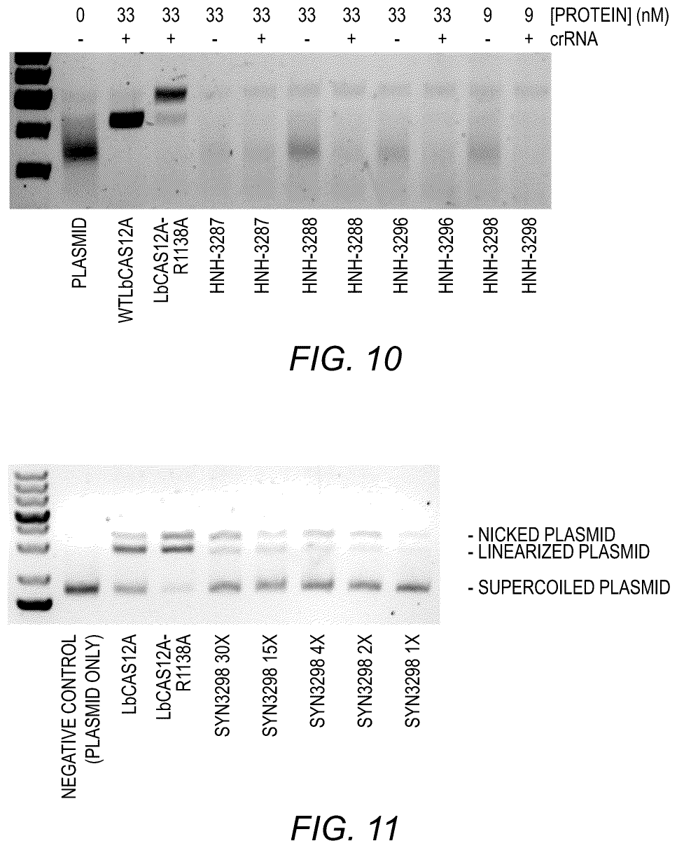

To ensure that the observed nicking and cleavage of the plasmid were guide-dependent and not due to random nuclease activity, the plasmid nickase assay was repeated for selected designs in both the presence of a targeting crRNA. Designs SYN3288, SYN3296, and SYN3298 all showed a reduction in the amount of uncleaved plasmid present in the presence of crRNA as can be seen in FIG. 10, indicating that their nuclease activity is RNA-dependent.

### Plasmid Nicking Activity of Purified Synthetic Nickase SYN3298.

Different quantities of protein+guide were tested relative to the concentration of LbCas12a control used (e.g. 30× indicates that 30 picomoles of protein and guide were included in the reaction). Nicking and, to a lesser extent, cleavage of the plasmid were observed at all tested concentrations of SYN3298 (FIG. 11), confirming that this design acts as a DNA nickase.

### Fluorescent Nickase Assay Using Purified Synthetic Nickases SYN3298 and SYN3289.

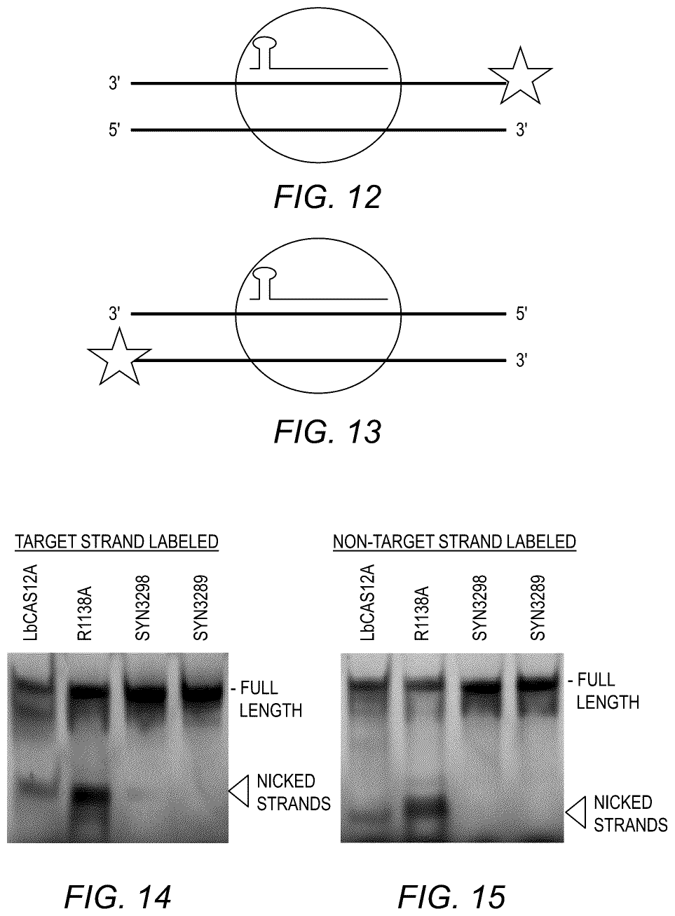

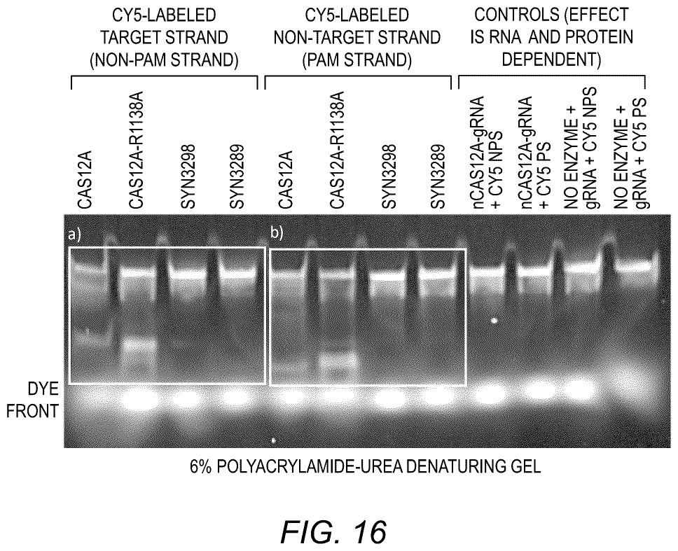

Substrates with a fluorescent Cy5 label on either the target (FIG. 12) or the nontarget (FIG. 13) were incubated with the designed nickases (which included an active HNH domain and an inactivated RuvC domain), LbCas12a, or the LbCas12a R1138A mutant (a nontarget strand nickase) and separated on a denaturing TBE-Urea gel. A shift in the position of the labeled band indicates that that strand was cleaved. FIG. 14 shows a portion of the gel for the samples incubated with the labeled target strand, FIG. 15 shows a portion of the gel for the samples incubated with the labeled non-target strand, and FIG. 16 shows the entire gel with lanes for the controls, the samples incubated with the labeled target strand (the boxed lanes denoted as “a)”), and the samples incubated with the labeled non-target strand (the boxed lanes denoted as “b)”). SYN3298 shows bands at the expected location for a cleaved substrate for the target DNA strand but not the nontarget DNA strand, indicating that it acts as a target strand nickase.

### Sequence-Based Strand-Specific Nickase Assay of Genomic DNA in HEK293T Cells.

Synthetic nickases were co-transfected with nearby Cas9 nickases (e.g., Cas9 (H840A) or Cas9 (D10A) cutting either the target strand (e.g., Cas9 (D10A)) or nontarget strand (e.g., Cas9 (H840A)). Information on the spacers used in the sequence based, strand specific nickase assay is provided in Table 2. Upstream guide refers to which spacer would be expected to cut closer to the 5′ end of the PAM-containing DNA strand. Estimated distance between the cut sites was determined based on the predicted cut site for each native nuclease domain.

TABLE 2
Spacer information for the sequence
based, strand specific nickase assay.

| | | Cas12a/ | Estimated | |
|---|---|---|---|---|
| | | synthetic | distance | |
| | Cas9 | nickase | between | |
| | spacer | spacer | cut | Upstream |
| Target | sequence | sequence | sites | guide |
| RUNX1 | GCATTTT | CAGGAGGAA | 7-12 bp | Cas9 |
| | CAGGAGG | GCGATGGCT | | |
| | AAGCGA | TCAGA | | |
| | (SEQ ID | (SEQ ID | | |
| | NO: 136) | NO: 137) | | |
| AAV1 | GTCCCCT | TCTGTCCCC | 2-7 bp | Cas12a/ |
| | CCACCCC | TCCACCCCA | | synthetic |
| | ACAGTG | CAGTG | | nuclease |
| | (SEQ ID | (SEQ ID | | |
| | NO: 138) | NO: 139) | | |

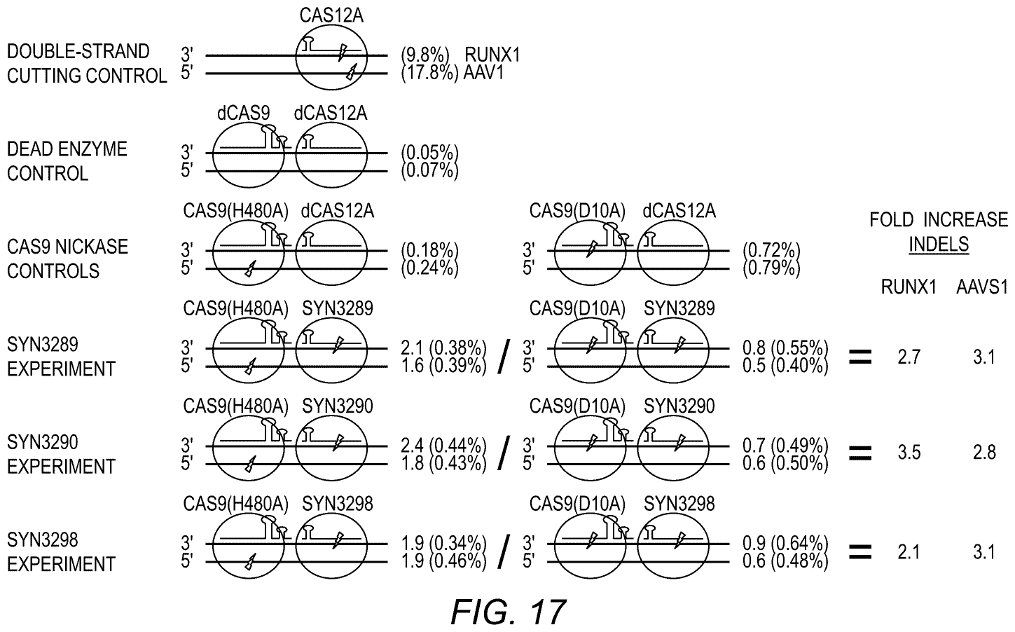

Editing efficiencies for each enzyme pair were normalized to the observed level of indels when the Cas9 nickase was paired with a nuclease-dead LbCas12a at the same target site (FIG. 17). Numbers in parentheses in FIG. 17 indicate the observed editing efficiencies prior to normalization. If the synthetic enzyme (SYN) (i.e., SYN3289, SYN3290, or SYN3298) preferentially cuts the target strand then (H480A::SYN)/(D10A::SYN)>1. If the synthetic enzyme (SYN) (i.e., SYN3289, SYN3290, or SYN3298) preferentially cuts the nontarget strand then (H480A::SYN)/(D10A::SYN)<1.

To determine which strand the designed proteins preferentially nick, pairs of guides were designed such that a Cas9 guide and a guide for the designed proteins on the same strand would cut close to each other (within ˜10 bp). Each tested design was paired with either a nuclease-dead SpCas9, a SpCas9 D10A target strand nickase, or a SpCas9 H840A nontarget strand nickase. If the synthetic nickase and its paired Cas9 nickase cut opposite strands, then a greater editing frequency was expected to be seen than if they cut the same strand due to the production of double-stranded breaks. Increased (about a 3-fold increase) indel frequency was consistently observed when all designed nickases were paired with a Cas9 nontarget strand nickase compared to a Cas9 target strand nickase, indicating that the designed nickases preferentially cut the target DNA strand.

### Example 5

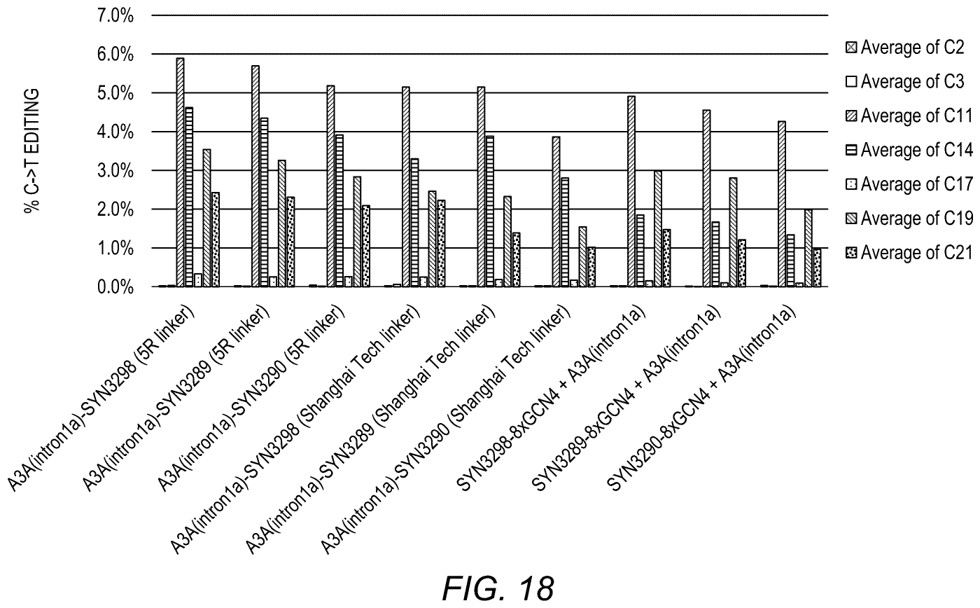

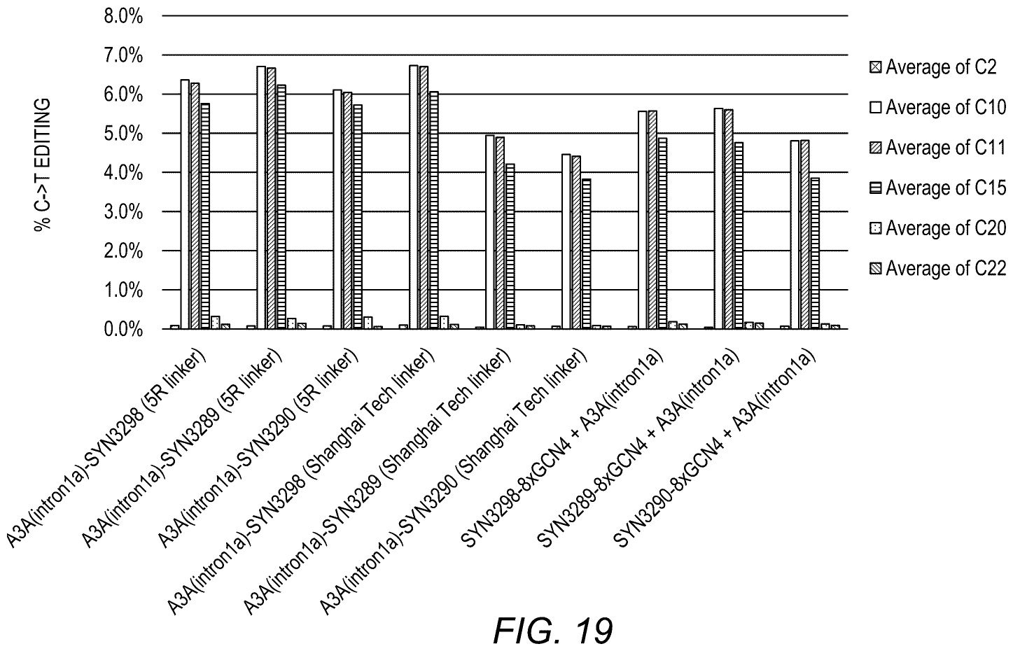

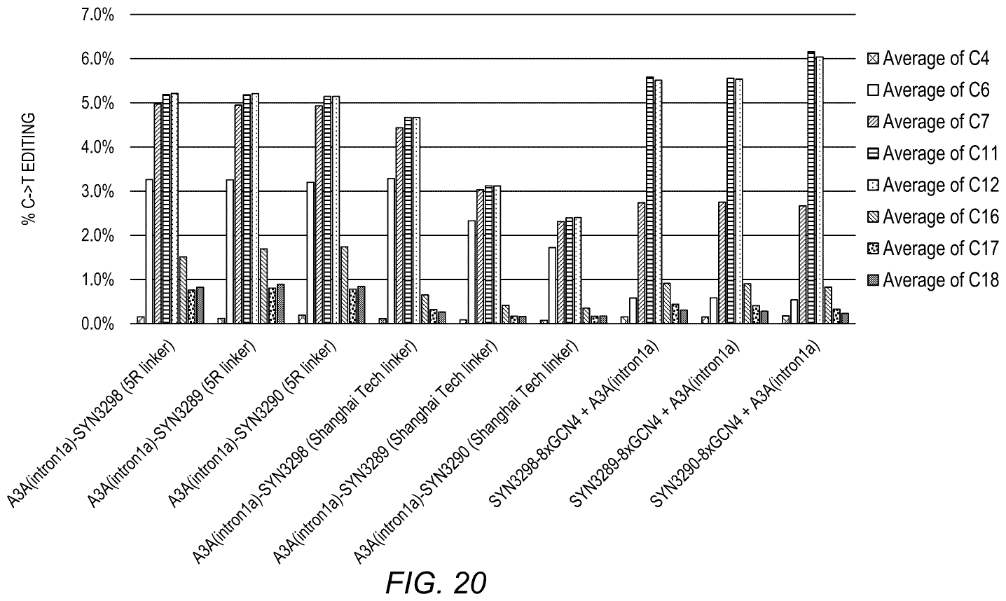

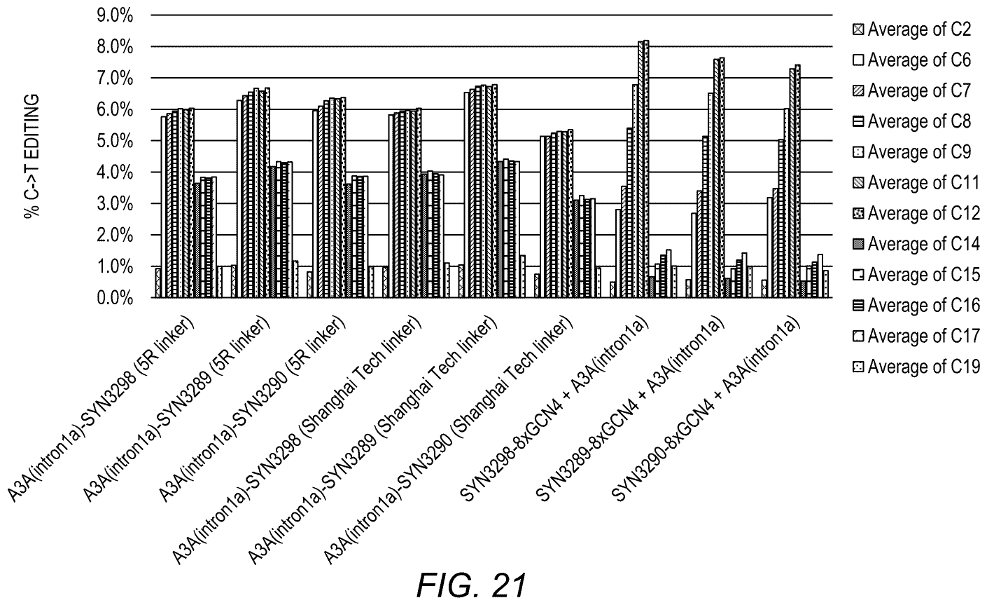

Cytosine base editing data for base editors combining the A3A cytosine deaminase (SEQ ID NO: 152) with SYN3289, SYN3290, or SYN3298 was obtained (FIGS. 18-21). Three architectures were tested for each enzyme: fusion of A3A to the N-terminus of the synthetic enzyme using a linker (SEQ ID NO: 22) along with fusion of UGI (SEQ ID NO: 104) to the C-terminus of the synthetic enzyme using a linker of SEQ ID NO: 45 to provide SEQ ID NOs: 160-162; fusion of A3A to the N terminus of the synthetic enzyme using a previously published linker (SEQ ID NO: 153; Li et al. Nat Biotechnol 36, 324-327 (2018)) along with fusion of UGI (SEQ ID NO: 104) to the C-terminus of the synthetic enzyme using a linker of SEQ ID NO: 154 to provide SEQ ID NOs: 163-165; or Suntag-based recruitment of UGI (SEQ ID NO: 104) fused to the C-terminus of A3A (SEQ ID NO: 152) to provide SEQ ID NO: 156 recruited to the peptide tagged synthetic enzyme of one of SEQ ID NOs: 157-159. All percentages shown in FIGS. 18-21 indicate averages across three data points. All of the tested enzymes demonstrated cytosine base editing in all three of the tested configurations. The spacer for FIG. 18 was SEQ ID NO: 144, the spacer for FIG. 19 was SEQ ID NO: 145, spacer for FIG. 20 was SEQ ID NO: 146, and the spacer for FIG. 21 was SEQ ID NO: 147.

### Example 6

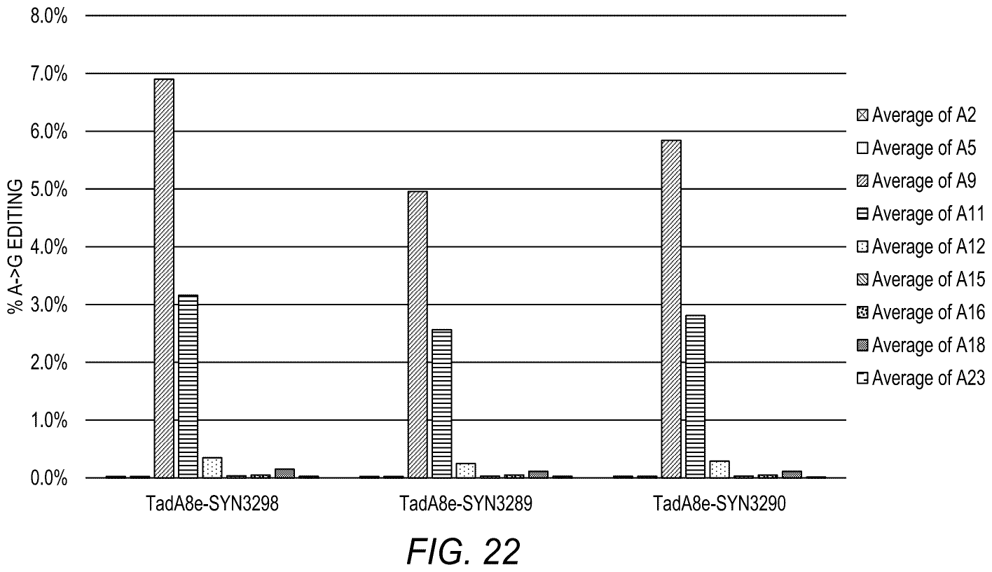

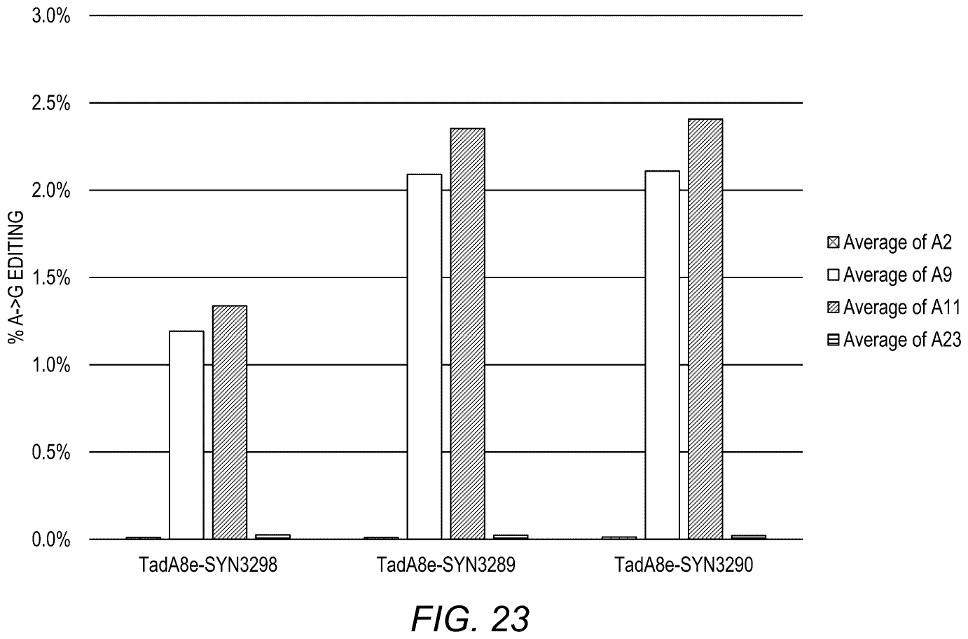

Adenine base editing data for synthetic enzymes SYN3289, SYN3290, and SYN3298 as N-terminal fusions to the TadA8e adenine deaminase was obtained (FIGS. 22-23). The synthetic enzymes were fused to TadA8e (SEQ ID NO: 155) using a linker (SEQ ID NO: 47) to provide SEQ ID NOs: 166-168. All percentages shown in FIGS. 22-23 indicate averages across three data points. The three tested designs all demonstrated adenine base editing activity when fused with TadA8e. The spacer for FIG. 22 was SEQ ID NO: 148 and the spacer for FIG. 23 was SEQ ID NO: 149.

### Example 7

The PAM specificity of the engineered proteins described herein will be tested using the PAM-SCANR assay (Leenay, R. T. et al. Mol Cell 62, 137-147 (2016)) Briefly, an inactivated version of the synthetic enzyme (e.g., SYN3298 having a H370A mutation (i.e., SYN3298 with an alanine at amino acid residue number 370 to provide an inactivating mutation in the HNH domain)) will be expressed in a library of bacteria containing all 256 possible 4-base 5′ PAMs in the promoter of a lacl gene and a GFP gene controlled by a lacZ promoter. In cells where the synthetic enzyme binds to the PAM, the lacl protein will not be expressed, allowing the expression of GFP, and the cells will fluoresce. Fluorescent cells will then be isolated by cell sorting and sequenced to determine which PAMs can be bound by the synthetic enzyme.

### Example 8

The PAM specificity for synthetic enzymes SYN3287, SYN3288, and SYN3298 and mutants thereof was tested with native TTTV PAM sequences (where V is A, G, or C) and/or non-native PAM sequences (e.g., CCCC, TCCA, TCCC, TCCG, TTCA, TTCC, TTCG, TATA, TATC, or TATG). Specifically, SYN3287, SYN3288, or SYN3298 with either G532R and K595R (RR) mutations with reference to position numbering of SEQ ID NO: 180 (LbCas12a); G532R, K538V, and Y542R (RVR) mutations with reference to position numbering of SEQ ID NO: 180 (LbCas12a); or D156R mutation were tested, and the indel generation frequency was determined. RR-LbCas12a has G532R and K595R mutations (SEQ ID NO: 181); 3298 RR has G680R and K743R mutations (SEQ ID NO: 184); 3287 RR has G676R and K739R mutations (SEQ ID NO: 187); 3288 RR has G678R and K741R mutations (SEQ ID NO: 190); RVR-LbCas12a has G532R, K538V, and Y542R mutations (SEQ ID NO: 182); 3298 RVR has G680R, K686V, and Y690R mutations (SEQ ID NO: 185); 3287 RVR has G676R, K682V, and Y686R mutations (SEQ ID NO: 188); 3288 RVR has G678R, K684V, and Y688R mutations (SEQ ID NO: 191); LbCas12a D156R has a D156R mutation (SEQ ID NO: 183); 3298 D156R has a D156R mutation (SEQ ID NO: 186); 3287 D156R has a D156R mutation (SEQ ID NO: 189); and 3288 D156R has a D156R mutation (SEQ ID NO: 192), each with reference to the referred to sequence's own position numbering.

### HEK293-T Cell Testing

HEK293T cells were seeded into 48-well collagen-coated plates (Corning) in the absence of antibiotic using DMEM media. At 70-80% confluency, cells were transfected with 1.5 μl of Lipofectamine 3000 (ThermoFisher Scientific) using 750 ng of editor plasmid and 250 ng of guide RNA plasmid according to manufacturer's protocol. After 3 days, cells were lysed, and DNA was extracted using MagMax DNA extraction kit (Applied Biosystems). Samples were sequenced using NGS, and editing efficiencies were calculated as the average percentage of reads containing desired edits across all available technical replicates.

### Plant Testing

For each PAM sequence to be tested, five spacers each in corn and soy were designed, each spacer targeting a unique locus. Plasmids expressing the editor and spacers to be tested were transformed into corn or soy dried excised embryos (DEEs) via a standard Agrobacterium transformation protocol. After 10 weeks, plantlets were sampled destructively, and all editing targets were sequenced using NGS. Editing efficiencies were calculated for each target as the percentage of analyzed samples containing desired edits comprising at least 1% or 10% of sequenced reads.

### Results

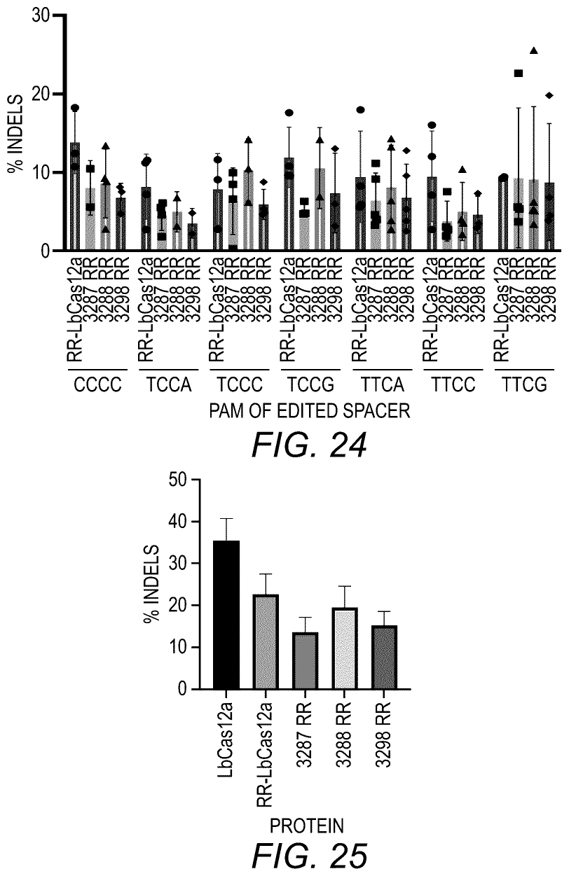

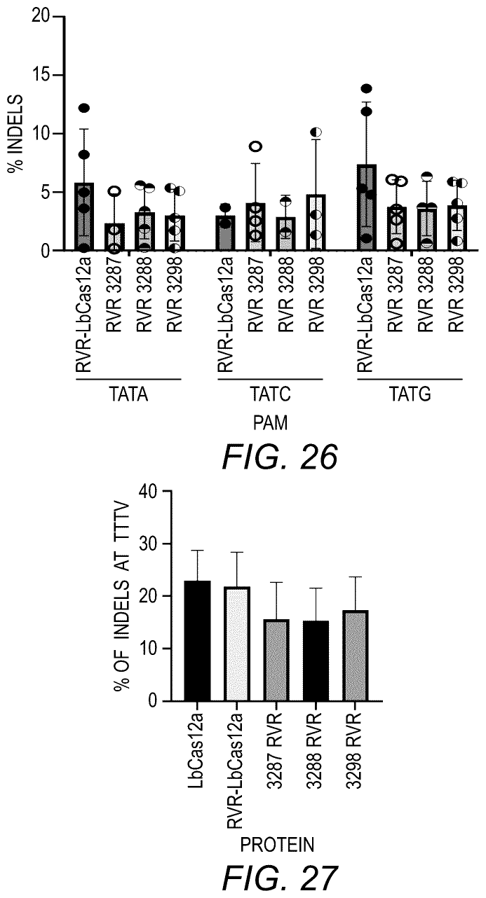

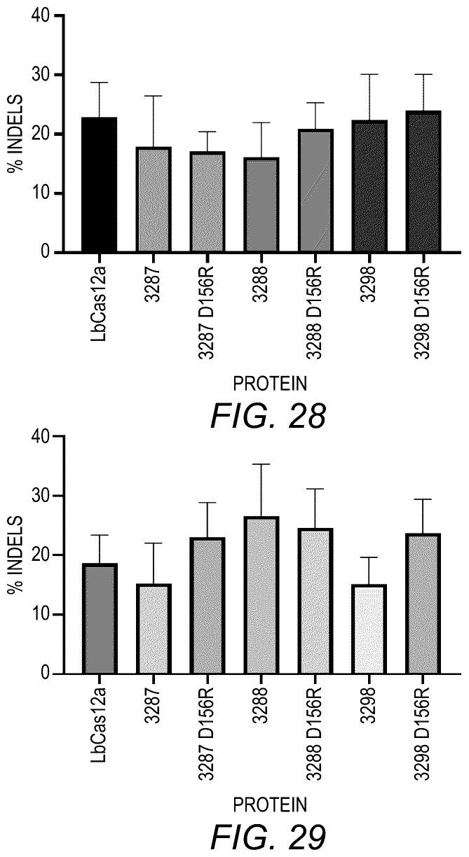

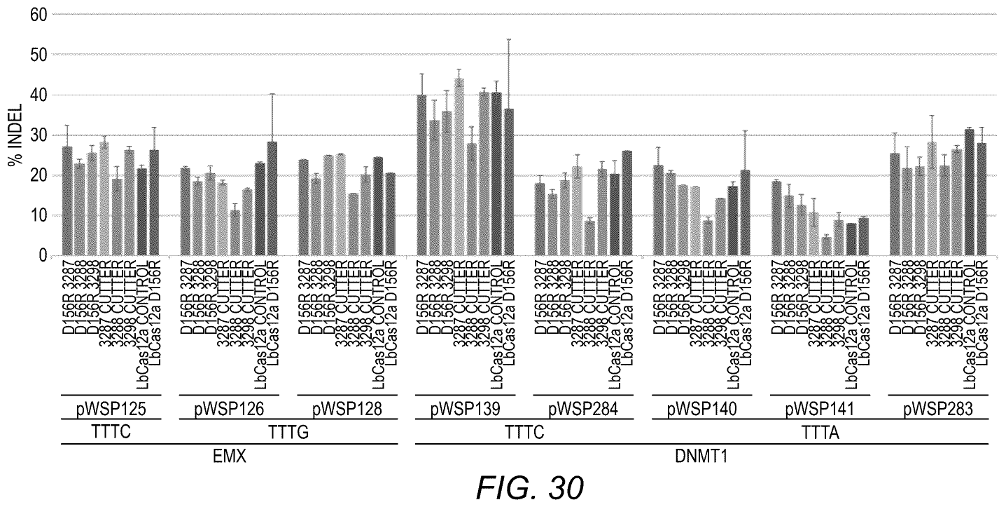

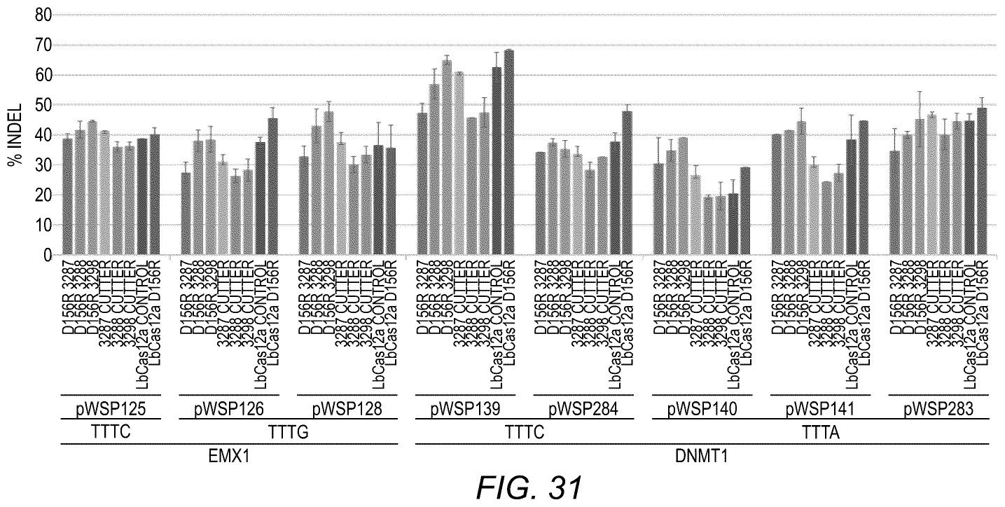

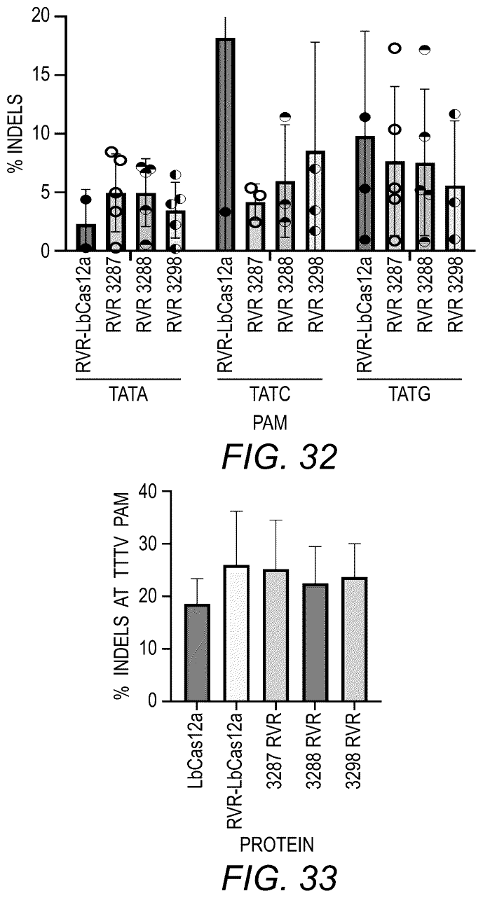

The results are provided in FIGS. 24-33 and Tables 3-10. All proteins tested, with the exception of SYN3288, appear to be as efficient as LbCas12a and D156R controls against all eight TTTV spacers tested. The D156R mutants did not significantly alter PAM specificity and did not negatively affect recognition of TTTV PAM sequences (FIGS. 28-29).

In soy and corn plants, the RR and RVR mutations in SYN3298 (SYN3298-RR and SYN3298-RVR) were shown to have binding affinity for native TTTV PAM sequences and for non-native PAM sequences (Tables 3-10). SYN3298 (SEQ ID NO: 107; SEQ ID NO: 131), SYN3298-RR (SEQ ID NO: 184), and SYN3298-RVR (SEQ ID NO: 185).

TABLE 3
Total counts of desired edit for five different spacers
testing SYN3298, SYN3298-RR, and SYN3298-RVR in soy.

| | | | Spacer | Spacer | Spacer | Spacer | Spacer |
|---|---|---|---|---|---|---|---|
| | PAM | PAM | 1 total | 2 total | 3 total | 4 total | 5 total |
| Protein | type | sequence | counts | counts | counts | counts | counts |
| SYN3298 | Native | TTTV | 174 | 118 | 148 | 134 | 151 |
| SYN3298- | Native | TTTA | 8 | 6 | 7 | 8 | 9 |
| RR | | TTTC | 12 | 7 | 12 | 8 | 10 |
| | | TTTG | 40 | 27 | 40 | 40 | 40 |
| | Non- | CCCC | 1 | 0 | 0 | 0 | 0 |
| | native | TCCA | 2 | 0 | 0 | 1 | 1 |
| | | TCCC | 40 | 38 | 36 | 39 | 31 |
| | | TCCG | 40 | 39 | 26 | 40 | 36 |
| | | TTCA | 1 | 17 | 1 | 47 | 3 |
| | | TTCC | 0 | 32 | 0 | 0 | 0 |
| | | TTCG | 39 | 40 | 17 | 40 | 39 |
| SYN3298- | Native | TTTG | 37 | 40 | 40 | 40 | 14 |
| RVR | Non- | TATA | 34 | 37 | 39 | 36 | 38 |
| | native | TATC | 29 | 40 | 0 | 20 | 35 |
| | | TATG | 0 | 39 | 28 | 36 | 40 |

TABLE 4
Percentage of samples with ≥ 1% editing for each of the five different
spacers used to test SYN3298, SYN3298-RR, and SYN3298-RVR in soy.

| | | % Samples with ≥ 1% editing | | | | |
|---|---|---|---|---|---|---|
| Enzyme | PAM | Spacer 1 | Spacer 2 | Spacer 3 | Spacer 4 | Spacer 5 |
| SYN3298 | TTTV | 64.9 | 78 | 3.4 | 26.9 | 68.5 |
| SYN3298- | TTTA | 87.5 | 100 | 14.3 | 62.5 | 77.8 |
| RR | TTTC | 0 | 0 | 0 | 50 | 100 |
| | TTTG | 2.5 | 77.8 | 80 | 0 | 2.5 |
| | CCCC | 100 | — | — | — | — |
| | TCCA | 0 | — | — | 0 | 0 |
| | TCCC | 75 | 79 | 33.3 | 30.8 | 0 |
| | TCCG | 100 | 33.3 | 19.2 | 42.5 | 0 |
| | TTCA | 0 | 58.8 | 0 | 23.4 | 66.7 |
| | TTCC | — | 19 | — | — | — |
| | TTCG | 51.3 | 0 | 0 | 0 | 20.5 |
| SYN3298- | TTTG | 2.7 | 60 | 57.5 | 2.5 | 0 |
| RVR | TATA | 2.9 | 0 | 2.6 | 0. | 0 |
| | TATC | 0.00 | 0 | — | 15 | 0 |
| | TATG | — | 0 | 0 | 0 | 0 |

A “—” indicates that no data were returned for a given spacer.

TABLE 5
Percentage of samples with ≥ 10% editing for each of the five different
spacers used to test SYN3298, SYN3298-RR, and SYN3298-RVR in soy.

| | | % Samples with ≥ 10% Editing | | | | |
|---|---|---|---|---|---|---|
| Enzyme | PAM | Spacer 1 | Spacer 2 | Spacer 3 | Spacer 4 | Spacer 5 |
| SYN3298 | TTTV | 29.2 | 44.7 | 1.6 | 8.9 | 31.4 |
| SYN3298- | TTTA | 75 | 66.7 | 0 | 0 | 22 |
| RR | TTTC | 0 | 0 | 0 | 25 | 10 |
| | TTTG | 2.5 | 14.8 | 30 | 0 | 0 |
| | CCCC | 10 | — | — | — | — |
| | TCCA | 0 | — | — | 0 | 0 |
| | TCCC | 22.5 | 18.4 | 2.8 | 2.6 | 0 |
| | TCCG | 30 | 15.4 | 0 | 12.5 | 0 |
| | TTCA | 0 | 29.4 | 0 | 2.1 | 0 |
| | TTCC | — | 9.4 | — | — | — |
| | TTCG | 2.6 | 0 | 0 | 0 | 2.6 |
| SYN3298- | TTTG | 0 | 22.5 | 17.5 | 0 | 0 |
| RVR | TATA | 0 | 0 | 0 | 0 | 0 |
| | TATC | 0 | 0 | — | 0 | 0 |
| | TATG | — | 0 | 0 | 0 | 0 |

A “—” indicates that no data were returned for a given spacer.

TABLE 6
Overall PAM specificity of SYN3298,
SYN3298-RR, and SYN3298-RVR in soy.

| | | | Samples with | Samples with | |
|---|---|---|---|---|---|
| | | | desired edits | desired edits | |
| | | | comprising at | comprising at | |
| | | PAM | least 1% of | least 10% of | |
| | PAM | se- | total sequence | total sequence | Total |
| Protein | type | quence | reads | reads | samples |
| SYN3298 | Native | TTTV | 48.2% | 19.6% | 725 |
| SYN3298- | Native | TTTA | 68.4% | 31.6% | 38 |
| RR | | TTTC | 28.6% | 6.1% | 49 |
| | | TTTG | 29.4% | 9.1% | 187 |
| | Non- | CCCC | 100% | 100% | 1 |
| | native | TCCA | 0% | 0% | 4 |
| | | TCCC | 45.7% | 9.8% | 184 |
| | | TCCG | 41.4% | 12.7% | 181 |
| | | TTCA | 33.3% | 8.7% | 69 |
| | | TTCC | 18.8% | 9.4% | 32 |
| | | TTCG | 16% | 1.1% | 175 |
| SYN3298- | Native | TTTG | 28.7% | 9.4% | 171 |
| RVR | Non- | TATA | 1.1% | 0% | 184 |
| | native | TATC | 2.4% | 0% | 124 |
| | | TATG | 0% | 0% | 143 |

TABLE 7
Total counts of desired edit for five different spacers
testing SYN3298, SYN3298-RR, and SYN3298-RVR in corn.

| | | | Spacer | Spacer | Spacer | Spacer | Spacer |
|---|---|---|---|---|---|---|---|
| | PAM | PAM | 1 total | 2 total | 3 total | 4 total | 5 total |
| Protein | type | sequence | counts | counts | counts | counts | counts |
| SYN3298 | Native | TTTV | 0 | 401 | 452 | 405 | 509 |
| SYN3298- | Native | TTTA | 83 | 77 | 107 | 97 | 89 |
| RR | | TTTC | 0 | 60 | 77 | 80 | 54 |
| | | TTTG | 39 | 47 | 48 | 47 | 44 |
| | Non- | TTCA | 91 | 36 | 65 | 0 | 62 |
| | native | TCCG | 34 | 20 | 0 | 27 | 2810 |
| | | CCCC | 58 | 37 | 81 | 91 | 0 |
| | | TCCA | 33 | 24 | 28 | 0 | 25 |
| | | TCCC | 18 | 30 | 39 | 0 | 37 |
| | | TTCC | 0 | 47 | 46 | 49 | 2 |
| | | TTCG | 33 | 39 | 13 | 15 | 0 |
| SYN3298- | Native | TTTA | 15 | 12 | 13 | 0 | 6 |
| RVR | | TTTC | 0 | 16 | 19 | 0 | 13 |
| | | TTTG | 17 | 16 | 0 | 20 | 22 |

TABLE 8
Percentage of samples with ≥ 1% editing for each of the five different
spacers used to test SYN3298, SYN3298-RR, and SYN3298-RVR in corn.

| | | % Samples with ≥ 1% editing | | | | |
|---|---|---|---|---|---|---|
| Protein | PAM | Spacer 1 | Spacer 2 | Spacer 3 | Spacer 4 | Spacer 5 |
| SYN3298 | TTTV | 509 | — | 20.5 | 0.9 | 18.8 |
| SYN3298- | TTTA | 89 | 12.1 | 32.5 | 32.7 | 0 |
| RR | TTTC | 54 | — | 3.3 | 9.1 | 8.8 |
| | TTTG | 44 | 0 | 0 | 6.3 | 4.3 |
| | CCCC | 0 | 12.1 | 0 | 0 | 4.4 |
| | TCCA | 25 | 9.1 | 0 | 0 | — |
| | TCCC | 37 | 16.7 | 16.7 | 2.6 | — |
| | TCCG | 28 | 0 | 5 | — | 3.7 |
| | TTCA | 62 | 7.7 | 2.8 | 9.2 | — |
| | TTCC | 2 | — | 34.1 | 52.2 | 10.2 |
| | TTCG | 0 | 3 | 10.3 | 30.8 | 0 |
| SYN3298- | TTTA | 6 | 0 | 0 | 7.7 | — |
| RVR | TTTC | 13 | — | 0.00 | 10.5 | — |
| | TTTG | 22 | 5.9 | 0 | — | 5 |

A “—” indicates that no data were returned for a given spacer.

TABLE 9
Percentage of samples with ≥ 10% editing for each of the five different
spacers used to test SYN3298, SYN3298-RR, and SYN3298-RVR in corn.

| | | % Samples with ≥ 10% Editing | | | | |
|---|---|---|---|---|---|---|
| Protein | PAM | Spacer 1 | Spacer 2 | Spacer 3 | Spacer 4 | Spacer 5 |
| SYN3298 | TTTV | 27.9 | — | 15.7 | 0.4 | 13.8 |
| SYN3298- | TTTA | 19.1 | 6 | 28.6 | 28 | 0 |
| RR | TTTC | 5.6 | — | 1.7 | 6.5 | 8.8 |
| | TTTG | 0 | 0 | 0 | 6.3 | 2 |
| | CCCC | — | 12.1 | 0 | 0 | 3.3 |
| | TCCA | 0 | 5.5 | 0 | 0 | — |
| | TCCC | 5.4 | 11.1 | 16.7 | 2.6 | — |
| | TCCG | 0 | 0 | 5 | — | 0 |
| | TTCA | 0 | 4.4 | 0 | 3.1 | — |
| | TTCC | 0 | — | 17.9 | 33.3 | 6.8 |
| | TTCG | — | 3 | 10.3 | 23.1 | 0 |
| SYN3298- | TTTA | 0 | 0 | 0 | 0 | — |
| RVR | TTTC | 7.7 | — | 0 | 10.5 | — |
| | TTTG | 0 | 5.9 | 0 | — | 5 |

A “—” indicates that no data were returned for a given spacer.

TABLE 10

| | | | Samples | Samples | |
|---|---|---|---|---|---|
| | | | with | with | |
| | | | desired | desired | |
| | | | edits | edits | |
| | | | comprising | comprising | |
| | | | at | at | |
| | | | least | least | |
| | | | 1% of | 10% of | |
| | | | total | total | |
| | PAM | PAM | sequence | sequence | Total |
| Protein | type | sequence | reads | reads | samples |
| SYN3298 | Native | TTTV | 17.2% | 13.9% | 1767 |
| SYN3298- | Native | TTTA | 19.2% | 15% | 453 |
| RR | | TTTC | 7% | 5.9% | 271 |
| | | TTTG | 2.2% | 1.8% | 225 |
| | Non- | TTCA | 5.5% | 2.4% | 254 |
| | native | TCCG | 1.8% | 0.9% | 109 |
| | | CCCC | 4.1% | 3.7% | 267 |
| | | TCCA | 2.7% | 1.6% | 110 |
| | | TCCC | 8.9% | 8.1% | 124 |
| | | TTCC | 31.3% | 18.9% | 144 |
| | | TTCG | 9% | 8% | 100 |
| SYN3298- | Native | TTTA | 2.2% | 0% | 46 |
| RVR | | TTTC | 6.3% | 6.3% | 48 |
| | | TTTG | 2.7% | 2.7% | 75 |

### Example 9. Directed Evolution

dLbCas12a Error-Prone PCR and Selection Via FACS

Directed evolution is a powerful technique that allows for selection of a protein or nucleic acid with improved properties from a large pool of variants. We decided to use a fluorescence-based selection to identify variants of LbCas12a that alter PAM recognition. To alter PAM recognition, we targeted the amino acid residues in and around the PAM interacting domain (PID) when introducing variation into LbCas12a, because altering sequence in the PID may result in increased binding affinity for a new PAM. For simplicity, we adapted the fluorescence-based screen used to evaluate rationally designed LbCas12a variants. Instead of evaluating a single rationally designed LbCas12a variant against 256 PAMs, we evaluated a pool of LbCas12a mutants against a single PAM of interest. To generate a library of dLbCas12a containing variation in the PID region and approximately 500,000-1,000,000 variants, error-prone PCR (epPCR) was used because the amino acids within the PID that impact PAM recognition could not be easily identified through visual analysis of the crystal structure. Error-prone PCR introduces variation into a sequence of interest by utilizing an error prone DNA polymerase and modified PCR buffer conditions, resulting in random mutations in the PCR product. The rate of error may be tuned by altering buffer conditions, PCR cycle number or template input quantity. An error rate of 4.5-9 mutations/kb was used. The epPCR products were then cloned into a plasmid backbone (either dLbCas12a or dLbCas12a-K595Y) to generate a libraries of variants. The libraries were then co-transformed with a PAM of interest. For the first selection experiment, the dLbCas12a variant library was co-transformed with one of 13 PAMs: AAAA, AACG, AAGC, AGCA, AGCG, CGCG, GAGC, TAGC, AGGC, ACGC, GAGG, GGCA, and TTTC (positive control). For the second selection experiment, the dLbCas12a (K595Y) library was co-transformed with one of the following 13 PAMs: AAAA, AACG, AAGC, AGCA, AGCG, CGCG, GAGC, TAGC, AGGC, ACGC, GAGG, GGCA, and TTTC (positive control). After co-transformation, a small portion of the transformation was plated to determine transformation efficiency; the remaining amount of transformation recovery was then diluted into media containing appropriate antibiotics and grown overnight. Transformations were then pre-screened for presence of GFP fluorescence in the population via flow cytometry and frozen for future cell sorting experiments.

For the first round of directed evolution, we used the second method (FACS) to identify clones that were capable of binding novel PAMs. To prepare the dLbCas12a EP-PCR libraries for FACS, 0.5 ODs of each library and controls were washed 3 times with 1×PBS buffer and stored on ice. Immediately prior to cell sorting, the libraries and controls were filtered using 35 μm mesh filter caps and diluted 1:4 with 1×PBS. A Beckman-Coulter MoFlo XDP was then used to analyze each of the controls and set gates. Once the gates were set, each library was cell sorted on single-cell purity, collecting the to 0.5-1% of fluorescent clones. Cells were collected into a falcon tube with 2-mL of LB with appropriate antibiotics. Once cell sorting was completed, they were returned to the lab, back-diluted with an additional 4-mL of LB with antibiotics, and grown overnight at 37° C. with 225 rpm shaking. The following day, the cell sorting process was repeated to further enrich GFP-positive clones. Table 11 below lists cell sorting information for each of the libraries in the first round of directed evolution.

TABLE 11
Cell Sorting Data for EP-PCR dLbCas12a libraries (first round of directed evolution).

| | | Total | No. | | | No. | | |
|---|---|---|---|---|---|---|---|---|
| | | No. | Aborted | GFP + | Total No. | Aborted | GFP + | |
| | | Events | Events | Events | Events | Events | Events | % GFP |
| Sample | PAM | (day1) | (day1) | (day1) | (day2) | (day2) | (day2) | Collected |
| Negative | TTTC | — | — | — | — | — | — | — |
| Control | | | | | | | | |
| Positive | TTTC | — | — | — | — | — | — | — |
| Control | | | | | | | | |
| dCas12a | TTTC | 2.32E⁶ | 18.36 k | 102,685 | 0.645E6 | 14.95 k | 101,019 | 5% |
| Library 3 | | | | | | | | |
| dCas12a | TCAA | 3.33E⁶ | 2.09 k | 11,762 | 6.45E6 | 1.28 k | 6,167 | 0.19% |
| Library 4 | | | | | | | | |
| dCas12a | TTGC | 4.76E⁶ | 11.16 k | 52,819 | 5.11E6 | 3.42 k | 25,055 | 1% |
| Library 5 | | | | | | | | |
| dCas12a | TTAG | 4.72E⁶ | 5.38 k | 30,088 | 7.86E6 | 423 k | 2,158 | 0.06% |
| Library 6 | | | | | | | | |

Note
that no cells were collected for the controls - they were only used to set the gates.

After the second day of cell sorting the entire sample was plated onto LB agar plates with antibiotics. Ten colonies from each of the plates were selected along with three from wild type controls. The colonies were subcultured, plasmid DNA was isolated, and the DNA was sequenced to check for amino acid mutations in the LbCas12a protein and to verify presence of the specific PAM reporter plasmid. 15 of 33 variants sent for sequencing had complete LbCas12a ORFs; the other 18 clones did not have a complete LbCas12a ORF (meaning that they were false positives) and were discarded. The remaining 15 variants were cured of their PAM-reporter plasmid by utilizing restriction enzymes to eliminate the PAM-reporter plasmid. Once isolated, the dLbCas12a variants were re-transformed with their corresponding PAM reporter plasmids to confirm that colonies were green (this step was done as another false-positive check). The results from isolating the 15 clones and re-transforming them with their PAM reporter plasmids further identified two false positive variants out of the 15 clones. See Table 12 below.

TABLE 12
False positive testing of 15 variants identified in round 1 of
Directed Evolution identified two additional false positives.

| Sample | False Positive (Y/N) | PAM Reporter |
|---|---|---|
| 3-1 | N | TTTC |
| 3-2 | N | TTTC |
| 3-3 | N | TTTC |
| 3-4 | N | TTTC |
| 5-3 | N | TTGC |
| 5-4 | N | TTGC |
| 5-6 | N | TTGC |
| 5-7 | N | TTGC |
| 5-8 | N | TTGC |
| 5-9 | N | TTGC |
| 6-1 | N | TTAG |
| 6-2 | Y | TTAG |
| 6-3 | N | TTAG |
| 6-4 | N | TTAG |
| 6-6 | Y | TTAG |

For the second round of directed evolution, using dLbCas12a (K595Y) was performed in a similar manner, with slight changes. Again, libraries were pre-screened on the CytoFLEX (Table XX above). Cells for FACS were then prepared in the same manner as they were for the first round of directed evolution. For the second set of directed evolution libraries with dLbCas12a (K595), only 1 day of cell sorting was performed. Instead of the Beckman-Coulter MoFlo-XDP, a BD biosciences FACS Ariall was used to perform the cell sorting. Again, the top 0.5-1% of the fluorescent population was collected on single-cell purity mode. See Table 13 for data on the FACS sorting of dLbCas12a (K595Y) libraries.

TABLE 13
Round 2 FACS sorting data for dLbCas12a(K595Y) EP-PCR Libraries.

| | | Events | Threshold Count | Collection | % GFP |
|---|---|---|---|---|---|
| Sample | Sample | Collected | (Total No. | Time | collected |
| No. | Descp | (max 100k) | sorted events) | (min:sec) | cells |
| 6 | K595Y Lib. + | 4344 | 15,218,388 | 20:24 | |
| | AAGC | | | | |
| 8 | K595Y Lib. + | 2003 | 21,464,270 | 25:58 | 0.0093 |
| | AGCG | | | | |
| 10 | K595Y Lib + | 6565 | 12,708,054 | 20:05 | 0.0517 |
| | GAGC | | | | |
| 11 | K595Y Lib + | 4409 | 12,141783 | 20:01 | 0.0363 |
| | TAGC | | | | |
| 13 | K595Y Lib + | 100,000 | 1,926,978 | 3:11 | 5.1895 |
| | ACGC | | | | |
| 14 | K595Y Lib + | 472 | 10,948,770 | 20:04 | 0.0043 |
| | GAGG | | | | |
| 15 | K595Y Lib + | 395 | 11,674,490 | 20:27 | 0.0034 |
| | GGCA | | | | |
| 1 | Negative | 100,000 | 105,587 | 00:09 | 94.7086 |
| | Ctrl | | | | |
| 3 | K595Y Lib + | 5684 | 11,110,934 | 19:55 | 0.0512 |
| | TTTC | | | | |
| 2 | WT + | 50,496 | 801,080 | | 6.3035 |
| | TTTC | | | | |

After FACS was completed, the libraries were back-diluted into LB with appropriate antibiotics and then grown up overnight at 37C with 225 rpm shaking. The following day, 6-mL of each culture was then used to isolate plasmid DNA. The DNA was cured of the PAM reporter plasmid by restriction enzyme digestion. This DNA was then used to re-transform with its matching PAM reporter plasmid and plated out onto LB agar plates with appropriate antibiotics.

To assess the PAM-binding strengths of the dLbCas12a (WT) and dLbCas12a (K595Y) variants in E. coli, an in vivo growth test was performed. The growth test was performed by growing up colonies E. coli containing the variant of interest and its corresponding PAM. After overnight growth, the cultures were measured for cell density and GFP fluorescence (see Table 14 and Table 15 below).

TABLE 14
Round 1 DE dLbCas12a Cell density measurements by OD600
nm and GFP fluorescence (Ex, Em 485/20, 528/20)

| | No. of AA | | |
|---|---|---|---|
| Clone | changes | PAM | GFP/OD600 |
| 3-1 | 4 | TTTC | 322836 |
| 3-2 | 6 | TTTC | 517668 |
| 3-3 | 6 | TTTC | 420812 |
| 3-4 | 1 | TTTC | 475946 |
| 5-3 | 1 | TTGC | 33239 |
| 5-4 | 1 | TTGC | 43493 |
| 5-6 | 2 | TTGC | 82035 |
| 5-7 | 4 | TTGC | 46281 |
| 5-8 | 2 | TTGC | 38212 |
| 5-9 | 1 | TTGC | 62414 |
| 6-1 | 8 | TTAG | 71851 |
| 6-3 | 5 | TTAG | 121065 |
| 6-4 | 1 | TTAG | 117275 |
| 6-2 | 11 | TTAG | 6677 |

TABLE 15
Round 2 DE dLbCas12a Cell density measurements by OD600 nm
and GFP fluorescence (Excitation/Emission λ 485/20, 528/20)

| | | | | False- |
|---|---|---|---|---|
| | No. AA | | | Positives |
| Clone | Changes | PAM | GFP/OD600 | (Y/N) |
| 6-1 | 4 | AAGC | 322836 | N |
| 6-2 | 2 | AAGC | 517668 | N |
| 6-3 | 4 | AAGC | 420812 | N |
| 6-20 | 5 | AAGC | 475946 | Y |
| 8-1 | 3 | AGCG | 2972580 | N |
| 11-1 | 2 | TAGC | 33239 | N |
| 11-2 | 5 | TAGC | 43493 | N |
| 11-4 | 3 | TAGC | 82035 | N |
| 11-12 | 2 | TAGC | 46281 | Y |
| 11-15 | 4 | TAGC | 38212 | Y |
| 12-1 | | AGGC | 1847893 | N |
| 12-2 | | AGGC | 3142139 | N |
| 12-3 | | AGGC | 3607182 | N |
| 12-4 | | AGGC | 1200856 | Y |
| 12-5 | 5 | AGGC | 5111568 | N |
| 12-6 | 6 | AGGC | 5866201 | N |
| 12-7 | | AGGC | 1842311 | Y |
| 12-8 | | AGGC | 3400834 | N |
| 12-9 | | AGGC | 2346790 | N |
| 12-10 | | AGGC | 1199643 | Y |
| 15-1 | 9 | GGCA | 3709073 | N |
| 15-2 | 6 | GGCA | 2527884 | N |
| 15-3 | | GGCA | 2817978 | N |
| 15-4 | | GGCA | 2678149 | N |
| 15-5 | | GGCA | 2685433 | N |
| 15-6 | | GGCA | 2640861 | N |
| 15-9 | | GGCA | 2721722 | N |
| 15-10 | | GGCA | 2824155 | N |

Differences in GFP fluorescence were observed among the variants. The variants were then rank-ordered from highest normalized fluorescence (binding strength of PAM) to lowest. This information was then used in evaluating which variants to test in a human cell cutting assay.

To test the mutations in a eukaryotic system, and as nucleases, cutting assays were performed in human cells (HEK293T). For each variant, 3-5 spacers were tested (each spacer had the desired novel PAM). In addition to testing mutants identified in directed evolution, the mutants were also stacked with other mutations known to expand PAM access (e.g., G532R+K595R (RR) or G532R+K538V+Y542R(RVR)).

TABLE 16
Amino acid residues that were changed
resulting in novel PAM recognition.

| Variant | AA mutations | PAM | pWISE |
|---|---|---|---|
| | Round 1 Below | | |
| 3-1 | K167I, Q231I, A454V, F657L | TTTC | Not tested |
| | | | in MCF |
| 3-2 | F337L, L459M, I507V, M531I, | TTTC | Not tested |
| | D654E, E659V | | in MCF |
| 3-3 | S170L, V245I, I425V, Y426D, | TTTC | Not tested |
| | V463A, L492P | | in MCF |
| 3-4 | K167N | TTTC | Not tested |
| | | | in MCF |
| 5-3 | K514N | TTGC | Not tested |
| | | | in MCF |
| 5-4 | E330V | TTGC | pWISE6649 |
| 5-6 | N263I, L498M | TTGC | pWISE6613 |
| 5-7 | N112D, E223V, Y426C, I455F | TTGC | Not tested |
| | | | in MCF |
| 5-8 | S117C, A239T | TTGC | Not tested |
| | | | in MCF |
| 5-9 | T295I | TTGC | pWISE6692 |
| 6-1 | N100S, K387E, A404V, D405G, | TTAG | pWISE6614 |
| | D423V, E484D, E539V, D572G | | |
| 6-3 | N211S, N527S, Y553H, Y554N, | TTAG | pWISE6616 |
| | L585Q | | |
| 6-4 | E125G | TTAG | pWISE6695 |
| | Round 2 below | | |
| 8-1 | I417F, K538N, K595Y | AGCG | pWISE7620 |
| 11-1 | D489Y, K595Y | TAGC | pWISE7621 |
| 11-2 | D367V, E379G, Y381H, N590Y, | TAGC | pWISE7622 |
| | K595Y | | |
| 11-4 | L324M, E484V, K595Y | TAGC | pWISE7623 |
| 6-2 | L321V, K595Y | AAGC | pWISE7624 |
| 12-5 | K153N, T307S, S345R, D489V, | AGGC | pWISE7769 |
| | K595Y | | |
| 12-6* | K167I, K192I, I344T, E443D, | AGGC | pWISE7768 |
| | K538N, K595Y | | |
| 15-1 | S168N, T246S, R359S, I369M, | GGCA | pWISE7767 |
| | F474L, E479V, K538N, Y542N, | | |
| | K595Y | | |
| 15-2* | K167I, K192I, I344T, E443D, | GGCA | pWISE7768 |
| | K538N, K595Y | | |
| n/a | K595Y | ACGC | pWISE2204 |

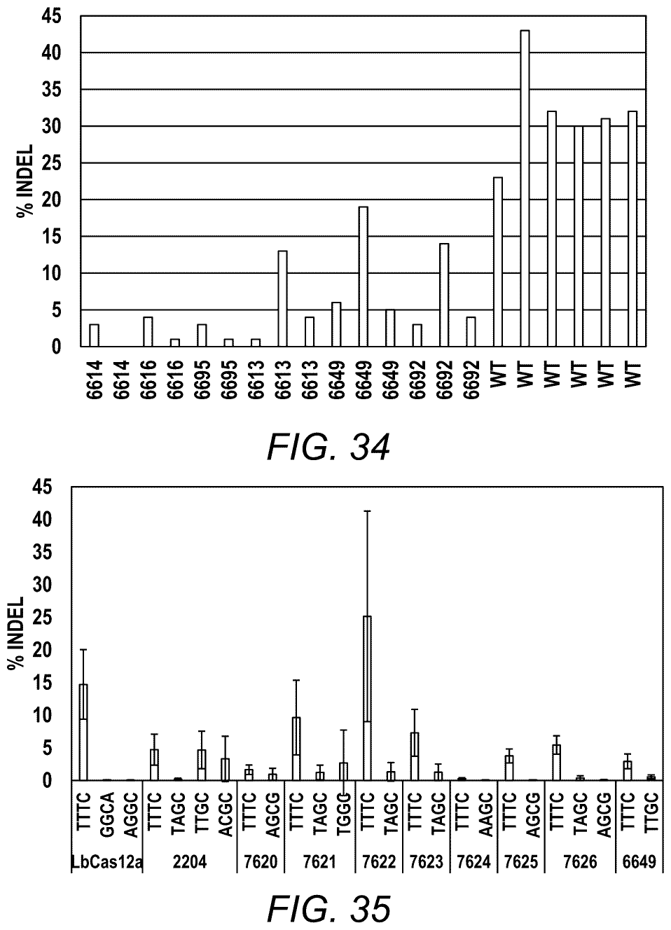

Variants 3-1 through 6-4 in Table 16 correspond to the first round of DE performed on wild type LbCas12a (see also FIG. 34). The second set of variants 8-1 through 15-2 provides the other round of DE using LbCas12a (K595Y) enzyme. Table 16 identifies all the mutations were found that passed all of the assay QC in E. coli (i.e., these are all the mutations found using DE). All variants that have a pWISE number were further tested in human cells.

TABLE 17
Additional Cas12a mutant constructs tested in mammalian
cells facility. The mutants were those identified
in both rounds of directed evolution (DE).

| | Editor | |
|---|---|---|
| | Plasmid | PAMs Testing |
| Cas12a Variant | pWISE# | Against |
| WT-LbCas12a | pWISE121 | TTTC, TAGC, TTGC |
| RR-LbCas12a | pWISE3559 | TTTC, TAGC, TTGC |
| RVR-LbCas12a | pWISE6648 | TTTC, TAGC, TTGC |
| LbCas12a (K595Y) | pWISE2204 | TTTC, TAGC, TTGC, |
| | | ACGC |
| LbCas12a (I417F + K538N + | pWISE7620 | AGCG, TTTC |
| K595Y) | | |
| LbCas12a (D489Y + K595Y) | pWISE7621 | TAGC, TGGC, TTTC |
| LbCas12a (D367V + E379G + | pWISE7622 | TAGC, TTTC |
| Y381H + N590Y + K595Y) | | |
| LbCas12a (L324M + E484V + | pWISE7623 | TAGC, TTTC |
| K595Y) | | |
| LbCas12a (L321V + K595Y) | pWISE7624 | AAGC, TTTC |
| LbCas12a (I417F) | pWISE7625 | AGCG, TTTC |
| LbCas12a (I417F + D489Y) | pWISE7626 | AGCG, TAGC, TTTC |
| RR-LbCas12a (E330V + I417F + | pWISE7627 | AGCG, TTGC, TTTC |
| K538N) | | |
| RR-LbCas12a (E330V) | pWISE7628 | Various RR, TTTC |
| RVR-LbCas12a (I417F) | pWISE7630 | various RVR, AGCG, |
| | | TTTC |
| RVR-LbCas12a (N263I + L498M) | pWISE7631 | various RVR, TTGC, |
| | | TTTC |
| RVR-LbCas12a (E330V) | pWISE7632 | Various RVR, TTTC |
| RVR-LbCas12a (T295I) | pWISE7633 | Various RVR, TTGC, |
| | | TTTC |
| LbCas12a (E330V) | pWISE6649 | TTGC (2 new spacers) |

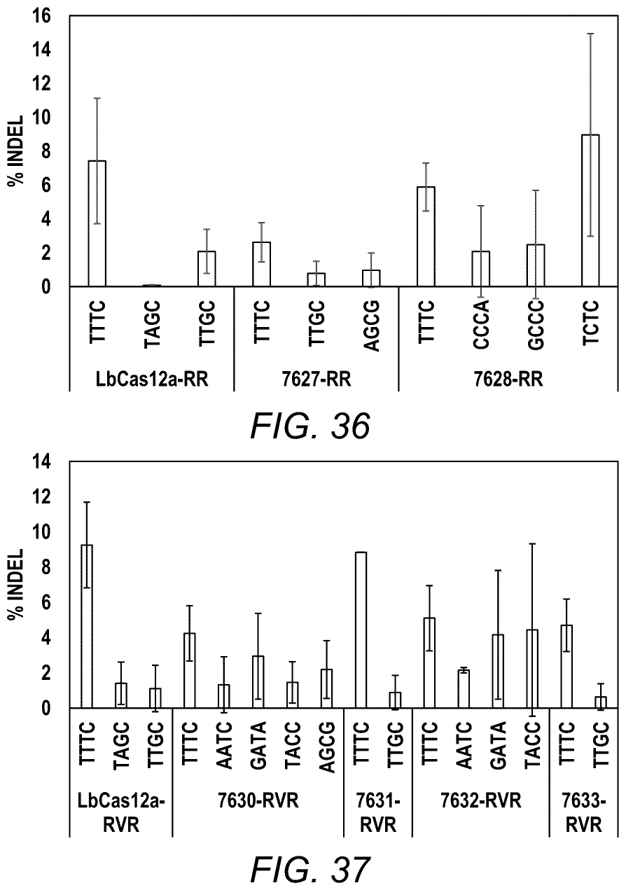

In addition to testing mutants that came out of directed evolution directly, we tested the variants described above in Table 17 (see also FIG. 35-37). These variants were selected using 3 criteria:
1. Mutations that arose in directed evolution that, upon inspection of the crystal structure were in close proximity the PAM or to the target DNA (an example of this is E330V or T295I)
2. Combinations of high-performing mutations
3. Combinations of high-performing mutations with previously identified mutants that expand PAM access (G532R+K595R and G532R+K538V+K595R)

TABLE 18
Constructs tested in mammalian cell facility from utilizing
mutations found from the second round of directed evolution.

| | Editor Plasmid | PAMs Testing |
|---|---|---|
| Cas12a Variant | pWISE# | Against |
| WT-LbCas12a | pWISE121 | TTTC, GGCA, |
| | | AGGC |
| G532R/K595R-LbCas12a | pWISE3559 | TTTC, GGCA, |
| | | AGGC |
| G532R/K538V/K595R-LbCas12a | pWISE6648 | TTTC, GGCA, |
| | | AGGC |
| LbCas12a (K595Y) | pWISE2204 | TTTC, GGCA, |
| | | AGGC |
| LbCas12a (K538N) | pWISE7763 | TTTC, GGCA |
| LbCas12a (K595Y + K583N) | pWISE7764 | TTTC, GGCA, |
| | | AGGC |
| LbCas12a (G532R + K538N + Y542R) | pWISE7765 | TTTC, GGCA |
| LbCas12a (G532R + K538N + Y542N) | pWISE7766 | TTTC, GGCA |
| LbCas12a Variant 15-1 (S168N, T246S, | pWISE7767 | TTTC, GGCA |
| R359S, I369M, F474L, E479V, K538N, | | |
| Y542N, K595Y) | | |
| LbCas12a Variant 15-2/12-6 (K167I, | pWISE7768 | TTTC, GGCA, |
| K192I, I344T, E443D, K538N, K595Y) | | AGGC |
| LbCas12a Variant 12-5 (K135N, | pWISE7769 | TTTC, AGGC |
| T307S, S345R, D489V, K595Y) | | |
| Variant 15-1 SHARC(3298) | pWISE7899 | TTTC, GGCA |
| Variant 15-2 SHARC(3298) | pWISE7900 | TTTC, GGCA, |
| | | AGGC |
| Variant 12-5 SHARC (3298) | pWISE7901 | TTTC, AGGC |

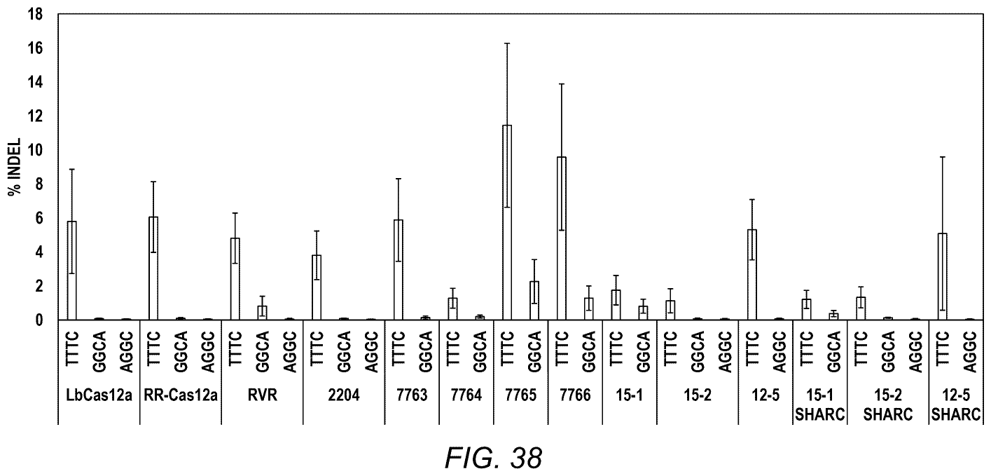

The Cas12a variants described in Table 18 (see also FIG. 38) were generated using the same criteria that was used to generate the variants in Table 17. Single mutants and combinations of mutations were tested. In addition, mutations were also transplanted onto SHARC, to test whether the PAM-expanding mutations identified in a Cas12a context also applied to SHARC.

TABLE 19
Performance of LbCas12a Directed Evolution variants mean across 3-spacers (MCF782)

| Description* | pWISE | D156R pWise | ACGC | AGCG | TAGC | TGGC | TTGC | TTTV |
|---|---|---|---|---|---|---|---|---|
| LbCas12a | pWISE121 | n/a | | | | | | 15.1 |
| LbCas12a (K595Y) | pWISE2204 | n/a | 6 | | | | | 16.5 |
| LbCas12a (D156R) | pWISE121 | pWISE8870 | 1.1 | | 3.1 | | 14 | 52.6 |
| LbCas12a (D156R + | pWISE2204 | pWISE8871 | 25.8 | | 18.3 | | 28.3 | 31.6 |
| K595Y) | | | | | | | | |
| LbCas12a | pWISE7621 | pWISE8874 | | | 20.4 | 13.6 | | 14.5 |
| (D156R + D489Y + | | | | | | | | |
| K595Y) | | | | | | | | |
| LbCas12a | pWISE7623 | pWISE8876 | | | 17 | | | 10.9 |
| D156R + L324M + | | | | | | | | |
| E484V + K595Y) | | | | | | | | |
| LbCas12a | pWISE7629 | pWISE8881 | | | 16.6 | | 13.3 | 33.5 |
| (D156R + E330V + | | | | | | | | |
| D489Y) | | | | | | | | |
| LbCas12a | pWISE7622 | pWISE8875 | | | 11.6 | | | 9.3 |
| (D156R + D367V + | | | | | | | | |
| E379G + Y381H + | | | | | | | | |
| N590Y + K595Y) | | | | | | | | |
| LbCas12a | pWISE7626 | pWISE8878 | | 0.04 | 6.6 | | | 12.3 |
| (D156R + I417F + | | | | | | | | |
| D489Y) | | | | | | | | |

Blank cells: PAM was not tested for that variant

To generate the variants tested in Table 19, mutants that displayed the highest nuclease activity at expanded PAMs were selected. We hypothesized that these mutants, while displaying nuclease activity at expanded PAMs, could be improved by adding a point mutation known to increase activity (D156R). For each of these Cas12a variants, we added in the D156R mutation in addition to the other mutations described. For each variant, the average INDEL rate in HEK293T cells across 3 spacers is shown.

### Example 10. RYY Variant and R2YY Variant

Additional Cas12a variants were generated which contained the amino acid variants of D156R, D489Y and K595Y, all with reference to position numbering of SEQ ID NO: 180; or T152R, D489Y and K595Y all with reference to position numbering of SEQ ID NO: 180. D156R, D489Y and K595Y is referred to as “RYY” while T152R, D489Y and K595Y is referred to as “R2YY”.

The RYY variant demonstrated increased PAM recognition as shown in Table 20. HEK293T cells were used as previously described in Example 9 to evaluate PAM access using 3 different spacers targeting three different genomic regions. Entries in Table 20 with an “*” (single asterisk) indicate that these entries are for one spacer while the “**” (two asterisks) indicates that these entries are for two spacers targeting two different genomic regions. Table 20 data represents an average of percentage of insertion/deletion for either the RYY or R2YY variant. A control is not shows as the wildtype, control Cas12a is unable to access any of the PAM sequences in Table 20. This data demonstrates that both RYY and R2YY have increased PAM access.

TABLE 20

| | RYY variant | R2YY variant |
|---|---|---|
| PAM | pWISE10212 | pWISE11357 |
| GAGC | 12.527 | 4.469 |
| GCGC** | 43.694 | 36.907 |
| GGGC* | 18.902 | 9.338 |
| GTGC** | 36.772 | 27.245 |
| TGGC** | 11.572 | 6.250 |
| CCCT | 7.435 | 3.289 |
| CCTA | 15.335 | 17.922 |
| GCCC | 32.359 | 23.940 |
| GCTA | 11.562 | 7.664 |
| GTCG | 12.035 | 6.523 |
| GTTA | 31.717 | 19.804 |
| TCCA | 18.692 | 13.925 |
| TCTT | 9.171 | 4.396 |
| TTCA | 40.804 | 34.209 |
| TTCT | 22.590 | 14.398 |
| TTTV | 49.507 | 47.036 |
| ATGA | 10.386 | 9.159 |
| ATGC | 10.240 | 8.453 |
| ATGG** | 12.113 | 15.931 |
| TAGA | 0.373 | 0.223 |
| TAGC** | 17.155 | 14.278 |
| TAGG** | 2.624 | 3.339 |
| ATCA | 31.381 | 21.252 |

### Example 11. R2YY in Soybean

The variant R2YY described in Example 10 was also evaluated in soybean cells to demonstrate enhanced PAM recognition. Soybean was transformed with a vector containing the variant of Cas12a and regenerated plants were screened for edits in the targeted gene. The transformation construct contained spacer sequences designed for a genomic soybean target gene. The R2YY variant of Cas12a was evaluated for editing at two different genomic regions using the PAM recognition sequence “ACGC” and for one genomic region using the PAM recognition sequence “TGGC”. As shown in Table 21, R2YY the soybean data demonstrates that the R2YY variant recognizes the non-cannonical PAM sequences of “ACGC” and “TGGC” indicating that this variant has expanded PAM access. The values in Table 21 are the average editing strength (meaning: edit with the highest percentage of an insertion/deletion detected in a given plant). The value range of +/− are the 95% confidence interval. N/A=no edits recovered at the indicated target.

TABLE 21

| Cas12a | PAM | Target M | Target P | Target Q |
|---|---|---|---|---|
| R2YY- | ACGC | 28.3 ± 3.6 | 30.8 ± 12 | N/A |
| R2YY- | TGGC | N/A | N/A | 13.5 |

The foregoing is illustrative of the present invention, and is not to be construed as limiting thereof. The invention is defined by the following claims, with equivalents of the claims to be included therein.

What is claimed is:

1. A CRISPR (Clustered Regularly Interspaced Short Palindromic Repeats) Cas12a polypeptide, wherein the Cas12a polypeptide comprises:
(a) an amino acid sequence having at least 95% identity to the amino acid sequence of SEQ ID NO: 180 and a mutation located at one or more than one of the following positions of K135, S168, T246, N263, T307, L324, E330, S345, R359, D367, I369, E379, Y381, I417, F474, E479, E484, D489, L498, N590, K167, Q231, A454, F657, F337, L459, I507, M531, D654, E659, S170, V245, I425, V463, L492, K514, N112, E223, Y426, I455, S117, A239, T295, N100, K387, A404, D405, D423, E539, D572, N211, N527, Y553, Y554, L321, K192, I344 and/or E443, optionally a mutation located at position K135, S168, T246, N263, T307, L324, E330, S345, R359, D367, I369, E379, Y381, I417, F474, E479, E484, D489, L498, and/or N590, and/or a mutation comprising one or more of K135N, S168N, T246S, N263I, T307S, L324M, E330V, S345R, R359S, D367V, I369M, E379G, Y381H, I417F, F474L, E479V, E484V, D489Y, L498M, N590Y, K167I, Q231, A454V, F657, F337L, L459M, I507V, M531I, D654E, E659V, S170L, V245I, I425V, V463A, L492P, K514N, N112D, E223V, Y426D, 1455F, S117C, A239T, T295I, N100S, K387E, A404V, D405G, D423V, E539V, D572G, N211S, N527S, Y553H, Y554N, L321V, K192I, I344T and/or E443D, optionally a mutation comprising one or more of K135N, S168N, T246S, N263I, T307S, L324M, E330V, S345R, R359S, D367V, 1369M, E379G, Y381H, I417F, F474L, E479V, E484V, D489Y, L498M, and/or N590Y, optionally the mutation of D489Y, L324M, E484V and/or E330V, with reference to position numbering of SEQ ID NO: 180, wherein the polypeptide exhibits a reduced PAM stringency and increased recognition of new protospacer adjacent motifs (PAMs) as compared to the amino acid sequence of SEQ ID NO: 180,
(b) an amino acid sequence having at least 95% identity to the amino acid sequence of SEQ ID NO: 51 and a mutation located at one or more than one of the following positions of E153, S186, R267, I285, Y336, L352, S358, E372, R386, E391, R393, K403, A405, A434, V486, E491, P494, R499, E508, A602, I185, Q252, E465, L698, F365, Q470, A517, A541, L695, P700, A188, S266, I442, L474, G502, K524, T114, P244, L443, I466, D119, Q260, K324, T101, R411, A427, G428, S440, E549, E584, S234, M537, Y563, Y564, A349, R210, L371, and/or K459, optionally a mutation at position E153, S186, R267, I285, Y336, L352, S358, E372, R386, E391, R393, K403, A405, A434, V486, E491, P494, R499, E508, and/or A602, optionally a mutation at position R499, L352, P494 and/or S358, with reference to position numbering of SEQ ID NO: 51, wherein the polypeptide exhibits a reduced PAM stringency and increased recognition of new protospacer adjacent motifs (PAMs) as compared to the amino acid sequence of SEQ ID NO: 51, or
(c) an amino acid sequence having at least 95% identity to the amino acid sequence of SEQ ID NO: 55 and a mutation located at one or more than one of the following positions of I162, P196, V290, S308, Q358, L377, K385, T400, Q423, D431, P433, K443, T445, A480, I454, D550, E559, V564, A573, N666, I195, Q275, A525, F741, Y392, L529, I582, A606, G738, D743, S198, F289, I488, T533, E567, K589, S116, A267, F489, A525, N121, T283, E343, T104, L451, R467, D468, P486, E614, E648, T243, N602, Y628, Y629, L374, S220, L399, and/or Q507, optionally a mutation located at I162, P196, V290, S308, Q358, L377, K385, T400, Q423, D431, P433, K443, T445, A480, I454, D550, E559, V564, A573, and/or N666, optionally a mutation at position V564, L377, E559 and/or K385, with reference to position numbering of SEQ ID NO: 55, wherein the polypeptide exhibits a reduced PAM stringency and increased recognition of new protospacer adjacent motifs (PAMs) as compared to the amino acid sequence of SEQ ID NO: 55.

2. The Cas12a polypeptide of claim 1, wherein the Cas12a polypeptide of (a) further comprises a mutation at one or more than one of the following positions of K116, K120, K121, D122, E125, T148, T149, T152, D156, E159, Q529, D535, K538, D541, Y542, L585, K591, N590, M592, K595, V596, S599, K600, K601, Y616, Y646, and/or W649 with reference to position numbering of SEQ ID NO: 180; the Cas12a polypeptide of (b) further comprises a mutation at one or more than one of the following positions of E127, K548, N552, F598, A602 and/or K607 with reference to position numbering of SEQ ID NO: 51; the Cas12a polypeptide of (c) further comprises a mutation at one or more than one of the following positions of D129, K613, N617, L661, N666 and/or K671 with reference to position numbering of SEQ ID NO: 55.

3. The Cas12a polypeptide of claim 2, wherein the Cas12a polypeptide comprises one or more than one or the amino acid mutations of K116R, K116N, K116D, K120R, K120H, K120N, K120T, K120Y, K120Q, K121S, K121T, K121H, K121R, K121G, K121D, K121Q, D122R, D122K, D122H, D122E, D122N, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D1561, D156V, D156L, D156E, E159K, E159R, E159H, E159Y, E159Q, Q529N, Q529T, Q529H, Q529A, Q529F, Q529G, Q529G, Q529S, Q529P, Q529W, Q529D, G532D, G532N, G532S, G532H, G532F, G532K, G532R, G532Q, G532A, G532L, G532C, D535N, D535H, D535V, D535T, D535,S D535A, D535W, D535K, K538R K538V, K538Q, K538W, K538Y, K538F, K538H, K538L, K538M, K538C, K538G, K538A, K538P, D541N, D541H, D541R, D541K, D541Y, D5411, D541A, D541S, D541E, Y542R, Y542K, Y542H, Y542Q, Y542F, Y542L, Y542M, Y542P, Y542V, Y542N, Y542T, L585G, L585H, L585F, K591W, K591F, K591Y, K591H, K591R, K591S, K591A, K591G, K591P, M592R, M592K, M592Q, M592E, M592A, K595R, K595Q, K595Y, K595L, K595W, K595H, K595E, K595S, K595D, K595M, V596T, V596H, V596G, V596A, S599G, S599H, S599N, S599D, K600R, K600H, K600G, K601R, K601H, K601Q, K601T, Y616K, Y616R, Y616E, Y616F, Y616H, Y646R, Y646E, Y646K, Y646H, Y646Q, Y646W, Y646N, W649H, W649K, W649Y, W649R, W649E, W649S, W649V, and/or W649T with reference to position numbering of SEQ ID NO: 180.

4. The Cas12a polypeptide of claim 3, wherein the Cas12a polypeptide of
(a) comprises any one of the following combination of mutations of (1) D156R, D489Y and K595Y, (2) D156R, L324M, E484V and K595Y, (3) D156R, E330V and D489Y, (4) D156R, D367V, E379G, Y381H, N590Y and K595Y, (5) D156R, I417F and D489Y, (6) D156R, I417F, K538N and K595Y, (7) D156R, E330V, I417F, and K538N, (8) D156R and E330V, (9) D156R and 1417F, (10) D156R, N263I and L498M, (11) K135N, D156R, T307S, S345R, D489V and K595Y, and/or (12) D156R, S168N, T246S, R359S, I369M, F474L, E479V, K538N, Y542N and K595Y with reference to position numbering of SEQ ID NO: 180;
(b) comprises any one of the following combination of mutations of (1) E174, R499 and K607, (2) E174, L352, P494 and K607, (3) E174, S358 and R499, (4) E174, E391, K403, A405, A602 and K607, (5) E174, A434 and R499, (6) E174, A434, K548 and K607, (7) E174, S358, A434 and K548, (8) E174 and S358, (9) E174 and A434, (10) E174, I285 and E508, (11) E153, E174, Y336, E372, R499 and K607, and/or (12) E174, S186, R267, R386, R393, V486, E491, K548, N552 and K607 with reference to position numbering of SEQ ID NO: 51; and/or
(c) comprises any one of the following combination of mutations of (1) E184, V564 and K671, (2) E184, L377, E559 and K671, (3) E184, K385 and V564, (4) E184, D431, K443, T445, N666 and K671, (5) E184, A480 and V564, (6) E184, A480, K613 and K671, (7) E184, K385, A480, and K613, (8) E184 and K385, (9) E184 and A480, (10) E184, S308, and A573, (11) I162, E184, Q358, T400, V564 and K671, or (12) E184, P196, V290, Q423, P433, I545, D550, K613, N617 and K671 with reference to position numbering of SEQ ID NO: 55.

5. The Cas12a polypeptide of any one of claims 1-4, further comprising a mutation in the RuvC domain.

6. A fusion protein comprising the Cas12a polypeptide of any one of claims 1-5, and a linker, optionally a peptide linker.

7. The fusion protein of claim 6, wherein the peptide linker is linked to the C-terminus and/or the N-terminus of the Cas12a polypeptide.

8. A fusion protein comprising the Cas12a polypeptide of any one of claims 1-7, and a polypeptide of interest.

9. The fusion protein of claim 8, wherein the polypeptide of interest is linked to the C-terminus and/or the N-terminus of the Cas12a polypeptide.

10. The fusion protein of claim 8 or claim 9, wherein the polypeptide of interest comprises at least one polypeptide or protein domain having deaminase (deamination) activity, nickase activity, recombinase activity, transposase activity, methylase activity, glycosylase (DNA glycosylase) activity, glycosylase inhibitor activity (e.g., uracil-DNA glycosylase inhibitor (UGI)), demethylase activity, transcription activation activity, transcription repression activity, transcription release factor activity, histone modification activity, nuclease activity, single-strand RNA cleavage activity, double-strand RNA cleavage activity, restriction endonuclease activity (e.g., Fok1), nucleic acid binding activity, methyltransferase activity, DNA repair activity, DNA damage activity, dismutase activity, alkylation activity, depurination activity, oxidation activity, pyrimidine dimer forming activity, integrase activity, transposase activity, polymerase activity, ligase activity, helicase activity, and/or photolyase activity.

11. The fusion protein of any one of claims 8-10, wherein the polypeptide of interest comprises at least one polypeptide or protein domain having deaminase activity, optionally wherein the at least one polypeptide or protein domain having deaminase activity is a cytosine deaminase domain or an adenine deaminase domain.

12. The fusion protein of any one of claims 8-11, wherein the at least one polypeptide has glycosylase inhibitor activity, optionally wherein the at least one polypeptide is a uracil-DNA glycosylase inhibitor (UGI).

13. A polynucleotide encoding the Cas12a polypeptide of any one of claims 1-5, or the fusion protein of any one of claims 6-12.

14. The polynucleotide of claim 13 wherein the polynucleotide is optimized for expression in an organism, optionally, wherein the organism is an animal, a plant, a fungus, an archaeon, or a bacterium.

15. A complex comprising the Cas12a polypeptide any one of claims 1-5, or the fusion protein of any one of claims 6-12, and a guide nucleic acid (e.g., CRISPR RNA, CRISPR DNA, crRNA, crDNA).

16. A nucleic acid construct encoding the complex of claim 15.

17. A composition comprising (a) the Cas12a polypeptide of any one of claims 1-5, or the fusion protein of any one of claims 6-12 and (b) a guide nucleic acid.

18. An expression cassette or vector comprising the polynucleotide of claim 13 or claim 14, or the nucleic acid construct of claim 16.

19. A Type V Clustered Regularly Interspaced Short Palindromic Repeats (CRISPR)-associated (Cas) (CRISPR-Cas) system comprising a guide nucleic acid (CRISPR RNA, CRISPR DNA, crRNA, crDNA) comprising a spacer sequence and a repeat sequence and
(a) a fusion protein comprising the Cas12a polypeptide of any one of claims 1-5 and a polypeptide of interest; or
(b) a nucleic acid encoding the Cas12a polypeptide of any one of claims 1-5, and a nucleic acid encoding the polypeptide of interest,
wherein the guide nucleic acid is capable of forming a complex with the modified LbCas12a polypeptide or the fusion protein and the spacer sequence is capable of hybridizing to a target nucleic acid, thereby guiding the Cas12a polypeptide and the polypeptide of interest to the target nucleic acid, whereby the target nucleic acid is modified (e.g., cleaving or editing) or modulated (e.g., modulating transcription).

20. The system of claim 18, wherein the Cas12a polypeptide comprises a mutation in the RuvC domain.

21. The system of claim 19 or claim 20, wherein the polypeptide of interest is linked to the C-terminus and/or the N-terminus of theCas12a polypeptide.

22. The system of any one of claims 19-21, wherein the polypeptide of interest comprises at least one polypeptide or protein domain having deaminase (deamination) activity, nickase activity, recombinase activity, transposase activity, methylase activity, glycosylase (DNA glycosylase) activity, glycosylase inhibitor activity (e.g., uracil-DNA glycosylase inhibitor (UGI)). demethylase activity, transcription activation activity, transcription repression activity, transcription release factor activity, histone modification activity, nuclease activity, single-strand RNA cleavage activity, double-strand RNA cleavage activity, restriction endonuclease activity (e.g., Fok1), nucleic acid binding activity, methyltransferase activity, DNA repair activity, DNA damage activity, dismutase activity, alkylation activity, depurination activity, oxidation activity, pyrimidine dimer forming activity, integrase activity, transposase activity, polymerase activity, ligase activity, helicase activity, and/or photolyase activity, optionally wherein the polypeptide of interest comprises at least one polypeptide or protein domain having deaminase activity.

23. The system of any one of claims 19-22, wherein the polypeptide of interest comprises a uracil-DNA glycosylase inhibitor (UGI).

24. A cell comprising the polynucleotide of claim 13 or claim 14, the nucleic acid construct of claim 16, the expression cassette or vector of claim 18, or the system of any one of claims 19-23.

25. A method of modifying a target nucleic acid comprising contacting the target nucleic acid with:
(a)(i) the Cas12a polypeptide of any one of claims 1-5, or the fusion protein of any one of claims 6-12, and (ii) a guide nucleic acid (e.g., CRISPR RNA, CRISPR DNA, crRNA, crDNA);
(b) the complex of claim 14 and a guide nucleic acid;
(c) a composition comprising (i) the Cas12a polypeptide of any one of claims 1-5, or the fusion protein of any one of claims 6-12, and (ii) a guide nucleic acid; and/or
(d) the system of any one of claims 19-23, thereby modifying the target nucleic acid.

26. A method of modifying a target nucleic acid, comprising contacting a cell or a cell free system comprising the target nucleic acid with:
(a)(i) the polynucleotide of claim 13 or claim 14, or an expression cassette or vector comprising the same, and (ii) a guide nucleic acid, or an expression cassette or vector comprising (a)(i) and (ii); and/or
(b) the nucleic acid construct of claim 16, or an expression cassette or vector comprising the same, thereby modifying the target nucleic acid.

27. A method of editing a target nucleic acid, comprising:
contacting the target nucleic acid with:
(a)(i) the fusion protein of any one of claims 6-12, and (a)(ii) a guide nucleic acid;
(b) a complex comprising the fusion protein of any one of claims 6-12, and a guide nucleic acid;
(c) a composition comprising the fusion protein of any one of claims 6-12 and a guide nucleic acid; and/or,
(d) the system of any one of claims 19-23, thereby editing the target nucleic acid.

28. A method of editing a target nucleic acid, comprising contacting a cell or a cell free system comprising the target nucleic acid with:
(a)(i) a polynucleotide encoding the fusion protein of any one of claims 6-12, or an expression cassette or vector comprising the same, and (a)(ii) a guide nucleic acid, or an expression cassette or vector comprising the same; and/or
(b) a nucleic acid construct encoding a complex comprising the fusion protein of any one of claims 6-12, and a guide nucleic acid, or an expression cassette or vector comprising the same; and/or
(c) the system of any one of claims 19-23, thereby editing the target nucleic acid.

29. A kit comprising the polynucleotide of claim 13 or claim 14, and/or an expression cassette or vector comprising the same, and optionally instructions for the use thereof, optionally, further comprising a CRISPR-Cas12a guide nucleic acid and/or expression cassette or vector comprising the same.

30. An engineered protein comprising:
a first polypeptide that is a first portion of a modified protein, wherein the modified protein comprises an amino acid sequence having at least 80%, 85%, 90%, or 95% identity to the amino acid sequence of SEQ ID NO: 180 (LbCas12a) and, with reference to the amino acid sequence of SEQ ID NO: 180, comprises a mutation at one or more of the following positions selected from the group consisting of: N100, K116, K120, K121, D122, E125, T148, T149, T152, D156, E159, N211, N263, T295, E330, K387, A404, D405, D423, E484, L498, N527, Q529, G532, D535, K538, E539, D541, Y542, Y553, Y554, D572, L585, K591, M592, K595, V596, S599, K600, K601, Y616, Y646, W649, N112, S117, K135, K167, S168, S170, K192, E223, Q231, A239, V245, T246, T307, L321, L324, F337, I344, S345, R359, D367, I369, E379, Y381, I417, I425, Y426, E443, A454, I455, L459, V463, F474, E479, D489, L492, I507, K514, M531, N590, F657, D654, E659 and any combination thereof with reference to position numbering of SEQ ID NO: 180; and
a second polypeptide that is heterologous to the first polypeptide and is not a Type V CRISPR-Cas effector polypeptide,
wherein the first polypeptide and second polypeptide are different from each other, and
wherein the engineered protein comprises the mutation.

31. The engineered protein of claim 30, wherein the modified protein comprises one or more amino acid mutations selected from the group consisting of: N100S, K116D, K116R, K116N, K120R, K120H, K120N, K120T, K120Y, K120Q, K121S, K121T, K121H, K121R, K121G, K121D, K121Q, D122R, D122K, D122H, D122E, D122N, E125G, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D1561, D156V, D156L, D156E, E159K, E159R, E159H, E159Y, E159Q, N211S, N263I, T295I, E330V, K387E, A404V, D405G, D423V, E484D, L498M, N527S, Q529N, Q529T, Q529H, Q529A, Q529F, Q529G, Q529S, Q529P, Q529W, Q529D, G532D, G532N, G532S, G532H, G532F, G532K, G532R, G532Q, G532A, G532L, G532C, D535N, D535H, D535V, D535T, D535S, D535A, D535W, D535K, K538R, K538V, K538Q, K538W, K538Y, K538F, K538H, K538L, K538M, K538C, K538G, K538A, K538P, E539V, D541N, D541H, D541R, D541K, D541Y, D5411, D541A, D541S, D541E, Y542R, Y542K, Y542H, Y542Q, Y542F, Y542L, Y542M, Y542P, Y542V, Y542N, Y542T, Y553H, Y554N, D572G, L585Q, L585G, L585H, L585F, K591W, K591F, K591Y, K591H, K591R, K591S, K591A, K591G, K591P, M592R, M592K, M592Q, M592E, M592A, K595R, K595Q, K595Y, K595L, K595W, K595H, K595E, K595S, K595D, K595M, V596T, V596H, V596G, V596A, S599G, S599H, S599N, S599D, K600R, K600H, K600G, K601R, K601H, K601Q, K601T, Y616K, Y616R, Y616E, Y616F, Y616H, Y646R, Y646E, Y646K, Y646H, Y646Q, Y646W, Y646N, W649H, W649K, W649Y, W649R, W649E, W649S, W649V, W649T, N112D, S117C, K135N, K167I, S168N, S170L, K192I, E223V, Q231, A239T, V245I, T246S, T307S, L321V, L324M, F337L, 1344T, S345R, R359S, D367V, I369M, E379G, Y381H, I417F, 1425V, Y426D, E443D, A454V, 1455F, L459M, F474L, E479V, D489Y, L492P, I507V, K514N, M531I, N590Y, D654E, F657, E659V, V463A and any combination thereof with reference to the position numbering of SEQ ID NO: 180, optionally wherein the modified protein comprises one or more amino acid mutations selected from the group consisting of: N100S, K116D, E125G, T152R, D156E, N211S, N263I, T295I, E330V, K387E, A404V, D405G, D423V, E484D, L498M, N527S, G532R, K538V, E539V, Y542R, Y553H, Y554N, D572G, L585Q, K595R, K595Y, N112D, S117C, K135N, K167I, S168N, S170L, K192I, E223V, Q231, A239T, V245I, T246S, T307S, L321V, L324M, F337L, 1344T, S345R, R359S, D367V, I369M, E379G, Y381H, I417F, 1425V, Y426D, E443D, A454V, 1455F, L459M, V463A F474L, E479V, D489Y, L492P, I507V, K514N, M531I, N590Y, D654E, F657, E659V and any combination thereof with reference to the position numbering of SEQ ID NO: 180.

32. The engineered protein of claim 31, wherein the modified protein comprises any one of the following combinations of mutations (1) D156R, D489Y and K595Y, (2) D156R, L324M, E484V and K595Y, (3) D156R, E330V and D489Y, (4) D156R, D367V, E379G, Y381H, N590Y and K595Y, (5) D156R, I417F and D489Y, (6) D156R, I417F, K538N and K595Y, (7) D156R, E330V, I417F, and K538N, (8) D156R and E330V, (9) D156R and 1417F, (10) D156R, N263I and L498M, (11) K135N, D156R, T307S, S345R, D489V and K595Y, and/or (12) D156R, S168N, T246S, R359S, I369M, F618L, E479V, K538N, Y686N and K595Y with reference to the position numbering of SEQ ID NO: 180.

33. The engineered protein of any one of claims 30-32, wherein the engineered protein has an altered PAM (protospacer adjacent motif) specificity compared to the PAM specificity for LbCas12a.

34. The engineered protein of any one of claims 30-33, further comprising a mutation in a nuclease active site (e.g., RuvC domain) (e.g., deadLbCas12a, dLbCas12a), optionally wherein the modified protein comprises an amino acid sequence having at least 80%, 85%, 90%, or 95% identity to the amino acid sequence of SEQ ID NO: 50.

35. The engineered protein of any one of claims 30-34, wherein the first polypeptide comprises amino acid residue 1 to about amino residue 250, 300, or 350 with reference to the position numbering of the modified protein, optionally wherein the first polypeptide comprises the mutation and/or the first polypeptide comprises amino acid residue 1 to about amino residue 282, 283, 284, 285, 286, 287, 288, 289, 290, 291, 292, or 293 with reference to the position numbering of the modified protein.

36. The engineered protein of any one of claims 30-35, wherein the second polypeptide has a length of about 10, 50, 100, or 150 to about 200, 250, or 300 amino acids and/or the first polypeptide has a length of about 100, 200, or 250 to about 300, 350, or 400 amino acids, optionally wherein the second polypeptide has a length of about 140 or 150 to about 160, 170, 180, 190, 200, 210, 220, 230, 240, or 250 amino acids and/or the first polypeptide has a length of about 250 or 275 to about 300 or 350 amino acids.

37. The engineered protein of any one of claims 30-36, wherein the second polypeptide comprises a first nuclease domain or a portion thereof.

38. The engineered protein of any one of claims 30-37, wherein the second polypeptide comprises a target strand nickase domain or a portion thereof, optionally wherein the second polypeptide comprises a target strand specific nickase domain, a nontarget strand specific nickase domain, or a target and nontarget strand nickase domain.

39. The engineered protein of any one of claims 30-38, further comprising a third polypeptide that comprises a second nuclease domain or a portion thereof, optionally wherein the third polypeptide comprises the mutation and/or wherein the first polypeptide and the third polypeptide are non-continuous (i.e., are separated from each (optionally by at least 10, 50, 100, or more amino acids) and are not directly attached to each other).

40. The engineered protein of claim 39, wherein the second polypeptide is heterologous to the third polypeptide.

41. The engineered protein of claim 39 or claim 40, wherein the third polypeptide is a Type V CRISPR-Cas effector polypeptide, optionally wherein the third polypeptide is a second portion of the modified protein and is different than the first polypeptide.

42. The engineered protein of any one of claims 39-41, wherein the second nuclease domain or a portion thereof is a nontarget and target strand nickase domain or a portion thereof.

43. The engineered protein of any one of claims 39-42, wherein the second nuclease domain is active.

44. The engineered protein of any one of claims 39-42, wherein the second nuclease domain is inactive.

45. The engineered protein of any one of claims 30-44, wherein the second polypeptide comprises a HNH domain, optionally wherein the HNH domain comprises a mutation that modifies the activity of the HNH domain (e.g., a H840A mutation).

46. The engineered protein of any one of claims 39-45, wherein the first and third polypeptides are each a portion of the modified protein and the second polypeptide is between and/or linked to (e.g., directly or indirectly) two amino acids that are two consecutive or nonconsecutive amino acids of the modified protein.

47. The engineered protein of claim 46, wherein the second polypeptide is positioned in the engineered protein in a location that corresponds to an interdomain linker region of the modified protein.

48. The engineered protein of any one of claims 30-48, wherein the second polypeptide comprises an amino acid sequence having at least 70%, 75%, 80%, 85%, 90%, 95%, 96%, 97%, 98%, 99%, or more sequence identity to one or more of SEQ ID NOs: 1 or 169-174, optionally wherein the second polypeptide comprises an amino acid sequence of any one of SEQ ID NOs: 1 or 169-174.

49. The engineered protein of any one of claims 39-48, wherein the engineered protein comprises, in the amino terminal to carboxy terminal direction, the first polypeptide, the second polypeptide, and the third polypeptide, optionally wherein the third polypeptide has a length of about 800 or 850 to about 900, 1,000, or 1,100 amino acids (e.g., about 900 to about 950 or 1,000 amino acids).

50. The engineered protein of any one of claims 30-49, further comprising all or a portion of a wedge domain, a Rec1 domain, a Rec2 domain, a PAM-interacting domain, a RuvC domain, a bridge helix, and/or a Nuc domain of the modified protein, optionally wherein the engineered protein comprises all or a portion of a wedge domain, a Rec1 domain, a Rec2 domain, a PAM-interacting domain, a RuvC domain, a bridge helix, and/or a Nuc domain of LbCas12a.

51. The engineered protein of claim 50, wherein the engineered protein comprises the Rec1 domain and the Rec2 domain and the second polypeptide is between the Rec1 domain and the Rec2 domain.

52. The engineered protein of any one of claims 30-51, wherein the engineered protein is devoid of at least a portion of the modified protein, optionally wherein the engineered protein is devoid of at least a portion of LbCas12a.

53. The engineered protein of any one of claims 39-52, further comprising a first linker between the first polypeptide and the second polypeptide and/or a second linker between the second polypeptide and the third polypeptide, optionally wherein the first linker and/or second comprises an amino acid sequence of (GGS)ₙ wherein n is an integer of 1-20; GS; SG; and/or an amino acid sequence of one of SEQ ID NOs: 18-47 or 176-179.

54. The engineered protein of claim 53, wherein the first linker and/or the second linker comprises 1 to 10 amino acids, optionally wherein the first linker and/or the second linker comprises 1, 2, 3, or 4 amino acids.

55. The engineered protein of claim 53 or claim 54, wherein the first linker and/or second linker comprises glycine and/or serine.

56. The engineered protein of any one of claims 30-55, wherein the engineered protein comprises an amino acid sequence having about 70%, 75%, 80%, 85%, 90%, 95%, 96%, 97%, 98%, 99%, or more sequence identity to the amino acid sequence of a wild-type CRISPR-Cas effector protein, optionally wherein the engineered protein comprises an amino acid sequence having at least 70%, 75%, 80%, 85%, 90%, 95%, 96%, 97%, 98%, 99%, or more sequence identity to one or more of SEQ ID NOs: 50-66 151, or 180.

57. The engineered protein of any one of claims 30-56, wherein, when the modified protein is optimally aligned to SEQ ID NO: 50, 58, or 180, the second polypeptide is between one of the following sets of amino acid residues that is present in the engineered protein:
amino acid residues 290 and 291 of SEQ ID NO: 50,
amino acid residues 291 and 292 of SEQ ID NO: 50,
amino acid residues 291 and 292 of SEQ ID NO: 58, or
amino acid residues 292 and 293 of SEQ ID NO: 58,
amino acid residues 290 and 291 of SEQ ID NO: 180,
amino acid residues 291 and 292 of SEQ ID NO: 180, or
amino acid residues 292 and 293 of SEQ ID NO: 180.

58. The engineered protein of any one of claims 30-57, wherein the engineered protein is a nuclease, optionally wherein the engineered protein is a target strand nickase, a nontarget strand nickase, or a target and nontarget strand nickase.

59. The engineered protein of any one of claims 30-58, wherein the engineered protein has increased efficiency in nicking the target strand and/or nontarget strand of a target nucleic acid compared to a CRISPR-Cas effector protein (e.g., a wild-type CRISPR-Cas effector protein and/or a protein having a sequence of one of SEQ ID NOs: 50-66 or 151, or 180).

60. The engineered protein of any one of claims 30-59, wherein the engineered protein recognizes a non-natural protospacer adjacent motif (PAM) site and/or sequence (e.g., the engineered protein comprises an altered PAM specificity compared to wild-type LbCas12a).

61. An engineered protein comprising:
an amino acid sequence having at least 80%, 85%, 90%, or 95% identity to the amino acid sequence of SEQ ID NO: 107 (SYN3298); and
a mutation at one or more of the following positions selected from the group consisting of: N100, K116, K120, K121, D122, E125, T148, T149, T152, D156, E159, N211, N263, T443, E478, K535, A552, D553, D571, E632, L646, N675, Q677, G680, D683, K686, E687, D689, Y690, Y701, Y702, D720, L733, K739, M740, K743, V744, S747, K748, K749, Y764, Y794, W797, N112, S117, K135, K167, S168, S170, K192, E223, Q231, A239, V245, T246, T455, L469, L472, F485, I492, S493, R507, D515, I517, E527, Y529, I565, I573, Y574, E591, A602, I603, L607, V611, F622, E627, D637, L640, I655, K662, M679, N738, D802, F805, E807 and any combination thereof with reference to residue position numbering of SEQ ID NO: 107 (SYN3298).

62. The engineered protein of claim 61, wherein the mutation is one or more amino acid mutations selected from the group consisting of: N100S, K116D, K116R, K116N, K120R, K120H, K120N, K120T, K120Y, K120Q, K121S, K121T, K121H, K121R, K121G, K121D, K121Q, D122R, D122K, D122H, D122E, D122N, E125G, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D1561, D156V, D156L, D156E, E159K, E159R, E159H, E159Y, E159Q, N211S, N263I, T4431, E478V, K535E, A552V, D553G, D571V, E632D, L646M, N675S, Q677N, Q677T, Q677H, Q677A, Q677F, Q677G, Q677S, Q677P, Q677W, Q677D, G680D, G680N, G680S, G680H, G680F, G680K, G680R, G680Q, G680A, G680L, G680C, D683N, D683H, D683V, D683T, D683S, D683A, D683W, D683K, K686R, K686V, K686Q, K686W, K686Y, K686F, K686H, K686L, K686M, K686C, K686G, K686A, K686P, E687V, D689N, D689H, D689R, D689K, D689Y, D6891, D689A, D689S, D689E, Y690R, Y690K, Y690H, Y690Q, Y690F, Y690L, Y690M, Y690P, Y690V, Y690N, Y690T, Y701H, Y702N, D720G, L733Q, L733G, L733H, L733F, K739W, K739F, K739Y, K739H, K739R, K739S, K739A, K739G, K739P, M740R, M740K, M740Q, M740E, M740A, K743R, K743Q, K743Y, K743L, K743W, K743H, K743E, K743S, K743D, K743M, V744T, V744H, V744G, V744A, S747G, S747H, S747N, S747D, K748R, K748H, K748G, K749R, K749H, K749Q, K749T, Y764K, Y764R, Y764E, Y764F, Y764H, Y794R, Y794E, Y794K, Y794H, Y794Q, Y794W, Y794N, W797H, W797K, W797Y, W797R, W797E, W797S, W797V, W797T, N112D, S117C, K135N, K167I, S168N, S170L, K192I, E223V, Q231, A239T, V245I, T246S, T455S, L321V, L324M, F337L, 1344T, S345R, R359S, D367V, I369M, E379G, Y381H, I417F, 1425V, Y426D, E443D, A454V, 1455F, L459M, V463A, F474L, E479V, D489Y, L492P, I507V, K514N, M531I, N590Y, D654E, F657, E659V, and any combination thereof with reference to the position numbering of SEQ ID NO: 107, optionally wherein the mutation is one or more amino acid mutations selected from the group consisting of: N100S, K116D, E125G, T152R, D156E, N211S, N263I, T4431, E478V, K535E, A552V, D553G, D571V, E632D, L646M, N675S, G680R, K686V, E687V, Y690R, Y701H, Y702N, D720G, L733Q, K743R, K743Y, N112D, S117C, K135N, K167I, S168N, S170L, K192I, E223V, Q231, A239T, V245I, T246S, T455S, L321V, L324M, F337L, 1344T, S345R, R359S, D367V, I369M, E379G, Y381H, I417F, 1425V, Y426D, E443D, A454V, 1455F, L459M, V463A, F474L, E479V, D489Y, L492P, I507V, K514N, M531I, N590Y, D654E, F657, E659V,
and any combination thereof with reference to the position numbering of SEQ ID NO: 107.

63. The engineered protein of claim 62, wherein the modified protein comprises any one of the following combinations of mutations: (1) D156R, D637Y and K743Y, (2) D156R, L472M, E632V and K743Y, (3) D156R, E478V, D637Y, (4) D156R, D515V, E527G, Y529H, N738Y and K743Y, (5) D156R, 1565F and D637Y, (6) D156R, I565F, K686N and K743Y, (7) D156R, E478V V, I565F, and K686N, (8) D156R and E478V, (9) D156R and I565F, (10) D156R, N263I and L646M, (11) K135N, D156R, T455S, S493R, D637V and K743Y, and/or (12) D156R, S168N, T246S, R507S, I517M, F474L, E627V, K686N, Y542N and K743Y with reference to the amino acid residue positions of SEQ ID NO: 107.

64. The engineered protein of claim 62, wherein the engineered protein has an altered PAM (protospacer adjacent motif) specificity compared to the PAM specificity for LbCas12a.

65. The engineered protein of any one of claims 61-64, wherein the mutation is in the nuclease active site (e.g., RuvC domain) (e.g., deadLbCas12a, dLbCas12a), optionally wherein the engineered protein comprises an amino acid sequence having at least 80%, 85%, 90%, or 95% identity to the amino acid sequence of SEQ ID NO: 50.

66. The engineered protein of any one of claims 61-65, wherein the engineered protein comprises a first polypeptide that comprises amino acid residue 1 to about amino residue 250, 300, or 350 with reference to the position numbering of the amino acid sequence of SEQ ID NO: 180 (LbCas12a) and optionally the mutation, optionally wherein the first polypeptide comprises amino acid residue 1 to about amino residue 282, 283, 284, 285, 286, 287, 288, 289, 290, 291, 292, or 293 with reference to the position numbering of the amino acid sequence of SEQ ID NO: 180 (LbCas12a) and optionally the mutation.

67. The engineered protein of claim 66, wherein the engineered protein comprises a second polypeptide that is heterologous to the first polypeptide and is not a Type V CRISPR-Cas effector polypeptide and the second polypeptide has a length of about 10, 50, 100, or 150 to about 200, 250, or 300 amino acids and/or the first polypeptide has a length of about 100, 200, or 250 to about 300, 350, or 400 amino acids, optionally wherein the second polypeptide has a length of about 140 or 150 to about 160, 170, 180, 190, 200, 210, 220, 230, 240, or 250 amino acids and/or the first polypeptide has a length of about 250 or 275 to about 300 or 350 amino acids.

68. The engineered protein of claim 67, wherein the second polypeptide comprises a first nuclease domain or a portion thereof.

69. The engineered protein of claim 67 or claim 68, wherein the second polypeptide comprises a target strand nickase domain or a portion thereof, optionally wherein the second polypeptide comprises a target strand specific nickase domain, a nontarget strand specific nickase domain, or a target and nontarget strand nickase domain.

70. The engineered protein of any one of claims 61-69, further comprising a third polypeptide that comprises a second nuclease domain or a portion thereof, optionally wherein the third polypeptide comprises the mutation and/or the first polypeptide and the third polypeptide are non-continuous (i.e., are separated from each (optionally by at least 10, 50, 100, or more amino acids) and are not directly attached to each other).

71. The engineered protein of claim 70, wherein the second polypeptide is heterologous to the third polypeptide.

72. The engineered protein of claim 70 or claim 71, wherein the third polypeptide is a Type V CRISPR-Cas effector polypeptide, optionally wherein the third polypeptide comprises the mutation and/or is a portion of the amino acid sequence of SEQ ID NO: 180 (LbCas12a).

73. The engineered protein of any one of claims 70-72, wherein the second nuclease domain or a portion thereof is a nontarget and target strand nickase domain or a portion thereof.

74. The engineered protein of any one of claims 70-73, wherein the second nuclease domain is active.

75. The engineered protein of any one of claims 70-73, wherein the second nuclease domain is inactive.

76. The engineered protein of any one of claims 67-75, wherein the second polypeptide comprises a HNH domain, optionally wherein the HNH domain comprises a mutation that modifies the activity of the HNH domain (e.g., a H840A mutation).

77. The engineered protein of any one of claims 70-76, wherein the first and third polypeptides are each a portion of a Type V CRISPR-Cas effector protein and the second polypeptide is between and/or linked to (e.g., directly or indirectly) two amino acids that are two consecutive or nonconsecutive amino acids of the Type V CRISPR-Cas effector protein.

78. The engineered protein of claim 77, wherein the second polypeptide is positioned in the engineered protein in a location that corresponds to an interdomain linker region of the Type V CRISPR-Cas effector protein.

79. The engineered protein of any one of claims 67-78, wherein the second polypeptide comprises an amino acid sequence having at least 70%, 75%, 80%, 85%, 90%, 95%, 96%, 97%, 98%, 99%, or more sequence identity to one or more of SEQ ID NOs: 1 or 169-174, optionally wherein the second polypeptide comprises an amino acid sequence of any one of SEQ ID NOs: 1 or 169-174.

80. The engineered protein of any one of claims 70-79, wherein the engineered protein comprises, in the amino terminal to carboxy terminal direction, the first polypeptide, the second polypeptide, and the third polypeptide, optionally wherein the third polypeptide has a length of about 800 or 850 to about 900, 1,000, or 1,100 amino acids (e.g., about 900 to about 950 or 1,000 amino acids).

81. The engineered protein of any one of claims 61-80, further comprising all or a portion of a wedge domain, a Rec1 domain, a Rec2 domain, a PAM-interacting domain, a RuvC domain, a bridge helix, and/or a Nuc domain of the Type V CRISPR-Cas effector protein, optionally wherein the engineered protein comprises all or a portion of a wedge domain, a Rec1 domain, a Rec2 domain, a PAM-interacting domain, a RuvC domain, a bridge helix, and/or a Nuc domain of a LbCas12a.

82. The engineered protein of claim 81, wherein the engineered protein comprises the Rec1 domain and the Rec2 domain and the second polypeptide is between the Rec1 domain and the Rec2 domain.

83. The engineered protein of any one of claims 61-82, wherein the engineered protein is devoid of at least a portion of the Type V CRISPR-Cas effector protein, optionally wherein the engineered protein is devoid of at least a portion of LbCas12a.

84. The engineered protein of any one of claims 61-83, further comprising a first linker between the first polypeptide and the second polypeptide and/or a second linker between the second polypeptide and the third polypeptide, optionally wherein the first linker and/or second comprises an amino acid sequence of (GGS), wherein n is an integer of 1-20; GS; SG; and/or an amino acid sequence of one of SEQ ID NOs: 18-47 or 176-179.

85. The engineered protein of claim 84, wherein the first linker and/or the second linker comprises 1 to 10 amino acids, optionally wherein the first linker and/or the second linker comprises 1, 2, 3, or 4 amino acids.

86. The engineered protein of claim 84 or claim 85, wherein the first linker and/or second linker comprises glycine and/or serine.

87. The engineered protein of any one of claims 61-86, wherein the engineered protein comprises an amino acid sequence having about 70%, 75%, 80%, 85%, 90%, 95%, 96%, 97%, 98%, 99%, or more sequence identity to the amino acid sequence of a wild-type CRISPR-Cas effector protein, optionally wherein the engineered protein comprises an amino acid sequence having at least 70%, 75%, 80%, 85%, 90%, 95%, 96%, 97%, 98%, 99%, or more sequence identity to any one of SEQ ID NOs: 50-66, 151, or 180.

88. The engineered protein of any one of claims 61-87, wherein the engineered protein is a nuclease.

89. The engineered protein of any one of claims 61-88, wherein the engineered protein is a target strand nickase, a nontarget strand nickase, or a target and nontarget strand nickase.

90. The engineered protein of any one of claims 61-89, wherein the engineered protein has increased efficiency in nicking the target strand and/or nontarget strand of a target nucleic acid compared to a CRISPR-Cas effector protein (e.g., a wild-type CRISPR-Cas effector protein and/or a protein having a sequence of one of SEQ ID NOs: 50-66, 151, or 180).

91. The engineered protein of any one of claims 61-90, wherein the engineered protein recognizes a non-natural PAM site and/or sequence (e.g., the engineered protein comprises an altered PAM specificity compared to wild-type LbCas12a).

92. The engineered protein of any one of claims 30-91, wherein the engineered protein comprises an amino acid sequence having at least 70%, 75%, 80%, 85%, 90%, 95%, 96%, 97%, 98%, 99%, or more sequence identity to any one of SEQ ID NOs: 181-192, optionally wherein the engineered protein comprises an amino acid sequence having at least 70%, 75%, 80%, 85%, 90%, 95%, 96%, 97%, 98%, 99%, or more sequence identity to any one of SEQ ID NOs: 184-192.

93. A complex comprising:
the engineered protein of any one of claims 30-92;
a guide nucleic acid (e.g., a guide RNA); and
optionally a deaminase.

94. A nucleic acid molecule encoding the engineered protein of any one of claims 30-92.

95. An expression cassette or vector comprising the nucleic acid molecule of claim 94 or a nucleotide sequence encoding the engineered protein of any one of claims 30-92.

96. A method of modifying a target nucleic acid, the method comprising:
contacting the target nucleic acid with:
the engineered protein of any one of claims 30-92, and
a guide nucleic acid (e.g., a guide RNA),
optionally wherein the engineered protein and the guide nucleic acid form a complex or are comprised in a complex, thereby modifying the target nucleic acid.

97. The method of claim 96, wherein the method has increased efficiency in modifying the target nucleic acid and/or in nicking the target strand and/or nontarget strand of a target nucleic acid compared to the efficiency of a control method (e.g., a method comprising contacting the target nucleic acid with a wild-type CRISPR-Cas effector protein).

98. A method of increasing the efficiency of modifying a target nucleic acid, the method comprising:
contacting the target nucleic acid with:
the engineered protein of any one of claims 30-92, and
a guide nucleic acid (e.g., a guide RNA),
optionally wherein the engineered protein and the guide nucleic acid form a complex or are comprised in a complex, thereby modifying the target nucleic acid, thereby increasing the efficiency of modifying the target nucleic acid compared to a control method (e.g., a method that comprises contacting the target nucleic acid with a wild-type CRISPR-Cas effector protein and that is devoid of the engineered protein).

99. The method of claim 96-98, wherein the target nucleic acid is present in a eukaryotic cell, optionally wherein the target nucleic acid is present in a plant cell.

100. The method of any one of claims 96-99, wherein the engineered protein provides a different editing profile of the target nucleic acid compared to the editing profile of the target nucleic acid provided by a wild-type CRISPR-Cas effector protein.

101. The method of any one of claims 96-99, wherein the engineered protein provides a different cleavage pattern of the target nucleic acid compared to the cleavage pattern of the target nucleic acid provided by a wild-type CRISPR-Cas effector protein.

102. The method of any one of claims 96-101, wherein the engineered protein recognizes a non-natural PAM site and/or sequence (e.g., the engineered protein comprises an altered PAM specificity compared to wild-type LbCas12a).
